(19) United States
(12) Patent Application Publication
(10) Pub. No.: US 2017/0325479 A1
(43) Pub. Date: Nov. 16, 2017

(54) PROLINE TOLERANT TRIPEPTIDYL PEPTIDASES AND USES THEREOF

(71) Applicant: DUPONT NUTRITION BIOSCIENCES APS, Copenhagen K (DK)

(72) Inventors: Karsten Matthias KRAGH, Viby J (DK); Ernst MEINJOHANNS, Frederiksberg C (DK); Andrei MIASNIKOV, Union City, CA (US); Maria MA, Fremont, CA (US); Thomas EISELE, Hørning (DK); Svend HAANING, Galten (DK); Peter Edvard DEGN, Egå (DK); Steffen Yde BAK, Aarhus N (DK)

(73) Assignee: DuPont Nutrition Biosciences APS, Copenhagen (DK)

(21) Appl. No.: 15/521,245

(22) PCT Filed: Oct. 23, 2015
(86) PCT No.: PCT/EP2015/074600

§ 371 Date: Apr. 21, 2017

Related U.S. Application Data
(60) Provisional application No. 62/068,282, filed on Oct. 24, 2014.
(60) Provisional application No. 62/068,264, filed on Oct. 24, 2014.
(60) Provisional application No. 62/068,243, filed on Oct. 24, 2014.
(60) Provisional application No. 62/093,301, filed on Dec. 17, 2014.

(51) Int. Cl. A23J 3/34; A23K 20/147; C12N 9/48; A23K 50/75; A23K 50/30; C12P 21/06; A23K 10/14

(52) U.S. Cl. CPC A23J 3/34; C12Y 304/14009; C12Y 304/14; C12P 21/06; A23K 20/147; A23K 10/14; A23K 50/75; A23K 50/30; C12N 9/485

(57) ABSTRACT

A method for the production of a hydrolysate comprising: (a) admixing at least one protein or a portion thereof with: (A) at least one endoprotease; and (B) (a′) at least one proline tolerant tripeptidyl peptidase or fermentate comprising a proline tolerant tripeptidyl peptidase predominantly having exopeptidase activity wherein said proline tolerant tripeptidyl peptidase is capable of cleaving tri-peptides from the N-terminus of peptides having: Proline at P1; and synthetic amino acids at P1; or (b′) at least one proline tolerant tripeptidyl peptidase having exopeptidase activity wherein said proline tolerant tripeptidyl peptidase is capable of cleaving tri-peptides from the N-terminus of peptides having: Proline at PV; and synthetic amino acids at PV; and (b) recovering the hydrolysate. The invention also relates to methods for producing a hydrolysate comprising the use of an endoprotease an exo-tripeptidyl peptidase of the S53 family and an aminopeptidase, to uses of a proline tolerant tripeptidyl peptidase, compositions, food and/or feed additive compositions comprising the same, as well as hydrolysates and uses of proline tolerant tripeptidyl peptidases.

### FIELD OF THE INVENTION

The present invention relates to proline tolerant tripeptidyl peptidases for use in the preparation of hydrolysates and in foods comprising said proline tolerant tripeptidyl peptidases or hydrolysates.

### BACKGROUND

Proteases (synonymous with peptidases) are enzymes that are capable of cleaving peptide bonds between amino acids in substrate peptides, oligopeptides and/or proteins.

Proteases are grouped into 7 families based on their catalytic reaction mechanism and the amino acid residue involved in the active site for catalysis. The serine proteases, aspartic acid proteases, cysteine proteases and metalloprotease are the 4 major families, whilst the threonine proteases, glutamic acid proteases and ungrouped proteases make up the remaining 3 families.

The substrate specificity of a protease is usually defined in terms of preferential cleavage of bonds between particular amino acid residues in a substrate. Typically, amino acid positions in a substrate peptide are defined relative to the location of the scissile bond (i.e. the position at which a protease cleaves):
NH₂— . . . P3-P2-P1*P1′-P2′-P3′ . . . —COOH

Illustrated using the hypothetical peptide above, the scissile bond is indicated by the asterisk (*) whilst amino acid residues are represented by the letter ‘P’, with the residues N-terminal to the scissile bond beginning at P1 and increasing in number when moving away from the scissile bond towards the N-terminus. Amino acid residues C-terminal to the scissile bond begin at P1′ and increase in number moving towards the C-terminal residue.

Proteases can be also generally subdivided into two broad groups based on their substrate-specificity. The first group is that of the endoproteases, which are proteolytic peptidases capable of cleaving internal peptide bonds of a peptide or protein substrate and tending to act away from the N-terminus or C-terminus. Examples of endoproteases include trypsin, chymotrypsin and pepsin. In contrast, the second group of proteases is the exopeptidases which cleave peptide bonds between amino acid residues located towards the C or N-terminus of a protein or peptide substrate.

Certain enzymes of the exopeptidase group may have tripeptidyl peptidase activity. Such enzymes are therefore capable of cleaving 3 amino acid fragments (tripeptides) from the unsubstituted N-terminus of substrate peptides, oligopeptides and/or proteins. Tripeptidyl peptidases are known to cleave tripeptide sequences from the N-terminus of a substrate except peptide bonds with proline at the P1 and/or P1′ position. Alternatively tripeptidyl peptidases may be proline-specific and only capable of cleaving substrates having a proline residue N-terminal to the scissile bond (i.e. in the P1 position).

Both exopeptidases and endoproteases have many applications both in the food and feed industries and in the production of hydrolysates.

### SUMMARY OF THE INVENTION

In a broad aspect the present invention provides a method for the production of a hydrolysate comprising: (a) admixing at least one protein or a portion thereof with:
(A) At least one endoprotease; and
   a tripeptidyl peptidase comprising one or more amino acid sequence selected from the group consisting of SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith; or a tripeptidyl peptidase expressed from one or more of the nucleotide sequences selected from the group consisting of: SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 98, SEQ ID No. 99 or a nucleotide sequence having at least 70% identity therewith, or which differs from these nucleotide sequences by the degeneracy of the genetic code, or which hybridises under medium or high stringency conditions.

According to a first aspect of the invention there is provided a method for the production of a hydrolysate comprising:

(a) admixing at least one protein or a portion thereof with:
(A) at least one endoprotease; and
(B) (a′) at least one proline tolerant tripeptidyl peptidase or fermentate comprising a proline tolerant tripeptidyl peptidase predominantly having exopeptidase activity wherein said proline tolerant tripeptidyl peptidase is capable of cleaving tri-peptides from the N-terminus of peptides having:
   Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
   (b′) at least one proline tolerant tripeptidyl peptidase having exopeptidase activity wherein said proline tolerant tripeptidyl peptidase is capable of cleaving tri-peptides from the N-terminus of peptides having:
   Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′; and

(b) recovering the hydrolysate.

In a second aspect there is provided a method for the production of a hydrolysate comprising:
(a) admixing at least one protein or a portion thereof selected from the group consisting of: a plant protein, a milk-based protein, or an egg protein with:
   (i) at least one endopeptidase;
   (ii) at least one exo-tripeptidyl peptidase of the S53 family; and
   (iii) one or more aminopeptidase, and
(b) recovering the hydrolysate.

In a third aspect there is provided the use of (i) at least one endopeptidase; (ii) at least one exo-tripeptidyl peptidase of the S53 family; and (iii) one or more aminopeptidase, in the manufacture of a hydrolysate from at least one protein or a portion thereof selected from the group consisting of: a plant protein, a milk based protein, or an egg protein, for reducing the immunogenicity in a subject predisposed to having an immune reaction to an untreated hydrolysate or for reducing bitterness of the hydrolysate.

In a fourth aspect there is provided an isolated nucleic acid comprising:

(a) a nucleotide sequence as shown herein as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 98 or SEQ ID No. 99; (b) a nucleotide sequence which has at least about 70% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 98 or SEQ ID No. 99; or

(c) a sequence that hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 98 or SEQ ID No. 99 under medium stringency conditions; or

(c) a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 98 or SEQ ID No. 99 due to degeneracy of the genetic code.

In a fifth aspect there is provided a vector (e.g. a plasmid) comprising a nucleic acid of the invention (suitably an isolated nucleic acid of the second aspect).

According to a sixth aspect of the invention there is provided a host cell comprising a nucleic acid sequence or vector according to the invention.

According to a seventh aspect of the invention there is provided a method for the expression of a proline tolerant tripeptidyl peptidase predominantly having exopeptidase activity wherein said proline tolerant tripeptidyl peptidase is capable of cleaving tri-peptides from the N-terminus of peptides having the consensus sequence cleavage sites:

(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or

(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′:

said method comprising:
(a) transforming a host cell with a nucleic acid or a vector comprising a nucleotide sequence encoding a proline tolerant tripeptidyl peptidase;
(b) expressing the nucleic acid sequence or vector of step (a); and
(c) obtaining the proline tolerant tripeptidyl peptidase or a fermentate comprising said proline tolerant tripeptidyl peptidase and optionally isolating and/or purifying and/or packaging.

In a eighth aspect there is provided the use of at least one endoprotease and at least one proline tolerant tripeptidyl peptidase or fermentate comprising a proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention in the manufacture of a hydrolysate for reducing the immunogenicity in a subject predisposed to having an immune reaction to an untreated hydrolysate or for reducing bitterness of the hydrolysate.

In a ninth aspect there is provided a hydrolysate comprising at least one endoprotease and a proline tolerant tripeptidyl peptidase predominantly having exopeptidase activity wherein said proline tolerant tripeptidyl peptidase is capable of cleaving tri-peptides from the N-terminus of peptides having:

(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or

(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention.

According to a tenth aspect there is provided a composition comprising at least one proline tolerant tripeptidyl peptidase predominantly having exopeptidase wherein said proline tolerant tripeptidyl peptidase is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention; and one or more ingredients selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other food grade salts; a preservative, e.g. sodium benzoate and/or potassium sorbate; or combinations thereof.

In an eleventh aspect there is provided a method for producing a feedstuff or foodstuff comprising contacting a feed component or food component with a hydrolysate or a composition of the invention.

In a twelfth aspect there is provided a food additive composition or feed additive composition comprising at least one proline tolerant tripeptidyl peptidase wherein said proline tolerant tripeptidyl peptidase predominantly has exopeptidase activity and is capable of cleaving tri-peptides from the N-terminus of peptides having:

(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or

(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention or a hydrolysate of the invention;

optionally further comprising one or more ingredients selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other food grade salts; a preservative, e.g. sodium benzoate and/or potassium sorbate; or combinations thereof.

In a thirteenth aspect there is provided a food additive or feed additive composition comprising a hydrolysate according to the invention.

In a fourteenth aspect there is provided a foodstuff or feedstuff comprising: at least one proline tolerant tripeptidyl peptidase comprising an amino acid sequence selected from SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity thereto, or obtainable by a method of the invention; or a hydrolysate of the invention and optionally at least one food or feed ingredient.

In a fifteenth aspect there is provided a kit comprising at least one proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention; at least one endoprotease; and instructions for co-administering same.

In a sixteenth aspect there is provided a kit comprising:

(i) at least one endopeptidase;

(ii) at least one exo-tripeptidyl peptidase of the S53 family; and

(iii) one or more aminopeptidase, and instructions for co-administering same.

In a seventeenth aspect the invention provides a nonfood product comprising the hydrolysate of the invention, wherein the nonfood product is a cosmetic, a lotion, or a cleanser for use on human skin.

In an eighteenth aspect the invention provides a hydrolysate enriched in:
(a) a tripeptide having a Proline at its N-terminus; or
(b) a tripeptide having a Proline at its N-terminus and at its C-terminus.

In any of the embodiments describe herein, hydrolysates produced by the protein hydrolysis processes described herein can be removed or permeated by ultrafiltration or any suitable membrane separation known in the art.

### BRIEF DESCRIPTION OF THE DRAWINGS

Embodiments of the invention will now be described, by way of example only, with reference to accompanying drawings, in which:

FIG. 1 shows a plasmid map of the expression vector pTTT-pyrG-TRI083.

FIG. 2 shows a pH profile for a proline tolerant tripeptidyl peptidase.

FIG. 3 shows a graph displaying the activity of a proline tolerant tripeptidyl peptidase at various temperatures.

FIG. 4 shows enzyme activity when Alphalase® AFP (herein referred to as AFP) (an acid protease) is used in combination with a proline tolerant tripeptidyl peptidase at different dosages of each enzyme.

FIG. 5 shows the ability of a proline tolerant tripeptidyl peptidase to cleave the substrate AAPPA over time.

FIG. 6 shows the production of the cleavage product AAP from an AAPPA substrate over time.

FIG. 7 shows a plasmid map of the expression vector pTTT-pyrG13-TRI071. The endogenous signal sequence was replaced by the secretion signal sequence from the Trichoderma reesei acidic fungal protease (AFP) and an intron from a Trichoderma reesei glycoamylase gene (TrGA1) (see lower portion of FIG. 7).

FIG. 8 shows dose response of Alphalase® AFP on protein hydrolysis of corn soy feed. Dashed line represents the control where only pepsin and pancreatin were used.

FIG. 9 shows the dependence of DH on enzyme composition in the feed sample.

FIG. 10 shows the effect of feed treatment by Alphalase® AFP and proline tolerant tripeptidyl peptidase at different conditions. Solid bars represent the treatment at 40° C. for 100 min. Empty bars represent the treatment at 40° C. for 200 min.

FIG. 11 shows the effect of commercial proteases compared to a proline tolerant tolerant tripeptidyl peptidase on ileal N digestibility %.

FIG. 12 shows the effect of commercial proteases compared to a proline tolerant tripeptidyl peptidase on ileal digestibility of energy (MJ/kg).

FIG. 13 shows the increase of serine equivalents of a 15% (w/w) WPI hydrolysis employing an endopeptidase and different endopeptidase/exopeptidases combinations (Endopeptidase (Endo): Alphalase® FP2, PepN: 197 nkat_{H-Ala-pNA}/mL in stock solution; a proline tolerant tripeptidyl peptidase (TRI083) 4,375 nkat_{H-Ala-Ala-Ala-pNA}/ML).

FIG. 14 shows the increase of serine equivalents of a 15% (w/w) WPI hydrolysis employing an endopeptidase and tripeptidyl peptidase (Endopeptidase (Endo): Alphalase® FP2, a proline tolerant tripeptidylpeptidase (TRI071=3PP_Fo) 4,375 nkat_{H-Ala-Ala-Ala-pNA}/mL).

FIG. 15 shows Michaelis-Menten plots of T. reesei proline tolerant tripeptidyl peptidase analysed using H-Ala-Ala-Ala-pNA analysed with and without pre-hydrolysed H-Ala-Ala-Ala-OH. The pre-hydrolysed inhibitor added contains Ala-Ala-Ala, Ala-Ala and Ala. The concentration is given as the concentration of H-Ala-Ala-Ala-OH before hydrolysis.

FIG. 16 shows the digestion of a fragment of β-lactoglobulin (peptide IIAEK) by a proline tolerant tripeptidyl peptidase measured with liquid chromatography-mass spectrometry (LC-MS).

FIG. 17 shows the digestion of a fragment of β-lactoglobulin (peptide ALPMHIR) by a proline tolerant tripeptidyl peptidase measured with LC-MS.

FIG. 18 shows the digestion of a fragment of β-lactoglobulin (peptide WENGECAQK) by a proline tolerant tripeptidyl peptidase measured with LC-MS.

FIG. 19 shows the digestion of a fragment of β-lactoglobulin (peptide VYVEELKPTPEGDLEILLQK) by a proline tolerant tripeptidyl peptidase measured with LC-MS.

FIG. 20 shows the further digestion of a proline tolerant tripeptidyl peptidase digestion product EELKPTPEGDLEILLQK of beta-lactoglobulin by a proline tolerant tripeptidyl peptidase measured with LC-MS.

FIG. 21 shows the digestion of a fragment of β-lactoglobulin (peptide VAGTWYSLAMAASDISLLDAQSAPLR) by a proline tolerant tripeptidyl peptidase measured with LC-MS.

FIG. 22 shows the digestion of a tryptic fragment of β-casein (GPFPIIV) by a proline tolerant tripeptidyl peptidase measured with LC-MS.

FIG. 23 shows the digestion of a tryptic fragment of β-casein (HKEMPFPK) by a proline tolerant tripeptidyl peptidase measured with LC-MS.

FIG. 24 shows the digestion of a bradykinin peptide by a proline tolerant tripeptidyl peptidase.

FIG. 25 shows alignments between a number of proline tolerant tripeptidyl peptidase amino acid sequences. The xEANLD, y′Tzx′G and QNFSV motifs are shown (boxed).

FIG. 26 shows the effect of pH on TRI045 (a tripeptidyl peptidase having pre-pro sequence SEQ ID No. 98 and mature protein SEQ ID No. 99) activity using AAF-pNA as substrate (values are the average of one test with 0.8 μl TRI045 (n=2).

FIG. 27 shows a plasmid map of the expression vector pTTT-pyrG13-TRI045.

FIG. 28 shows a pH profile for the tripeptidyl peptidase TRI045.

### DETAILED DESCRIPTION

A seminal finding of the present invention is that a tripeptidyl peptidase can have exopeptidase activity on a substrate having proline at P1 and/or P1′ as well as any other amino acid at P1 and/or P1′. This is highly surprising as tripeptidyl peptidases that have been documented in the art typically are inhibited when proline is at P1 or are active when proline is at P1 but inactive when an amino acid other than proline is present at position P1 in the substrate, this is sometimes referred to herein as a proline-specific tripeptidyl peptidase. The inventors have shown for the first time that a proline tolerant tripeptidyl peptidase according to the present invention is highly advantageous for use in the preparation of hydrolysates and confers advantages to subjects fed the hydrolysate or a food and/or feed comprising the hydrolysate and/or a food or feed additive composition comprising the proline tolerant tripeptidyl peptidase (optionally in combination with at least one endoprotease). Alternatively or additionally, the hydrolysate produced using an endoprotease and tripeptidyl peptidase may also have reduced bitterness when compared to an untreated hydrolysate. Advantageously, a proline tolerant tripeptidyl peptidase taught for use in the present invention is capable of acting on a wide range of peptide and/or protein substrates and due to having such a broad substrate-specificity is not readily inhibited from cleaving substrates enriched in certain amino acids (e.g. proline and/or lysine and/or arginine and/or glycine). The use of such a proline tolerant tripeptidyl peptidase therefore may efficiently and/or rapidly breakdown protein substrates (e.g. present in a substrate for preparation of a hydrolysate). Based on these findings, there is provided a method for the production of a hydrolysate comprising:

(a) admixing at least one protein or a portion thereof with:
(A) at least one endoprotease; and
(B) (a′) at least one proline tolerant tripeptidyl peptidase or fermentate comprising a proline tolerant tripeptidyl peptidase predominantly having exopeptidase activity wherein said proline tolerant tripeptidyl peptidase is capable of cleaving tri-peptides from the N-terminus of peptides having:
   Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
   (b′) at least one proline tolerant tripeptidyl peptidase having exopeptidase activity wherein said proline tolerant tripeptidyl peptidase is capable of cleaving tri-peptides from the N-terminus of peptides having:
   Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′; and

(b) recovering the hydrolysate.

Suitably the method may comprise a further step of admixing the hydrolysate recovered in step (b) with at least one food or feed ingredient.

Suitably step (A) and step (B) of the method for production of a hydrolysate may be carried out simultaneously (e.g. at the same time).

Suitably, the protein or portion thereof may be admixed with the endoprotease before adding the proline tolerant tripeptidyl peptidase. Suitably the protein or portion thereof may be admixed with the endoprotease before adding the proline tolerant tripeptidyl peptidase and one or more further protease(s) as detailed herein.

There is also provided in a second aspect a method for the production of a hydrolysate comprising:
(a) admixing at least one protein or a portion thereof selected from the group consisting of: a plant protein, a milk-based protein, or an egg protein with:
   (i) at least one endopeptidase;
   (ii) at least one exo-tripeptidyl peptidase of the S53 family; and
   (iii) one or more aminopeptidase, and
(b) recovering the hydrolysate.

Suitably, (i) (ii) and/or (iii) may be admixed simultaneously with the protein or portion thereof. Alternatively, the order of admixing in step (a) may be carried out in a specific order namely (i) may be carried out before (ii) and/or (iii).

In another aspect there is also provided the use of (i) at least one endopeptidase; (ii) at least one exo-tripeptidyl peptidase of the S53 family; and (iii) one or more aminopeptidase, in the manufacture of a hydrolysate from at least one protein or a portion thereof selected from the group consisting of: a plant protein, a milk based protein, or an egg protein, for reducing the immunogenicity in a subject predisposed to having an immune reaction to an untreated hydrolysate or for reducing bitterness of the hydrolysate.

In any of the embodiments describe herein ultrafiltration or membrane separation can be used to improve the protein hydrolysis processes described herein. This can increase the production of short peptides with highly reduced allergenicity.

In one embodiment the at least one protein for use in the methods and/or uses of the present invention may be selected from the group consisting of: a plant protein, preferably wherein the protein is one or more of a gliadin, an immunogenic fragment of a gliadin, a grain protein, gluten, a soy protein.

In another embodiment the at least one protein for use in the methods and/or uses of the present invention may be selected from the group consisting of: a milk-based protein, preferably wherein the protein is one or more of a casein, e.g. beta-casein; a lactoglobulin, e.g. beta-lactoglobulin; or a whey protein.

Suitably the at least one protein for use in the methods and/or uses of the present invention may be an egg protein.

The term “exo-tripeptidyl peptidase of the S53 family” as used herein refers to a protease predominantly having exopeptidase activity as well as the ability to cleave tripeptides from the N-terminus of a protein and/or peptide substrate. The S53 family peptidases broadly encompass a class of serine proteases. Although the S53 family includes both endoproteases and exopeptidases it is intended herein that this definition refers only to those tripeptidyl peptidases predominantly having exopeptidase activity.

An “exo-tripeptidyl peptidase of the S53 family” has an activity of at least about 50 nkat per mg of protein in the “Exopeptidase Broad-Specificity Assay” (EBSA) taught herein. Suitably an “exo-tripeptidyl peptidase of the S53 family” in accordance with the present invention has an activity of between about 50-2000 nkat per mg of protein in the EBSA activity assay taught herein.

The combination of an endoprotease, an exo-tripeptidyl peptidase of the S53 family and an aminopeptidase may be referred to herein as a “triple enzyme composition”. The term “triple enzyme composition” refers to a combination of at least: an endoprotease, an exo-tripeptidyl peptidase of the S53 family and an aminopeptidase. In other words the term “triple enzyme composition” may also include one or more further enzyme activities and/or further constituents.

In one embodiment the exo-tripeptidyl peptidase of the S53 family may be a proline tolerant tripeptidyl peptidase.

The term “aminopeptidase” as used herein refers to an exopeptidase which is able to cleave single amino acids from the N-terminus of a protein and/or peptide substrate.

The aminopeptidase may be obtainable (e.g. obtained) from Lactobacillus.

Suitably the aminopeptidase may be obtainable from Lactobacillus helveticus. More suitably the aminopeptidase may be obtainable from Lactobacillus helveticus ATCC 12046.

The aminopeptidase for use in combination with an endoprotease and exo-tripeptidyl peptidase in accordance with the present invention may be an aminopeptidase N (e.g. PepN) (EC 3.4.11.2).

In one embodiment the aminopeptidase used in combination with an endoprotease and exo-tripeptidyl peptidase of the S53 family may comprise the sequence shown as:

MAVKRFYKTFHPEHYDLRINVNRKNKTINGTSTITGDVIENPVFINQKFM
TIDSVKVDGKNVDFDVIEKDEAIKIKTGVIGKAVIEIAYSAPLIDTMMGI
YPSYYELEGKKKQIIGTQFETTFARQAFPCVDEPEAKATFSLALKWDEQD
GEVALANMPEVEVDKDGYHHFEETVRMSSYLVAFAFGELQSKTTHTKDGV
LIGVYATKAHKPKELDFALDIAKRAIEFYEEFYQTKYPLPQSLQLALPDF
SAGAMENWGLVTYREAYLLLDPDNTSLEMKKLVATVITHELAHQWFGDLV
TMKWWDNLWLNESFANMMEYLSVDGLEPDWHIWEMFQTSEAASALNRDAT
DGVQPIQMEINDPADIDSVEDGAIVYAKGSRMLVMVRSLLGDDALRKGLK
YYFDHHKFGNATGDDLWDALSTATDLDIGKIMHSWLKQPGYPVVNAFVAE
DGHLKLTQKQFFIGEGEDKGRQWQIPLNANFDAPKIMSDKEIDLGNYKVL
REEAGHPLRLNVGNNSHFIVEYDKTLLDDILSDVNELDPIDKLQLLQDLR
LLAEGKQISYASIVPLLVKFADSKSSLVINALYTTAAKLRQFVEPESNEE
KNLKKLYDLLSKDQVARLGWEVKPGESDEDVQIRPYELSASLYAENADSI
KAAHQIFTENEDNLEALNADIRPYVLINEVKNFGNAELVDKLIKEYQRTA
DPSYKVDLRSAVTSTKDLAAIKAIVGDFENADVVKPQDLCDWYRGLLANH
YGQQAAWDWIREDWDWLDKTVGGDMEFAKFITVTAGVFHTPERLKEFKEF
FEPKINVPLLSREIKMDVKVIESKVNLIEAEKDAVNDAVAKAID

The term “admixing” as used herein refers to the mixing of one or more ingredients and/or enzymes where the one or more ingredients or enzymes are added in any order and in any combination. Suitably, admixing may relate to mixing one or more ingredients and/or enzymes simultaneously or sequentially.

In one embodiment the one or more ingredients and/or enzymes may be mixed sequentially. Preferably, the one or more ingredients and/or enzymes may be mixed simultaneously.

The term “recovering a hydrolysate” as used herein refers to the isolation of a hydrolysate. In some embodiments this may involve separating the hydrolysed matter from unhydrolysed protein and/or peptide substrates. In other embodiments it may additionally or alternatively involve separating the hydrolysed matter away from a proline tolerant tripeptidyl peptidase and/or endoprotease or triple enzyme composition of the invention used for preparing said hydrolysate. In one embodiment the hydrolysate may comprise hydrolysed matter with a purity of at least 80%, more suitably at least 90%, even more suitably at least 95%.

Preferably the hydrolysate may comprise hydrolysed matter with a purity of at least about 99%.

In one embodiment a proline tolerant tripeptidyl peptidase or triple enzyme composition for use in the methods and/or uses of the present invention may be incubated with a substrate (e.g. a protein and/or peptide substrate) at a temperature of at least about 40° C. In other words the method of the present invention may be carried out at a temperature of at least about 40° C.

Suitably the proline tolerant tripeptidyl peptidase or triple enzyme composition may be incubated with a substrate at a temperature of at least about 45° C., suitably at least about 50° C.

Preferably the proline tolerant tripeptidyl peptidase or triple enzyme composition may be incubated with a substrate at a temperature of at least about 55° C.

In another embodiment the proline tolerant tripeptidyl peptidase or triple enzyme composition for use in the methods and/or uses of the present invention may be incubated with a substrate (e.g. a protein and/or peptide substrate) at a temperature of between about 40° C. to about 70° C. In other words the method of the present invention may be carried out at a temperature of between about 40° C. to about 70° C.

Suitably the proline tolerant tripeptidyl peptidase or triple enzyme composition may be incubated with a substrate at a temperature of between about 40° C. to about 60° C.; even more suitably at a temperature of between about 45° C. to about 65° C.

Preferably the proline tolerant tripeptidyl peptidase or triple enzyme composition may be incubated with a substrate at a temperature of between about 50° C. to about 60° C.

Suitably the proline tolerant tripeptidyl peptidase for use in the methods and/or uses or comprised in any of the products of the present invention may be capable of cleaving tri-peptides from the N-terminus of peptides having proline at P1; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1.

Alternatively or additionally, the proline tolerant tripeptidyl peptidase for use in the methods and/or uses or comprised in any of the products of the present invention may be capable of cleaving tri-peptides from the N-terminus of peptides having proline at P1′; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

The term “proline tolerant tripeptidyl peptidase” as used herein relates to an exopeptidase which can cleave tripeptides from the N-terminus of a peptide, oligopeptide and/or protein substrate. A “proline tolerant tripeptidyl peptidase”, also referred to herein as 3PP, is capable of cleaving peptide bonds where proline is at position P1 as well as cleaving peptide bonds where an amino acid other than proline is at P1 and/or capable of cleaving peptide bonds where proline is at position P1′ as well as cleaving peptide bonds where an amino acid other than proline is at P1′.

In one embodiment the proline tolerant tripeptidyl peptidase and/or exo-tripeptidyl peptidase of the S53 family is not an endoprotease.

In another embodiment the proline tolerant tripeptidyl peptidase and/or exo-tripeptidyl peptidase of the S53 family is not an enzyme which cleaves tetrapeptides from the N-terminus of a substrate.

In a further embodiment the proline tolerant tripeptidyl peptidase and/or exo-tripeptidyl peptidase of the S53 family is not an enzyme which cleaves dipeptides from the N-terminus of a substrate.

In a yet further embodiment the proline tolerant tripeptidyl peptidase and/or exo-tripeptidyl peptidase of the S53 family is not an enzyme which cleaves single amino acids from the N-terminus of a substrate.

In one embodiment the proline tolerant tripeptidyl peptidase may be capable of cleaving peptide bonds where proline is at position P1 as well as cleaving peptide bonds where an amino acid other than proline is at P1.

In another embodiment the proline tolerant tripeptidyl peptidase may be capable of cleaving peptide bonds where proline is at position P1′ as well as cleaving peptide bonds where an amino acid other than proline is at P1′.

Suitably, the proline tolerant tripeptidyl peptidase may also be able to cleave peptide bonds where the proline present at position P1 and/or P1′ is present in its cis or trans configuration.

Suitably an “amino acid other than proline” may be an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids.

In another embodiment the “amino acid other than proline” may be an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine or valine.

Suitably, in such an embodiment synthetic amino acids may be excluded.

Preferably, the proline tolerant tripeptidyl peptidase may be able to cleave peptide bonds where proline is present at position P1 and P1′.

It is surprising that a tripeptidyl peptidase can act on a substrate having proline at position P1 and/or P1′. It is even more surprising that in addition to this activity a tripeptidyl peptidase may also have activity when an amino acid other than proline is present at position P1 and/or P1′.

In addition to having activity on any of the various substrates as described above the proline tolerant tripeptidyl peptidase for use in the present invention may additionally be tolerant of proline at one or more positions selected from the group consisting of: P2, P2′, P3 and P3′. Suitably the proline tolerant tripeptidyl peptidase in addition to having the activities described above may be tolerant of proline at position P2, P2′, P3 and P3′.

This is advantageous as it allows the efficient cleavage of peptide and/or protein substrates having stretches of proline and allows cleavage of a wide range of peptide and/or protein substrates.

Preferably the proline tolerant tripeptidyl peptidase may have a high activity on peptides and/or proteins having one or more of lysine, arginine or glycine in the P1 position. Without wishing to be bound by theory peptide and/or protein substrates comprising these amino acids at the P1 position may be difficult to digest for many tripeptidyl peptidases and/or proteases in general and upon encountering such residues cleavage of the peptide and/or protein substrate by a tripeptidyl peptidase and/or protease may halt or slow. Advantageously, by using a proline tolerant tripeptidyl peptidase of the invention it is possible to digest protein and/or peptide substrates comprising lysine, arginine and/or glycine at P1 efficiently.

Suitably the proline tolerant tripeptidyl peptidase may have a preferential activity on peptides and/or proteins having lysine at the P1 position. Advantageously this allows the efficient cleavage of substrates having high lysine content, such as whey protein.

In one embodiment the proline tolerant tripeptidyl peptidase or exo-tripeptidyl peptidase of the S53 family may comprise a catalytic triad of the amino acids serine, aspartate and histidine.

The proline tolerant tripeptidyl peptidase or exo-tripeptidyl peptidase of the S53 family for use in the present invention may be a thermostable proline tolerant tripeptidyl peptidase or exo-tripeptidyl peptidase of the S53 family.

The term “thermostable” means that an enzyme retains its activity when heated to temperatures of up to about 60° C. Suitably “thermostable” may mean that an enzyme retains its activity when heated to about 65° C., more suitably about 70° C.

Advantageously, a thermostable proline tolerant tripeptidyl peptidase or exo-tripeptidyl peptidase of the S53 family is less prone to being denatured and/or will retain its activity for a longer period of time in e.g. an animal when compared to a non-thermostable variant.

The proline tolerant tripeptidyl peptidase or exo-tripeptidyl peptidase of the S53 family may have activity in a range of about pH 2 to about pH 7. Suitably, the proline tolerant tripeptidyl peptidase or exo-tripeptidyl peptidase of the S53 family may have activity in a range of about pH 4 to about pH 7, more suitably in a range of about pH 4.5 to about pH 6.5. Suitably the method of the present invention, in particular the hydrolysis step, may be carried out at a pH of between 2 to about 7.

In one embodiment the method of the present invention, in particular the hydrolysis step may be carried out at a pH of between about 4 to about 7, e.g. 4.5 to 6.5.

Using a proline tolerant tripeptidyl peptidase or exo-tripeptidyl peptidase of the S53 family having activity in a pH range between about pH 4 to about pH 7 is advantageous as it allows the proline tolerant tripeptidyl peptidase or exo-tripeptidyl peptidase of the S53 family to be used with one more endoproteases having activity in this pH range.

When a proline tolerant tripeptidyl peptidase or exo-tripeptidyl peptidase of the S53 family having activity in a pH range between about pH 4 to about pH 7 is used, suitably it may be used in combination with a neutral or an alkaline endoprotease.

Advantageously this means that changing the pH of the reaction medium comprising the protein and/or peptide substrate for hydrolysate production is not necessary between enzyme treatments. In other words it allows the proline tolerant tripeptidyl peptidase and the endoprotease or the constituents of the triple enzyme composition to be added to a reaction simultaneously, which may make the process for producing the hydrolysate quicker and/or more efficient and/or more cost-effective. Moreover, this allows for a more efficient reaction as at lower pH values the substrate may precipitate out of solution and therefore not be cleaved.

Any suitable alkaline endoprotease may be used in the present invention. Suitably, the alkaline endoprotease may be one or more selected from the group consisting of: a trypsin, a chymotrypsin, or a subtilisin.

In another embodiment the proline tolerant tripeptidyl peptidase or exo-tripeptidyl peptidase of the S53 family may have activity at an acidic pH (suitably, the proline tolerant tripeptidyl peptidase may have optimum activity at acidic pH). The proline tolerant tripeptidyl peptidase or exo-tripeptidyl peptidase of the S53 family may have activity at a pH of less than about pH 6, more suitably less than about pH 5. Preferably, the proline tolerant tripeptidyl peptidase may have activity at a pH of between about 2.5 to about pH 4.0, more suitably at between about 3.0 to about 3.3.

Suitably the method of the present invention, in particular the hydrolysis step, may be carried out at a pH of between 2 to about 4, e.g. 3 to 3.3. In one embodiment the proline tolerant tripeptidyl peptidase or exo-tripeptidyl peptidase of the S53 family may have activity at a pH around 2.5.

A proline tolerant tripeptidyl peptidase or exo-tripeptidyl peptidase of the S53 family having activity at an acidic pH can be used in combination with an acid endoprotease and advantageously does not require the pH of the reaction medium comprising the protein and/or peptide substrate for hydrolysate production to be changed between enzyme treatments. In other words it allows the proline tolerant tripeptidyl peptidase and the endoprotease or the constituents of the triple enzyme composition to be added to a reaction simultaneously, which may make the process for producing the hydrolysate quicker and/or more efficient and/or more cost-effective.

At least one endoprotease may be used in combination with the proline tolerant tripeptidyl peptidase or exo-tripeptidyl peptidase of the S53 family for any of the applications herein. For example at least one endoprotease may be comprised in the composition and/or food additive composition and/or nonfood product of the invention.

The term “endoprotease” as used herein is synonymous with the term “endopeptidase” and refers to an enzyme which is a proteolytic peptidase capable of cleaving internal peptide bonds of a peptide or protein substrate (e.g. not located towards the C or N-terminus of the peptide or protein substrate). Such endoproteases may be defined as ones that tend to act away from the N-terminus or C-terminus.

In one embodiment the endoprotease may be one or more selected from the group consisting of: a serine protease, an aspartic acid protease, a cysteine protease, a metalloprotease, a threonine protease, a glutamic acid protease and a protease selected from the family of ungrouped proteases.

In one embodiment the endoprotease may be one or more selected from the group consisting of: an acid fungal protease, a subtilisin, a chymotrypsin, a trypsin and a pepsin or from the group of commercial protease products Alphalase® AFP, Alphalase® FP2, Alphalase® NP, FoodPro® Alkaline Protease, FoodPro® PXT, FoodPro® PBR, FoodPro® 30L, FoodPro® PHT or FoodPro® 51FP.

In one embodiment, the endoprotease may be an acid endoprotease. Suitably, the endoprotease may be an acid fungal protease.

Advantageously, the use of an endoprotease in combination with a proline tolerant tripeptidyl peptidase or exo-tripeptidyl peptidase of the S53 family can increase the efficiency of substrate cleavage. Without wishing to be bound by theory, it is believed that an endoprotease is able to cleave a peptide and/or protein substrate at multiple regions away from the C or N-terminus, thereby producing more N-terminal ends for the proline tolerant tripeptidyl peptidase to use as a substrate, thereby advantageously increasing reaction efficiency and/or reducing reaction times and/or more effectively removing antigens (and thereby reducing immunogenicity).

The proline tolerant tripeptidyl peptidase or exo-tripeptidyl peptidase of the S53 family for use in accordance with the present invention may be an “in-food” proline tolerant tripeptidyl peptidase.

The term “in-food” or “in-feed” as used herein means that the enzyme (e.g. the proline tolerant tripeptidyl peptidase and/or endoprotease and/or exo-tripeptidyl peptidase of the S53 family and/or triple enzyme composition) is functional, preferably primarily functional, more preferably solely functional, in the gastrointestinal tract (GIT) of a subject. In other words, the term “in-food” as used herein means that the enzyme is substantially inactive (or is inactive) in the composition and/or food additive composition and/or feed additive composition and/or foodstuff and/or feedstuff prior to consumption of the composition and/or food additive composition and/or feed additive composition and/or foodstuff and/or feedstuff by a subject. There term “primarily functional” means that the enzyme mainly functions on its substrate once it enters the GIT. In other words, prior to entering the GIT the level of enzyme activity defined as the amount of cleavage of peptide and/or protein substrates to tripeptides is less than about 20%, suitably less than about 10%, preferably less than about 5%, of the level of enzyme activity after it enters the GIT.

The term “solely functional” as used herein means that the enzyme is inactive before entering the GIT and is activated upon entering the GIT.

The term “inactive” as used herein means that the enzyme is not active. This may mean that the enzyme's activity is somehow inhibited or that the enzyme is in an environment in which it is inactive or that the enzyme is presented to its substrate immediately prior to feeding to the animal such that there is not enough time to be active. The “inactivity” of the enzyme may be in any event reversible once it enters the GIT of a subject.

Therefore, suitably the proline tolerant tripeptidyl peptidase (optionally in combination with an endoprotease) and/or exo-tripeptidyl peptidase of the S53 family and/or triple enzyme composition may be admixed with the at least one protein or portion thereof immediately prior to consumption of same by the subject.

The term “substantially inactive” as used herein means that the enzyme has low activity compared with its activity once it has entered the GIT. For instance, substantially inactive may mean that the enzyme in the feed additive composition and/or food additive composition and/or feed additive composition and/or foodstuff and/or feedstuff has less than 10% of its activity when compared with its activity in the GIT.

Maintaining the “in-food” enzyme in an inactive or substantially inactive state in the composition and/or food additive composition and/or feed additive composition and/or foodstuff and/or feedstuff can be achieved in a number of ways known to one skilled in the art.

By way of example only maintaining the water content (wt %) of the composition and/or food additive composition and/or feed additive composition and/or foodstuff and/or feedstuff at less than 15%, preferably less than 10%, is sufficient to ensure that the in-food enzyme is inactive or substantially inactive in the composition and/or food additive composition and/or feed additive composition and/or foodstuff and/or feedstuff.

In one embodiment the composition and/or food additive composition and/or feed additive composition and/or foodstuff and/or feedstuff, post-admixing the in-food enzyme, are (maintained and/or stored) in a dry state or substantially dry state.

The term “dry state” as used herein means that the proline tolerant tripeptidyl peptidase (optionally in combination with an endoprotease) and/or the composition and/or food additive composition and/or feed additive composition and/or foodstuff and/or feedstuff contains no or only a very low amount of water. In other words the term “dry state” as used herein may mean that the proline tolerant tripeptidyl peptidase (optionally in combination with an endoprotease) and/or exo-tripeptidyl peptidase of the S53 family and/or triple enzyme composition and/or the composition and/or food additive composition and/or feed additive composition and/or foodstuff and/or feedstuff comprises less than 5%, preferably less than 1%, water content (wt %).

In one embodiment the proline tolerant tripeptidyl peptidase and/or endoprotease and/or exo-tripeptidyl peptidase of the S53 family and/or triple enzyme composition for use in the methods and/or uses and/or products of the present invention may in a dry or substantially dry state.

In another embodiment a proline tolerant tripeptidyl peptidase for use in any of the methods and/or uses and/or products of the invention may be admixed with a composition comprising at least one protein or at least a portion of a protein, wherein the composition, the proline tolerant tripeptidyl peptidase or combinations thereof and/or exo-tripeptidyl peptidase of the S53 family and/or triple enzyme composition are in a dry or substantially dry state when admixed. Suitably an endoprotease may be further admixed.

The term “dry or substantially dry state” when used herein means that the composition, proline tolerant tripeptidyl peptidase, endoprotease or combinations thereof contains only a very low amount of water. In other words the term “substantially dry state” as used herein may mean that the composition, proline tolerant tripeptidyl peptidase, endoprotease or combinations thereof and/or exo-tripeptidyl peptidase of the S53 family and/or triple enzyme composition comprises less than 15%, preferably less than 10%, water content (wt %).

In one embodiment, the composition, proline tolerant tripeptidyl peptidase, endoprotease or combinations thereof and/or exo-tripeptidyl peptidase of the S53 family and/or triple enzyme composition may be dried prior to, during or after (preferably prior to) use in the methods and/or uses of the invention.

In another embodiment the composition, proline tolerant tripeptidyl peptidase, endoprotease or combinations thereof and/or exo-tripeptidyl peptidase of the S53 family and/or triple enzyme composition either before or after use in the methods and/or uses of the invention comprises less than 15 wt % moisture content.

In another embodiment the composition, proline tolerant tripeptidyl peptidase, endoprotease or combinations thereof and/or exo-tripeptidyl peptidase of the S53 family and/or triple enzyme composition either before or after use in the methods and/or uses of the invention comprises less than 10 wt % moisture content; more suitably less than 5 wt % moisture content, and even more suitably less than 1 wt % moisture content.

The proline tolerant tripeptidyl peptidase (e.g. “in-food” proline tolerant tripeptidyl peptidase), endoprotease or combinations thereof may be maintained in an inactive or substantially inactive state in the composition and/or food additive composition and/or feed additive composition and/or foodstuff and/or feedstuff by physically preventing the enzyme from interacting with its substrate. For example the proline tolerant tripeptidyl peptidase (e.g. “in-food” proline tolerant tripeptidyl peptidase), endoprotease or combinations thereof and/or exo-tripeptidyl peptidase of the S53 family and/or triple enzyme composition may be encapsulated prior to its use in the methods and/or uses composition and/or food additive composition and/or feed additive composition and/or foodstuff and/or feedstuff of the invention.

When the proline tolerant tripeptidyl peptidase (e.g. “in-food” proline tolerant tripeptidyl peptidase), endoprotease or combinations thereof and/or exo-tripeptidyl peptidase of the S53 family and/or triple enzyme composition is physically prevented from interacting with its substrate in the composition and/or food additive composition and/or feed additive composition and/or foodstuff and/or feedstuff, then once in the GIT the physical barrier is removed thus allowing the interaction of the proline tolerant tripeptidyl peptidase (e.g. “in-food” proline tolerant tripeptidyl peptidase), endoprotease or combinations thereof and/or exo-tripeptidyl peptidase of the S53 family and/or triple enzyme composition with its substrate.

By way of example only, the encapsulation may be removed by passage of the encapsulated proline tolerant tripeptidyl peptidase (e.g. “in-food” proline tolerant tripeptidyl peptidase), endoprotease or combinations thereof and/or exo-tripeptidyl peptidase of the S53 family and/or triple enzyme composition through the gizzard, proventriculus or stomach of a subject. The gizzard, proventriculus or stomach of a subject is at very low (acidic) pH (e.g. pH 2-4). This acidity can be used to activate encapsulated enzymes.

In one embodiment the enzyme may be encapsulated by a polymer, such as chitin or chitosans, gelatin, gum arabic or wax for example. By way of example only the polymer may be a gelatin or gum arabic as taught in Xue et al Food Funct. 2013, Apr. 25; 6 February (epub); 4 (4) 610-7. Alternatively, the polymer may a chitosan-based hydrogel as taught in Zhang et al Biomacromolecules 2011, 12, 2894-2901.

In one embodiment the proline tolerant tripeptidyl peptidase (e.g., “in-food” proline tolerant tripeptidyl peptidase), endoprotease or combinations thereof and/or exo-tripeptidyl peptidase of the S53 family and/or triple enzyme composition may be activated by consumption of the enzyme by a subject.

The term “inactive” as used herein may mean that the enzyme is presented to its substrate immediately prior to consumption of the enzyme by a subject such that there is not enough time to be active before it enters the GIT of the subject.

In some embodiment inactivation of the enzyme is a permanent deactivation, e.g. by heat, chemical or pH denaturing. Thus in such embodiments the “inactive” may be permanently inactive, e.g. including in the GIT of a subject.

The present enzymes, including combinations of 3PP and an endoprotease, are also useful in a starch conversion process, particularly in a saccharification and fermentation process of gelatinized, raw, and or granular starch that has undergone liquefaction. The desired end-product (often referred to as an “end-of-fermentation” or “EOF” product) may be any product that may be produced by the enzymatic conversion of the starch substrate. For example, the desired product may be a syrup rich in glucose and maltose, which can be used in other processes, such as the preparation of high-fructose corn syrup (HFCS), or which can be converted into a number of other useful products, such as an ascorbic acid intermediates (e.g., gluconate; 2-keto-L-gulonic acid; 5-keto-gluconate; and 2,5-diketogluconate); 1,3-propanediol; amino acids (e.g., tyrosine, serine, lysine, glutamic acid, glycine, phenylalanine and tryptophan); organic acids (e.g., lactate, pyruvate, succinate, citrate, isocitrate, gluconate, itaconate, and oxaloacetate); antibiotics; antimicrobials; enzymes; vitamins; hormones; ethanol, butanol, and other alcohols; glucono delta-lactone; sodium erythorbate; omega-3 fatty acid; isoprene; and other biochemicals and biomaterials. One skilled in the art is aware of various fermentation conditions that may be used in the production of these EOF products.

Those of skill in the art are well aware of available methods that may be used to prepare starch substrates for conversion. Useful starch substrates may be obtained from tubers, roots, stems, legumes, cereals or whole grain. More specifically, the granular starch may be obtained from corn, cobs, wheat, barley, rye, triticale, milo, sago, millet, cassava, tapioca, sorghum, rice, peas, bean, banana, or potatoes. The starch substrate can be a crude starch from milled whole grain, which contains non-starch fractions, e.g., germ residues and fibers. Liquefaction generally involves gelatinization of starch simultaneously with or followed by the addition of an α-amylase, although additional liquefaction-inducing enzymes optionally may be added. In some cases, liquefaction of starch is performed at or below the gelatinization temperature typically requires similar classes of enzymes with different performance criteria. The liquefied starch can be saccharified into a syrup with a lower degree of polymerization (DP) using α-amylases, optionally in the presence of other enzymes. The exact composition of the products of saccharification depends on the combination of enzymes used, as well as the type of granular starch processed.

The present enzymes may be added during liquefaction and/or saccharification as an isolated enzyme solution, dried or granular enzyme, clarified broth, ultrafiltrate concentrate, or whole cell broth, optionally as part of a blend. The present enzymes can be also added in the form of a cultured cell material produced by host cells expressing the enzymes. The present enzymes may also be secreted by a host cell into the reaction medium during the fermentation or simultaneous saccharification and fermentation (SSF) process, such that the enzyme is provided continuously into the reaction (see, below). The host cell producing and secreting the present enzymes may also express an additional enzyme, such as a glucoamylase and/or α-amylase. The host cell can be engineered to express a broad spectrum of various saccharolytic enzymes.

The soluble starch hydrolysate produced by treatment with amylase can be converted into high fructose starch-based syrup, such as high fructose corn syrup (HFCS). This conversion can be achieved using a glucose isomerase, particularly a glucose isomerase immobilized on a solid support.

Soluble starch hydrolysate, particularly a glucose rich syrup, can be fermented by contacting the starch hydrolysate with a fermenting organism. Ethanologenic microorganisms include yeast, such as Saccharomyces cerevisiae and bacteria, e.g., Zymomonas mobilis, expressing alcohol dehydrogenase and pyruvate decarboxylase. Commercial sources of yeast include ETHANOL REDÒ (LeSaffre); THERMOSACCÒ (Lallemand); RED STARÒ (Red Star); FERMIOLÒ (DSM Specialties); and SUPERSTARTÒ (Alltech). Microorganisms that produce other EOF (such as those mentioned, above) are also known in the art. As mentioned, above, the saccharification and fermentation processes may be carried out as an SSF process, wherein the fermenting organism expresses the present enzymes, optionally with one or more additional enzymes, such as a glucoamylase and/or α-amylase. Fermentation may comprise subsequent enrichment, purification, and recovery of the EOF.

Hydrolysates

The invention also provides a hydrolysate obtainable (e.g. obtained) by a method of the invention.

Suitably such a hydrolysate may be enriched in tripeptides. Suitably, the hydrolysate may be enriched in one or more of a tripeptide having a proline at its N-terminus and/or a tripeptide having a proline at its N-terminus and C-terminus.

Preferably such a hydrolysate may be enriched in one or more tripeptides having a proline at its N-terminus and a proline at its C-terminus.

Alternatively or additionally, the hydrolysate may comprise a proline tolerant tripeptidyl peptidase (optionally in combination with an endoprotease) and/or exo-tripeptidyl peptidase of the S53 family and/or triple enzyme composition described herein, such as any one of the amino acid sequences described herein or a proline tolerant tripeptidyl peptidase obtainable (e.g. obtained) by any of the methods herein.

The term “hydrolysate” as used herein has its usual meaning in the art and refers to a product resulting from the treatment of a protein or portion thereof with a proline tolerant tripeptidyl peptidase and an endoprotease. The extent of proteolytic cleavage of the protein or portion thereof can range from minimal (e.g. cleavage of a single peptide bond on a single protein) to extensive depending on, for example, the conditions of the treatment, such as the length of the treatment, the temperature, the concentration of the protein, and the purity, concentration, and activity of the proline tolerant tripeptidyl peptidase and endoprotease.

A “hydrolysate” therefore typically comprises a mixture of short peptides obtainable by cleaving a peptide and/or protein substrate with at least one protease (suitably a proline tolerant tripeptidyl peptidase). Suitably such a hydrolysate may be substantially enriched in tripeptides, and optionally also in single amino acids and/or dipeptides.

Advantageously, enrichment in tripeptides, dipeptides and/or single amino acids may result in more efficient uptake of the hydrolysate by an animal and/or human.

Hydrolysates with a high content of tripeptides as well as dipeptides are nutritionally advantageous because the intestinal absorption of di- and tripeptides happens via the PepT1 receptors, which function independently from the uptake of free amino acids (Gilbert et al. 2008, J ANIM SCI, 86:2135-2155).

The term “substantially enriched in tripeptides” as used herein means that of the total peptide concentration measured by any method known in the art (e.g. liquid chromatography-mass spectrometry (LC-MS)) at least about 20% (suitably at least about 30%) of those peptides are tripeptides. Suitably, at least about 40% of those peptides are tripeptides, more suitably at least about 50%.

In one embodiment the term “substantially enriched in tripeptides” as used herein means that of the total peptide concentration measured by any method known in the art (e.g. liquid chromatography-mass spectrometry (LC-MS)) at least about 70% of those peptides are tripeptides.

Advantageously ultrafiltration can be used during protein hydrolysis. Enzymatic membrane reactors have at least one ultrafiltration membrane coupled to the hydrolysis reactor. Thereby ultrafiltration can be used during the hydrolysis to remove enzyme inhibiting peptides and/or to produce peptide fractions with molecular masses determined by the molecular weight cut-off of the membrane. This is useful for production of short peptides with highly reduced allergenicity. Enzyme membrane reactors can also facilitate higher conversion of the protein substrate into the desired peptides and re-use of the proteolytic enzymes applied. Examples of use of enzyme membrane reactor are given in Eisele et al. (2013), European Food Research and Technology 236, 483-490 and Cheison S C, Wang Z, Xu S Y (2007) J Agric Food Chem 55, 3896-3904.

In one embodiment the hydrolysate may comprise less than about 20%, suitably less than about 10% of the full-length starting substrate (e.g. at least one protein). Suitably, the hydrolysate may comprise less than about 5%, more suitably less than about 1% of the full-length starting substrate (e.g. at least one protein).

In some embodiments the hydrolysate may comprise no, or substantially no, full-length starting substrate.

The term “substantially no” as used in this context may mean less than about 0.5%, suitably less than about 0.1% of full-length starting substrate.

Where an endoprotease, exo-tripeptidyl peptidase of the S53 family and aminopeptidase have been used in the manufacture of a hydrolysate it is believed that such a hydrolysate will be enriched in single amino acids, dipeptides and tripeptides.

In one embodiment the single amino acids, dipeptides and tripeptides present in such a hydrolysate may be quantified in terms of molarity of each stated. In one embodiment the molar ratio of single amino acids, dipeptides and tripeptides in a hydrolysate may be at least about 20% single amino acids to at least about 10% dipeptides to at least about 10% tripeptides.

In another embodiment the molar ratio of single amino acids, dipeptides and tripeptides in a hydrolysate may be at least about 10% single amino acids to at least about 20% dipeptides to at least about 20% tripeptides.

The hydrolysate obtainable according to the methods of the present invention or for use in any of the applications taught herein may have a reduced immunogenicity in a subject predisposed to having an immune response to the at least one protein or a portion thereof that formed the substrate for digestion for the production of the hydrolysate.

The hydrolysate of the invention is produced by admixing at least one protein or a portion thereof with an endoprotease and a proline tolerant tripeptidyl peptidase.

The protein or portion thereof used as the substrate in manufacture of the hydrolysate may be an animal protein or a plant protein (e.g. a vegetable protein).

Suitably the protein or portion thereof may be one or more selected from the group consisting of: a gliadin, a beta-casein, a beta-lactoglobulin or an immunogenic fragment of a gliadin, a beta-casein, a beta-lactoglobulin, glycinin, beta-conglycinin, cruciferin, napin, collagen, whey protein, fish protein, meat protein, egg protein, soy protein, a hordein and a grain protein.

In one embodiment, preferably the protein or portion thereof is a plant protein, a milk based protein, an egg protein or a combination thereof.

In one embodiment, preferably the protein or portion thereof is a plant protein, preferably wherein the protein is one or more of a gliadin, an immunogenic fragment of a gliadin, a grain protein, gluten, a soy protein.

In one embodiment, preferably the protein or portion thereof is a milk-based protein, preferably wherein the protein is one or more of a casein, e.g. beta-casein; a lactoglobulin, e.g. beta-lactoglobulin; or a whey protein.

In one embodiment, preferably the protein or portion thereof is an egg protein.

The protein or portion thereof may be comprised in corn, soybean meal, corn dried distillers grains with solubles (DDGS), wheat, wheat proteins (including gluten), wheat byproducts, wheat bran, corn by products including corn gluten meal, barley, oat, rye, triticale, full fat soy, animal by-product meals, an alcohol-soluble protein (preferably a zein (e.g. a maize zein maize) and/or a kafirin (e.g. from sorghum)), a protein from oil seeds (preferably from soybean seed proteins, sun flower seed proteins, rapeseed proteins, canola seed proteins or combinations thereof) or a combination thereof.

In one embodiment the protein or portion thereof may be a protein-meal. In one embodiment this may be a protein-meal from fish or a protein-meal from an animal (e.g. such as a non-human mammal).

In some embodiments the protein or a portion thereof for use in accordance with the present invention may be an animal by-product.

Such by-products may include tissues from animal production and processing which are not utilized in human food and are processed into an array of protein meals used in animal feeds and pet food. In one embodiment the animal protein by-products may be meat and bone meal, meat meal, blood meal, poultry by-product meal, poultry meal, feather meal, and fish meal.

In another embodiment the protein or a portion thereof for use in the present invention may be a microbial protein. Microbial proteins, such as e.g. yeast extracts, are typically made by extracting the cell contents from microbial cultures; they may be used as food additives or flavourings, or as nutrients for microbial culture media.

In yet further embodiments the protein or a portion thereof for use in the present invention may be an invertebrate protein, suitably an insect protein.

Insects/invertebrates possess enormous biodiversity and represent a large biomass (95% of the animal kingdom), and thus offer alternative protein sources.

In one embodiment the protein or portion thereof may be one or more selected from the group consisting of: a wheat protein, portions of a wheat protein, a dairy protein and portions of a dairy protein.

The wheat protein or portion thereof may be obtainable from wheat, wheat products (e.g. wheat flour), wheat byproducts and/or wheat bran. Suitably, the wheat protein may be one or more selected from the group consisting of: gliadin, portions of gliadin, gluten and portions of gluten.

The dairy protein or a portion thereof may be milk protein. Suitably the milk protein may be one or more selected from the group consisting of: a beta-casein, a beta-lactoglobulin and a whey protein.

The “portion thereof” in relation to a protein or portion thereof used for the manufacture of a hydrolysate may be an immunogenic fragment of a protein. An “immunogenic fragment” is any portion that is capable of eliciting an immune response in a sensitive individual. The “immunogenic fragment” or “portion thereof” is a region of a full-length protein comprising or consisting of at least 10 amino acids, suitably at least 20 amino acids, more suitably at least 30 amino acids.

In some embodiments the immunogenic fragment or portion thereof may be at least about 50 amino acids; more suitably at least about 100 amino acids, and even more suitably at least about 200 amino acids.

As used herein, “milk protein” encompasses any naturally-occurring protein in the normal secretion of the mammary gland of a postpartum female mammal, or products derived therefrom, such as fractions thereof, or components made therefrom or thereof. The milk can be from any mammalian species including but not limited to cow, goat, sheep, buffalo, yak, camel, llama, alpaca, and human. Milk proteins from those mammals whose milk is used commercially or widely in various cultures and countries are preferred. It is to be noted that “milk protein” as used herein encompasses both the singular and the plural of the word “protein”, thus, the term “milk protein” may refer to a single protein, or any mixture of one or more proteins, except as otherwise indicated.

The term “whey protein” encompasses any protein found in any amount in “whey”; the liquid by-product of cheese making that is separated from the curd. The whey resulting from the production of many cheeses is particularly low in micellar milk proteins, such as caseins, but relatively enriched in soluble proteins such as alpha lactalbumin and beta-lactoglobulin. As with “milk protein” above, the term “whey protein” as used herein encompasses both the singular and the plural of the word “protein”, thus, the term “whey protein” also may refer to a single protein, or any mixture of one or more whey proteins, except as otherwise indicated. It will be understood by the skilled artisan that whey proteins are in fact a subclass of milk proteins, and thus the term “milk protein” may include one or more whey proteins, except as otherwise indicated herein. Whey compositions may include, for example, milk, cream, and cheese whey. Whey derived from any cheese type may be used. Whey protein may be derived from any methods such as filtration, dialysis, evaporation, and reverse osmosis of cheese whey, or by any other process which results in the proteins typically described as “whey proteins”.

In a preferred embodiment, the hydrolysate obtainable (e.g. obtained) by the method of the present invention may be a milk protein hydrolysate, a wheat protein hydrolysate (e.g. a gliadin and/or gluten hydrolysate), a soy protein hydrolysate or combinations thereof. Therefore, there is provided the use of at least one endoprotease and at least one proline tolerant tripeptidyl peptidase or fermentate comprising a proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention; in the manufacture of a hydrolysate for reducing the immunogenicity in a subject predisposed to having an immune reaction to an untreated hydrolysate.

Suitably, the proline tolerant tripeptidyl peptidase may be capable of cleaving tri-peptides from the N-terminus of peptides having: Proline at P1; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; and Proline at P1′; and

an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

As used herein, “reduced immunogenicity” refers to any reduction, decrease, or amelioration of a measurable immunological response. The measurement of such response may be assessed in vitro or in vivo. For example, the response may be measured directly or indirectly in a biological sample comprising tissue, cells, or fluid, or the like, or any combination thereof from an individual or it may be assessed in the individual, either directly or indirectly. While in various embodiments provided herein, any mathematical decrease (or reduction) in such response, whether measured in vitro or in vivo, will suffice, it is preferred that the decrease be a more substantial one. Of course, the skilled artisan will appreciate that biological data such as a measurement of an immunological response, are subject to potentially large variation within an individual, and from individual to individual. For example where the response is in a “sensitive” individual, the immune response is preferably substantially reduced (e.g. by at least about 50%, 60%, or 70%, or even about 80% or more). More preferably, only small or minimal differences are seen in such the sensitive individual with the protein hydrolysates described herein, as compared to a measure of the immunological response from an individual who is not sensitive to one or more proteins or portions thereof. This is particularly preferable where the immunological response is deemed adverse, for example an allergic response. In such cases the decrease in the sensitive individual's immunological response (or measure thereof) may be at least about 85% to about 90%, more preferably about 90% to about 95%, or even more. In some case, a sensitive individual's response to the protein hydrolysates described herein is not significantly different, statistically, from the response of an individual who is not sensitive. In yet other cases, the reduction in immunological response may be many-fold over that seen with the unhydrolysed protein in a “sensitive” individual. For example, there may be about a 10-fold to 100-fold or even 1000-fold reduction in response. More preferably reductions of about 1000-fold to 10,000-fold or even 100,000-fold or greater reduction in a measurement of an immunological response from an individual consuming or exposed to the protein hydrolysate compositions as disclosed herein, as compared to that individual's response to the unmodified proteins.

As used herein, a “sensitive” individual is an individual predisposed to having an immune response or reaction to the protein in an unhydrolysed form. Such immune response or reaction as a direct or indirect result of the consumption of, or exposure to, for example one or more protein or portion thereof, is a measure of the immunogenicity of those proteins. Such proteins will demonstrate little to no immunogenicity in an individual who is not predisposed to having such an immune response to the protein, such an individual is sometimes referred to herein as “insensitive” or “not sensitive” to the one or more proteins or portions thereof. Preferably, such an individual will not have a significant immune reaction (immunological response) to either the exposure to or consumption of the protein.

Sensitive individuals having a reaction to wheat proteins (in particular gluten and/or gliadin) may present with symptoms of coeliac disease (e.g. celiac sprue). Symptoms include pain and discomfort in the digestive tract, chronic constipation and diarrheoa, failure to thrive (in children), anemia and fatigue, but these may be absent, and symptoms in other organ systems have been described. Vitamin deficiencies are often noted in people with coeliac disease owing to the reduced ability of the small intestine to properly absorb nutrients from food. Without wishing to be bound by theory it is believed that upon exposure to gliadin that the enzyme tissue transglutaminases modifies the gliadin resulting in cross-reaction of the immune system with the bowel tissue causing an inflammatory reaction in the affected individual.

Sensitive individuals having a reaction to milk proteins may present with symptoms of a milk allergy which is caused by an adverse immune reaction to one or more of the proteins or immunogenic fragments thereof in the milk. The disorder can be either antibody-mediated or non-antibody-mediated. Antibody-mediated milk allergies are typically rapid in onset and may result in the individual displaying gastrointestinal, dermatological and/or respiratory symptoms. Such symptoms may further manifest as: skin rashes, hives, vomiting, and gastric distress, such as diarrhea, rhinitis, stomach pain, wheezing, or anaphylactic reactions. Non-antibody-mediated is typically believed to be mediated by T-lymphocytes and not caused by antibodies. Symptoms of this form are typically gastrointestinal and dermatological.

Other sensitive individuals may have a reaction to soy which results in a range of symptoms, the most severe being anaphylaxis.

The hydrolysates and/or food and/or feed and/or comprising such hydrolysates or compositions of the invention may be particularly suitable for administering to a subject suffering from coeliac disease, a milk protein allergy and/or a soy protein allergy.

Advantageously, the endoprotease in combination with a proline tolerant tripeptidyl peptidase is capable of cleaving protein substrates associated with causing an immune response in sensitive individuals suffering from a disease, such as a milk protein allergy and/or a soy protein allergy.

Therefore in one embodiment there is provided a food additive or feed additive composition comprising a hydrolysate produced in accordance with the methods and/or uses of the present invention.

In another aspect of the invention there is also provided the use of at least one endoprotease and at least one proline tolerant tripeptidyl peptidase or fermentate comprising a proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention; in the manufacture of a hydrolysate for reducing bitterness of the hydrolysate.

When the protein or portion thereof substrate for hydrolysate production is rich in hydrophobic L-amino acids the protein hydrolysate may have a bitter taste. Without wishing to be bound by theory it is believed that the bitterness of a peptide is dependent on its peptide length and the average hydrophobicity of the L-amino acid residues therein.

The “reduced bitterness” of a hydrolysate can be measured objectively using a tasting panel of individuals who are asked to rate the bitterness of a hydrolysate. A bitterness index can be used to rate the bitterness of the hydrolysate. For example, a bitterness index can be used that rates substances relative to quinine which is given a reference index of 1, alternatively a bitterness index may be used having a scale from 0 (not bitter) to 10 (bitter). Suitably any tasting of hydrolysates may be done using the appropriate controls, such as blind testing. Additionally or alternatively, the cleavage of known bitter peptides may be monitored via LC-MS or other suitable techniques known in the art.

The endoprotease and proline tolerant tripeptidyl peptidase may be used to cleave bitter peptides.

Advantageously, this can be achieved due to the tolerance of a wide variety of amino acids at positions P1, P2, P3, P1′, P2′ and/or P3′ by the proline tolerant tripeptidyl peptidase.

The proline tolerant tripeptidyl peptidase may be capable of cleaving a protein having the sequence GPFPIIV, VIPVPQK and/or GPFPVI. GPFPVI is known to be a highly bitter peptide (Matoba et al (1970), Agric. Biol. Chem. 34, 321).

Surprisingly, the proline tolerant tripeptidyl peptidase may be capable of cleaving peptides having proline at the P1 and/or P1′ position, thus facilitating the cleavage of such bitter peptides.

In one embodiment debittered hydrolysates may be used in the preparation of food and or foodstuffs.

In one embodiment the hydrolysate may comprise a proline tolerant tripeptidyl peptidase comprising one or more amino acid sequence selected from:

SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith.

Suitably the proline tolerant tripeptidyl peptidase may comprise one or more amino acid sequence selected from SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28 or a functional fragment thereof or an amino acid sequence having at least 70% identity therewith.

Suitably the proline tolerant tripeptidyl peptidase may comprise one or more amino acid sequence selected from SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith.

Kits

In one aspect there is provided a kit comprising at least one proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;

or obtainable by any one of methods of the invention; at least one endoprotease; and instructions for co-administering same.

Suitably the proline tolerant tripeptidyl peptidase may be capable of cleaving tri-peptides from the N-terminus of peptides having:
(i) (A) Proline at P1; and
   (B) An amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; and
(ii) (a′) Proline at P1′; and
   (b′) An amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

More suitably, the proline tolerant tripeptidyl peptidase may be capable of cleaving tri-peptides from the N-terminus of peptides having proline at position P1 and at position P1′.

The present invention also provides a kit comprising: (i) at least one endopeptidase;

(ii) at least one exo-tripeptidyl peptidase of the S53 family; and (iii) one or more aminopeptidase, and instructions for co-administering same

The endoprotease may be compartmentalised separately to the proline tolerant tripeptidyl peptidase or the two enzymes may be mixed.

The proline tolerant tripeptidyl peptidase and/or endoprotease may be formulated in any manner as described herein or known to the person skilled in the art.

The term “co-administering” as used herein means administering one or more ingredients and/or enzyme either separately (e.g. sequentially) or together (e.g. simultaneously).

Activity and Assays

The proline tolerant tripeptidyl peptidase for use in the present invention predominantly has exopeptidase activity.

The term “exopeptidase” activity as used herein means that the proline tolerant tripeptidyl peptidase is capable of cleaving tri-peptides from the N-terminus of a substrate, such as a protein and/or peptide substrate.

The term “predominantly has exopeptidase activity” as used herein means that the tripeptidyl peptidase has no or substantially no endoprotease activity.

“Substantially no endoprotease activity” means that the proline tolerant tripeptidyl peptidase or exo-peptidase of the S53 family has less than about 100 U endoprotease activity in the “Endoprotease Assay” taught herein when compared to 1000 nkat of exopeptidase activity in the “Exopeptidase Broad-Specificity Assay (EBSA)” taught herein. Suitably, “substantially no endoprotease activity” means that the proline tolerant tripeptidyl peptidase has less than about 100 U endoprotease activity in the “Endoprotease Assay” taught herein when compared to 1000 nkat of exopeptidase activity in the “Exopeptidase Broad-Specificity Assay” taught herein.

Preferably the proline tolerant tripeptidyl peptidase or exo-peptidase of the S53 family may have less than about 10 U endoprotease activity in the “Endoprotease Assay” taught herein when compared to 1000 nkat of exopeptidase activity in the “Exopeptidase Broad-Specificity Assay” taught herein, more preferably less than about 1 U endoprotease activity in the “Endoprotease Assay” taught herein when compared to 1000 nkat of exopeptidase activity in the “Exopeptidase Broad-Specificity Assay” taught herein. Even more preferably the proline tolerant tripeptidyl peptidase or exo-tripeptidyl peptidase may have less than about 0.1 U endoprotease activity in the “Endoprotease Assay” taught herein when compared to 1000 nkat of exopeptidase activity in the “Exopeptidase Broad-Specificity Assay” taught herein.

“Endoprotease Assay”

Azoscasein Assay for Endoprotease Activity

A modified version of the endoprotease assay described by Iversen and Jørgensen, 1995 (Biotechnology Techniques 9, 573-576) is used. An enzyme sample of 50 μl is added to 250 μl of azocasein (0.25% w/v; from Sigma) in 4 times diluted Mcllvaine buffer, pH 5 and incubated for 15 min at 40° C. with shaking (800 rpm). The reaction is terminated by adding 50 μl of 2 M trichloroacetic acid (TCA) (from Sigma Aldrich, Denmark) and centrifugation for 5 min at 20,000 g. To a 195 μl sample of the supernatant 65 μl of 1 M NaOH is added and absorbance at 450 nm is measured. One unit of endoprotease activity is defined as the amount which yields an increase in absorbance of 0.1 in 15 min at 40° C. at 450 nm.

“Exopeptidase Assay”

There are Two Parts to the Assay:

Part 1—“Exopeptidase Broad-Specificity Assay” (EBSA)

10 μL of the chromogenic peptide solution (10 mM H-Ala-Ala-Ala-pNA dissolved in dimethyl sulfoxide (DMSO); MW=387.82; Bachem, Switzerland) were added to 130 μl Na-acetate (20 mM, adjusted to pH 4.0 with acetic acid) in a microtiter plate and heated for 5 minutes at 40° C. 10 μL of appropriately diluted enzyme was added and the absorption was measured in a MTP reader (Versa max, Molecular Devices, Denmark) at 405 nm. One katal of proteolytic activity was defined as the amount of enzyme required to release 1 mole of p-nitroaniline per second.

Part 2 (i)—P1 Proline Assay

(a) Dissolve the substrate H-Arg-Gly-Pro-Phe-Pro-Ile-Ile-Val (MW=897.12; from Schafer-N, Copenhagen in 10 times diluted Mcllvain buffer, pH=4.5 at 1 mg/ml concentration.

(b) Incubate 1000 ul of the substrate solution with 10 ug of proline tolerant tripeptidyl peptidase solution at 40° C.

(c) Take 100 ul samples at seven time points (0, 30, 60, 120, 720 and 900 min), dilute with 50 ul 5% TFA, heat inactivate (10 min at 80° C.) and keep at −20° C. until LC-MS analysis;

(d) Perform LC-MC/MS analysis using an Agilent 1100 Series Capillary HPLC system (Agilent Technologies, Santa Clara, Calif.) interfaced to a LTQ Orbitrap Classic hybrid mass spectrometer (Thermo Scientific, Bremen, Germany);

(e) Load samples onto a 50 mm Fortis™ C18 column with an inner diameter of 2.1 mm and a practical size of 1.7 μm

(f) Perform separation at a flow rate of 200 μL/min using a 14 min gradient of 2-28% Solvent B (H2O/CH3CN/HCOOH (50/950/0.65 v/v/v)) into the IonMAX source—The LTQ Orbitrap Classic instrument was operated in a data-dependent MS/MS mode;

(g) Measure the peptide masses by the Orbitrap (obtain MS scans with a resolution of 60.000 at m/z 400), and select up to 2 of the most intense peptide m/z and subject to fragmentation using CID in the linear ion trap (LTQ). Enable dynamic exclusion with a list size of 500 masses, duration of 40 s, and an exclusion mass width of ±10 ppm relative to masses on the list;

(h) Use the open source program Skyline 1.4.0.4421 (available from MacCoss Lab Software, University of Washington, Department of Genome Sciences, 3720 15^{th} Ave NE Seattle, Wash., US) to access the RAW files and extract MS1 intensities to build chromatograms. Set the precursor isotopic import filter to a count of three, (M, M+1, and M+2) at a resolution of 60,000 and use the most intense charge state;

(i) Peptide sequences of the substrate and cleavage products were typed into Skyline and intensities were calculated in each sample (0, 30, 60, 120, 720 and 900 min hydrolysis).

(j) One unit of activity is defined as the amount of enzyme which in this assay will hydrolyse 50% of the substrate within 720 min while releasing Arg-Gly-Pro.

Part 2 (ii)—P1′ Proline Assay

(a) Dissolve the peptide H-Ala-Ala-Phe-Pro-Ala-NH2 (MW=474.5; from Schafer-N, Copenhagen) in 10 times diluted Mcllvain buffer, pH=4.5 at 0.1 mg/ml concentration.

(b) Incubate 1000 ul of the substrate solution with 10 ug proline tolerant tripeptidyl peptidase solution at 40° C.

(c) Take 100 ul samples at seven time points (0, 30, 60, 120, 720 and 900 min), dilute with 50 ul 5% TFA, heat inactivate (10 min at 80° C.) and keep at −20° C. until LC-MS analysis;

(d) Perform LC-MC/MS analysis using a Agilent 1100 Series Capillary HPLC system (Agilent Technologies, Santa Clara, Calif.) interfaced to a LTQ Orbitrap Classic hybrid mass spectrometer (Thermo Scientific, Bremen, Germany);

(e) Load samples onto a 50 mm Fortis™ C18 column with an inner diameter of 2.1 mm and a practical size of 1.7 μm

(f) Perform separation at a flow rate of 200 μL/min using a 14 min gradient of 2-28% Solvent B (H2O/CH3CN/HCOOH (50/950/0.65 v/v/v)) into the lonMAX source—The LTQ Orbitrap Classic instrument was operated in a data-dependent MS/MS mode;

(g) Measure the peptide masses by the Orbitrap (obtain MS scans with a resolution of 60.000 at m/z 400), and select up to 2 of the most intense peptide m/z and subject to fragmentation using CID in the linear ion trap (LTQ). Enable dynamic exclusion with a list size of 500 masses, duration of 40 s, and an exclusion mass width of ±10 ppm relative to masses on the list.

(h) Use the open source program Skyline 1.4.0.4421 (available from MacCoss Lab Software, University of Washington, Department of Genome Sciences, 3720 15^{th} Ave NE Seattle, Wash., US) to access the RAW files and extract MS1 intensities to build chromatograms. Set the precursor isotopic import filter to a count of three, (M, M+1, and M+2) at a resolution of 60,000 and use the most intense charge state;

(i) Peptide sequences of the substrate as well as cleavage products were typed into Skyline and intensities were calculated in each sample.

(j) One unit of activity is defined as the amount of enzyme which in this assay will hydrolyse 50% of the substrate within 720 min while releasing Ala-Ala-Phe.

In one embodiment a proline tolerant tripeptidyl peptidase in accordance with the present invention has an activity of at least 50 nkat in Part 1 of the activity taught herein and at least 100 U activity in Part 2(i) or Part 2(ii) of the assay taught herein per mg of protein.

In one embodiment a proline tolerant tripeptidyl peptidase in accordance with the present invention has an activity of between about 50-2000 nkat in Part 1 of the activity taught herein and between about 1-500 units activity in Part 2(i) or Part 2(ii) of the assay taught herein per mg of protein. Note the protein measurement is described in Example 2.

“P1 and P1′ Proline Activity Assay”

Suitably the tripeptidyl peptidase for use in the present invention may be able to cleave substrates having proline at position P1 and P1′. This can be assessed using the assay taught below.

In this assay a tripeptidyl peptidase is examined for its ability to hydrolyse a synthetic substrate AAPPA by LC-MS and label free quantification.

(a) Dissolve the peptide H-AAPPA-NH2 (MW=424.3, from Schafer-N, Copenhagen) in 20 mM MES buffer, pH=4.0 (1 mg/ml);

(b) Incubate 1000 ul of the H-AAPPA-NH2 solution with 200 ul proline tolerant tripeptidyl peptidase solution (40 ug/ml) (substrate/enzyme 100:0.8) at room temperature;

(c) Take 100 ul samples at seven time points (0, 5, 15, 60, 180, 720 and 1440 min), dilute with 50 ul 5% TFA, heat inactivate (10 min at 80° C.) and keep at −20° C. until LC-MS analysis;

(d) Perform Nano LC-MS/MS analyses using an Easy LC system (Thermo Scientific, Odense, DK) interfaced to a LTQ Orbitrap Classic hybrid mass spectrometer (Thermo Scientific, Bremen, Germany);

(e) Load samples onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle;

(f) Perform separation at a flow rate of 300 nL/min using a 10 min gradient of 0-34% Solvent B (H2O/CH3CN/TFE/HCOOH (100/800//100/1 v/v/v/v)) into the nanoelectrospray ion source (Thermo Scientific, Odense, DK)—operate the LTQ Orbitrap Classic instrument in a data-dependent MS/MS mode;

(g) Measure the peptide masses by the Orbitrap (obtain MS scans with a resolution of 60 000 at m/z 400), and select up to 2 of the most intense peptide m/z and subject to fragmentation using CID in the linear ion trap (LTQ). Enable dynamic exclusion with a list size of 500 masses, duration of 40 s, and an exclusion mass width of ±10 ppm relative to masses on the list;

(h) Use the open source program Skyline 1.4.0.4421 (available from MacCoss Lab Software, University of Washington, Department of Genome Sciences, 3720 15^{th} Ave NE Seattle, Wash., US) to access the RAW files which program can use the MS1 intensities to build chromatograms. Set the precursor isotopic import filter to a count of three, (M, M+1, and M+2) at a resolution of 60,000 and use the most intense charge state;

(i) Peptide sequences of the substrate as well as cleavage products were typed into Skyline and intensities were calculated in each sample.

(j) One unit of activity is defined as the amount of enzyme which in this assay will hydrolyse 50% of the substrate within 24 h while releasing AAP.

In one embodiment a proline tolerant tripeptidyl peptidase in accordance with the present invention has an activity of at least 50 nkat in Part 1 of the activity taught herein and at least 100 U activity in Part 2(i) or Part 2(ii) of the assay taught herein per mg of protein. In one embodiment a proline tolerant tripeptidyl peptidase in accordance with the present invention has an activity of between about 50-2000 nkat in Part 1 of the activity taught herein and between about 1-500 units activity in Part 2(i) or Part 2(ii) of the assay taught herein per mg of protein (protein concentration is calculated as in Example 2).

In one embodiment a proline tolerant tripeptidyl peptidase for use in the present invention may have at least 10 U activity in the “P1 and P1′ Proline Activity Assay” taught herein per mg of protein.

In one embodiment a proline tolerant tripeptidyl peptidase in accordance with the present invention has an activity of between about 1 U-500 U activity in the “P1 and P1′ Proline Activity Assay” taught herein per mg of protein.

In addition to the above, the proline tolerant tripeptidyl peptidase may also have activity in accordance with Part 1 of the “Exopeptidase Activity Assay” taught above.

In one embodiment the proline tolerant tripeptidyl peptidase for use in the present invention may have at least 10 U activity in the “P1 and P1′ Proline Activity Assay” taught herein and at least 50 nkatal in Part 1 of the “Exopeptidase Activity Assay” taught herein per mg of protein. In another embodiment a proline tolerant tripeptidyl peptidase in accordance with the present invention has an activity of between about 1 U-500 U activity in the “P1 and P1′ Proline Activity Assay” taught herein and between about 50 U-2000 U katal in Part 1 of the “Exopeptidase Activity Assay” taught herein per mg of protein.

Amino Acid and Nucleotide Sequences

The proline tolerant tripeptidyl peptidase for use in accordance with the present invention may be obtainable (e.g. obtained) from any source so long as it has the activity described herein.

In one embodiment the proline tolerant tripeptidyl peptidase for use in accordance with the present invention may be obtainable (e.g. obtained) from Trichoderma.

Suitably from Trichoderma reesei, more suitably, Trichoderma reesei QM6A.

Suitably from Trichoderma virens, more suitably, Trichoderma virens Gv29-8.

Suitably from Trichoderma atroviride. More suitably, Trichoderma atroviride IMI 206040.

In one embodiment the proline tolerant tripeptidyl peptidase for use in accordance with the present invention may be obtainable (e.g. obtained) from Aspergillus.

Suitably from Aspergillus fumigatus, more suitably Aspergillus fumigatus CAE17675.

Suitably from Aspergillus kawachii, more suitably from Aspergillus kawachii IFO 4308.

Suitably from Aspergillus nidulans, more suitably from Aspergillus nidulans FGSC A4.

Suitably from Aspergillus oryzae, more suitably Aspergillus oryzae RIB40.

Suitably from Aspergillus ruber, more suitably Aspergillus ruber CBS135680.

Suitably from Aspergillus terreus, more suitably from Aspergillus terreus NIH2624.

In one embodiment the proline tolerant tripeptidyl peptidase for use in accordance with the present invention may be obtainable (e.g. obtained) from Bipolaris, suitably from Bipolaris maydis, more suitably Bipolaris maydis C5.

In one embodiment the proline tolerant tripeptidyl peptidase for use in accordance with the present invention may be obtainable (e.g. obtained) from Togninia, suitably from Togninia minima more suitably Togninia minima UCRPA7.

In one embodiment the proline tolerant tripeptidyl peptidase for use in accordance with the present invention may be obtainable (e.g. obtained) from Talaromyces, suitably from Talaromyces stipitatus more suitably Talaromyces stipitatus ATCC 10500.

In one embodiment the proline tolerant tripeptidyl peptidase for use in accordance with the present invention may be obtainable (e.g. obtained) from Arthroderma, suitably from Arthroderma benhamiae more suitably Arthroderma benhamiae CBS 112371.

In one embodiment the proline tolerant tripeptidyl peptidase for use in accordance with the present invention may be obtainable (e.g. obtained) from Magnaporthe, suitably from Magnaporthe oryzae more suitably Magnaporthe oryzae 70-1.

In another embodiment the proline tolerant tripeptidyl peptidase for use in accordance with the present invention may be obtainable (e.g. obtained) from Fusarium.

Suitably from Fusarium oxysporum, more suitably from Fusarium oxysporum f. sp. cubense race 4.

Suitably from Fusarium graminearum, more suitably Fusarium graminearum PH-1.

In a further embodiment the proline tolerant tripeptidyl peptidase for use in accordance with the present invention may be obtainable (e.g. obtained) from Phaeosphaeria, suitably from Phaeosphaeria nodorum more suitably Phaeosphaeria nodorum SN15.

In a yet further embodiment the proline tolerant tripeptidyl peptidase for use in accordance with the present invention may be obtainable (e.g. obtained) from Agaricus, suitably from Agaricus bisporus more suitably Agaricus bisporus var. burnettii JB137-S8.

In a yet further embodiment the proline tolerant tripeptidyl peptidase for use in accordance with the present invention may be obtainable (e.g. obtained) from Acremonium, suitably from Acremonium alcalophilum.

In a yet further embodiment the proline tolerant tripeptidyl peptidase for use in accordance with the present invention may be obtainable (e.g. obtained) from Sodiomyces, suitably from Sodiomyces alkalinus.

In one embodiment the proline tolerant tripeptidyl peptidase for use in accordance with the present invention may be obtainable (e.g. obtained) from Penicillium.

Suitably the proline tolerant tripeptidyl peptidase may be obtainable from Penicillium digitatum, more suitably from Penicillium digitatum Pd1.

Suitably the proline tolerant tripeptidyl peptidase may be obtainable from Penicillium oxalicum, more suitably from Penicillium oxalicum 114-2.

Suitably the proline tolerant tripeptidyl peptidase may be obtainable from Penicillium roqueforti, more suitably from Penicillium roqueforti FM164.

Suitably the proline tolerant tripeptidyl peptidase may be obtainable from Penicillium rubens, more suitably from Penicillium rubens Wisconsin 54-1255.

In another embodiment the proline tolerant tripeptidyl peptidase for use in accordance with the present invention may be obtainable (e.g. obtained) from Neosartorya.

Suitably the proline tolerant tripeptidyl peptidase may be obtainable from Neosartorya fischeri, more suitably from Neosartorya fischeri NRRL181.

In one embodiment the tripeptidyl peptidase (e.g. proline tolerant tripeptidyl peptidase) for use in accordance with the present invention is not obtainable (e.g. obtained) from Aspergillus niger.

| SEQ | | |
|---|---|---|
| ID | | |
| No.: | Sequence | Origin |
| 1 | MAKLSTLRLASLLSLVSVQVSASVHLLESLEKLPHGWKAAETPSPSSQIV | Trichoderma |
| | LQVALTQQNIDQLESRLAAVSTPTSSTYGKYLDVDEINSIFAPSDASSSAV | reesei QM6a |
| | ESWLQSHGVTSYTKQGSSIWFQTNISTANAMLSTNFHTYSDLTGAKKVR | |
| | TLKYSIPESLIGHVDLISPTTYFGTTKAMRKLKSSGVSPAADALAARQEPS | |
| | SCKGTLVFEGETFNVFQPDCLRTEYSVDGYTPSVKSGSRIGFGSFLNES | |
| | ASFADQALFEKHFNIPSQNFSVVLINGGTDLPQPPSDANDGEANLDAQTI | |
| | LTIAHPLPITEFITAGSPPYFPDPVEPAGTPNENEPYLQYYEFLLSKSNAEI | |
| | PQVITNSYGDEEQTVPRSYAVRVCNLIGLLGLRGISVLHSSGDEGVGASC | |
| | VATNSTTPQFNPIFPATCPYVTSVGGTVSFNPEVAWAGSSGGFSYYFSR | |
| | PVVYQQEAVGTYLEKYVSAETKKYYGPYVDFSGRGFPDVAAHSVSPDYP | |
| | VFQGGELTPSGGTSAASPVVAAIVALLNDARLREGKPTLGFLNPLIYLHA | |
| | SKGFTDITSGQSEGCNGNNTQTGSPLPGAGFIAGAHWNATKGWDPTTG | |
| | FGVPNLKKLLALVRF | |
| 2 | SVHLLESLEKLPHGWKAAETPSPSSQIVLQVALTQQNIDQLESRLAAVST | Trichoderma |
| | PTSSTYGKYLDVDEINSIFAPSDASSSAVESWLQSHGVTSYTKQGSSIWF | reesei QM6a |
| | QTNISTANAMLSTNFHTYSDLTGAKKVRTLKYSIPESLIGHVDLISPTTYFG | |
| | TTKAMRKLKSSGVSPAADALAARQEPSSCKGTLVFEGETFNVFQPDCLR | |
| | TEYSVDGYTPSVKSGSRIGFGSFLNESASFADQALFEKHFNIPSQNFSVV | |
| | LINGGTDLPQPPSDANDGEANLDAQTILTIAHPLPITEFITAGSPPYFPDPV | |
| | EPAGTPNENEPYLQYYEFLLSKSNAEIPQVITNSYGDEEQTVPRSYAVRV | |
| | CNLIGLLGLRGISVLHSSGDEGVGASCVATNSTTPQFNPIFPATCPYVTS | |
| | VGGTVSFNPEVAWAGSSGGFSYYFSRPVVYQQEAVGTYLEKYVSAETKK | |
| | YYGPYVDFSGRGFPDVAAHSVSPDYPVFQGGELTPSGGTSAASPVVAAI | |
| | VALLNDARLREGKPTLGFLNPLIYLHASKGFTDITSGQSEGCNGNNTQTG | |
| | SPLPGAGFIAGAHWNATKGWDPTTGFGVPNLKKLLALVRF | |
| 3 | EAFEKLSAVPKGWHYSSTPKGNTEVCLKIALAQKDAAGFEKTVLEMSDP | Aspergillus |
| | DHPSYGQHFTTHDEMKRMLLPRDDTVDAVRQWLENGGVTDFTQDADW | oryzae RIB40 |
| | INFCTTVDTANKLLNAQFKWYVSDVKHIRRLRTLQYDVPESVTPHINTIQP | |
| | TTRFGKISPKKAVTHSKPSQLDVTALAAAVVAKNISHCDSIITPTCLKELYN | |
| | IGDYQADANSGSKIAFASYLEEYARYADLENFENYLAPWAKGQNFSVTT | |
| | FNGGLNDQNSSSDSGEANLDLQYILGVSAPLPVTEFSTGGRGPLVPDLT | |
| | QPDPNSNSNEPYLEFFQNVLKLDQKDLPQVISTSYGENEQEIPEKYARTV | |
| | CNLIAQLGSRGVSVLFSSGDSGVGEGCMTNDGTNRTHFPPQFPAACPW | |
| | VTSVGATFKTTPERGTYFSSGGFSDYWPRPEWQDEAVSSYLETIGDTFK | |
| | GLYNSSGRAFPDVAAQGMNFAVYDKGTLGEFDGTSASAPAFSAVIALLN | |
| | DARLRAGKPTLGFLNPWLYKTGRQGLQDITLGASIGCTGRARFGGAPDG | |
| | GPVVPYASWNATQGWDPVTGLGTPDFAELKKLALGN | |
| 4 | EPFEKLFSTPEGWKMQGLATNEQIVKLQIALQQGDVAGFEQHVIDISTPS | Phaeosphaer |
| | HPSYGAHYGSHEEMKRMIQPSSETVASVSAWLKAAGINDAEIDSDVVVTF | ia nodorum |
| | KTTVGVANKMLDTKFAVVYVSEEAKPRKVLRTLEYSVPDDVAEHINLIQPT | SN15 |
| | TRFAAIRQNHEVAHEIVGLQFAALANNTVNCDATITPQCLKTLYKIDYKAD | |
| | PKSGSKVAFASYLEQYARYNDLALFEKAFLPEAVGQNFSVVQFSGGLND | |
| | QNTTQDSGEANLDLQYIVGVSAPLPVTEFSTGGRGPWVADLDQPDEAD | |
| | SANEPYLEFLQGVLKLPQSELPQVISTSYGENEQSVPKSYALSVCNLFAQ | |
| | LGSRGVSVIFSSGDSGPGSACQSNDGKNTTKFQPQYPAACPFVTSVGS | |
| | TRYLNETATGFSSGGFSDYWKRPSYQDDAVKAYFHHLGEKFKPYFNRH | |
| | GRGFPDVATQGYGFRVYDQGKLKGLQGTSASAPAFAGVIGLLNDARLK | |
| | AKKPTLGFLNPLLYSNSDALNDIVLGGSKGCDGHARFNGPPNGSPVIPYA | |
| | GWNATAGWDPVTGLGTPNFPKLLKAAVPSRYRA | |
| 5 | NAAVLLDSLDKVPVGWQAASAPAPSSKITLQVALTQQNIDQLESKLAAVS | Trichoderma |
| | TPNSSNYGKYLDVDEINQIFAPSSASTAAVESWLKSYGVDYKVQGSSIW | atroviride IMI |
| | FQTDVSTANKMLSTNFHTYTDSVGAKKVRTLQYSVPETLADHIDLISPTT | 206040 |
| | YFGTSKAMRALKIQNAASAVSPLAARQEPSSCKGTIEFENRTFNVFQPD | |
| | CLRTEYSVNGYKPSAKSGSRIGFGSFLNQSASSSDLALFEKHFGFASQG | |
| | FSVELINGGSNPQPPTDANDGEANLDAQNIVSFVQPLPITEFIAGGTAPYF | |
| | PDPVEPAGTPDENEPYLEYYEYLLSKSNKELPQVITNSYGDEEQTVPQA | |
| | YAVRVCNLIGLMGLRGISILESSGDEGVGASCLATNSTTTPQFNPIFPATC | |
| | PYVTSVGGTVSFNPEVAWDGSSGGFSYYFSRPWYQEAAVGTYLNKYVS | |
| | EETKEYYKSYVDFSGRGFPDVAAHSVSPDYPVFQGGELTPSGGTSAAS | |
| | PIVASVIALLNDARLRAGKPALGFLNPLIYGYAYKGFTDITSGQAVGCNGN | |
| | NTQTGGPLPGAGVIPGAFWNATKGWDPTTGFGVPNFKKLLELVRY | |
| 6 | KPTPGASHKVIEHLDFVPEGWQMVGAADPAAIIDFWLAIERENPEKLYDTI | Arthroderma |
| | YDVSTPGRAQYGKHLKREELDDLLRPRAETSESIINWLTNGGVNPQHIR | benhamiae |
| | DEGDVVVRFSTNVKTAETLMNTRFNVFKDNLNSVSKIRTLEYSVPVAISAH | CBS 112371 |
| | VQMIQPTTLFGRQKPQNSLILNPLTKDLESMSVEEFAASQCRSLVTTACL | |
| | RELYGLGDRVTQARDDNRIGVSGFLEEYAQYRDLELFLSRFEPSAKGFN | |
| | FSEGLIAGGKNTQGGPGSSTEANLDMQYVVGLSHKAKVTYYSTAGRGP | |
| | LIPDLSQPSQASNNNEPYLEQLRYLVKLPKNQLPSVLTTSYGDTEQSLPA | |
| | SYTKATCDLFAQLGTMGVSVIFSSGDTGPGSSCQTNDGKNATRFNPIYP | |
| | ASCPFVTSIGGTVGTGPERAVSFSSGGFSDRFPRPQYQDNAVKDYLKIL | |
| | GNQWSGLFDPNGRAFPDIAAQGSNYAVYDKGRMTGVSGTSASAPAMA | |
| | AIIAQLNDFRLAKGSPVLGFLNPWIYSKGFSGFTDIVDGGSRGCTGYDIYS | |
| | GLKAKKVPYASWNATKGWDPVTGFGTPNFQALTKVLP | |
| 7 | KSYSHHAEAPKGWKVDDTARVASTGKQQVFSIALTMQNVDQLESKLLDL | Fusarium |
| | SSPDSKNYGQWMSQKDVTTAFYPSKEAVSSVTKWLKSKGVKHYNVNG | graminearum |
| | GFIDFALDVKGANALLDSDYQYYTKEGQTKLRTLSYSIPDDVAEHVQFVD | PH-1 |
| | PSTNFGGTLAFAPVTHPSRTLTERKNKPTKSTVDASCQTSITPSCLKQMY | |
| | NIGDYTPKVESGSTIGFSSFLGESAIYSDVFLFEEKFGIPTQNFTTVLINNG | |
| | TDDQNTAHKNFGEADLDAENIVGIAHPLPFTQYITGGSPPFLPNIDQPTAA | |
| | DNQNEPYVPFFRYLLSQKEVPAVVSTSYGDEEDSVPREYATMTCNLIGL | |
| | LGLRGISVIFSSGDIGVGAGCLGPDHKTVEFNAIFPATCPYLTSVGGTVDV | |
| | TPEIAWEGSSGGFSKYFPRPSYQDKAVKTYMKTVSKQTKKYYGPYTNW | |
| | EGRGFPDVAGHSVSPNYEVIYAGKQSASGGTSAAAPVWAAIVGLLNDAR | |
| | FRAGKPSLGWLNPLVYKYGPKVLTDITGGYAIGCDGNNTQSGKPEPAGS | |
| | GIVPGARWNATAGWDPVTGYGTPDFGKLKDLVLSF | |
| 8 | AVVIRAAVLPDAVKLMGKAMPDDIISLQFSLKQQNIDQLETRLRAVSDPSS | Acremonium |
| | PEYGQYMSESEVNEFFKPRDDSFAEVIDVVVAASGFQDIHLTPQAAAINLA | alcalophilum |
| | ATVETADQLLGANFSWFDVDGTRKLRTLEYTIPDRLADHVDLISPTTYFG | |
| | RARLDGPRETPTRLDKRQRDPVADKAYFHLKWDRGTSNCDLVITPPCLE | |
| | AAYNYKNYMPDPNSGSRVSFTSFLEQAAQQSDLTKFLSLTGLDRLRPPS | |
| | SKPASFDTVLINGGETHQGTPPNKTSEANLDVQWLAAVIKARLPITQWIT | |
| | GGRPPFVPNLRLRHEKDNTNEPYLEFFEYLVRLPARDLPQVISNSYAEDE | |
| | QTVPEAYARRVCNLIGIMGLRGVTVLTASGDSGVGAPCRANDGSDRLEF | |
| | SPQFPTSCPYITAVGGTEGWDPEVAWEASSGGFSHYFLRPVVYQANAVE | |
| | KYLDEELDPATRAYYDGNGFVQFAGRAYPDLSAHSSSPRYAYIDKLAPG | |
| | LTGGTSASCPVVAGIVGLLNDARLRRGLPTMGFINPWLYTRGFEALQDV | |
| | TGGRASGCQGIDLQRGTRVPGAGIIPWASWNATPGWDPATGLGLPDFW | |
| | AMRGLALGRGT | |
| 9 | AVVIRAAPLPESVKLVRKAAAEDGINLQLSLKRQNMDQLEKFLRAVSDPF | Sodiomyces |
| | SPKYGQYMSDAEVHEIFRPTEDSFDQVIDWLTKSGFGNLHITPQAAAINV | alkalinus |
| | ATTVETADQLFGANFSWFDVDGTPKLRTGEYTIPDRLVEHVDLVSPTTYF | |
| | GRMRPPPRGDGVNDWITENSPEQPAPLNKRDTKTESDQARDHPSWDS | |
| | RTPDCATIITPPCLETAYNYKGYIPDPKSGSRVSFTSFLEQAAQQADLTKF | |
| | LSLTRLEGFRTPASKKKTFKTVLINGGESHEGVHKKSKTSEANLDVQWLA | |
| | AVTQTKLPITQWITGGRPPFVPNLRIPTPEANTNEPYLEFLEYLFRLPDKD | |
| | LPQVISNSYAEDEQSVPEAYARRVCGLLGIMGLRGVTVLTASGDSGVGA | |
| | PCRANDGSGREEFSPQFPSSCPYITTVGGTQAWDPEVAWKGSSGGFS | |
| | NYFPRPWYQVAAVEKYLEEQLDPAAREYYEENGFVRFAGRAFPDLSAH | |
| | SSSPKYAYVDKRVPGLTGGTSASCPVVAGIVGLLNDARLRRGLPTMGFI | |
| | NPWLYAKGYQALEDVTGGAAVGCQGIDIQTGKRVPGAGIIPGASWNATP | |
| | DWDPATGLGLPNFWAMRELALED | |
| 10 | WHEKLAAVPSGWHHLEDAGSDHQISLSIALARKNLDQLESKLKDLSTPG | Aspergillus |
| | ESQYGQWLDQEEVDTLFPVASDKAVISWLRSANITHIARQGSLVNFATTV | kawachii IFO |
| | DKVNKLLNTTFAYYQRGSSQRLRTTEYSIPDDLVDSIDLISPTTFFGKEKT | 4308 |
| | SAGLTQRSQKVDNHVAKRSNSSSCADTITLSCLKEMYNFGNYTPSASSG | |
| | SKLGFASFLNESASYSDLAKFERLFNLPSQNFSVELINGGVNDQNQSTAS | |
| | LTEADLDVELLVGVGHPLPVTEFITSGEPPFIPDPDEPSAADNENEPYLQ | |
| | YYEYLLSKPNSALPQVISNSYGDDEQTVPEYYAKRVCNLIGLVGLRGISVL | |
| | ESSGDEGIGSGCRTTDGTNSTQFNPIFPATCPYVTAVGGTMSYAPEIAW | |
| | EASSGGFSNYFERAWFQKEAVQNYLANHITNETKQYYSQFANFSGRGF | |
| | PDVSAHSFEPSYEVIFYGARYGSGGTSAACPLFSALVGMLNDARLRAGK | |
| | STLGFLNPLLYSKGYKALTDVTAGQSIGCNGIDPQSDEAVAGAGIIPWAH | |
| | WNATVGWDPVTGLGLPDFEKLRQLVLSL | |
| 11 | AAALVGHESLAALPVGWDKVSTPAAGTNIQLSVALALQNIEQLEDHLKSV | Talaromyces |
| | STPGSASYGQYLDSDGIAAQYGPSDASVEAVTNWLKEAGVTDIYNNGQ | stipitatus |
| | SIHFATSVSKANSLLGADFNYYSDGSATKLRTLAYSVPSDLKEAIDLVSPT | ATCC 10500 |
| | TYFGKTTASRSIQAYKNKRASTTSKSGSSSVQVSASCQTSITPACLKQMY | |
| | NVGNYTPSVAHGSRVGFGSFLNQSAIFDDLFTYEKVNDIPSQNFTKVIIAN | |
| | ASNSQDASDGNYGEANLDVQNIVGISHPLPVTEFLTGGSPPFVASLDTPT | |
| | NQNEPYIPYYEYLLSQKNEDLPQVISNSYGDDEQSVPYKYAIRACNLIGLT | |
| | GLRGISVLESSGDLGVGAGCRSNDGKNKTQFDPIFPATCPYVTSVGGTQ | |
| | SVTPEIAVVVASSGGFSNYFPRTVVYQEPAIQTYLGLLDDETKTYYSQYTN | |
| | FEGRGFPDVSAHSLTPDYQVVGGGYLQPSGGTSAASPVFAGIIALLNDA | |
| | RLAAGKPTLGFLNPFFYLYGYKGLNDITGGQSVGCNGINGQTGAPVPGG | |
| | GIVPGAAWNSTTGWDPATGLGTPDFQKLKELVLSF | |
| 12 | KSFSHHAEAPQGWQVQKTAKVASNTQHVFSLALTMQNVDQLESKLLDL | Fusarium |
| | SSPDSANYGNWLSHDELTSTFSPSKEAVASVTKWLKSKGIKHYKVNGAF | oxysporum f. |
| | IDFAADVEKANTLLGGDYQYYTKDGQTKLRTLSYSIPDDVAGHVQFVDPS | sp. cubense |
| | TNFGGTVAFNPVPHPSRTLQERKVSPSKSTVDASCQTSITPSCLKQMYNI | race 4 |
| | GDYTPDAKSGSEIGFSSFLGQAAIYSDVFKFEELFGIPKQNYTTILINNGT | |
| | DDQNTAHGNFGEANLDAENIVGIAHPLPFKQYITGGSPPFVPNIDQPTEK | |
| | DNQNEPYVPFFRYLLGQKDLPAVISTSYGDEEDSVPREYATLTCNMIGLL | |
| | GLRGISVIFSSGDIGVGSGCLAPDYKTVEFNAIFPATCPYLTSVGGTVDVT | |
| | PEIAWEGSSGGFSKYFPRPSYQDKAIKKYMKTVSKETKKYYGPYTNWE | |
| | GRGFPDVAGHSVAPDYEVIYNGKQARSGGTSAAAPVWAAIVGLLNDAR | |
| | FKAGKKSLGWLNPLIYKHGPKVLTDITGGYAIGCDGNNTQSGKPEPAGS | |
| | GLVPGARWNATAGWDPTTGYGTPNFQKLKDLVLSL | |
| 13 | SVLVESLEKLPHGWKAASAPSPSSQITLQVALTQQNIDQLESRLAAVSTP | Trichoderma |
| | NSKTYGNYLDLDEINEIFAPSDASSAAVESWLHSHGVTKYTKQGSSIWFQ | virens Gv29- |
| | TEVSTANAMLSTNFHTYSDAAGVKKLRTLQYSIPESLVGHVDLISPTTYF | 8 |
| | GTSNAMRALRSKSVASVAQSVAARQEPSSCKGTLVFEGRTFNVFQPDC | |
| | LRTEYNVNGYTPSAKSGSRIGFGSFLNQSASFSDLALFEKHFGFSSQNF | |
| | SVVLINGGTDLPQPPSDDNDGEANLDVQNILTIAHPLPITEFITAGSPPYFP | |
| | DPVEPAGTPDENEPYLQYFEYLLSKPNRDLPQVITNSYGDEEQTVPQAY | |
| | AVRVCNLIGLMGLRGISILESSGDEGVGASCVATNSTTPQFNPIFPATCPY | |
| | VTSVGGTVNFNPEVAWDGSSGGFSYYFSRPVVYQEEAVGNYLEKHVSA | |
| | ETKKYYGPYVDFSGRGFPDVAAHSVSPDYPVFQGGQLTPSGGTSAASP | |
| | VVASIIALLNDARLREGKPTLGFLNPLIYQYAYKGFTDITSGQSDGCNGNN | |
| | TQTDAPLPGAGVVLGAHWNATKGWDPTTGFGVPNFKKLLELIRYI | |
| 14 | AVLVESLKQVPNGWNAVSTPDPSTSIVLQIALAQQNIDELEWRLAAVSTP | Trichoderma |
| | NSGNYGKYLDIGEIEGIFAPSNASYKAVASWLQSHGVKNFVKQAGSIWF | atroviride IMI |
| | YTTVSTANKMLSTDFKHYSDPVGIEKLRTLQYSIPEELVGHVDLISPTTYF | 206040 |
| | GNNHPATARTPNMKAINVTYQIFHPDCLKTKYGVDGYAPSPRCGSRIGF | |
| | GSFLNETASYSDLAQFEKYFDLPNQNLSTLLINGAIDVQPPSNKNDSEAN | |
| | MDVQTILTFVQPLPITEFVVAGIPPYIPDAALPIGDPVQNEPWLEYFEFLM | |
| | SRTNAELPQVIANSYGDEEQTVPQAYAVRVCNQIGLLGLRGISVIASSGD | |
| | TGVGMSCMASNSTTPQFNPMFPASCPYITTVGGTQHLDNEIAWELSSG | |
| | GFSNYFTRPVVYQEDAAKTYLERHVSTETKAYYERYANFLGRGFPDVAAL | |
| | SLNPDYPVIIGGELGPNGGTSAAAPVVASIIALLNDARLCLGKPALGFLNP | |
| | LIYQYADKGGFTDITSGQSWGCAGNTTQTGPPPPGAGVIPGAHWNATK | |
| | GWDPVTGFGTPNFKKLLSLALSV | |
| 15 | SPLARRWDDFAEKHAVVVEVPRGWEMVSEAPSDHTFDLRIGVKSSGME | Agaricus |
| | LIENLMQTSDPTHSRYGQHLSKEELHDFVQPHPDSTGAVEAWLEDFG1 | bisporus var. |
| | SDDFIDRTGSGNVVVTVRVSVAQAERMLGTKYNVYRHSESGESVVRTMS | burnettii |
| | YSLPSELHSHIDVVAPTTYFGTMKSMRVTSFLQPEIEPVDPSAKPSAAPA | JB137-S8 |
| | SCLSTTVITPDCLRDLYNTADYVPSATSRNAIGIAGYLDRSNRADLQTFFR | |
| | RFRPDAVGFNYTTVQLNGGGDDQNDPGVEANLDIQYAAGIAFPTPATY | |
| | WSTGGSPPFIPDTQTPTNTNEPYLDWINFVLGQDEIPQVISTSYGDDEQT | |
| | VPEDYATSVCNLFAQLGSRGVTVFFSSGDFGVGGGDCLTNDGSNQVLF | |
| | QPAFPASCPFVTAVGGTVRLDPEIAVSFSGGGFSRYFSRPSYQNQTVAQ | |
| | FVSNLGNTFNGLYNKNGRAYPDLAAQGNGFQVVIDGIVRSVGGTSASSP | |
| | TVAGIFALLNDFKLSRGQSTLGFINPLIYSSATSGFNDIRAGTNPGCGTRG | |
| | FTAGTGWDPVTGLGTPDFLRLQGLI | |
| 16 | RVFDSLPHPPRGWSYSHAAESTEPLTLRIALRQQNAAALEQVVLQVSNP | Magnaporthe |
| | RHANYGQHLTRDELRSYTAPTPRAVRSVTSWLVDNGVDDYTVEHDVVVT | oryzae 70-15 |
| | LRTTVGAADRLLGADFAVVYAGPGETLQLRTLSYGVDDSVAPHVDLVQPT | |
| | TRFGGPVGQASHIFKQDDFDEQQLKTLSVGFQVMADLPANGPGSIKAAC | |
| | NESGVTPLCLRTLYRVNYKPATTGNLVAFASFLEQYARYSDQQAFTQRV | |
| | LGPGVPLQNFSVETVNGGANDQQSKLDSGEANLDLQYVMAMSHPIPILE | |
| | YSTGGRGPLVPTLDQPNANNSSNEPYLEFLTYLLAQPDSAIPQTLSVSYG | |
| | EEEQSVPRDYAIKVCNMFMQLGARGVSVMFSSGDSGPGNDCVRASDN | |
| | ATFFGSTFPAGCPYVTSVGSTVGFEPERAVSFSSGGFSIYHARPDYQNE | |
| | VVPKYIESIKASGYEKFFDGNGRGIPDVAAQGARFVVIDKGRVSLISGTSA | |
| | SSPAFAGMVALVNAARKSKDMPALGFLNPMLYQNAAAMTDIVNGAGIGC | |
| | RKQRTEFPNGARFNATAGWDPVTGLGTPLFDKLLAVGAPGVPNA | |
| 17 | SDVVLESLREVPQGWKRLRDADPEQSIKLRIALEQPNLDLFEQTLYDISS | Togninia |
| | PDHPKYGQHLKSHELRDIMAPREESTAAVIAWLQDAGLSGSQIEDDSDW | minima |
| | INIQTTVAQANDMLNTTFGLFAQEGTEVNRIRALAYSVPEEIVPHVKMIAPI | UCRPA7 |
| | IRFGQLRPQMSHIFSHEKVEETPSIGTIKAAAIPSVDLNVTACNASITPECL | |
| | RALYNVGDYEADPSKKSLFGVCGYLEQYAKHDQLAKFEQTYAPYAIGAD | |
| | FSVVTINGGGDNQTSTIDDGEANLDMQYAVSMAYKTPITYYSTGGRGPL | |
| | VPDLDQPDPNDVSNEPYLDFVSYLLKLPDSKLPQTITTSYGEDEQSVPRS | |
| | YVEKVCTMFGALGARGVSVIFSSGDTGVGSACQTNDGKNTTRFLPIFPA | |
| | ACPYVTSVGGTRYVDPEVAVSFSSGGFSDIFPTPLYQKGAVSGYLKILGD | |
| | RWKGLYNPHGRGFPDVSGQSVRYHVFDYGKDVMYSGTSASAPMFAAL | |
| | VSLLNNARLAKKLPPMGFLNPWLYTVGFNGLTDIVHGGSTGCTGTDVYS | |
| | GLPTPFVPYASWNATVGWDPVTGLGTPLFDKLLNLSTPNFHLPHIGGH | |
| 18 | STTSHVEGEVVERLHGVPEGWSQVGAPNPDQKLRFRIAVRSADSELFE | Bipolaris |
| | RTLMEVSSPSHPRYGQHLKRHELKDLIKPRAKSTSNILNWLQESGIEARD | maydis C5 |
| | IQNDGEWISFYAPVKRAEQMMSTTFKTYQNEARANIKKIRSLDYSVPKHI | |
| | RDDIDIIQPTTRFGQIQPERSQVFSQEEVPFSALVVNATCNKKITPDCLAN | |
| | LYNFKDYDASDANVTIGVSGFLEQYARFDDLKQFISTFQPKAAGSTFQVT | |
| | SVNAGPFDQNSTASSVEANLDIQYTTGLVAPDIETRYFTVPGRGILIPDLD | |
| | QPTESDNANEPYLDYFTYLNNLEDEELPDVLTTSYGESEQSVPAEYAKK | |
| | VCNLIGQLGARGVSVIFSSGDTGPGSACQTNDGKNTTRFLPIFPASCPYV | |
| | TSVGGTVGVEPEKAVSFSSGGFSDLWPRPAYQEKAVSEYLEKLGDRWN | |
| | GLYNPQGRGFPDVAAQGQGFQVFDKGRLISVGGTSASAPVFASVVALL | |
| | NNARKAAGMSSLGFLNPWIYEQGYKGLTDIVAGGSTGCTGRSIYSGLPA | |
| | PLVPYASWNATEGWDPVTGYGTPDFKQLLTLATAPKSGERRVRRGGLG | |
| | GQA | |
| 19 | MLSSFLSQGAAVSLALLSLLPSPVAAEIFEKLSGVPNGWRYANNPHGNE | Aspergillus |
| | VIRLQIALQQHDVAGFEQAVMDMSTPGHADYGKHFRTHDEMKRMLLPS | kawachii IFO |
| | DTAVDSVRDWLESAGVHNIQVDADVVVKFHTTVNKANALLDADFKWYVS | 4308 |
| | EAKHIRRLRTLQYSIPDALVSHINMIQPTTRFGQIQPNRATMRSKPKHADE | |
| | TFLTAATLAQNTSHCDSIITPHCLKQLYNIGDYQADPKSGSKVGFASYLEE | |
| | YARYADLERFEQHLAPNAIGQNFSVVQFNGGLNDQLSLSDSGEANLDLQ | |
| | YILGVSAPVPVTEYSTGGRGELVPDLSSPDPNDNSNEPYLDFLQGILKLD | |
| | NSDLPQVISTSYGEDEQTIPVPYARTVCNLYAQLGSRGVSVIFSSGDSGV | |
| | GAACLTNDGTNRTHFPPQFPASCPWVTSVGATSKTSPEQAVSFSSGGF | |
| | SDLWPRPSYQQAAVQTYLTQHLGNKFSGLFNASGRAFPDVAAQGVNYA | |
| | VYDKGMLGQFDGTSCSAPTFSGVIALLNDARLRAGLPVMGFLNPFLYGV | |
| | GSESGALNDIVNGGSLGCDGRNRFGGTPNGSPVVPFASWNATTGWDP | |
| | VSGLGTPDFAKLRGVALGEAKAYGN | |
| 20 | MAATGRFTAFWNVASVPALIGILPLAGSHLRAVLCPVCIWRHSKAVCAPD | Aspergillus |
| | TLQAMRAFTRVTAISLAGFSCFAAAAAAAFESLRAVPDGWIYESTPDPNQ | nidulans |
| | PLRLRIALKQHNVAGFEQALLDMSTPGHSSYGQHFGSYHEMKQLLLPTE | FGSC A4 |
| | EASSSVRDWLSAAGVEFEQDADWINFRTTVDQANALLDADFLWYTTTG | |
| | STGNPTRILRTLSYSVPSELAGYVNMIQPTTRFGGTHANRATVRAKPIFLE | |
| | TNRQLINAISSGSLEHCEKAITPSCLADLYNTEGYKASNRSGSKVAFASFL | |
| | EEYARYDDLAEFEETYAPYAIGQNFSVISINGGLNDQDSTADSGEANLDL | |
| | QYIIGVSSPLPVTEFTTGGRGKLIPDLSSPDPNDNTNEPFLDFLEAVLKLD | |
| | QKDLPQVISTSYGEDEQTIPEPYARSVCNLYAQLGSRGVSVLFSSGDSG | |
| | VGAACQTNDGKNTTHFPPQFPASCPVVVTAVGGTNGTAPESGVYFSSG | |
| | GFSDYWARPAYQNAAVESYLRKLGSTQAQYFNRSGRAFPDVAAQAQN | |
| | FAVVDKGRVGLFDGTSCSSPVFAGIVALLNDVRLKAGLPVLGFLNPWLY | |
| | QDGLNGLNDIVDGGSTGCDGNNRFNGSPNGSPVIPYAGWNATEGWDP | |
| | VTGLGTPDFAKLKALVLDA | |
| 21 | MLSFVRRGALSLALVSLLTSSVAAEVFEKLHVVPEGWRYASTPNPKQPIR | Aspergillus |
| | LQIALQQHDVTGFEQSLLEMSTPDHPNYGKHFRTHDEMKRMLLPNENA | ruber CBS |
| | VHAVREWLQDAGISDIEEDADVVVRFHTTVDQANDLLDANFLWYAHKSH | 135680 |
| | RNTARLRTLEYSIPDSIAPQVNVIQPTTRFGQIRANRATHSSKPKGGLDEL | |
| | AISQAATADDDSICDQITTPHCLRKLYNVNGYKADPASGSKIGFASFLEEY | |
| | ARYSDLVLFEENLAPFAEGENFTVVMYNGGKNDQNSKSDSGEANLDLQ | |
| | YIVGMSAGAPVTEFSTAGRAPVIPDLDQPDPSAGTNEPYLEFLQNVLHM | |
| | DQEHLPQVISTSYGENEQTIPEKYARTVCNMYAQLGSRGVSVIFSSGDS | |
| | GVGSACMTNDGTNRTHFPPQFPASCPVVVTSVGATEKMAPEQATYFSS | |
| | GGFSDLFPRPKYQDAAVSSYLQTLGSRYQGLYNGSNRAFPDVSAQGTN | |
| | FAVYDKGRLGQFDGTSCSAPAFSGIIALLNDVRLQNNKPVLGFLNPWLY | |
| | GAGSKGLNDVVHGGSTGCDGQERFAGKANGSPVVPYASWNATQGWD | |
| | PVTGLGTPDFGKLKDLALSA | |
| 22 | MLPSLVNNGALSLAVLSLLTSSVAGEVFEKLSAVPKGWHFSHAAQADAPI | Aspergillus |
| | NLKIALKQHDVEGFEQALLDMSTPGHENYGKHFHEHDEMKRMLLPSDS | terreus |
| | AVDAVQTWLTSAGITDYDLDADWINLRTTVEHANALLDTQFGWYENEVR | NIH2624 |
| | HITRLRTLQYSIPETVAAHINMVQPTTRFGQIRPDRATFHAHHTSDARILS | |
| | ALAAASNSTSCDSVITPKCLKDLYKVGDYEADPDSGSQVAFASYLEEYA | |
| | RYADMVKFQNSLAPYAKGQNFSVVLYNGGVNDQSSSADSGEANLDLQT | |
| | IMGLSAPLPITEYITGGRGKLIPDLSQPNPNDNSNEPYLEFLQNILKLDQD | |
| | ELPQVISTSYGEDEQTIPRGYAESVCNMLAQLGSRGVSVVFSSGDSGVG | |
| | AACQTNDGRNQTHFNPQFPASCPVVVTSVGATTKTNPEQAVYFSSGGFS | |
| | DFWKRPKYQDEAVAAYLDTLGDKFAGLFNKGGRAFPDVAAQGMNYAIY | |
| | DKGTLGRLDGTSCSAPAFSAIISLLNDARLREGKPTMGFLNPWLYGEGR | |
| | EALNDVVVGGSKGCDGRDRFGGKPNGSPVVPFASWNATQGWDPVTGL | |
| | GTPNFAKMLELAP | |
| 23 | MIASLFNRRALTLALLSLFASSATADVFESLSAVPQGWRYSRTPSANQPL | Penicillium |
| | KLQIALAQGDVAGFEAAVIDMSTPDHPSYGNHFNTHEEMKRMLQPSAES | digitatum |
| | VDSIRNWLESAGISKIEQDADWMTFYTTVKTANELLAANFQFYINGVKKIE | Pd1 |
| | RLRTLKYSVPDALVSHINMIQPTTRFGQLRAQRAILHTEVKDNDEAFRSN | |
| | AMSANPDCNSIITPQCLKDLYSIGDYEADPTNGNKVAFASYLEEYARYSD | |
| | LALFEKNIAPFAKGQNFSVVQYNGGGNDQQSSSGSSEANLDLQYIVGVS | |
| | SPVPVTEFSTGGRGELVPDLDQPNPNDNNNEPYLEFLQNVLKLHKKDLP | |
| | QVISTSYGEDEQSVPEKYARAVCNLYSQLGSRGVSVIFSSGDSGVGAAC | |
| | QTNDGRNATHFPPQFPAACPVVVTSVGATTHTAPERAVYFSSGGFSDLW | |
| | DRPTWQEDAVSEYLENLGDRWSGLFNPKGRAFPDVAAQGENYAIYDKG | |
| | SLISVDGTSCSAPAFAGVIALLNDARIKANRPPMGFLNPWLYSEGRSGLN | |
| | DIVNGGSTGCDGHGRFSGPTNGGTSIPGASWNATKGWDPVSGLGSPN | |
| | FAAMRKLANAE | |
| 24 | MHVPLLNQGALSLAVVSLLASTVSAEVFDKLVAVPEGWRFSRTPSGDQP | Penicillium |
| | IRLQVALTQGDVEGFEKAVLDMSTPDHPNYGKHFKSHEEVKRMLQPAG | oxalicum |
| | ESVEAIHQWLEKAGITHIQQDADWMTFYTTVEKANNLLDANFQYYLNEN | 114-2 |
| | KQVERLRTLEYSVPDELVSHINLVTPTTRFGQLHAEGVTLHGKSKDVDE | |
| | QFRQAATSPSSDCNSAITPQCLKDLYKVGDYKASASNGNKVAFTSYLEQ | |
| | YARYSDLALFEQNIAPYAQGQNFTVIQYNGGLNDQSSPADSSEANLDLQ | |
| | YlIGTSSPVPVTEFSTGGRGPLVPDLDQPDINDNNNEPYLDFLQNVIKMS | |
| | DKDLPQVISTSYGEDEQSVPASYARSVCNLIAQLGGRGVSVIFSSGDSG | |
| | VGSACQTNDGKNTTRFPAQFPAACPWVTSVGATTGISPERGVFFSSGG | |
| | FSDLWSRPSWQSHAVKAYLHKLGKRQDGLFNREGRAFPDVSAQGENY | |
| | AIYAKGRLGKVDGTSCSAPAFAGLVSLLNDARIKAGKSSLGFLNPWLYSH | |
| | PDALNDITVGGSTGCDGNARFGGRPNGSPVVPYASWNATEGWDPVTG | |
| | LGTPNFQKLLKSAVKQK | |
| 25 | MIASLFSRGALSLAVLSLLASSAAADVFESLSAVPQGWRYSRRPRADQP | Penicillium |
| | LKLQIALTQGDTAGFEEAVMEMSTPDHPSYGHHFTTHEEMKRMLQPSA | roqueforti |
| | ESAESIRDWLEGAGITRIEQDADWMTFYTTVETANELLAANFQFYVSNVR | FM164 |
| | HIERLRTLKYSVPKALVPHINMIQPTTRFGQLRAHRGILHGQVKESDEAFR | |
| | SNAVSAQPDCNSIITPQCLKDIYNIGDYQANDTNGNKVGFASYLEEYARY | |
| | SDLALFEKNIAPSAKGQNFSVTRYNGGLNDQSSSGSSSEANLDLQYIVG | |
| | VSSPVPVTEFSVGGRGELVPDLDQPDPNDNNNEPYLEFLQNVLKLDKKD | |
| | LPQVISTSYGEDEQSIPEKYARSVCNLYSQLGSRGVSVIFSSGDSGVGSA | |
| | CLTNDGRNATRFPPQFPAACPWVTSVGATTHTAPEQAVYFSSGGFSDL | |
| | WARPKWQEEAVSEYLEILGNRWSGLFNPKGRAFPDVTAQGRNYAIYDK | |
| | GSLTSVDGTSCSAPAFAGVVALLNDARLKVNKPPMGFLNPWLYSTGRA | |
| | GLKDIVDGGSTGCDGKSRFGGANNGGPSIPGASWNATKGWDPVSGLG | |
| | SPNFATMRKLANAE | |
| 26 | MIASLFNRGALSLAVLSLLASSASADVFESLSAVPQGWRYSRRPRADQP | Penicillium |
| | LKLQIALAQGDTAGFEEAVMDMSTPDHPSYGNHFHTHEEMKRMLQPSA | rubens |
| | ESADSIRDWLESAGINRIEQDADWMTFYTTVETANELLAANFQFYANSAK | Wisconsin |
| | HIERLRTLQYSVPEALMPHINMIQPTTRFGQLRVQGAILHTQVKETDEAF | 54-1255 |
| | RSNAVSTSPDCNSIITPQCLKNMYNVGDYQADDDNGNKVGFASYLEEYA | |
| | RYSDLELFEKNVAPFAKGQNFSVIQYNGGLNDQHSSASSSEANLDLQYI | |
| | VGVSSPVPVTEFSVGGRGELVPDLDQPDPNDNNNEPYLEFLQNVLKME | |
| | QQDLPQVISTSYGENEQSVPEKYARTVCNLFSQLGSRGVSVIFASGDSG | |
| | VGAACQTNDGRNATRFPAQFPAACPWVTSVGATTHTAPEKAVYFSSGG | |
| | FSDLWDRPKWQEDAVSDYLDTLGDRWSGLFNPKGRAFPDVSAQGQNY | |
| | AIYDKGSLTSVDGTSCSAPAFAGVIALLNDARLKANKPPMGFLNPWLYST | |
| | GRDGLNDIVHGGSTGCDGNARFGGPGNGSPRVPGASWNATKGWDPV | |
| | SGLGSPNFATMRKLANGE | |
| 27 | MLSSTLYAGLLCSLAAPALGVVHEKLSAVPSGWTLVEDASESDTTTLSIA | Neosartorya |
| | LARQNLDQLESKLTTLATPGNAEYGKWLDQSDIESLFPTASDDAVIQWLK | fischeri |
| | DAGVTQVSRQGSLVNFATTVGTANKLFDTKFSYYRNGASQKLRTTQYSI | NRRL 181 |
| | PDSLTESIDLIAPTVFFGKEQDSALPPHAVKLPALPRRAATNSSCANLITP | |
| | DCLVEMYNLGDYKPDASSGSRVGFGSFLNQSANYADLAAYEQLFNIPPQ | |
| | NFSVELINGGANDQNWATASLGEANLDVELIVAVSHALPVVEFITGGSPP | |
| | FVPNVDEPTAADNQNEPYLQYYEYLLSKPNSHLPQVISNSYGDDEQTVP | |
| | EYYARRVCNLIGLMGLRGITVLESSGDTGIGSACMSNDGTNTPQFTPTFP | |
| | GTCPFITAVGGTQSYAPEVAWDASSGGFSNYFSRPWYQYFAVENYLNN | |
| | HITKDTKKYYSQYTNFKGRGFPDVSAHSLTPDYEVVLTGKHYKSGGTSA | |
| | ACPVFAGIVGLLNDARLRAGKSTLGFLNPLLYSILAEGFTDITAGSSIGCN | |
| | GINPQTGKPVPGGGIIPYAHWNATAGWDPVTGLGVPDFMKLKELVLSL | |
| 28 | MLSSTLYAGWLLSLAAPALCVVQEKLSAVPSGWTLIEDASESDTITLSIAL | Aspergillus |
| | ARQNLDQLESKLTTLATPGNPEYGKWLDQSDIESLFPTASDDAVLQWLK | fumigatus |
| | AAGITQVSRQGSLVNFATTVGTANKLFDTKFSYYRNGASQKLRTTQYSIP | CAE17675 |
| | DHLTESIDLIAPTVFFGKEQNSALSSHAVKLPALPRRAATNSSCANLITPD | |
| | CLVEMYNLGDYKPDASSGSRVGFGSFLNESANYADLAAYEQLFNIPPQN | |
| | FSVELINRGVNDQNWATASLGEANLDVELIVAVSHPLPVVEFITGALPPVL | |
| | RVLALQTQLPSSSGDFQLTVPEYYARRVCNLIGLMGLRGITVLESSGDTG | |
| | IGSACMSNDGTNKPQFTPTFPGTCPFITAVGGTQSYAPEVAWDGSSGG | |
| | FSNYFSRPVVYQSFAVDNYLNNHITKDTKKYYSQYTNFKGRGFPDVSAHS | |
| | LTPYYEVVLTGKHYKSGGTSAASPVFAGIVGLLNDARLRAGKSTLGFLNP | |
| | LLYSILAEGFTDITAGSSIGCNGINPQTGKPVPGGGIIPYAHWNATAGWDP | |
| | VTGLGVPDFMKLKELVLSL | |
| 29 | QEPSSCKGTLVFEGETFNVFQPDCLRTEYSVDGYTPSVKSGSRIGFGSF | Trichoderma |
| | LNESASFADQALFEKHFNIPSQNFSVVLINGGTDLPQPPSDANDGEANLD | reesei QM6a |
| | AQTILTIAHPLPITEFITAGSPPYFPDPVEPAGTPNENEPYLQYYEFLLSKS | |
| | NAEIPQVITNSYGDEEQTVPRSYAVRVCNLIGLLGLRGISVLHSSGDEGV | |
| | GASCVATNSTTPQFNPIFPATCPYVTSVGGTVSFNPEVAWAGSSGGFSY | |
| | YFSRPWYQQEAVGTYLEKYVSAETKKYYGPYVDFSGRGFPDVAAHSVS | |
| | PDYPVFQGGELTPSGGTSAASPVVAAIVALLNDARLREGKPTLGFLNPLI | |
| | YLHASKGFTDITSGQSEGCNGNNTQTGSPLPGAGFIAGAHWNATKGWD | |
| | PTTGFGVPNLKKLLALVRF | |
| 30 | CDSIITPTCLKELYNIGDYQADANSGSKIAFASYLEEYARYADLENFENYL | Aspergillus |
| | APWAKGQNFSVTTFNGGLNDQNSSSDSGEANLDLQYILGVSAPLPVTEF | oryzae RIB40 |
| | STGGRGPLVPDLTQPDPNSNSNEPYLEFFQNVLKLDQKDLPQVISTSYG | |
| | ENEQEIPEKYARTVCNLIAQLGSRGVSVLFSSGDSGVGEGCMTNDGTNR | |
| | THFPPQFPAACPVVVTSVGATFKTTPERGTYFSSGGFSDYWPRPEWQD | |
| | EAVSSYLETIGDTFKGLYNSSGRAFPDVAAQGMNFAVYDKGTLGEFDGT | |
| | SASAPAFSAVIALLNDARLRAGKPTLGFLNPWLYKTGRQGLQDITLGASI | |
| | GCTGRARFGGAPDGGPVVPYASWNATQGWDPVTGLGTPDFAELKKLA | |
| 31 | CDATITPQCLKTLYKIDYKADPKSGSKVAFASYLEQYARYNDLALFEKAFL | Phaeosphaer |
| | PEAVGQNFSVVQFSGGLNDQNTTQDSGEANLDLQYIVGVSAPLPVTEFS | ia nodorum |
| | TGGRGPWVADLDQPDEADSANEPYLEFLQGVLKLPQSELPQVISTSYGE | SN15 |
| | NEQSVPKSYALSVCNLFAQLGSRGVSVIFSSGDSGPGSACQSNDGKNT | |
| | TKFQPQYPAACPFVTSVGSTRYLNETATGFSSGGFSDYWKRPSYQDDA | |
| | VKAYFHHLGEKFKPYFNRHGRGFPDVATQGYGFRVYDQGKLKGLQGTS | |
| | ASAPAFAGVIGLLNDARLKAKKPTLGFLNPLLYSNSDALNDIVLGGSKGC | |
| | DGHARFNGPPNGSPVIPYAGWNATAGWDPVTGLGTPNFPKLLKAA | |
| 32 | VFQPDCLRTEYSVNGYKPSAKSGSRIGFGSFLNQSASSSDLALFEKHFG | Trichoderma |
| | FASQGFSVELINGGSNPQPPTDANDGEANLDAQNIVSFVQPLPITEFIAG | atroviride IMI |
| | GTAPYFPDPVEPAGTPDENEPYLEYYEYLLSKSNKELPQVITNSYGDEE | 206040 |
| | QTVPQAYAVRVCNLIGLMGLRGISILESSGDEGVGASCLATNSTTTPQFN | |
| | PIFPATCPYVTSVGGTVSFNPEVAWDGSSGGFSYYFSRPVVYQEAAVGT | |
| | YLNKYVSEETKEYYKSYVDFSGRGFPDVAAHSVSPDYPVFQGGELTPS | |
| | GGTSAASPIVASVIALLNDARLRAGKPALGFLNPLIYGYAYKGFTDITSGQ | |
| | AVGCNGNNTQTGGPLPGAGVIPGAFWNATKGWDPTTGFGVPNFKKLLE | |
| | LV | |
| 33 | CRSLVTTACLRELYGLGDRVTQARDDNRIGVSGFLEEYAQYRDLELFLS | Arthroderma |
| | RFEPSAKGFNFSEGLIAGGKNTQGGPGSSTEANLDMQYVVGLSHKAKV | benhamiae |
| | TYYSTAGRGPLIPDLSQPSQASNNNEPYLEQLRYLVKLPKNQLPSVLTTS | CBS 112371 |
| | YGDTEQSLPASYTKATCDLFAQLGTMGVSVIFSSGDTGPGSSCQTNDG | |
| | KNATRFNPIYPASCPFVTSIGGTVGTGPERAVSFSSGGFSDRFPRPQYQ | |
| | DNAVKDYLKILGNQWSGLFDPNGRAFPDIAAQGSNYAVYDKGRMTGVS | |
| | GTSASAPAMAAIIAQLNDFRLAKGSPVLGFLNPWIYSKGFSGFTDIVDGG | |
| | SRGCTGYDIYSGLKAKKVPYASWNATKGWDPVTGFGTPNFQALTKVL | |
| 34 | CQTSITPSCLKQMYNIGDYTPKVESGSTIGFSSFLGESAIYSDVFLFEEKF | Fusarium |
| | GIPTQNFTTVLINNGTDDQNTAHKNFGEADLDAENIVGIAHPLPFTQYITG | graminearum |
| | GSPPFLPNIDQPTAADNQNEPYVPFFRYLLSQKEVPAVVSTSYGDEEDS | PH-1 |
| | VPREYATMTCNLIGLLGLRGISVIFSSGDIGVGAGCLGPDHKTVEFNAIFP | |
| | ATCPYLTSVGGTVDVTPEIAWEGSSGGFSKYFPRPSYQDKAVKTYMKTV | |
| | SKQTKKYYGPYTNWEGRGFPDVAGHSVSPNYEVIYAGKQSASGGTSAA | |
| | APVWAAIVGLLNDARFRAGKPSLGWLNPLVYKYGPKVLTDITGGYAIGCD | |
| | GNNTQSGKPEPAGSGIVPGARWNATAGWDPVTGYGTPDFGKLKDLVLS | |
| 35 | CDLVITPPCLEAAYNYKNYMPDPNSGSRVSFTSFLEQAAQQSDLTKFLSL | Acremonium |
| | TGLDRLRPPSSKPASFDTVLINGGETHQGTPPNKTSEANLDVQWLAAVIK | alcalophilum |
| | ARLPITQWITGGRPPFVPNLRLRHEKDNTNEPYLEFFEYLVRLPARDLPQ | |
| | VISNSYAEDEQTVPEAYARRVCNLIGIMGLRGVTVLTASGDSGVGAPCR | |
| | ANDGSDRLEFSPQFPTSCPYITAVGGTEGWDPEVAWEASSGGFSHYFL | |
| | RPVVYQANAVEKYLDEELDPATRAYYDGNGFVQFAGRAYPDLSAHSSSP | |
| | RYAYIDKLAPGLTGGTSASCPVVAGIVGLLNDARLRRGLPTMGFINPWLY | |
| | TRGFEALQDVTGGRASGCQGIDLQRGTRVPGAGIIPWASWNATPGWDP | |
| | ATGLGLPDFWAMRGL | |
| 36 | CATIITPPCLETAYNYKGYIPDPKSGSRVSFTSFLEQAAQQADLTKFLSLT | Sodiomyces |
| | RLEGFRTPASKKKTFKTVLINGGESHEGVHKKSKTSEANLDVQWLAAVT | alkalinus |
| | QTKLPITQWITGGRPPFVPNLRIPTPEANTNEPYLEFLEYLFRLPDKDLPQ | |
| | VISNSYAEDEQSVPEAYARRVCGLLGIMGLRGVTVLTASGDSGVGAPCR | |
| | ANDGSGREEFSPQFPSSCPYITTVGGTQAWDPEVAWKGSSGGFSNYFP | |
| | RPVVYQVAAVEKYLEEQLDPAAREYYEENGFVRFAGRAFPDLSAHSSSP | |
| | KYAYVDKRVPGLTGGTSASCPVVAGIVGLLNDARLRRGLPTMGFINPWL | |
| | YAKGYQALEDVTGGAAVGCQGIDIQTGKRVPGAGIIPGASWNATPDWDP | |
| | ATGLGLPNFWAMRELA | |
| 37 | CADTITLSCLKEMYNFGNYTPSASSGSKLGFASFLNESASYSDLAKFERL | Aspergillus |
| | FNLPSQNFSVELINGGVNDQNQSTASLTEADLDVELLVGVGHPLPVTEFI | kawachii IFO |
| | TSGEPPFIPDPDEPSAADNENEPYLQYYEYLLSKPNSALPQVISNSYGDD | 4308 |
| | EQTVPEYYAKRVCNLIGLVGLRGISVLESSGDEGIGSGCRTTDGTNSTQF | |
| | NPIFPATCPYVTAVGGTMSYAPEIAWEASSGGFSNYFERAWFQKEAVQ | |
| | NYLANHITNETKQYYSQFANFSGRGFPDVSAHSFEPSYEVIFYGARYGS | |
| | GGTSAACPLFSALVGMLNDARLRAGKSTLGFLNPLLYSKGYKALTDVTA | |
| | GQSIGCNGIDPQSDEAVAGAGIIPWAHWNATVGWDPVTGLGLPDFEKLR | |
| | QLVLS | |
| 38 | CQTSITPACLKQMYNVGNYTPSVAHGSRVGFGSFLNQSAIFDDLFTYEK | Talaromyces |
| | VNDIPSQNFTKVIIANASNSQDASDGNYGEANLDVQNIVGISHPLPVTEFL | stipitatus |
| | TGGSPPFVASLDTPTNQNEPYIPYYEYLLSQKNEDLPQVISNSYGDDEQS | ATCC 10500 |
| | VPYKYAIRACNLIGLTGLRGISVLESSGDLGVGAGCRSNDGKNKTQFDPI | |
| | FPATCPYVTSVGGTQSVTPEIAVVVASSGGFSNYFPRTVVYQEPAIQTYLG | |
| | LLDDETKTYYSQYTNFEGRGFPDVSAHSLTPDYQVVGGGYLQPSGGTS | |
| | AASPVFAGIIALLNDARLAAGKPTLGFLNPFFYLYGYKGLNDITGGQSVGC | |
| | NGINGQTGAPVPGGGIVPGAAWNSTTGWDPATGLGTPDFQKLKELVLS | |
| 39 | CQTSITPSCLKQMYNIGDYTPDAKSGSEIGFSSFLGQAAIYSDVFKFEELF | Fusarium |
| | GIPKQNYTTILINNGTDDQNTAHGNFGEANLDAENIVGIAHPLPFKQYITG | oxysporum f. |
| | GSPPFVPNIDQPTEKDNQNEPYVPFFRYLLGQKDLPAVISTSYGDEEDSV | sp. cubense |
| | PREYATLTCNMIGLLGLRGISVIFSSGDIGVGSGCLAPDYKTVEFNAIFPA | race 4 |
| | TCPYLTSVGGTVDVTPEIAWEGSSGGFSKYFPRPSYQDKAIKKYMKTVS | |
| | KETKKYYGPYTNWEGRGFPDVAGHSVAPDYEVIYNGKQARSGGTSAAA | |
| | PVWAAIVGLLNDARFKAGKKSLGWLNPLIYKHGPKVLTDITGGYAIGCDG | |
| | NNTQSGKPEPAGSGLVPGARWNATAGWDPTTGYGTPNFQKLKDLVLS | |
| 40 | VFQPDCLRTEYNVNGYTPSAKSGSRIGFGSFLNQSASFSDLALFEKHFG | Trichoderma |
| | FSSQNFSVVLINGGTDLPQPPSDDNDGEANLDVQNILTIAHPLPITEFITA | virens Gv29- |
| | GSPPYFPDPVEPAGTPDENEPYLQYFEYLLSKPNRDLPQVITNSYGDEE | 8 |
| | QTVPQAYAVRVCNLIGLMGLRGISILESSGDEGVGASCVATNSTTPQFNP | |
| | IFPATCPYVTSVGGTVNFNPEVAWDGSSGGFSYYFSRPVVYQEEAVGNY | |
| | LEKHVSAETKKYYGPYVDFSGRGFPDVAAHSVSPDYPVFQGGQLTPSG | |
| | GTSAASPVVASIIALLNDARLREGKPTLGFLNPLIYQYAYKGFTDITSGQS | |
| | DGCNGNNTQTDAPLPGAGVVLGAHWNATKGWDPTTGFGVPNFKKLLE | |
| | LI | |
| 41 | QIFHPDCLKTKYGVDGYAPSPRCGSRIGFGSFLNETASYSDLAQFEKYF | Trichoderma |
| | DLPNQNLSTLLINGAIDVQPPSNKNDSEANMDVQTILTFVQPLPITEFVVA | atroviride IMI |
| | GIPPYIPDAALPIGDPVQNEPWLEYFEFLMSRTNAELPQVIANSYGDEEQ | 206040 |
| | TVPQAYAVRVCNQIGLLGLRGISVIASSGDTGVGMSCMASNSTTPQFNP | |
| | MFPASCPYITTVGGTQHLDNEIAWELSSGGFSNYFTRPVVYQEDAAKTYL | |
| | ERHVSTETKAYYERYANFLGRGFPDVAALSLNPDYPVIIGGELGPNGGTS | |
| | AAAPVVASIIALLNDARLCLGKPALGFLNPLIYQYADKGGFTDITSGQSWG | |
| | CAGNTTQTGPPPPGAGVIPGAHWNATKGWDPVTGFGTPNFKKLLSLAL | |
| | S | |
| 42 | TVITPDCLRDLYNTADYVPSATSRNAIGIAGYLDRSNRADLQTFFRRFRP | Agaricus |
| | DAVGFNYTTVQLNGGGDDQNDPGVEANLDIQYAAGIAFPTPATYWSTG | bisporus var. |
| | GSPPFIPDTQTPTNTNEPYLDWINFVLGQDEIPQVISTSYGDDEQTVPED | burnettii |
| | YATSVCNLFAQLGSRGVTVFFSSGDFGVGGGDCLTNDGSNQVLFQPAF | JB137-S8 |
| | PASCPFVTAVGGTVRLDPEIAVSFSGGGFSRYFSRPSYQNQTVAQFVSN | |
| | LGNTFNGLYNKNGRAYPDLAAQGNGFQVVIDGIVRSVGGTSASSPTVAG | |
| | IFALLNDFKLSRGQSTLGFINPLIYSSATSGFNDIRAGTNPGCGTRGFTAG | |
| | TGWDPVTGLGTPDFLRLQ | |
| 43 | GVTPLCLRTLYRVNYKPATTGNLVAFASFLEQYARYSDQQAFTQRVLGP | Magnaporthe |
| | GVPLQNFSVETVNGGANDQQSKLDSGEANLDLQYVMAMSHPIPILEYST | oryzae 70-15 |
| | GGRGPLVPTLDQPNANNSSNEPYLEFLTYLLAQPDSAIPQTLSVSYGEEE | |
| | QSVPRDYAIKVCNMFMQLGARGVSVMFSSGDSGPGNDCVRASDNATF | |
| | FGSTFPAGCPYVTSVGSTVGFEPERAVSFSSGGFSIYHARPDYQNEVVP | |
| | KYIESIKASGYEKFFDGNGRGIPDVAAQGARFVVIDKGRVSLISGTSASSP | |
| | AFAGMVALVNAARKSKDMPALGFLNPMLYQNAAAMTDIVNGAGIGCRK | |
| | QRTEFPNGARFNATAGWDPVTGLGTPLFDKLLA | |
| 44 | CNASITPECLRALYNVGDYEADPSKKSLFGVCGYLEQYAKHDOLAKFEQ | Togninia |
| | TYAPYAIGADFSVVTINGGGDNQTSTIDDGEANLDMQYAVSMAYKTPITY | minima |
| | YSTGGRGPLVPDLDQPDPNDVSNEPYLDFVSYLLKLPDSKLPOTITTSYG | UCRPA7 |
| | EDEQSVPRSYVEKVCTMFGALGARGVSVIFSSGDTGVGSACQTNDGKN | |
| | TTRFLPIFPAACPYVTSVGGTRYVDPEVAVSFSSGGFSDIFPTPLYQKGA | |
| | VSGYLKILGDRWKGLYNPHGRGFPDVSGQSVRYHVFDYGKDVMYSGTS | |
| | ASAPMFAALVSLLNNARLAKKLPPMGFLNPWLYTVGFNGLTDIVHGGST | |
| | GCTGTDVYSGLPTPFVPYASWNATVGWDPVTGLGTPLFDKLLNL | |
| 45 | CNKKITPDCLANLYNFKDYDASDANVTIGVSGFLEQYARFDDLKQFISTF | Bipolaris |
| | QPKAAGSTFQVTSVNAGPFDQNSTASSVEANLDIQYTTGLVAPDIETRYF | maydis C5 |
| | TVPGRGILIPDLDQPTESDNANEPYLDYFTYLNNLEDEELPDVLTTSYGE | |
| | SEQSVPAEYAKKVCNLIGOLGARGVSVIFSSGDTGPGSACQTNDGKNTT | |
| | RFLPIFPASCPYVTSVGGTVGVEPEKAVSFSSGGFSDLWPRPAYQEKAV | |
| | SEYLEKLGDRWNGLYNPOGRGFPDVAAQGQGFQVFDKGRLISVGGTSA | |
| | SAPVFASVVALLNNARKAAGMSSLGFLNPWIYEQGYKGLTDIVAGGSTG | |
| | CTGRSIYSGLPAPLVPYASWNATEGWDPVTGYGTPDFKOLLTLAT | |
| 46 | CDSIITPHCLKQLYNIGDYQADPKSGSKVGFASYLEEYARYADLERFEQH | Aspergillus |
| | LAPNAIGQNFSVVQFNGGLNDQLSLSDSGEANLDLQYILGVSAPVPVTEY | kawachii IFO |
| | STGGRGELVPDLSSPDPNDNSNEPYLDFLQGILKLDNSDLPQVISTSYGE | 4308 |
| | DEQTIPVPYARTVCNLYAQLGSRGVSVIFSSGDSGVGAACLTNDGTNRT | |
| | HFPPQFPASCPVVVTSVGATSKTSPEQAVSFSSGGFSDLWPRPSYQQAA | |
| | VQTYLTQHLGNKFSGLFNASGRAFPDVAAQGVNYAVYDKGMLGQFDGT | |
| | SCSAPTFSGVIALLNDARLRAGLPVMGFLNPFLYGVGSESGALNDIVNGG | |
| | SLGCDGRNRFGGTPNGSPVVPFASWNATTGWDPVSGLGTPDFAKLRG | |
| | V | |
| 47 | CEKAITPSCLADLYNTEGYKASNRSGSKVAFASFLEEYARYDDLAEFEET | Aspergillus |
| | YAPYAIGQNFSVISINGGLNDQDSTADSGEANLDLQYIIGVSSPLPVTEFT | nidulans |
| | TGGRGKLIPDLSSPDPNDNTNEPFLDFLEAVLKLDQKDLPQVISTSYGED | FGSC A4 |
| | EQTIPEPYARSVCNLYAQLGSRGVSVLFSSGDSGVGAACQTNDGKNTT | |
| | HFPPQFPASCPVVVTAVGGTNGTAPESGVYFSSGGFSDYWARPAYQNA | |
| | AVESYLRKLGSTQAQYFNRSGRAFPDVAAQAQNFAVVDKGRVGLFDGT | |
| | SCSSPVFAGIVALLNDVRLKAGLPVLGFLNPWLYQDGLNGLNDIVDGGST | |
| | GCDGNNRFNGSPNGSPVIPYAGWNATEGWDPVTGLGTPDFAKLKALVL | |
| 48 | CDQITTPHCLRKLYNVNGYKADPASGSKIGFASFLEEYARYSDLVLFEEN | Aspergillus |
| | LAPFAEGENFTVVMYNGGKNDQNSKSDSGEANLDLQYIVGMSAGAPVT | ruber CBS |
| | EFSTAGRAPVIPDLDQPDPSAGTNEPYLEFLONVLHMDOEHLPQVISTSY | 135680 |
| | GENEQTIPEKYARTVCNMYAOLGSRGVSVIFSSGDSGVGSACMTNDGT | |
| | NRTHFPPQFPASCPVVVTSVGATEKMAPEQATYFSSGGFSDLFPRPKYQ | |
| | DAAVSSYLQTLGSRYQGLYNGSNRAFPDVSAQGTNFAVYDKGRLGQFD | |
| | GTSCSAPAFSGIIALLNDVRLQNNKPVLGFLNPWLYGAGSKGLNDVVHG | |
| | GSTGCDGQERFAGKANGSPVVPYASWNATQGWDPVTGLGTPDFGKLK | |
| | DLAL | |
| 49 | CDSVITPKCLKDLYKVGDYEADPDSGSQVAFASYLEEYARYADMVKFQN | Aspergillus |
| | SLAPYAKGQNFSVVLYNGGVNDQSSSADSGEANLDLOTIMGLSAPLPIT | terreus |
| | EYITGGRGKLIPDLSQPNPNDNSNEPYLEFLONILKLDQDELPQVISTSYG | NIH2624 |
| | EDEQTIPRGYAESVCNMLAQLGSRGVSVVFSSGDSGVGAACQTNDGRN | |
| | QTHFNPQFPASCPVVVTSVGATTKTNPEQAVYFSSGGFSDFWKRPKYQ | |
| | DEAVAAYLDTLGDKFAGLFNKGGRAFPDVAAQGMNYAIYDKGTLGRLD | |
| | GTSCSAPAFSAIISLLNDARLREGKPTMGFLNPWLYGEGREALNDVVVG | |
| | GSKGCDGRDRFGGKPNGSPVVPFASWNATQGWDPVTGLGTPNFAKML | |
| | ELA | |
| 50 | CNSIITPQCLKDLYSIGDYEADPTNGNKVAFASYLEEYARYSDLALFEKNI | Penicillium |
| | APFAKGQNFSVVQYNGGGNDQQSSSGSSEANLDLQYIVGVSSPVPVTE | digitatum |
| | FSTGGRGELVPDLDQPNPNDNNNEPYLEFLQNVLKLHKKDLPQVISTSY | Pd1 |
| | GEDEQSVPEKYARAVCNLYSQLGSRGVSVIFSSGDSGVGAACQTNDGR | |
| | NATHFPPQFPAACPWVTSVGATTHTAPERAVYFSSGGFSDLWDRPTW | |
| | QEDAVSEYLENLGDRWSGLFNPKGRAFPDVAAQGENYAIYDKGSLISVD | |
| | GTSCSAPAFAGVIALLNDARIKANRPPMGFLNPWLYSEGRSGLNDIVNG | |
| | GSTGCDGHGRFSGPTNGGTSIPGASWNATKGWDPVSGLGSPNFAAMR | |
| | KLA | |
| 51 | CNSAITPQCLKDLYKVGDYKASASNGNKVAFTSYLEQYARYSDLALFEQ | Penicillium |
| | NIAPYAQGQNFTVIQYNGGLNDQSSPADSSEANLDLQYIIGTSSPVPVTE | oxalicum |
| | FSTGGRGPLVPDLDQPDINDNNNEPYLDFLQNVIKMSDKDLPQVISTSYG | 114-2 |
| | EDEQSVPASYARSVCNLIAQLGGRGVSVIFSSGDSGVGSACQTNDGKN | |
| | TTRFPAQFPAACPVVVTSVGATTGISPERGVFFSSGGFSDLWSRPSWQS | |
| | HAVKAYLHKLGKRQDGLFNREGRAFPDVSAQGENYAIYAKGRLGKVDG | |
| | TSCSAPAFAGLVSLLNDARIKAGKSSLGFLNPWLYSHPDALNDITVGGST | |
| | GCDGNARFGGRPNGSPVVPYASWNATEGWDPVTGLGTPNFQKLLKSA | |
| | V | |
| 52 | CNSIITPQCLKDIYNIGDYQANDTNGNKVGFASYLEEYARYSDLALFEKNI | Penicillium |
| | APSAKGQNFSVTRYNGGLNDQSSSGSSSEANLDLQYIVGVSSPVPVTEF | roqueforti |
| | SVGGRGELVPDLDQPDPNDNNNEPYLEFLQNVLKLDKKDLPQVISTSYG | FM164 |
| | EDEQSIPEKYARSVCNLYSQLGSRGVSVIFSSGDSGVGSACLTNDGRNA | |
| | TRFPPQFPAACPVVVTSVGATTHTAPEQAVYFSSGGFSDLWARPKWQE | |
| | EAVSEYLEILGNRWSGLFNPKGRAFPDVTAQGRNYAIYDKGSLTSVDGT | |
| | SCSAPAFAGVVALLNDARLKVNKPPMGFLNPWLYSTGRAGLKDIVDGGS | |
| | TGCDGKSRFGGANNGGPSIPGASWNATKGWDPVSGLGSPNFATMRKL | |
| | A | |
| 53 | CNSIITPQCLKNMYNVGDYQADDDNGNKVGFASYLEEYARYSDLELFEK | Penicillium |
| | NVAPFAKGQNFSVIQYNGGLNDQHSSASSSEANLDLQYIVGVSSPVPVT | rubens |
| | EFSVGGRGELVPDLDQPDPNDNNNEPYLEFLQNVLKMEQQDLPQVISTS | Wisconsin |
| | YGENEQSVPEKYARTVCNLFSQLGSRGVSVIFASGDSGVGAACQTNDG | 54-1255 |
| | RNATRFPAQFPAACPVVVTSVGATTHTAPEKAVYFSSGGFSDLWDRPKW | |
| | QEDAVSDYLDTLGDRWSGLFNPKGRAFPDVSAQGQNYAIYDKGSLTSV | |
| | DGTSCSAPAFAGVIALLNDARLKANKPPMGFLNPWLYSTGRDGLNDIVH | |
| | GGSTGCDGNARFGGPGNGSPRVPGASWNATKGWDPVSGLGSPNFAT | |
| | MRKLA | |
| 54 | CANLITPDCLVEMYNLGDYKPDASSGSRVGFGSFLNQSANYADLAAYEQ | Neosartorya |
| | LFNIPPQNFSVELINGGANDQNWATASLGEANLDVELIVAVSHALPVVEFI | fischeri |
| | TGGSPPFVPNVDEPTAADNQNEPYLQYYEYLLSKPNSHLPQVISNSYGD | NRRL 181 |
| | DEQTVPEYYARRVCNLIGLMGLRGITVLESSGDTGIGSACMSNDGTNTP | |
| | QFTPTFPGTCPFITAVGGTQSYAPEVAWDASSGGFSNYFSRPWYQYFA | |
| | VENYLNNHITKDTKKYYSQYTNFKGRGFPDVSAHSLTPDYEVVLTGKHY | |
| | KSGGTSAACPVFAGIVGLLNDARLRAGKSTLGFLNPLLYSILAEGFTDITA | |
| | GSSIGCNGINPQTGKPVPGGGIIPYAHWNATAGWDPVTGLGVPDFMKLK | |
| | ELVLS | |
| 55 | CANLITPDCLVEMYNLGDYKPDASSGSRVGFGSFLNESANYADLAAYEQ | Aspergillus |
| | LFNIPPQNFSVELINRGVNDQNWATASLGEANLDVELIVAVSHPLPVVEFI | fumigatus |
| | TGALPPVLRVLALQTQLPSSSGDFQLTVPEYYARRVCNLIGLMGLRGITV | CAE17675 |
| | LESSGDTGIGSACMSNDGTNKPQFTPTFPGTCPFITAVGGTQSYAPEVA | |
| | WDGSSGGFSNYFSRPVVYQSFAVDNYLNNHITKDTKKYYSQYTNFKGRG | |
| | FPDVSAHSLTPYYEVVLTGKHYKSGGTSAASPVFAGIVGLLNDARLRAG | |
| | KSTLGFLNPLLYSILAEGFTDITAGSSIGCNGINPQTGKPVPGGGIIPYAH | |
| | WNATAGWDPVTGLGVPDFMKLKELVLS | |
| 56 | ATGGCAAAGTTGAGCACTCTCCGGCTTGCGAGCCTTCTTTCCCTTGT | Trichoderma |
| | CAGTGTGCAGGTATCTGCCTCTGTCCATCTATTGGAGAGTCTGGAGA | reesei QM6a |
| | AGCTGCCTCATGGATGGAAAGCAGCTGAAACCCCGAGCCCTTCGTCT | |
| | CAAATCGTCTTGCAGGTTGCTCTGACGCAGCAGAACATTGACCAGCT | |
| | TGAATCGAGGCTCGCAGCTGTATCCACACCCACTTCTAGCACCTACG | |
| | GCAAATACTTGGATGTAGACGAGATCAACAGCATCTTCGCTCCAAGT | |
| | GATGCTAGCAGTTCTGCCGTCGAGTCTTGGCTTCAGTCCCACGGAGT | |
| | GACGAGTTACACCAAGCAAGGCAGCAGCATTTGGTTTCAAACAAACA | |
| | TCTCCACTGCAAATGCGATGCTCAGCACCAATTTCCACACGTACAGC | |
| | GATCTCACCGGCGCGAAGAAGGTGCGCACTCTCAAGTACTCGATCCC | |
| | GGAGAGCCTCATCGGCCATGTCGATCTCATCTCTCCCACGACCTATT | |
| | TTGGCACGACAAAGGCCATGAGGAAGTTGAAATCCAGTGGCGTGAG | |
| | CCCAGCCGCTGATGCTCTAGCCGCTCGCCAAGAACCTTCCAGCTGCA | |
| | AAGGAACTCTAGTCTTTGAGGGAGAAACGTTCAATGTCTTTCAGCCAG | |
| | ACTGTCTCAGGACCGAGTATAGTGTTGATGGATACACCCCGTCTGTC | |
| | AAGTCTGGCAGCAGAATTGGGTTTGGTTCCTTTCTCAATGAGAGCGC | |
| | AAGCTTCGCAGATCAAGCACTCTTTGAGAAGCACTTCAACATCCCCA | |
| | GTCAAAACTTCTCCGTTGTCCTGATCAACGGTGGAACGGATCTCCCT | |
| | CAGCCGCCTTCTGACGCCAACGATGGCGAAGCCAACCTGGACGCTC | |
| | AAACCATTTTGACCATCGCACATCCTCTCCCCATCACCGAATTCATCA | |
| | CCGCCGGCAGTCCGCCATACTTCCCCGATCCAGTTGAACCTGCGGG | |
| | AACACCCAACGAGAACGAGCCTTATTTACAGTATTACGAATTTCTGTT | |
| | GTCCAAGTCCAACGCTGAAATTCCGCAAGTCATTACCAACTCCTACG | |
| | GCGACGAGGAGCAAACTGTGCCGCGGTCATATGCCGTTCGAGTTTG | |
| | CAATCTGATTGGTCTGCTAGGACTACGCGGTATCTCTGTCCTTCATTC | |
| | CTCGGGCGACGAGGGTGTGGGCGCCTCTTGCGTTGCTACCAACAGC | |
| | ACCACGCCTCAGTTTAACCCCATCTTTCCTGTAGGTCTTCTACGTCAA | |
| | CACTTCCAGACAACCATTTTCTCCTACTAACCACTCTACCCTACTCTCT | |
| | GTTCACATAGGCTACATGTCCTTATGTTACAAGTGTTGGCGGAACCGT | |
| | GAGCTTCAATCCCGAGGTTGCCTGGGCTGGTTCATCTGGAGGTTTCA | |
| | GCTACTACTTCTCTAGACCCTGGTACCAGCAGGAAGCTGTGGGTACT | |
| | TACCTTGAGAAATATGTCAGTGCTGAGACAAAGAAATACTATGGACCT | |
| | TATGTCGATTTCTCCGGACGAGGTTTCCCCGATGTTGCAGCCCACAG | |
| | CGTCAGCCCCGAGTGAGTTCTATTCCTACCTATGCAAATCATAGAATG | |
| | TATGCTAACTCGCCATGAAGCTATCCTGTGTTTCAGGGCGGTGAACT | |
| | CACCCCAAGCGGAGGCACTTCAGCAGCCTCTCCTGTCGTAGCAGCC | |
| | ATCGTGGCGCTGTTGAACGATGCCCGTCTCCGCGAAGGAAAACCCA | |
| | CGCTTGGATTTCTCAATCCGCTGATTTACCTACACGCCTCCAAAGGGT | |
| | TCACCGACATCACCTCGGGCCAATCTGAAGGGTGCAACGGCAATAAC | |
| | ACCCAGACGGGCAGTCCTCTCCCAGGAGTATGCAGAACATCAAGAAG | |
| | CCTTCTATCAGACGCCAATGCTAACTTGTGGATAGGCCGGCTTCATT | |
| | GCAGGCGCACACTGGAACGCGACCAAGGGATGGGACCCGACGACT | |
| | GGATTTGGTGTTCCAAACCTCAAAAAGCTCCTCGCACTTGTCCGGTTC | |
| | TAA | |
| 57 | ATGTTCTTCAGTCGTGGAGCGCTTTCGCTCGCAGTGCTTTCACTGCT | Aspergillus |
| | CAGCTCCTCCGCCGCAGGGGAGGCTTTTGAGAAGCTGTCTGCCGTT | oryzae RIB40 |
| | CCAAAGGGATGGCACTATTCTAGTACCCCTAAAGGCAACACTGAGGT | |
| | TTGTCTGAAGATCGCCCTCGCGCAGAAGGATGCTGCTGGGTTCGAAA | |
| | AGACCGTCTTGGAGATGTCGGATCCCGACCACCCCAGCTACGGCCA | |
| | GCACTTCACCACCCACGACGAGATGAAGCGCATGCTTCTTCCCAGAG | |
| | ATGACACCGTTGATGCCGTTCGACAATGGCTCGAAAACGGCGGCGT | |
| | GACCGACTTTACCCAGGATGCCGACTGGATCAACTTCTGTACTACCG | |
| | TCGATACCGCGAACAAACTCTTGAATGCCCAGTTCAAATGGTACGTCA | |
| | GCGATGTGAAGCACATCCGCCGTCTCAGAACACTGCAGTACGACGTC | |
| | CCCGAGTCGGTCACCCCTCACATCAACACCATCCAACCGACCACCCG | |
| | TTTTGGCAAGATTAGCCCCAAGAAGGCCGTTACCCACAGCAAGCCCT | |
| | CCCAGTTGGACGTGACCGCCCTTGCTGCCGCTGTCGTTGCAAAGAAC | |
| | ATCTCGCACTGTGATTCTATCATTACCCCCACCTGTCTGAAGGAGCTT | |
| | TACAACATTGGTGATTACCAGGCCGATGCAAACTCGGGCAGCAAGAT | |
| | CGCCTTCGCCAGCTATCTGGAGGAGTACGCGCGCTACGCTGACCTG | |
| | GAGAACTTTGAGAACTACCTTGCTCCCTGGGCTAAGGGCCAGAACTT | |
| | CTCCGTTACCACCTTCAACGGCGGTCTCAATGATCAGAACTCCTCGT | |
| | CCGATAGCGGTGAGGCCAACCTGGACCTGCAGTACATTCTTGGTGTC | |
| | AGCGCTCCACTGCCCGTTACTGAATTCAGCACCGGAGGCCGTGGTC | |
| | CCCTCGTTCCTGATCTGACCCAGCCGGATCCCAACTCTAACAGCAAT | |
| | GAGCCGTACCTTGAGTTCTTCCAGAATGTGTTGAAGCTCGACCAGAA | |
| | GGACCTCCCCCAGGTCATCTCGACCTCCTATGGAGAGAACGAACAG | |
| | GAAATCCCCGAAAAGTACGCTCGCACCGTCTGCAACCTGATCGCTCA | |
| | GCTTGGCAGCCGCGGTGTCTCCGTTCTCTTCTCCTCCGGTGACTCTG | |
| | GTGTTGGCGAGGGCTGCATGACCAACGACGGCACCAACCGGACTCA | |
| | CTTCCCACCCCAGTTCCCCGCCGCTTGCCCGTGGGTCACCTCCGTC | |
| | GGCGCCACCTTCAAGACCACTCCCGAGCGCGGCACCTACTTCTCCTC | |
| | GGGCGGTTTCTCCGACTACTGGCCCCGTCCCGAATGGCAGGATGAG | |
| | GCCGTGAGCAGCTACCTCGAGACGATCGGCGACACTTTCAAGGGCC | |
| | TCTACAACTCCTCCGGCCGTGCTTTCCCCGACGTCGCAGCCCAGGG | |
| | CATGAACTTCGCCGTCTACGACAAGGGCACCTTGGGCGAGTTCGAC | |
| | GGCACCTCCGCCTCCGCCCCGGCCTTCAGCGCCGTCATCGCTCTCC | |
| | TGAACGATGCCCGTCTCCGCGCCGGCAAGCCCACTCTCGGCTTCCT | |
| | GAACCCCTGGTTGTACAAGACCGGCCGCCAGGGTCTGCAAGATATCA | |
| | CCCTCGGTGCTAGCATTGGCTGCACCGGTCGCGCTCGCTTCGGCGG | |
| | CGCCCCTGACGGTGGTCCCGTCGTGCCTTACGCTAGCTGGAACGCT | |
| | ACCCAGGGCTGGGATCCCGTCACTGGTCTCGGAACTCCCGATTTCG | |
| | CCGAGCTCAAGAAGCTTGCCCTTGGCAACTAA | |
| 58 | ATGGCGCCCATCCTCTCGTTCCTTGTTGGCTCTCTCCTGGCGGTTCG | Phaeosphaer |
| | CGCTCTTGCTGAGCCATTTGAGAAGCTGTTCAGCACCCCGGAAGGAT | ia nodorum |
| | GGAAGATGCAAGGTCTTGCTACCAATGAGCAGATCGTCAAGCTCCAG | SN15 |
| | ATTGCTCTTCAGCAAGGCGATGTTGCAGGTTTCGAGCAACATGTGATT | |
| | GACATCTCAACGCCTAGCCACCCGAGCTATGGTGCTCACTATGGCTC | |
| | GCATGAGGAGATGAAGAGGATGATCCAGCCAAGCAGCGAGACAGTC | |
| | GCTTCTGTGTCTGCATGGCTGAAGGCCGCCGGTATCAACGACGCTGA | |
| | GATTGACAGCGACTGGGTCACCTTCAAGACGACCGTTGGCGTTGCCA | |
| | ACAAGATGCTCGACACCAAGTTCGCTTGGTACGTGAGCGAGGAGGC | |
| | CAAGCCCCGCAAGGTCCTTCGCACACTCGAGTACTCTGTACCAGATG | |
| | ATGTTGCAGAACACATCAACTTGATCCAGCCCACTACTCGGTTTGCTG | |
| | CGATCCGCCAAAACCACGAGGTTGCGCACGAGATTGTTGGTCTTCAG | |
| | TTCGCTGCTCTTGCCAACAACACCGTTAACTGCGATGCCACCATCACT | |
| | CCCCAGTGCTTGAAGACTCTTTACAAGATTGACTACAAGGCCGATCC | |
| | CAAGAGTGGTTCCAAGGTCGCTTTTGCTTCGTATTTGGAGCAGTACG | |
| | CGCGTTACAATGACCTCGCCCTCTTCGAGAAGGCCTTCCTCCCCGAA | |
| | GCAGTTGGCCAGAACTTCTCTGTCGTCCAGTTCAGCGGCGGTCTCAA | |
| | CGACCAGAACACCACGCAAGACAGTGGCGAGGCCAACTTGGACTTG | |
| | CAGTACATTGTCGGTGTCAGCGCTCCTCTTCCCGTCACCGAGTTCAG | |
| | CACCGGTGGTCGCGGCCCATGGGTCGCTGACCTAGACCAACCTGAC | |
| | GAGGCGGACAGCGCCAACGAGCCCTACCTTGAATTCCTTCAGGGTG | |
| | TGCTCAAACTTCCCCAGTCTGAGCTACCTCAGGTCATCTCCACATCCT | |
| | ATGGCGAGAATGAGCAGAGTGTACCTAAGTCATACGCTCTCTCCGTC | |
| | TGCAACTTGTTCGCCCAACTCGGTTCCCGTGGCGTCTCCGTCATCTT | |
| | CTCTTCTGGTGACAGCGGCCCTGGATCCGCATGCCAGAGCAACGAC | |
| | GGCAAGAACACGACCAAGTTCCAGCCTCAGTACCCCGCTGCCTGCC | |
| | CCTTTGTCACCTCGGTTGGATCGACTCGCTACCTCAACGAGACCGCA | |
| | ACCGGCTTCTCATCTGGTGGTTTCTCCGACTACTGGAAGCGCCCATC | |
| | GTACCAGGACGATGCTGTTAAGGCGTATTTCCACCACCTCGGTGAGA | |
| | AATTCAAGCCATACTTCAACCGCCACGGCCGTGGATTCCCCGACGTT | |
| | GCAACCCAGGGATATGGCTTCCGCGTCTACGACCAGGGCAAGCTCA | |
| | AGGGTCTCCAAGGTACTTCTGCCTCCGCGCCTGCATTCGCCGGTGTG | |
| | ATTGGTCTCCTCAACGACGCGCGATTGAAGGCGAAGAAGCCTACCTT | |
| | GGGATTCCTAAACCCACTGCTTTACTCTAACTCAGACGCGCTAAATGA | |
| | CATTGTTCTCGGTGGAAGCAAGGGATGCGATGGTCATGCTCGCTTTA | |
| | ACGGGCCGCCAAATGGCAGCCCAGTAATCCCATATGCGGGATGGAA | |
| | CGCGACTGCTGGGTGGGATCCAGTGACTGGTCTTGGAACGCCGAAC | |
| | TTCCCCAAGCTTCTTAAGGCTGCGGTGCCTAGCCGGTACAGGGCGT | |
| | GA | |
| 59 | ATGGCGAAACTGACAGCTCTTGCCGGTCTCCTGACCCTTGCCAGCGT | Trichoderma |
| | GCAGGCAAATGCCGCCGTGCTCTTGGACAGCCTCGACAAGGTGCCT | atroviride IMI |
| | GTTGGATGGCAGGCTGCTTCGGCCCCGGCCCCGTCATCCAAGATCA | 206040 |
| | CCCTCCAAGTTGCCCTCACGCAGCAGAACATTGATCAGTTGGAATCA | |
| | AAGCTCGCTGCCGTCTCCACGCCCAACTCCAGCAACTATGGAAAGTA | |
| | CCTGGATGTCGATGAGATTAACCAAATCTTCGCTCCCAGCAGCGCCA | |
| | GCACCGCTGCTGTTGAGTCCTGGCTCAAGTCGTACGGCGTGGACTA | |
| | CAAGGTGCAGGGCAGCAGCATCTGGTTCCAGACGGATGTCTCCACG | |
| | GCCAACAAGATGCTCAGCACAAACTTCCACACTTACACCGACTCGGT | |
| | TGGTGCCAAGAAAGTGCGAACTCTCCAGTACTCGGTCCCCGAGACCC | |
| | TGGCCGACCACATCGATCTGATTTCGCCCACAACCTACTTTGGCACG | |
| | TCCAAGGCCATGCGGGCGTTGAAGATCCAGAACGCGGCCTCTGCCG | |
| | TCTCGCCCCTGGCTGCTCGTCAGGAGCCCTCCAGCTGCAAGGGCAC | |
| | AATTGAGTTTGAGAACCGCACATTCAACGTCTTCCAGCCCGACTGTCT | |
| | CAGGACCGAGTACAGCGTCAACGGATACAAGCCCTCAGCCAAGTCC | |
| | GGTAGCAGGATTGGCTTCGGCTCTTTCCTGAACCAGAGCGCCAGCTC | |
| | CTCAGATCTCGCTCTGTTCGAGAAGCACTTTGGCTTTGCCAGCCAGG | |
| | GCTTCTCCGTCGAGCTCATCAATGGCGGATCAAACCCCCAGCCGCCC | |
| | ACAGACGCCAATGACGGCGAGGCCAACCTGGACGCCCAGAACATTG | |
| | TGTCGTTTGTGCAGCCTCTGCCCATCACCGAGTTTATTGCTGGAGGA | |
| | ACTGCGCCGTACTTCCCAGACCCCGTTGAGCCGGCTGGAACTCCCG | |
| | ATGAGAACGAGCCTTACCTCGAGTACTACGAGTACCTGCTCTCCAAG | |
| | TCAAACAAGGAGCTTCCCCAAGTCATCACCAACTCCTACGGTGATGA | |
| | GGAGCAGACTGTTCCCCAGGCATATGCCGTCCGCGTGTGCAACCTC | |
| | ATTGGATTGATGGGCCTTCGTGGTATCTCTATCCTCGAGTCATCCGGT | |
| | GATGAGGGTGTTGGTGCCTCTTGTCTCGCTACCAACAGCACCACCAC | |
| | TCCCCAGTTCAACCCCATCTTCCCGGCTACATGCCCCTATGTCACCA | |
| | GTGTTGGTGGAACCGTCAGCTTCAACCCCGAGGTTGCCTGGGACGG | |
| | CTCATCCGGAGGCTTCAGCTACTACTTCTCAAGACCTTGGTACCAGG | |
| | AGGCCGCAGTCGGCACATACCTTAACAAGTATGTCAGCGAGGAGACC | |
| | AAGGAATACTACAAGTCGTATGTCGACTTTTCCGGACGTGGCTTCCC | |
| | CGATGTTGCAGCTCACAGCGTGAGCCCCGATTACCCCGTGTTCCAAG | |
| | GCGGCGAGCTTACCCCCAGCGGCGGTACTTCTGCGGCCTCTCCCAT | |
| | CGTGGCCAGTGTTATTGCCCTCCTGAACGATGCTCGTCTCCGTGCAG | |
| | GCAAGCCTGCTCTCGGATTCTTGAACCCTCTGATCTACGGATATGCC | |
| | TACAAGGGCTTTACCGATATCACGAGTGGCCAAGCTGTCGGCTGCAA | |
| | CGGCAACAACACTCAAACTGGAGGCCCTCTTCCTGGTGCGGGTGTTA | |
| | TTCCAGGTGCTTTCTGGAACGCGACCAAGGGCTGGGATCCTACAACT | |
| | GGATTCGGTGTCCCCAACTTCAAGAAGCTGCTTGAGCTTGTCCGATA | |
| | CATTTAG | |
| 60 | ATGCGTCTTCTCAAATTTGTGTGCCTGTTGGCATCAGTTGCCGCCGC | Arthroderma |
| | AAAGCCTACTCCAGGGGCGTCACACAAGGTCATTGAACATCTTGACT | ben hamiae |
| | TTGTTCCAGAAGGATGGCAGATGGTTGGTGCCGCGGACCCTGCTGC | CBS 112371 |
| | TATCATTGATTTCTGGCTTGCCATCGAGCGCGAAAACCCAGAAAAGCT | |
| | CTACGACACCATCTATGACGTCTCCACCCCTGGACGCGCACAATATG | |
| | GCAAACATTTGAAGCGTGAGGAATTGGATGACTTACTACGCCCAAGG | |
| | GCAGAGACGAGTGAGAGCATCATCAACTGGCTCACCAATGGTGGAGT | |
| | CAACCCACAACATATTCGGGATGAAGGGGACTGGGTCAGATTCTCTA | |
| | CCAATGTCAAGACTGCCGAAACGTTGATGAATACCCGCTTCAACGTC | |
| | TTCAAGGACAACCTAAATTCCGTTTCAAAAATTCGAACTTTGGAGTATT | |
| | CCGTCCCTGTAGCTATATCAGCTCATGTCCAAATGATCCAGCCAACTA | |
| | CCTTATTTGGACGACAGAAGCCACAGAACAGTTTGATCCTAAACCCCT | |
| | TGACCAAGGATCTAGAATCCATGTCCGTTGAAGAATTTGCTGCTTCTC | |
| | AGTGCAGGTCCTTAGTGACTACTGCCTGCCTTCGAGAATTGTACGGA | |
| | CTTGGTGACCGTGTCACTCAGGCTAGGGATGACAACCGTATTGGAGT | |
| | ATCCGGCTTTTTGGAGGAGTACGCCCAATACCGCGATCTTGAGCTCT | |
| | TCCTCTCTCGCTTTGAGCCATCCGCCAAAGGATTTAATTTCAGTGAAG | |
| | GCCTTATTGCCGGAGGAAAGAACACTCAGGGTGGTCCTGGAAGCTCT | |
| | ACTGAGGCCAACCTTGATATGCAATATGTCGTCGGTCTGTCCCACAA | |
| | GGCAAAGGTCACCTATTACTCCACCGCTGGCCGTGGCCCATTAATTC | |
| | CCGATCTATCTCAGCCAAGCCAAGCTTCAAACAACAACGAACCATAC | |
| | CTTGAACAGCTGCGGTACCTCGTAAAGCTCCCCAAGAACCAGCTTCC | |
| | ATCTGTATTGACAACTTCCTATGGAGACACAGAACAGAGCTTGCCCG | |
| | CCAGCTATACCAAAGCCACTTGCGACCTCTTTGCTCAGCTAGGAACT | |
| | ATGGGTGTGTCTGTTATCTTCAGCAGTGGTGATACCGGGCCCGGAAG | |
| | CTCATGCCAGACCAACGATGGCAAGAATGCGACTCGCTTCAACCCTA | |
| | TCTACCCAGCTTCTTGCCCGTTTGTGACCTCCATCGGTGGAACCGTT | |
| | GGTACCGGTCCTGAGCGTGCAGTTTCATTCTCCTCTGGTGGCTTCTC | |
| | AGACAGGTTCCCCCGCCCACAATATCAGGATAACGCTGTTAAAGACT | |
| | ACCTGAAAATTTTGGGCAACCAGTGGAGCGGATTGTTTGACCCCAAC | |
| | GGCCGTGCTTTCCCAGATATCGCAGCTCAGGGATCAAATTATGCTGT | |
| | CTATGACAAGGGAAGGATGACTGGAGTCTCCGGCACCAGTGCATCC | |
| | GCCCCTGCCATGGCTGCCATCATTGCCCAGCTTAACGATTTCCGACT | |
| | GGCAAAGGGCTCTCCTGTGCTGGGATTCTTGAACCCATGGATATATT | |
| | CCAAGGGTTTCTCTGGCTTTACAGATATTGTTGATGGCGGTTCCAGG | |
| | GGTTGCACTGGTTACGATATATACAGCGGCTTGAAAGCGAAGAAGGT | |
| | TCCCTACGCAAGCTGGAATGCAACTAAGGGATGGGACCCAGTAACG | |
| | GGATTTGGTACTCCCAACTTCCAAGCTCTCACTAAAGTGCTGCCCTAA | |
| 61 | ATGTATATCACCTCATCCCGCCTCGTGCTGGCCTTAGCGGCACTTCC | Fusarium |
| | GACAGCATTTGGTAAATCATACTCCCACCATGCCGAAGCACCAAAGG | graminearum |
| | GATGGAAGGTCGACGACACCGCTCGTGTTGCCTCCACCGGTAAACAA | PH-1 |
| | CAGGTCTTCAGCATCGCACTGACCATGCAAAATGTTGATCAGCTCGA | |
| | GTCCAAGCTCCTTGACCTCTCCAGCCCCGACAGCAAGAACTATGGCC | |
| | AGTGGATGTCTCAAAAGGACGTAACAACTGCTTTCTATCCTTCGAAAG | |
| | AAGCTGTTTCCAGTGTGACAAAGTGGCTCAAGTCCAAGGGTGTCAAG | |
| | CACTACAACGTCAACGGTGGTTTCATTGACTTTGCTCTCGATGTCAAG | |
| | GGTGCCAATGCGCTACTTGATAGTGACTATCAATACTACACCAAAGAG | |
| | GGCCAGACCAAGTTGCGAACTCTGTCTTACTCTATCCCTGATGATGTA | |
| | GCCGAACACGTTCAGTTCGTCGACCCAAGCACCAACTTTGGCGGCAC | |
| | ACTGGCTTTCGCCCCTGTCACTCACCCATCGCGTACTCTAACCGAGC | |
| | GCAAGAACAAGCCCACCAAGAGCACAGTCGATGCTTCATGCCAAACC | |
| | AGCATCACACCCTCATGCTTGAAGCAGATGTACAACATTGGTGACTAC | |
| | ACTCCCAAGGTCGAGTCTGGAAGCACTATTGGTTTCAGCAGCTTCCT | |
| | TGGCGAGTCCGCCATCTACTCCGATGTTTTCCTGTTTGAGGAGAAGT | |
| | TTGGAATTCCCACGCAGAACTTTACCACTGTTCTCATCAACAACGGCA | |
| | CTGATGACCAGAACACTGCTCACAAGAACTTTGGCGAGGCTGACTTG | |
| | GATGCCGAGAACATTGTTGGAATTGCCCACCCTCTTCCCTTCACCCA | |
| | GTACATCACTGGCGGTTCACCACCTTTTCTTCCCAACATCGATCAGCC | |
| | AACTGCTGCCGATAACCAGAACGAGCCTTATGTGCCTTTCTTCCGCTA | |
| | CCTTCTATCGCAGAAGGAAGTCCCTGCAGTTGTCTCTACCTCGTATG | |
| | GTGACGAAGAAGATAGCGTCCCTCGCGAATATGCTACCATGACCTGC | |
| | AACCTGATTGGTCTTCTCGGACTTCGAGGAATCAGTGTCATCTTCTCC | |
| | TCTGGCGATATCGGCGTTGGTGCTGGATGTCTCGGCCCTGACCACAA | |
| | GACTGTCGAGTTCAACGCCATCTTCCCTGCCACCTGCCCTTACCTCA | |
| | CCTCCGTCGGCGGTACCGTTGATGTCACCCCCGAAATCGCCTGGGA | |
| | AGGTTCTTCTGGTGGTTTCAGCAAGTACTTCCCCCGACCCAGCTACC | |
| | AGGACAAGGCTGTCAAGACGTACATGAAGACTGTCTCCAAGCAGACA | |
| | AAGAAGTACTACGGCCCTTACACCAACTGGGAAGGCCGAGGCTTCCC | |
| | TGATGTTGCTGGCCACAGTGTCTCTCCCAACTATGAGGTTATCTATGC | |
| | TGGTAAGCAGAGTGCAAGCGGAGGTACCAGTGCTGCTGCTCCTGTTT | |
| | GGGCTGCCATTGTCGGTCTGCTCAACGATGCCCGTTTCAGAGCTGG | |
| | GAAGCCAAGCTTGGGATGGTTGAACCCTCTTGTTTACAAGTATGGAC | |
| | CAAAGGTGTTGACTGACATCACTGGTGGTTACGCCATTGGATGTGAT | |
| | GGCAACAACACCCAGTCCGGAAAGCCTGAGCCTGCAGGATCCGGTA | |
| | TTGTGCCCGGTGCCAGATGGAATGCCACTGCCGGATGGGATCCTGT | |
| | CACTGGTTATGGTACACCCGACTTTGGAAAGTTGAAGGATTTGGTTCT | |
| | TAGCTTCTAA | |
| 62 | ATGCGTTCCTCCGGTCTTTACGCAGCACTGCTGTGCTCTCTGGCCGC | Aspergillus |
| | ATCGACCAACGCAGTTGTTCATGAGAAGCTCGCCGCGGTCCCCTCG | kawachii IFO |
| | GGCTGGCACCATCTCGAAGATGCTGGCTCCGATCACCAGATTAGCCT | 4308 |
| | GTCGATCGCATTGGCACGCAAGAACCTCGATCAGCTTGAATCCAAGC | |
| | TGAAAGACTTGTCCACACCAGGTGAATCGCAGTATGGCCAGTGGCTG | |
| | GATCAAGAGGAAGTCGACACACTGTTCCCAGTGGCCAGCGACAAGG | |
| | CCGTGATCAGCTGGTTGCGCAGCGCCAACATCACCCATATTGCCCGG | |
| | CAGGGCAGCTTGGTGAACTTTGCGACCACCGTCGACAAGGTGAACAA | |
| | GCTTCTCAACACCACTTTTGCTTACTACCAAAGAGGTTCTTCCCAGAG | |
| | ACTGCGCACGACAGAGTACTCCATTCCCGATGATCTGGTCGACTCGA | |
| | TCGACCTCATCTCCCCGACAACCTTTTTCGGCAAGGAAAAGACCAGT | |
| | GCTGGCCTGACCCAGCGGTCGCAGAAAGTCGACAACCATGTGGCCA | |
| | AACGCTCCAACAGCTCGTCCTGCGCCGATACCATCACGTTATCCTGC | |
| | CTGAAGGAGATGTACAACTTTGGCAACTACACTCCCAGCGCCTCGTC | |
| | AGGAAGCAAGCTGGGATTCGCCAGCTTCCTGAACGAGTCCGCCTCG | |
| | TATTCCGATCTTGCCAAGTTCGAGAGACTGTTCAACTTGCCGTCTCAG | |
| | AACTTCTCCGTGGAGCTGATCAACGGCGGCGTCAATGACCAGAACCA | |
| | ATCGACGGCTTCTCTGACCGAGGCTGACCTCGATGTGGAATTGCTCG | |
| | TTGGCGTAGGTCATCCTCTTCCGGTGACCGAGTTTATCACTTCTGGC | |
| | GAACCTCCTTTCATTCCCGACCCCGATGAGCCGAGTGCCGCCGATAA | |
| | TGAGAATGAGCCTTACCTTCAGTACTACGAGTACCTCCTCTCCAAGCC | |
| | CAACTCGGCCCTGCCCCAAGTGATTTCCAACTCCTACGGTGACGACG | |
| | AACAGACCGTTCCAGAATACTACGCCAAGCGAGTCTGCAACCTGATC | |
| | GGACTGGTCGGCCTGCGCGGCATCAGCGTCCTGGAATCATCCGGTG | |
| | ACGAAGGAATTGGATCTGGCTGCCGCACCACCGACGGCACTAACAG | |
| | CACCCAATTCAATCCCATCTTCCCCGCCACCTGTCCCTACGTGACCG | |
| | CCGTAGGAGGCACCATGTCCTACGCGCCCGAAATTGCCTGGGAAGC | |
| | CAGTTCCGGTGGTTTCAGCAACTACTTCGAGCGAGCCTGGTTCCAGA | |
| | AGGAAGCCGTGCAGAACTACCTGGCGAACCACATCACCAACGAGAC | |
| | GAAGCAGTATTACTCACAATTCGCTAACTTTAGCGGTCGCGGATTTCC | |
| | CGATGTTTCGGCCCATAGCTTTGAGCCTTCGTACGAAGTTATCTTCTA | |
| | CGGCGCCCGTTACGGCTCCGGCGGTACTTCCGCCGCATGTCCTCTG | |
| | TTCTCTGCGCTAGTGGGCATGTTGAACGATGCTCGTCTGCGGGCGG | |
| | GCAAGTCCACGCTTGGTTTCTTGAACCCCCTGCTGTACAGTAAGGGG | |
| | TACAAGGCGCTGACAGATGTCACGGCGGGACAATCGATCGGGTGCA | |
| | ATGGCATTGATCCGCAGAGTGATGAGGCTGTTGCGGGCGCGGGCAT | |
| | TATCCCGTGGGCGCATTGGAATGCCACAGTCGGATGGGATCCGGTG | |
| | ACGGGATTGGGACTTCCTGATTTTGAGAAGTTGAGGCAGTTGGTGCT | |
| | GTCGTTGTAG | |
| 63 | ATGAGTCGAAATCTCCTCGTTGGTGCTGGCCTGTTGGCCCTCGCCCA | Talaromyces |
| | ATTGAGCGGTCAAGCTCTCGCTGCCGCTGCCCTCGTCGGCCATGAAT | stipitatus |
| | CCCTAGCTGCGCTGCCAGTTGGCTGGGATAAGGTCAGCACGCCAGC | ATCC 10500 |
| | TGCAGGGACGAACATTCAATTGTCCGTCGCCCTCGCTCTGCAAAACA | |
| | TCGAGCAGCTGGAAGACCACTTGAAGTCTGTGTCAACCCCCGGTTCT | |
| | GCCAGCTACGGTCAGTACCTGGATTCCGACGGTATTGCCGCTCAATA | |
| | CGGTCCCAGCGACGCATCCGTTGAGGCTGTCACCAACTGGCTGAAG | |
| | GAGGCCGGTGTCACTGACATCTACAACAACGGCCAGTCGATTCACTT | |
| | CGCAACCAGTGTCAGCAAGGCCAACAGCTTGCTCGGGGCCGATTTC | |
| | AACTACTATTCTGATGGTAGTGCGACCAAGTTGCGTACCTTAGCTTAT | |
| | TCCGTTCCCAGTGACCTCAAAGAGGCCATCGACCTTGTCTCGCCCAC | |
| | CACCTATTTCGGCAAGACCACTGCTTCTCGTAGCATCCAGGCTTACAA | |
| | GAACAAGCGCGCCTCTACTACTTCCAAGTCTGGATCGAGCTCTGTGC | |
| | AAGTATCTGCTTCCTGCCAGACCAGCATCACTCCTGCCTGCTTGAAA | |
| | CAGATGTACAATGTTGGCAACTACACACCCAGCGTCGCTCACGGCAG | |
| | TCGTGTCGGATTCGGTAGCTTCTTGAATCAATCTGCCATCTTTGACGA | |
| | CTTGTTCACCTACGAAAAGGTCAATGATATTCCATCACAGAATTTCAC | |
| | TAAGGTGATTATTGCAAATGCATCCAACAGCCAAGATGCCAGCGATG | |
| | GCAACTACGGCGAAGCCAACCTTGACGTGCAAAACATTGTCGGCATC | |
| | TCTCATCCTCTCCCCGTGACTGAATTCCTCACTGGTGGCTCACCTCC | |
| | CTTCGTTGCTAGCCTCGACACCCCTACCAACCAGAACGAGCCATATA | |
| | TTCCTTACTACGAATATCTTTTGTCTCAGAAGAACGAGGATCTCCCCC | |
| | AGGTCATTTCCAACTCTTACGGAGACGACGAGCAGTCTGTGCCGTAC | |
| | AAGTATGCCATCCGTGCATGCAACCTGATCGGCCTGACAGGTTTACG | |
| | AGGTATCTCGGTCTTGGAATCCAGCGGTGATCTCGGCGTTGGAGCC | |
| | GGCTGTCGCAGCAACGATGGCAAGAACAAGACTCAATTTGACCCCAT | |
| | CTTCCCTGCCACTTGCCCCTACGTTACCTCTGTTGGTGGTACCCAATC | |
| | CGTTACCCCTGAAATTGCCTGGGTCGCCAGCTCCGGTGGTTTCAGCA | |
| | ACTACTTCCCTCGTACCTGGTACCAGGAACCCGCAATTCAGACCTAT | |
| | CTCGGACTCCTTGACGATGAGACCAAGACATACTATTCTCAATACACC | |
| | AACTTTGAAGGCCGTGGTTTCCCCGATGTTTCCGCCCACAGCTTGAC | |
| | CCCTGATTACCAGGTCGTCGGTGGTGGCTATCTCCAGCCAAGCGGT | |
| | GGTACTTCCGCTGCTTCTCCTGTCTTTGCCGGCATCATTGCGCTTTTG | |
| | AACGACGCTCGTCTCGCTGCTGGCAAGCCCACTCTTGGCTTCTTGAA | |
| | CCCGTTCTTCTACCTTTATGGATACAAGGGTTTAAACGATATCACTGG | |
| | AGGACAGTCAGTGGGTTGCAACGGTATCAACGGCCAAACTGGGGCT | |
| | CCTGTTCCCGGTGGTGGCATTGTTCCTGGAGCGGCCTGGAACTCTAC | |
| | TACTGGCTGGGACCCAGCCACTGGTCTCGGAACACCCGACTTCCAG | |
| | AAGTTGAAAGAACTCGTACTTAGCTTTTAA | |
| 64 | ATGTATATCTCCTCCCAAAATCTGGTACTCGCCTTATCGGCGCTGCCT | Fusarium |
| | TCAGCATTTGGCAAATCCTTCTCTCACCATGCTGAAGCTCCTCAAGGC | oxysporum f. |
| | TGGCAAGTCCAAAAGACTGCCAAAGTCGCTTCCAACACGCAGCATGT | sp. cubense |
| | CTTCAGTCTTGCACTAACCATGCAAAACGTGGATCAGCTCGAATCCAA | race 4 |
| | GCTTCTTGACCTCTCCAGCCCCGACAGCGCCAACTACGGTAACTGGC | |
| | TCTCCCACGATGAGCTCACAAGCACTTTCTCTCCTTCCAAGGAGGCG | |
| | GTGGCTAGTGTGACAAAGTGGCTCAAGTCAAAGGGCATCAAGCACTA | |
| | CAAGGTCAACGGTGCTTTCATTGACTTTGCTGCTGATGTTGAGAAGG | |
| | CCAATACGCTTCTCGGAGGTGATTACCAGTACTACACTAAGGATGGT | |
| | CGGTCACGTTCAATTTGTTGATCCTAGCACAAACTTCGGTGGCACCG | |
| | TTGCGTTCAACCCTGTGCCTCACCCCTCGCGCACCCTCCAAGAGCGC | |
| | AAGGTCTCTCCCTCCAAGAGCACCGTTGATGCTTCATGCCAGACAAG | |
| | CATCACCCCTTCTTGCCTCAAGCAGATGTACAACATTGGAGACTACAC | |
| | TCCCGATGCCAAGTCTGGAAGTGAGATTGGTTTCAGCAGCTTTCTCG | |
| | GCCAGGCTGCTATTTACTCTGATGTCTTCAAGTTTGAGGAGCTGTTTG | |
| | GTATTCCTAAGCAGAACTACACCACTATTCTGATCAACAATGGCACCG | |
| | ATGATCAGAATACTGCGCATGGAAACTTTGGAGAGGCTAACCTTGAT | |
| | GCTGAGAACATTGTTGGAATCGCTCATCCTCTTCCTTTCAAGCAGTAC | |
| | ATTACTGGAGGTTCACCACCTTTCGTTCCCAACATCGATCAGCCCACC | |
| | GAGAAGGATAACCAGAACGAGCCCTACGTGCCTTTCTTCCGTTACCT | |
| | CTTGGGCCAGAAGGATCTCCCAGCCGTCATCTCCACTTCCTACGGCG | |
| | ATGAAGAAGACAGCGTTCCTCGTGAGTATGCTACACTCACCTGCAAC | |
| | ATGATCGGTCTTCTCGGTCTCCGTGGCATCAGTGTCATCTTCTCTTCC | |
| | GGTGACATCGGTGTCGGTTCCGGCTGCCTTGCTCCCGACTACAAGAC | |
| | CGTCGAGTTCAATGCCATCTTCCCCGCCACATGCCCCTACCTCACCT | |
| | CCGTCGGCGGTACCGTCGACGTCACCCCCGAGATCGCCTGGGAGG | |
| | GATCCTCCGGCGGATTCAGCAAGTACTTCCCCCGACCCAGCTACCAG | |
| | GACAAGGCCATCAAGAAGTACATGAAGACAGTCTCCAAGGAGACCAA | |
| | GAAGTACTACGGCCCTTACACCAACTGGGAGGGCCGAGGTTTCCCT | |
| | GATGTCGCTGGACACAGTGTTGCGCCTGACTACGAGGTTATCTACAA | |
| | TGGTAAGCAGGCTCGAAGTGGAGGTACCAGCGCTGCTGCCCCTGTT | |
| | TGGGCTGCTATCGTTGGTCTGTTGAACGATGCCCGCTTCAAGGCTGG | |
| | TAAGAAGAGCTTGGGATGGTTGAACCCTCTTATCTACAAGCATGGAC | |
| | CCAAGGTCTTGACTGACATCACCGGTGGCTATGCTATTGGATGTGAC | |
| | GGTAACAACACTCAGTCTGGAAAGCCCGAGCCCGCTGGATCTGGTCT | |
| | TGTTCCCGGTGCTCGATGGAACGCCACAGCTGGATGGGATCCTACCA | |
| | CTGGCTATGGAACTCCCAACTTCCAGAAGTTGAAGGACTTGGTTCTC | |
| | AGCTTGTAA | |
| 65 | ATGCCTAAGTCCACAGCGCTTCGGCTTGTTAGCCTCCTTTCCCTGGC | Trichoderma |
| | CAGTGTGCCGATATCTGCCTCCGTCCTTGTGGAAAGTCTCGAAAAGC | virens Gv29- |
| | TGCCTCACGGATGGAAAGCTGCTTCGGCTCCTAGCCCTTCCTCCCAG | 8 |
| | ATAACCCTACAAGTCGCTCTTACGCAGCAGAACATCGATCAGCTGGA | |
| | ATCGAGGCTCGCGGCTGTATCCACACCAAATTCCAAGACATACGGAA | |
| | ATTATCTGGATCTTGATGAGATCAATGAGATCTTCGCGCCAAGCGATG | |
| | CCAGCAGCGCAGCCGTGGAGTCTTGGCTCCATTCTCACGGTGTGAC | |
| | AAAATACACGAAGCAAGGCAGCAGTATCTGGTTCCAAACCGAAGTTT | |
| | CTACAGCAAATGCAATGTTGAGCACAAACTTCCACACTTACAGTGATG | |
| | CTGCTGGCGTTAAGAAGTTGCGAACTCTTCAGTATTCAATTCCGGAGA | |
| | GTCTTGTGGGCCATGTCGATCTCATCTCACCCACGACCTACTTTGGC | |
| | ACCTCTAACGCTATGAGAGCTTTGAGATCTAAAAGCGTGGCTTCAGTT | |
| | GCTCAAAGTGTGGCAGCCCGCCAAGAACCTTCTAGCTGCAAGGGAA | |
| | CTCTGGTTTTCGAAGGAAGAACGTTCAATGTCTTCCAACCAGATTGTC | |
| | TTAGGACAGAGTACAATGTCAATGGATACACTCCATCAGCCAAGTCTG | |
| | GTAGTAGAATAGGATTTGGTTCCTTCTTAAACCAAAGTGCAAGCTTTT | |
| | CAGACCTCGCACTCTTTGAAAAACACTTTGGGTTTTCCAGCCAAAATT | |
| | TCTCCGTCGTTCTGATCAATGGTGGAACGGACCTGCCCCAACCACCC | |
| | TCTGACGACAACGATGGCGAGGCCAATTTGGATGTCCAAAACATTTT | |
| | GACAATCGCACACCCTCTGCCCATCACTGAATTCATCACTGCCGGAA | |
| | GCCCGCCGTACTTCCCAGATCCCGTTGAACCTGCAGGAACTCCCGAT | |
| | GAGAACGAGCCTTACTTGCAGTACTTTGAGTATCTGTTGTCGAAGCC | |
| | CAACAGAGATCTTCCTCAGGTCATTACCAACTCTTACGGTGATGAGGA | |
| | GCAAACAGTACCTCAGGCTTATGCTGTCCGAGTGTGCAACCTAATTG | |
| | GATTGATGGGACTGCGTGGTATCAGTATCCTCGAGTCCTCCGGCGAT | |
| | GAGGGAGTGGGTGCTTCCTGCGTTGCTACCAACAGCACCACTCCTCA | |
| | ATTTAACCCCATTTTCCCGGCAACATGCCCCTATGTCACTAGCGTAGG | |
| | TGGAACTGTGAACTTCAACCCAGAAGTTGCCTGGGACGGTTCATCTG | |
| | GAGGTTTCAGCTACTATTTCTCCAGGCCATGGTACCAAGAGGAAGCA | |
| | GTTGGAAACTACCTAGAGAAGCATGTCAGCGCCGAAACAAAGAAGTA | |
| | CTACGGGCCTTATGTCGATTTCTCTGGACGTGGCTTCCCTGATGTTG | |
| | CAGCTCACAGCGTGAGCCCCGATTATCCTGTGTTCCAAGGCGGCCA | |
| | GCTCACTCCTAGCGGAGGCACTTCTGCGGCTTCTCCCGTCGTAGCCA | |
| | GTATCATTGCCCTTCTGAACGATGCACGCCTCCGTGAAGGCAAGCCC | |
| | ACACTTGGGTTCCTGAACCCGCTGATTTACCAATATGCTTACAAGGGT | |
| | TTCACGGATATCACATCCGGCCAGTCTGATGGCTGCAATGGCAACAA | |
| | CACCCAAACGGATGCCCCTCTTCCTGGAGCTGGCGTTGTCCTAGGAG | |
| | CACACTGGAATGCGACCAAAGGATGGGATCCTACGACAGGATTTGGT | |
| | GTCCCTAACTTTAAGAAGCTACTCGAGCTGATCCGATATATATAG | |
| 66 | ATGGCTAAACTGACGGCACTTCGGCTCGTCAGCCTTCTTTGCCTTGC | Trichoderma |
| | GGCTGCGCAGGCCTCTGCTGCTGTGCTCGTGGAAAGCCTCAAACAA | atroviride IMI |
| | GTGCCCAACGGGTGGAATGCAGTCTCGACCCCAGACCCTTCGACAT | 206040 |
| | CGATTGTCTTGCAAATCGCCCTCGCGCAACAGAATATCGATGAATTG | |
| | GAATGGCGTCTCGCGGCTGTATCCACGCCCAACTCTGGCAATTATGG | |
| | CAAATACCTGGATATTGGAGAGATTGAAGGAATTTTCGCCCCAAGCAA | |
| | TGCCTCTTACAAAGCCGTGGCATCGTGGCTCCAGTCTCATGGGGTGA | |
| | AGAACTTCGTCAAACAAGCCGGCAGTATTTGGTTCTACACTACTGTCT | |
| | CTACCGCAAACAAGATGCTTAGCACAGATTTCAAACACTATAGCGATC | |
| | CTGTTGGCATTGAGAAGCTGCGTACTCTTCAGTACTCGATCCCAGAA | |
| | GAACTAGTCGGCCATGTTGATCTCATCTCGCCTACAACATATTTTGGA | |
| | AACAACCACCCCGCGACAGCGAGAACACCCAACATGAAGGCCATTAA | |
| | CGTAACCTACCAAATCTTTCACCCAGACTGCCTTAAAACGAAATACGG | |
| | CGTTGATGGCTATGCCCCATCTCCAAGATGTGGCAGCAGGATTGGTT | |
| | TTGGCTCATTCCTCAACGAAACTGCCAGTTATTCGGATCTTGCGCAGT | |
| | TTGAGAAGTACTTTGACCTTCCCAACCAAAACCTTTCCACCTTATTGAT | |
| | CAATGGCGCAATCGACGTTCAGCCACCTTCCAACAAAAACGACAGCG | |
| | AGGCCAACATGGACGTTCAGACCATCTTGACCTTTGTCCAACCTCTTC | |
| | CTATTACTGAGTTTGTTGTTGCCGGAATCCCGCCGTATATTCCTGATG | |
| | CGGCTTTGCCGATCGGCGACCCTGTCCAAAACGAGCCGTGGCTGGA | |
| | ATACTTTGAGTTTTTGATGTCCAGGACCAACGCAGAGCTTCCCCAGGT | |
| | CATTGCCAACTCATACGGTGACGAGGAACAAACGGTACCACAGGCGT | |
| | ATGCCGTCCGAGTATGCAACCAGATTGGGCTGTTGGGCCTTCGCGGT | |
| | ATATCCGTTATCGCATCATCTGGCGATACGGGTGTTGGAATGTCTTGT | |
| | ATGGCTTCGAACAGCACTACTCCTCAGTTTAACCCCATGTTCCCGGCT | |
| | TCGTGTCCTTATATCACCACTGTCGGTGGAACTCAGCACCTTGATAAT | |
| | GAGATTGCTTGGGAGCTTTCATCGGGAGGCTTCAGTAACTATTTCACA | |
| | AGGCCATGGTATCAAGAAGACGCAGCCAAAACATATCTTGAACGTCA | |
| | TGTCAGCACCGAGACAAAGGCATATTACGAACGTTACGCCAATTTCTT | |
| | GGGACGCGGCTTTCCCGACGTTGCAGCACTTAGTCTCAACCCCGATT | |
| | ATCCAGTGATTATTGGCGGAGAACTTGGTCCCAATGGAGGCACTTCT | |
| | GCGGCCGCACCCGTCGTCGCTAGTATTATTGCACTCTTGAACGATGC | |
| | ACGCCTTTGCCTAGGCAAACCTGCCCTTGGGTTCTTGAACCCCCTGA | |
| | TCTATCAATATGCTGATAAGGGTGGCTTCACGGATATCACGTCCGGC | |
| | CAGTCTTGGGGCTGTGCCGGAAATACCACTCAGACGGGGCCTCCTC | |
| | CCCCTGGAGCTGGTGTCATTCCGGGGGCACACTGGAATGCGACCAA | |
| | GGGATGGGATCCTGTAACAGGATTTGGAACCCCGAACTTCAAGAAAT | |
| | TACTCTCACTGGCCCTGTCCGTCTAA | |
| 67 | ATGTTTTGGCGTCCAGCTTTTGTCCTTCTTCTCGCTCAGCTTGTCACT | Agaricus |
| | GCTAGTCCTTTAGCTCGACGCTGGGATGATTTCGCAGAAAAACATGC | bisporus var. |
| | CTGGGTTGAAGTTCCTCGCGGGTGGGAAATGGTCTCCGAGGCTCCC | burnettii |
| | AGTGACCATACCTTTGATCTTCGCATTGGAGTAAAGTCAAGTGGCATG | JB137-S8 |
| | GAGCAGCTCATTGAAAACTTGATGCAAACCAGCGATCCTACTCATTCC | |
| | AGATATGGTCAACATCTTAGTAAAGAAGAGCTCCATGATTTCGTTCAG | |
| | CCTCATCCTGATTCTACCGGAGCGGTCGAAGCATGGCTTGAAGATTT | |
| | CGGTATCTCCGATGATTTCATTGATCGTACTGGAAGTGGCAACTGGG | |
| | TTACTGTTCGAGTTTCAGTAGCCCAGGCTGAACGTATGCTTGGTACCA | |
| | AGTATAACGTCTACCGCCATTCTGAATCAGGGGAATCGGTTGTACGA | |
| | ACAATGTCTTATTCGCTTCCCAGCGAACTTCACTCCCACATAGATGTT | |
| | GTCGCACCCACCACTTATTTCGGCACGATGAAAAGCATGCGGGTGAC | |
| | CAGCTTCTTACAGCCGGAAATAGAGCCTGTTGACCCAAGCGCTAAAC | |
| | CATCGGCTGCTCCAGCTTCCTGTTTGAGTACCACTGTCATAACCCCC | |
| | GATTGCCTCCGTGACCTTTATAATACGGCTGACTACGTTCCTTCCGCC | |
| | ACTTCACGGAATGCCATTGGTATTGCTGGGTACTTGGATCGTTCAAAT | |
| | CGTGCAGATCTTCAGACTTTCTTCCGACGCTTCCGGCCCGATGCCGT | |
| | TGGCTTCAATTACACGACTGTCCAACTAAATGGCGGAGGAGACGACC | |
| | AGAATGATCCCGGTGTAGAGGCCAACCTCGATATTCAATACGCCGCT | |
| | GGTATTGCTTTCCCCACACCAGCTACATACTGGAGTACTGGCGGCTC | |
| | TCCACCTTTCATTCCAGATACTCAAACCCCGACAAACACCAATGAGCC | |
| | CTACCTGGATTGGATCAATTTTGTCCTAGGCCAGGACGAGATTCCAC | |
| | AGGTGATTTCAACGTCCTATGGTGACGACGAGCAAACAGTTCCTGAA | |
| | GATTACGCTACTAGCGTGTGTAATCTCTTCGCGCAACTCGGCAGCCG | |
| | TGGCGTTACAGTATTCTTCTCCAGCGGTGACTTTGGTGTTGGTGGTG | |
| | GAGATTGCCTCACGAATGATGGCTCAAACCAAGTCCTTTTCCAGCCG | |
| | GCTTTCCCCGCTTCCTGCCCATTCGTAACAGCTGTTGGCGGAACTGT | |
| | CAGGCTTGATCCTGAGATTGCTGTCAGTTTCTCTGGAGGAGGCTTTT | |
| | CCCGTTACTTCTCCAGGCCATCGTACCAGAATCAAACTGTGGCTCAAT | |
| | TTGTTTCTAATCTTGGGAATACATTCAACGGACTCTACAATAAAAATGG | |
| | AAGGGCCTACCCAGATCTTGCAGCACAGGGCAATGGCTTCCAAGTTG | |
| | TTATAGACGGCATCGTCCGTTCGGTTGGAGGGACCAGCGCCAGCTCT | |
| | CCGACGGTTGCCGGTATCTTTGCGCTTTTGAATGACTTCAAGCTCTCA | |
| | AGAGGCCAGTCGACACTCGGATTTATCAACCCACTTATATACTCCTCC | |
| | GCTACATCCGGCTTCAATGACATCAGGGCGGGTACAAACCCTGGTTG | |
| | TGGTACTCGCGGATTTACCGCTGGTACTGGTTGGGATCCGGTCACTG | |
| | GTCTGGGCACTCCCGATTTTTTGAGGCTTCAGGGACTTATTTAA | |
| 68 | ATGCTCGACCGTATCCTTCTCCCCCTCGGCCTCCTGGCCTCCCTTGC | Magnaporthe |
| | CACCGCTCGTGTCTTTGACAGCCTACCCCACCCTCCCCGAGGCTGGT | oryzae 70-15 |
| | CATACTCGCACGCGGCGGAATCGACGGAGCCGCTGACCCTGCGCAT | |
| | CGCCCTCCGCCAGCAAAATGCCGCCGCCCTGGAGCAGGTGGTGCTG | |
| | CAGGTCTCGAACCCCAGGCACGCCAATTACGGCCAGCACCTGACGC | |
| | GCGACGAGCTGCGCAGCTACACGGCGCCCACGCCGCGGGCCGTCC | |
| | GCAGCGTGACGTCGTGGCTGGTCGACAACGGCGTCGACGACTACAC | |
| | GGTCGAGCACGACTGGGTGACGCTGCGCACGACGGTCGGGGCCGC | |
| | GGACAGGCTGCTCGGCGCAGACTTTGCCTGGTATGCCGGCCCGGGC | |
| | GAGACGCTGCAGCTGCGGACGCTCTCGTACGGCGTCGACGACTCGG | |
| | TGGCGCCGCACGTCGACCTCGTGCAGCCCACGACGCGGTTTGGCGG | |
| | TCCCGTCGGGCAGGCGTCGCACATCTTCAAGCAGGACGACTTTGAC | |
| | GAGCAGCAGCTCAAGACCTTGTCGGTGGGGTTCCAGGTCATGGCTG | |
| | ACCTGCCGGCCAACGGGCCTGGGTCGATCAAGGCGGCATGTAACGA | |
| | GTCTGGCGTGACGCCCCTGTGCCTGCGAACTCTGTACAGGGTCAACT | |
| | ACAAGCCGGCAACCACGGGGAACCTGGTCGCTTTCGCGTCGTTCCT | |
| | GGAGCAGTACGCCAGGTACAGTGATCAGCAGGCATTCACTCAGCGG | |
| | GTCCTTGGCCCTGGTGTTCCGTTGCAGAACTTTTCGGTCGAAACGGT | |
| | CAACGGTGGAGCCAATGACCAGCAGAGCAAACTTGACAGCGGCGAG | |
| | GCGAACCTCGATCTGCAGTACGTCATGGCAATGAGCCACCCTATTCC | |
| | AATTTTGGAGTACAGCACTGGAGGCAGAGGACCCCTCGTCCCAACTC | |
| | TGGACCAGCCCAACGCCAACAACAGCAGCAATGAGCCTTACCTGGA | |
| | GTTCCTGACGTACCTCCTGGCCCAACCCGACTCAGCCATCCCTCAGA | |
| | CCCTGTCGGTGTCGTATGGCGAGGAGGAACAGTCGGTGCCGCGCGA | |
| | CTACGCCATCAAGGTTTGCAACATGTTCATGCAGCTCGGCGCCCGCG | |
| | GCGTGTCGGTTATGTTTTCGTCGGGCGACTCGGGCCCGGGTAATGA | |
| | CTGTGTTCGAGCCTCGGACAACGCAACCTTTTTTGGCTCAACATTCCC | |
| | CGCAGGCTGCCCCTACGTCACGTCGGTGGGCTCCACCGTCGGCTTC | |
| | GAGCCGGAGCGCGCCGTCTCCTTTTCCTCGGGCGGCTTCAGCATTTA | |
| | CCACGCTCGCCCCGACTACCAAAACGAAGTGGTCCCCAAGTACATTG | |
| | AATCGATCAAGGCTTCGGGCTACGAAAAGTTCTTTGACGGCAACGGC | |
| | CGCGGAATTCCCGACGTGGCTGCCCAGGGCGCCCGCTTCGTCGTCA | |
| | TCGACAAGGGCCGCGTTTCTCTAATCTCGGGGACCAGCGCCAGCTC | |
| | ACCTGCGTTTGCTGGCATGGTGGCGCTCGTCAACGCCGCCCGCAAG | |
| | TCAAAGGACATGCCGGCCTTGGGCTTCCTCAACCCCATGCTGTACCA | |
| | GAACGCCGCGGCCATGACGGACATTGTCAACGGCGCTGGCATCGGC | |
| | TGCAGGAAGCAACGTACAGAATTCCCGAATGGCGCCAGGTTCAACGC | |
| | CACGGCCGGCTGGGATCCCGTCACAGGGCTGGGGACGCCGTTGTTT | |
| | GACAAGCTGCTGGCTGTTGGCGCACCTGGAGTTCCCAACGCGTGA | |
| 69 | ATGCGTAGCCAGTTGCTCTTCTGCACAGCATTTGCTGCTCTCCAGTC | Togninia |
| | GCTTGTGGAGGGCAGCGATGTGGTGTTGGAGTCATTGCGAGAGGTC | minima |
| | CCTCAGGGCTGGAAGAGGCTTCGAGATGCGGACCCCGAGCAGTCCA | UCRPA7 |
| | TCAAGCTGCGCATTGCGCTTGAGCAGCCTAACCTGGACCTGTTCGAG | |
| | CAGACCCTCTACGACATCTCGTCACCGGATCACCCAAAATATGGCCA | |
| | GCATCTCAAGAGCCACGAGTTACGGGATATTATGGCACCTCGCGAGG | |
| | AGTCAACTGCTGCTGTCATCGCTTGGCTGCAAGACGCTGGGCTTTCT | |
| | GGCTCGCAGATTGAGGACGACAGCGACTGGATCAACATCCAGACGA | |
| | CAGTCGCCCAAGCCAACGACATGCTGAACACGACTTTCGGTCTCTTC | |
| | GCCCAGGAAGGCACCGAGGTCAATCGAATTCGAGCTCTGGCATATTC | |
| | CGTGCCTGAGGAGATCGTCCCTCACGTCAAGATGATTGCTCCCATCA | |
| | TCCGCTTCGGTCAGTTGAGACCTCAGATGAGCCACATCTTCTCGCAT | |
| | GAGAAAGTCGAGGAGACCCCGTCTATTGGCACCATCAAGGCCGCCG | |
| | CTATCCCATCTGTGGATCTTAACGTCACCGCTTGCAATGCCAGCATCA | |
| | CCCCCGAGTGCCTCCGAGCGCTTTACAACGTTGGTGATTACGAGGC | |
| | GGACCCATCGAAGAAGTCTCTTTTCGGAGTCTGTGGCTACTTGGAGC | |
| | AATATGCCAAGCACGATCAGCTGGCCAAGTTTGAGCAGACCTACGCT | |
| | CCGTATGCTATCGGTGCCGACTTCAGCGTCGTGACCATCAATGGCGG | |
| | AGGCGACAACCAGACCAGTACGATCGATGATGGAGAAGCCAACCTG | |
| | GATATGCAGTATGCTGTCAGCATGGCATACAAGACGCCAATCACATA | |
| | CTATTCAACTGGGGGTCGAGGACCTCTTGTTCCAGATCTCGACCAAC | |
| | CTGATCCCAACGACGTCTCAAACGAGCCGTACCTTGATTTTGTGAGC | |
| | TACCTTCTCAAGCTGCCCGACTCCAAATTGCCGCAGACCATCACAAC | |
| | TTCGTACGGAGAGGATGAGCAATCCGTTCCACGCTCCTACGTGGAGA | |
| | AGGTCTGCACCATGTTCGGCGCGCTCGGTGCCCGAGGCGTGTCTGT | |
| | GATCTTCTCCTCTGGTGATACCGGTGTCGGCTCAGCGTGCCAGACCA | |
| | ACGACGGCAAGAACACCACCCGCTTCCTGCCTATATTCCCTGCTGCG | |
| | TGCCCTTATGTGACCTCGGTTGGAGGCACTCGCTATGTCGACCCGGA | |
| | AGTCGCTGTGTCCTTCTCGTCTGGAGGCTTCTCGGACATCTTCCCTA | |
| | CGCCACTCTACCAGAAGGGCGCTGTCTCTGGCTACCTGAAGATCCTC | |
| | GGCGATCGCTGGAAGGGCCTCTATAACCCTCACGGCCGCGGTTTCC | |
| | CTGACGTCTCCGGACAGAGTGTCAGATACCACGTCTTCGACTACGGC | |
| | AAGGACGTCATGTACTCTGGCACAAGTGCCTCTGCACCGATGTTCGC | |
| | CGCGCTTGTCTCGCTGCTGAACAACGCCCGTCTCGCAAAGAAGTTGC | |
| | CGCCCATGGGATTCCTGAATCCCTGGCTGTATACCGTTGGTTTTAAC | |
| | GGGCTGACGGATATTGTGCACGGTGGATCTACTGGGTGCACTGGCA | |
| | CAGACGTGTACAGCGGCCTGCCCACACCTTTCGTTCCGTATGCGTCT | |
| | TGGAACGCAACCGTGGGATGGGACCCCGTTACTGGACTTGGCACGC | |
| | CTCTCTTTGATAAGCTGCTCAATTTGAGCACGCCAAACTTCCACTTGC | |
| | CGCACATTGGCGGTCACTAG | |
| 70 | ATGAAGTACAACACACTCCTCACCGGCCTGCTGGCTGTTGCCCATGG | Bipolaris |
| | CAGTGCCGTTTCCGCTTCAACTACTTCACATGTCGAGGGTGAAGTTG | maydis C5 |
| | TCGAGCGACTTCATGGCGTTCCTGAGGGTTGGAGTCAAGTGGGCGC | |
| | CCCCAATCCAGACCAGAAGCTGCGCTTTCGCATCGCAGTACGCTCGG | |
| | TGAGTAATTGCTTTTGTGAACCCATGTTTGAATCTTGCGGTGCTTTTTA | |
| | CTGAACATAACAGGCGGATAGCGAGCTGTTTGAGAGGACGCTTATGG | |
| | AGGTTTCTTCTCCCAGCCATCCTCGCTACGGACAGCACCTAAAGCGA | |
| | CACGAACTCAAGGACCTCATCAAACCGCGCGCCAAGTCAACTTCAAA | |
| | CATCCTGAACTGGCTGCAAGAGTCTGGAATTGAGGCCAGAGATATCC | |
| | AGAACGATGGCGAGTGGATCAGCTTCTATGCTCCGGTTAAACGTGCC | |
| | GAGCAAATGATGAGCACTACATTCAAGACCTATCAGAACGAGGCCCG | |
| | AGCGAATATCAAGAAGATCCGCTCTCTAGACTACTCGGTGCCGAAGC | |
| | ACATTCGAGATGACATCGACATCATCCAGCCTACGACTCGCTTCGGC | |
| | CAGATCCAACCGGAGCGTAGCCAAGTCTTTAGTCAAGAAGAGGTCCC | |
| | ATTCTCAGCGCTTGTTGTCAATGCGACGTGTAACAAGAAAATCACTCC | |
| | CGACTGCCTCGCCAACCTCTACAACTTCAAAGACTATGATGCCAGCG | |
| | ATGCCAATGTCACTATCGGAGTCAGCGGCTTCCTGGAGCAATATGCT | |
| | CGCTTTGACGACTTGAAGCAATTCATCAGCACTTTCCAACCAAAAGCA | |
| | GCTGGTTCCACATTCCAAGTTACATCTGTCAATGCAGGGCCTTTTGAC | |
| | CAGAACTCGACAGCCAGCAGTGTTGAAGCCAATCTTGACATTCAGTA | |
| | CACAACAGGTCTTGTTGCGCCCGACATTGAAACCCGCTACTTCACTG | |
| | TTCCCGGTCGCGGTATCCTGATCCCTGATCTGGACCAGCCTACGGAG | |
| | AGCGACAACGCTAATGAGCCGTATCTGGATTACTTTACATATCTTAAT | |
| | AACCTCGAAGACGAAGAACTCCCCGACGTGCTGACCACATCTTACGG | |
| | CGAGAGCGAGCAGAGTGTACCCGCCGAATATGCAAAAAAGGTGTGC | |
| | AATTTGATCGGCCAGTTGGGTGCTCGTGGTGTGTCCGTCATCTTCTC | |
| | CAGCGGTGATACTGGCCCTGGCTCTGCATGCCAAACCAATGATGGAA | |
| | AAAACACGACACGTTTCTTGCCCATCTTCCCTGCTTCTTGCCCCTACG | |
| | TCACTTCGGTTGGCGGCACTGTTGGTGTTGAGCCCGAAAAGGCTGTC | |
| | AGCTTCTCTTCGGGCGGCTTTTCTGACCTATGGCCTCGACCCGCTTA | |
| | TCAAGAGAAGGCCGTATCAGAATATCTTGAAAAGCTCGGAGACCGCT | |
| | GGAACGGGCTTTACAACCCTCAAGGACGCGGATTTCCTGATGTAGCT | |
| | GCTCAGGGCCAAGGCTTCCAGGTGTTTGACAAGGGCAGGCTGATTTC | |
| | GGTCGGAGGAACGAGCGCTTCAGCTCCTGTTTTCGCATCCGTAGTCG | |
| | CACTCCTGAACAATGCTCGCAAGGCTGCCGGCATGTCTTCACTCGGC | |
| | TTCTTGAACCCATGGATCTACGAGCAAGGCTACAAGGGCTTGACCGA | |
| | TATCGTTGCTGGAGGCTCGACAGGATGCACAGGAAGATCCATCTATT | |
| | CAGGCCTCCCAGCACCACTCGTGCCGTATGCTTCTTGGAATGCCACC | |
| | GAAGGATGGGATCCGGTGACGGGCTATGGTACACCTGATTTCAAGCA | |
| | ATTGCTCACCCTCGCGACGGCACCCAAGTCTGGCGAGCGTCGCGTT | |
| | CGTCGTGGCGGTCTCGGTGGCCAGGCTTAG | |
| 71 | ATGTTATCTTCCTTCCTTAGCCAGGGAGCAGCCGTATCCCTCGCGTTA | Aspergillus |
| | TTGTCGCTGCTCCCTTCGCCTGTAGCCGCGGAGATCTTCGAGAAGCT | kawachii IFO |
| | GTCCGGCGTCCCCAATGGCTGGAGATACGCCAACAATCCTCACGGC | 4308 |
| | AACGAGGTCATTCGCCTTCAAATCGCCCTTCAGCAGCACGATGTTGC | |
| | CGGTTTCGAACAAGCCGTGATGGACATGTCCACCCCCGGTCACGCC | |
| | GACTATGGAAAGCATTTCCGCACACATGATGAGATGAAGCGCATGCT | |
| | GCTCCCCAGCGACACTGCCGTCGACTCAGTTCGCGACTGGCTGGAA | |
| | TCCGCCGGAGTCCACAATATCCAGGTCGACGCCGACTGGGTCAAGTT | |
| | CCATACCACCGTCAACAAGGCCAATGCCCTGCTGGATGCCGACTTCA | |
| | AGTGGTATGTCAGCGAGGCCAAGCACATTCGTCGTCTACGCACCCTG | |
| | CAATACTCCATCCCCGACGCCCTGGTCTCGCACATCAACATGATCCA | |
| | GCCCACCACTCGCTTTGGCCAGATCCAGCCGAACCGTGCCACCATG | |
| | CGCAGCAAGCCCAAGCACGCCGACGAGACATTCCTGACCGCAGCCA | |
| | CCTTGGCCCAGAACACCTCCCACTGCGACTCCATCATCACGCCGCAC | |
| | TGTCTGAAGCAGCTCTACAACATCGGTGACTACCAGGCCGACCCCAA | |
| | GTCCGGTAGCAAGGTCGGCTTCGCCAGCTACCTCGAAGAATACGCC | |
| | CGGTATGCCGATCTCGAAAGGTTCGAGCAGCACCTGGCTCCCAACG | |
| | CCATCGGCCAGAACTTCAGCGTCGTTCAATTCAACGGCGGCCTCAAC | |
| | GACCAGCTTTCATTGAGCGACAGCGGCGAAGCCAACCTCGACCTGC | |
| | AGTACATCCTGGGCGTCAGCGCTCCCGTCCCGGTCACTGAATACAGC | |
| | ACTGGCGGACGCGGCGAACTGGTCCCCGACCTGAGCTCCCCGGACC | |
| | CCAACGACAACAGCAACGAGCCCTACCTCGACTTCCTCCAGGGTATT | |
| | CTCAAACTCGACAATTCCGACCTCCCCCAAGTCATCTCTACCTCCTAC | |
| | GGCGAAGACGAACAGACCATCCCCGTCCCCTACGCCCGCACAGTCT | |
| | GCAATCTCTACGCCCAACTCGGCAGCCGCGGTGTCTCCGTGATCTTC | |
| | TCGAGCGGCGACTCCGGCGTCGGCGCCGCCTGCCTCACCAACGAC | |
| | GGCACCAACCGCACCCACTTCCCTCCTCAATTCCCGGCCTCCTGCCC | |
| | CTGGGTAACCTCCGTCGGTGCCACCAGCAAAACCTCCCCGGAGCAA | |
| | GCCGTCTCCTTCTCCTCAGGAGGCTTCTCCGACCTCTGGCCCCGCCC | |
| | CTCCTACCAACAGGCTGCCGTCCAAACCTACCTCACCCAGCACCTGG | |
| | GCAACAAGTTCTCAGGCCTCTTCAACGCCTCCGGCCGCGCCTTCCCC | |
| | GACGTCGCCGCGCAGGGCGTCAACTACGCCGTCTACGACAAGGGCA | |
| | TGCTTGGCCAGTTCGATGGAACCAGTTGCTCCGCGCCGACGTTCAGT | |
| | GGTGTCATTGCCTTGTTGAATGACGCCAGACTGAGGGCGGGTTTGCC | |
| | CGTTATGGGATTCCTGAACCCGTTCCTCTATGGAGTTGGTAGTGAGA | |
| | GTGGCGCGTTGAATGATATTGTCAACGGCGGGAGCCTGGGTTGTGAT | |
| | GGTAGGAATCGATTTGGAGGCACGCCCAATGGAAGTCCCGTTGTGC | |
| | CGTTTGCTAGTTGGAATGCGACCACCGGGTGGGATCCGGTTTCTGG | |
| | GCTGGGAACGCCGGATTTTGCGAAGTTGAGGGGTGTGGCGTTGGGT | |
| | GAAGCTAAGGCGTATGGTAATTAA | |
| 72 | AUGGCAGCGACUGGACGAUUCACUGCCUUCUGGAAUGUCGCGAGC | Aspergillus |
| | GUGCCCGCCUUGAUUGGCAUUCUCCCCCUUGCUGGAUCUCAUUUA | nidulans |
| | AGAGCUGUCCUUUGCCCUGUCUGUAUCUGGCGUCACUCGAAGGCC | FGSC A4 |
| | GUUUGUGCACCAGACACUUUGCAAGCCAUGCGCGCCUUCACCCGU | |
| | GUAACGGCCAUCUCCCUGGCCGGUUUCUCCUGCUUCGCUGCUGCG | |
| | GCGGCUGCGGCUUUUGAGAGCCUGCGAGCUGUCCCUGACGGCUG | |
| | GAUCUACGAGAGCACCCCCGACCCUAACCAACCGCUGCGUCUACGC | |
| | AUCGCGCUGAAACAGCACAAUGUCGCCGGCUUCGAGCAGGCACUG | |
| | CUGGAUAUGUCCACACCCGGUCACUCCAGCUACGGGCAGCAUUUC | |
| | GGCUCCUACCACGAGAUGAAGCAGCUGCUUCUCCCUACCGAGGAG | |
| | GCGUCCUCCUCGGUGCGAGACUGGCUCUCGGCGGCGGGCGUUGA | |
| | GUUCGAACAGGACGCCGACUGGAUCAACUUCCGCACGACCGUCGA | |
| | CCAGGCUAACGCCCUCCUCGACGCCGAUUUCCUCUGGUACACAAC | |
| | GACCGGCUCGACGGGCAACCCGACGCGGAUCCUCCGAACCCUCUC | |
| | CUACAGCGUUCCCAGCGAGCUCGCUGGAUACGUCAACAUGAUCCA | |
| | GCCGACUACGCGUUUCGGCGGCACGCAUGCCAACCGGGCCACCGU | |
| | UCGCGCGAAGCCGAUCUUCCUCGAGACCAACCGGCAGCUCAUCAA | |
| | CGCCAUCUCCUCUGGCUCGCUCGAGCACUGCGAGAAGGCCAUCAC | |
| | CCCAUCGUGCCUGGCGGAUCUGUACAACACUGAAGGGUACAAGGC | |
| | GUCCAACCGCAGCGGGAGCAAGGUGGCCUUUGCCUCCUUCCUCGA | |
| | AGAGUACGCGCGCUACGACGAUCUCGCCGAGUUCGAGGAGACCUA | |
| | CGCUCCCUAUGCGAUCGGGCAGAACUUCUCGGUUAUCUCCAUCAA | |
| | CGGCGGCCUCAACGACCAGGACUCCACGGCCGACAGCGGCGAGGC | |
| | GAACCUCGACCUGCAGUACAUCAUCGGCGUCUCGUCGCCGCUACC | |
| | UGUGACCGAGUUCACAACCGGUGGCCGCGGCAAGCUCAUUCCUGA | |
| | CCUCUCCUCCCCCGACCCGAAUGACAACACCAACGAGCCUUUCCUU | |
| | GACUUCCUUGAGGCCGUCCUCAAGCUCGAUCAGAAAGACCUGCCC | |
| | CAGGUCAUCUCGACCUCCUACGGCGAGGACGAGCAGACAAUCCCU | |
| | GAGCCGUACGCCCGCUCCGUCUGCAACCUGUACGCUCAGCUCGGU | |
| | UCCCGCGGCGUGUCUGUGCUCUUCUCCUCGGGUGACUCUGGCGU | |
| | CGGCGCCGCCUGCCAGACCAACGAUGGCAAAAACACGACGCACUUC | |
| | CCGCCGCAGUUCCCGGCCUCUUGCCCCUGGGUGACCGCCGUCGG | |
| | CGGCACGAACGGCACAGCGCCCGAAUCCGGUGUAUACUUCUCCAG | |
| | CGGCGGGUUCUCCGACUACUGGGCGCGCCCGGCGUACCAGAACGC | |
| | CGCGGUUGAGUCAUACCUGCGCAAACUCGGUAGCACACAGGCGCA | |
| | GUACUUCAACCGCAGCGGACGCGCCUUCCCGGACGUCGCAGCGCA | |
| | GGCGCAGAACUUCGCUGUCGUCGACAAGGGCCGUGUCGGUCUCUU | |
| | CGACGGAACGAGCUGCAGUUCGCCUGUAUUUGCGGGCAUCGUGGC | |
| | GUUGCUCAACGACGUGCGUCUGAAGGCAGGCCUGCCCGUGCUGGG | |
| | AUUCCUCAACCCUUGGCUCUACCAGGAUGGCCUGAACGGGCUCAA | |
| | CGAUAUCGUGGAUGGAGGGAGCACCGGCUGCGACGGGAACAACCG | |
| | GUUUAACGGAUCGCCAAAUGGGAGCCCCGUAAUCCCGUAUGCGGG | |
| | UUGGAACGCGACGGAGGGGUGGGAUCCUGUGACGGGGCUGGGAA | |
| | CGCCGGAUUUCGCGAAGCUGAAAGCGCUCGUGCUUGAUGCUUAG | |
| 73 | ATGTTGTCATTTGTTCGTCGGGGAGCTCTCTCCCTCGCTCTCGTTTCG | Aspergillus |
| | CTGTTGACCTCGTCTGTCGCCGCCGAGGTCTTCGAGAAGCTGCATGT | ruber CBS |
| | TGTGCCCGAAGGTTGGAGATATGCCTCCACTCCTAACCCCAAACAAC | 135680 |
| | CCATTCGTCTTCAGATCGCTCTGCAGCAGCACGATGTCACCGGTTTC | |
| | GAACAGTCCCTCTTGGAGATGTCGACTCCCGACCATCCCAACTACGG | |
| | AAAACACTTCCGCACCCACGATGAGATGAAGCGCATGCTTCTCCCCA | |
| | ATGAAAATGCCGTTCACGCCGTCCGCGAATGGCTGCAAGACGCCGG | |
| | AATCAGCGACATCGAAGAAGACGCCGATTGGGTCCGTTTCCACACCA | |
| | CCGTGGACCAGGCCAACGACCTCCTCGACGCCAACTTCCTCTGGTAC | |
| | GCGCACAAGAGCCATCGTAACACGGCGCGTCTCCGCACTCTCGAGT | |
| | ACTCGATCCCAGACTCTATTGCGCCGCAGGTCAACGTGATCCAGCCA | |
| | ACCACGCGATTCGGACAGATCCGTGCCAACCGGGCTACGCATAGCA | |
| | GCAAGCCCAAGGGTGGGCTTGACGAGTTGGCTATCTCGCAGGCAGC | |
| | TACGGCGGATGATGATAGCATTTGTGACCAGATCACCACCCCACACT | |
| | GTCTGCGGAAGCTGTACAATGTCAATGGCTACAAGGCCGATCCCGCT | |
| | AGTGGTAGCAAGATCGGTTTTGCTAGTTTCCTGGAGGAATACGCGCG | |
| | GTACTCTGATCTGGTACTGTTCGAGGAGAACCTGGCACCGTTTGCGG | |
| | AGGGTGAGAACTTTACTGTCGTCATGTACAACGGCGGCAAGAATGAC | |
| | CAGAACTCCAAGAGCGACAGCGGCGAGGCCAACCTCGATCTGCAGT | |
| | ACATCGTGGGAATGAGCGCGGGCGCGCCCGTGACCGAGTTCAGCAC | |
| | CGCCGGTCGCGCACCCGTCATCCCGGACCTGGACCAGCCCGACCCC | |
| | AGCGCCGGTACCAACGAGCCGTACCTCGAGTTCCTGCAGAACGTGC | |
| | TACACATGGACCAGGAGCACCTGCCGCAGGTGATCTCTACTTCCTAC | |
| | GGTGAGAACGAACAGACCATCCCCGAAAAGTACGCCCGCACCGTTT | |
| | GCAACATGTACGCGCAGCTGGGCAGCCGCGGTGTGTCGGTGATTTT | |
| | CTCGTCGGGCGACTCCGGCGTCGGCTCTGCCTGTATGACCAACGAC | |
| | GGTACAAACCGCACCCACTTCCCCCCGCAGTTCCCGGCGTCCTGCC | |
| | CCTGGGTGACATCGGTCGGGGCCACTGAGAAGATGGCCCCCGAGCA | |
| | AGCGACATATTTCTCCTCGGGCGGCTTCTCTGACCTCTTCCCGCGCC | |
| | CAAAGTACCAGGACGCTGCTGTCAGCAGCTACCTTCAGACCCTCGGA | |
| | TCCCGGTACCAGGGCTTGTACAACGGTTCCAACCGTGCATTCCCTGA | |
| | CGTCTCGGCGCAGGGTACCAACTTTGCTGTGTACGACAAGGGCCGT | |
| | CTAGGCCAGTTCGATGGTACTTCTTGCTCTGCTCCCGCGTTTAGCGG | |
| | TATCATCGCCTTGCTCAACGACGTCCGTCTCCAGAACAACAAGCCCG | |
| | TCCTGGGCTTCTTGAACCCCTGGTTGTATGGCGCTGGGAGCAAGGG | |
| | CCTGAACGACGTCGTGCACGGTGGCAGTACAGGATGCGATGGACAG | |
| | GAGCGGTTTGCAGGAAAGGCCAATGGAAGCCCCGTCGTGCCGTACG | |
| | CTAGCTGGAATGCTACGCAAGGCTGGGATCCAGTCACTGGCCTTGGA | |
| | ACGCCGGATTTCGGCAAGTTGAAGGATTTGGCTCTGTCGGCTTAA | |
| 74 | AUGUUGCCCUCUCUUGUAAACAACGGGGCGCUGUCCCUGGCUGUG | Aspergillus |
| | CUUUCGCUGCUCACCUCGUCCGUCGCCGGCGAGGUGUUUGAGAAG | terreus |
| | CUGUCGGCCGUGCCGAAAGGAUGGCACUUCUCCCACGCUGCCCAG | NIH2624 |
| | GCCGACGCCCCCAUCAACCUGAAGAUCGCCCUGAAGCAGCAUGAU | |
| | GUCGAGGGCUUCGAGCAGGCCCUGCUGGACAUGUCCACCCCGGGC | |
| | CACGAGAACUACGGCAAGCACUUCCACGAGCACGACGAGAUGAAAC | |
| | GCAUGCUGCUCCCCAGCGACUCCGCCGUCGACGCCGUCCAGACCU | |
| | GGCUGACCUCCGCCGGCAUCACCGACUACGACCUCGACGCCGACU | |
| | GGAUCAACCUGCGCACCACCGUCGAGCACGCCAACGCCCUGCUGG | |
| | ACACGCAGUUCGGCUGGUACGAGAACGAAGUGCGCCACAUCACGC | |
| | GCCUGCGCACCCUGCAAUACUCCAUCCCCGAGACCGUCGCCGCGC | |
| | ACAUCAACAUGGUGCAGCCGACCACGCGCUUUGGCCAGAUCCGGC | |
| | CCGACCGCGCGACCUUCCACGCGCACCACACCUCCGACGCGCGCA | |
| | UCCUGUCCGCCCUGGCCGCCGCCAGCAACAGCACCAGCUGCGACU | |
| | CAGUCAUCACCCCCAAGUGCCUCAAGGACCUCUACAAGGUCGGCGA | |
| | CUACGAGGCCGACCCGGACUCGGGCAGCCAGGUCGCCUUCGCCAG | |
| | CUACCUCGAGGAAUACGCCCGCUACGCCGACAUGGUCAAGUUCCA | |
| | GAACUCGCUCGCCCCCUACGCCAAGGGCCAGAACUUCUCGGUCGU | |
| | CCUGUACAACGGCGGCGUCAACGACCAGUCGUCCAGCGCCGACUC | |
| | CGGCGAGGCCAACCUCGACCUGCAGACCAUCAUGGGCCUCAGCGC | |
| | GCCGCUCCCCAUCACCGAGUACAUCACCGGCGGCCGCGGCAAGCU | |
| | CAUCCCCGAUCUCAGCCAGCCCAACCCCAACGACAACAGCAACGAG | |
| | CCCUACCUCGAGUUCCUCCAGAACAUCCUCAAGCUGGACCAGGACG | |
| | AGCUGCCGCAGGUGAUCUCGACCUCCUACGGCGAGGACGAGCAGA | |
| | CAAUCCCCCGUGGCUACGCCGAAUCCGUCUGCAACAUGCUGGCCC | |
| | AGCUCGGCAGCCGCGGCGUGUCGGUGGUCUUCUCGUCAGGCGAU | |
| | UCGGGCGUCGGCGCCGCCUGCCAGACCAACGACGGCCGCAACCAA | |
| | ACCCACUUCAACCCGCAGUUCCCGGCCAGCUGCCCGUGGGUGACG | |
| | UCGGUCGGGGCCACGACCAAGACCAACCCGGAGCAGGCGGUGUAC | |
| | UUCUCGUCGGGCGGGUUCUCGGACUUCUGGAAGCGCCCGAAGUAC | |
| | CAGGACGAGGCGGUGGCCGCGUACCUGGACACGCUGGGCGACAAG | |
| | UUCGCGGGGCUGUUCAACAAGGGCGGGCGCGCGUUCCCGGACGU | |
| | CGCGGCGCAGGGCAUGAACUACGCCAUCUACGACAAGGGCACGCU | |
| | GGGCCGGCUGGACGGCACCUCGUGCUCGGCGCCGGCCUUCUCGG | |
| | CCAUCAUCUCGCUGCUGAACGAUGCGCGCCUGCGCGAGGGUAAGC | |
| | CGACCAUGGGCUUCUUGAACCCGUGGCUGUAUGGUGAGGGCCGCG | |
| | AGGCGCUGAAUGAUGUUGUCGUGGGUGGGAGCAAGGGCUGUGAU | |
| | GGGCGCGACCGGUUUGGCGGCAAGCCCAAUGGGAGCCCUGUCGU | |
| | GCCUUUUGCUAGCUGGAAUGCUACGCAGGGCUGGGACCCGGUUAC | |
| | UGGGCUGGGGACGCCGAACUUUGCGAAGAUGUUGGAGCUGGCGC | |
| | CAUAG | |
| 75 | ATGATTGCATCATTATTCAACCGTAGGGCATTGACGCTCGCTTTATTG | Penicillium |
| | TCACTTTTTGCATCCTCTGCCACAGCCGATGTTTTTGAGAGTTTGTCT | digitatum |
| | GCTGTTCCTCAGGGATGGAGATATTCTCGCACACCGAGTGCTAATCA | Pd1 |
| | GCCCTTGAAGCTACAGATTGCTCTGGCTCAGGGAGATGTTGCTGGGT | |
| | TCGAGGCAGCTGTGATCGATATGTCAACCCCCGACCACCCCAGTTAC | |
| | GGGAACCACTTCAACACCCACGAGGAAATGAAGCGGATGCTGCAGC | |
| | CTAGCGCGGAGTCCGTAGACTCGATCCGTAACTGGCTCGAAAGTGC | |
| | CGGTATTTCCAAGATCGAACAGGACGCTGACTGGATGACCTTCTATA | |
| | CCACCGTGAAGACAGCGAATGAGCTGCTGGCAGCCAACTTCCAGTTC | |
| | TACATCAATGGAGTCAAGAAAATAGAGCGTCTCCGCACACTCAAGTA | |
| | CTCTGTCCCGGACGCTTTGGTGTCCCACATTAACATGATCCAGCCAA | |
| | CCACCCGTTTCGGCCAGCTGCGCGCCCAGCGCGCCATTTTACACAC | |
| | CGAGGTCAAGGATAACGACGAGGCTTTCCGCTCAAATGCCATGTCCG | |
| | CTAATCCGGACTGCAACAGCATCATCACTCCCCAGTGTCTCAAGGAT | |
| | TTGTACAGTATCGGTGACTATGAGGCCGACCCCACCAATGGGAACAA | |
| | GGTCGCGTTTGCCAGCTACCTAGAGGAGTATGCCCGATACTCCGATC | |
| | TCGCATTATTTGAGAAAAACATCGCCCCCTTTGCCAAGGGACAGAATT | |
| | TCTCCGTTGTCCAGTATAACGGCGGTGGTAATGATCAACAATCGAGC | |
| | AGTGGCAGTAGTGAGGCGAATCTTGACTTGCAGTACATCGTTGGAGT | |
| | CAGCTCTCCTGTTCCCGTTACAGAGTTTAGCACTGGAGGTCGCGGTG | |
| | AACTTGTTCCGGATCTCGACCAGCCGAATCCCAATGACAACAACAAC | |
| | GAGCCATACCTTGAATTCCTCCAGAACGTGCTCAAGTTGCACAAGAA | |
| | GGACCTCCCCCAGGTGATTTCCACCTCTTATGGCGAGGACGAGCAGA | |
| | GCGTTCCAGAGAAGTACGCCCGCGCCGTTTGCAACCTGTACTCCCAA | |
| | CTCGGTAGCCGTGGTGTGTCCGTAATCTTTTCATCCGGCGACTCTGG | |
| | CGTTGGCGCCGCGTGTCAGACGAACGACGGCCGGAACGCGACCCA | |
| | CTTCCCACCCCAGTTCCCGGCCGCCTGCCCCTGGGTGACATCAGTC | |
| | GGTGCGACAACCCACACTGCGCCCGAACGAGCCGTTTACTTCTCATC | |
| | TGGCGGTTTCTCCGATCTCTGGGATCGCCCTACGTGGCAAGAAGATG | |
| | CTGTGAGTGAGTACCTCGAGAACCTGGGCGACCGCTGGTCTGGCCT | |
| | CTTCAACCCTAAGGGCCGTGCCTTCCCCGACGTCGCAGCCCAGGGT | |
| | GAAAACTACGCCATCTACGATAAGGGTTCTTTGATCAGCGTCGATGG | |
| | CACCTCTTGCTCGGCACCTGCGTTTGCCGGAGTCATCGCCCTCCTCA | |
| | ACGACGCCCGCATCAAGGCCAATAGACCACCCATGGGCTTCCTCAAC | |
| | CCTTGGCTGTACTCTGAAGGCCGCAGCGGCCTAAACGACATTGTCAA | |
| | CGGCGGTAGCACTGGCTGCGACGGTCATGGCCGCTTCTCCGGCCCC | |
| | ACTAACGGTGGTACGTCGATTCCAGGTGCCAGCTGGAACGCTACTAA | |
| | GGGCTGGGACCCTGTCTCCGGTCTTGGATCGCCCAACTTTGCTGCCA | |
| | TGCGCAAACTCGCCAACGCTGAGTAG | |
| 76 | ATGCATGTTCCTCTGTTGAACCAAGGCGCGCTGTCGCTGGCCGTCGT | Penicillium |
| | CTCGCTGTTGGCCTCCACGGTCTCGGCCGAAGTATTCGACAAGCTTG | oxalicum |
| | TCGCTGTCCCTGAAGGATGGCGATTCTCCCGCACTCCCAGTGGAGAC | 114-2 |
| | CAGCCCATCCGACTGCAGGTTGCCCTCACACAGGGTGACGTTGAGG | |
| | GCTTCGAGAAGGCCGTTCTGGACATGTCAACTCCCGACCACCCCAAC | |
| | TATGGCAAGCACTTCAAGTCACACGAGGAAGTTAAGCGCATGCTGCA | |
| | GCCTGCAGGCGAGTCCGTCGAAGCCATCCACCAGTGGCTCGAGAAG | |
| | GCCGGCATCACCCACATTCAACAGGATGCCGACTGGATGACCTTCTA | |
| | CACCACCGTTGAGAAGGCCAACAACCTGCTGGATGCCAACTTCCAGT | |
| | ACTACCTCAACGAGAACAAGCAGGTCGAGCGTCTGCGCACCTTGGA | |
| | GTACTCGGTTCCTGACGAGCTCGTCTCGCACATTAACCTTGTCACCC | |
| | CGACCACTCGCTTCGGCCAGCTGCACGCCGAGGGTGTGACGCTGCA | |
| | CGGCAAGTCTAAGGACGTCGACGAGCAATTCCGCCAGGCTGCTACTT | |
| | CCCCTAGCAGCGACTGCAACAGTGCTATCACCCCGCAGTGCCTCAAG | |
| | GACCTGTACAAGGTCGGCGACTACAAGGCCAGTGCCTCCAATGGCA | |
| | ACAAGGTCGCCTTCACCAGCTACCTGGAGCAGTACGCCCGGTACTC | |
| | GGACCTGGCTCTGTTTGAGCAGAACATTGCCCCCTATGCTCAGGGCC | |
| | AGAACTTCACCGTTATCCAGTACAACGGTGGTCTGAACGACCAGAGC | |
| | TCGCCTGCGGACAGCAGCGAGGCCAACCTGGATCTCCAGTACATTAT | |
| | CGGAACGAGCTCTCCCGTCCCCGTGACTGAGTTCAGCACCGGTGGT | |
| | CGTGGTCCCTTGGTCCCCGACTTGGACCAGCCTGACATCAACGACAA | |
| | CAACAACGAGCCTTACCTCGACTTCTTGCAGAATGTCATCAAGATGAG | |
| | CGACAAGGATCTTCCCCAGGTTATCTCCACCTCGTACGGTGAGGACG | |
| | AGCAGAGCGTCCCCGCAAGCTACGCTCGTAGCGTCTGCAACCTCATC | |
| | GCTCAGCTCGGCGGCCGTGGTGTCTCCGTGATCTTCTCATCTGGTGA | |
| | TTCCGGTGTGGGCTCTGCCTGTCAGACCAACGACGGCAAGAACACC | |
| | ACTCGCTTCCCCGCTCAGTTCCCCGCCGCCTGCCCCTGGGTGACCT | |
| | CTGTTGGTGCTACTACCGGTATCTCCCCCGAGCGCGGTGTCTTCTTC | |
| | TCCTCCGGTGGCTTCTCCGACCTCTGGAGCCGCCCCTCGTGGCAAA | |
| | GCCACGCCGTCAAGGCCTACCTTCACAAGCTTGGCAAGCGTCAAGAC | |
| | GGTCTCTTCAACCGCGAAGGCCGTGCGTTCCCCGACGTGTCAGCCC | |
| | AGGGTGAGAACTACGCTATCTACGCGAAGGGTCGTCTCGGCAAGGTT | |
| | GACGGCACTTCCTGCTCGGCTCCCGCTTTCGCCGGTCTGGTTTCTCT | |
| | GCTGAACGACGCTCGCATCAAGGCGGGCAAGTCCAGCCTCGGCTTC | |
| | CTGAACCCCTGGTTGTACTCGCACCCCGATGCCTTGAACGACATCAC | |
| | CGTCGGTGGAAGCACCGGCTGCGACGGCAACGCTCGCTTCGGTGGT | |
| | CGTCCCAACGGCAGTCCCGTCGTCCCTTACGCTAGCTGGAACGCTAC | |
| | TGAGGGCTGGGACCCCGTCACCGGTCTGGGTACTCCCAACTTCCAG | |
| | AAGCTGCTCAAGTCTGCCGTTAAGCAGAAGTAA | |
| 77 | ATGATTGCATCCCTATTTAGTCGTGGAGCATTGTCGCTCGCGGTCTTG | Penicillium |
| | TCGCTTCTCGCGTCCTCTGCTGCAGCCGATGTATTTGAGAGTTTGTCT | roqueforti |
| | GCTGTTCCTCAAGGATGGAGATATTCTCGCAGGCCGCGTGCTGATCA | FM164 |
| | GCCCTTGAAGTTACAGATCGCTCTGACACAGGGGGATACTGCCGGCT | |
| | TCGAAGAGGCTGTGATGGAGATGTCAACCCCCGATCACCCTAGCTAC | |
| | GGGCACCACTTCACCACCCACGAAGAAATGAAGCGGATGCTACAGC | |
| | CCAGTGCGGAGTCCGCGGAGTCAATCCGTGACTGGCTCGAAGGCGC | |
| | GGGTATTACCAGGATCGAACAGGATGCAGATTGGATGACCTTCTACA | |
| | CCACCGTGGAGACGGCAAATGAGCTGCTGGCAGCCAATTTCCAGTTC | |
| | TACGTCAGTAATGTCAGGCACATTGAGCGTCTTCGCACACTCAAGTA | |
| | CTCAGTCCCGAAGGCTCTGGTGCCACACATCAACATGATCCAGCCAA | |
| | CCACCCGTTTCGGCCAGCTGCGCGCCCATCGGGGCATATTACACGG | |
| | CCAGGTCAAGGAATCCGACGAGGCTTTCCGCTCAAACGCCGTGTCC | |
| | GCTCAGCCGGATTGCAACAGTATCATCACTCCTCAGTGTCTCAAGGA | |
| | TATATATAATATCGGTGATTACCAGGCCAATGATACCAATGGGAACAA | |
| | GGTCGGGTTTGCCAGCTACCTAGAGGAGTATGCACGATACTCCGATC | |
| | TGGCACTATTTGAGAAAAATATCGCGCCCTCTGCCAAGGGCCAGAAC | |
| | TTCTCCGTCACCAGGTACAACGGCGGTCTTAATGATCAAAGTTCCAG | |
| | CGGTAGCAGCAGCGAGGCGAACCTGGACTTGCAGTACATTGTTGGA | |
| | GTCAGCTCTCCTGTTCCCGTCACCGAATTTAGCGTTGGCGGCCGTGG | |
| | TGAACTTGTTCCCGATCTCGACCAGCCTGATCCCAATGATAACAACAA | |
| | CGAGCCATACCTTGAATTCCTCCAGAACGTGCTCAAGCTGGACAAAA | |
| | AGGACCTTCCCCAGGTGATTTCTACCTCCTATGGTGAGGACGAGCAG | |
| | AGCATTCCCGAGAAGTACGCCCGCAGTGTTTGCAACTTGTACTCGCA | |
| | GCTCGGTAGCCGTGGTGTATCCGTCATTTTCTCATCTGGCGACTCCG | |
| | GCGTTGGGTCCGCGTGCCTGACGAACGACGGCAGGAACGCGACCC | |
| | GCTTCCCACCCCAGTTCCCCGCCGCCTGCCCGTGGGTGACATCAGT | |
| | CGGCGCGACAACCCATACCGCGCCCGAACAGGCCGTGTACTTCTCG | |
| | TCCGGCGGCTTTTCCGATCTCTGGGCTCGCCCGAAATGGCAAGAGG | |
| | AGGCCGTGAGTGAGTACCTCGAGATCCTGGGTAACCGCTGGTCTGG | |
| | CCTCTTCAACCCTAAGGGTCGTGCCTTCCCCGATGTCACAGCCCAAG | |
| | GTCGCAATTACGCTATATACGATAAGGGCTCGTTGACCAGCGTCGAC | |
| | GGCACCTCCTGCTCGGCACCTGCCTTCGCCGGAGTCGTCGCCCTCC | |
| | TCAACGACGCTCGCCTCAAAGTCAACAAACCACCAATGGGCTTCCTT | |
| | AATCCTTGGCTGTACTCGACAGGGCGCGCCGGCCTAAAGGACATTGT | |
| | CGATGGCGGCAGCACGGGTTGCGATGGCAAGAGCCGCTTCGGTGGT | |
| | GCCAATAACGGTGGTCCGTCGATCCCAGGTGCTAGCTGGAACGCTA | |
| | CTAAGGGTTGGGACCCTGTTTCTGGTCTCGGGTCGCCCAACTTTGCT | |
| | ACCATGCGCAAGCTTGCGAACGCTGAGTAG | |
| 78 | AUGAUUGCAUCUCUAUUUAACCGUGGAGCAUUGUCGCUCGCGGUA | Penicillium |
| | UUGUCGCUUCUCGCGUCUUCGGCUUCCGCUGAUGUAUUUGAGAGU | rubens |
| | UUGUCUGCUGUUCCUCAAGGAUGGAGAUAUUCUCGCAGACCGCGU | Wisconsin |
| | GCUGAUCAGCCCCUGAAGCUACAGAUUGCUCUGGCACAAGGGGAU | 54-1255 |
| | ACUGCCGGAUUCGAAGAGGCUGUGAUGGACAUGUCAACCCCUGAU | |
| | CACCCCAGCUACGGGAACCACUUCCACACCCACGAGGAAAUGAAGC | |
| | GGAUGCUGCAGCCCAGCGCGGAGUCCGCAGACUCGAUCCGUGACU | |
| | GGCUUGAAAGUGCGGGUAUCAAUAGAAUUGAACAGGAUGCCGACU | |
| | GGAUGACAUUCUACACCACCGUCGAGACGGCAAAUGAGCUGCUGG | |
| | CAGCCAAUUUCCAGUUCUAUGCCAACAGUGCCAAGCACAUUGAGCG | |
| | UCUUCGCACACUCCAGUACUCCGUCCCGGAGGCUCUGAUGCCACA | |
| | CAUCAACAUGAUCCAGCCAACCACUCGUUUCGGCCAGCUGCGCGU | |
| | CCAGGGGGCCAUAUUGCACACCCAGGUCAAGGAAACCGACGAGGC | |
| | UUUCCGCUCAAACGCCGUGUCCACUUCACCGGACUGCAACAGUAUC | |
| | AUCACUCCUCAGUGUCUCAAGAAUAUGUACAAUGUGGGUGACUACC | |
| | AGGCCGACGACGACAAUGGGAACAAGGUCGGAUUUGCCAGCUACC | |
| | UAGAGGAGUAUGCACGGUACUCCGAUUUGGAACUAUUUGAGAAAAA | |
| | UGUCGCACCCUUCGCCAAGGGCCAGAACUUCUCCGUCAUCCAGUA | |
| | UAACGGCGGUCUUAACGAUCAACACUCGAGUGCUAGCAGCAGCGA | |
| | GGCGAACCUUGACUUACAGUACAUUGUUGGAGUUAGCUCUCCUGU | |
| | UCCAGUUACAGAGUUUAGCGUUGGCGGUCGUGGUGAACUUGUUCC | |
| | CGAUCUUGACCAGCCUGAUCCCAAUGAUAACAACAACGAGCCAUAC | |
| | CUUGAAUUCCUCCAGAACGUGCUCAAGAUGGAACAACAGGACCUCC | |
| | CCCAGGUGAUUUCCACCUCUUAUGGCGAGAACGAGCAGAGUGUUC | |
| | CCGAGAAAUACGCCCGCACCGUAUGCAACUUGUUCUCGCAGCUUG | |
| | GCAGCCGUGGUGUGUCCGUCAUCUUCGCAUCUGGCGACUCCGGCG | |
| | UUGGCGCCGCGUGCCAGACGAAUGACGGCAGGAACGCGACCCGCU | |
| | UCCCGGCCCAGUUCCCUGCUGCCUGCCCAUGGGUGACAUCGGUCG | |
| | GCGCGACAACCCACACCGCGCCCGAGAAGGCCGUGUACUUCUCGU | |
| | CCGGUGGCUUCUCCGAUCUUUGGGAUCGCCCGAAAUGGCAAGAAG | |
| | ACGCCGUGAGUGACUACCUCGACACCCUGGGCGACCGCUGGUCCG | |
| | GCCUCUUCAAUCCUAAGGGCCGUGCCUUCCCCGACGUCUCAGCCC | |
| | AAGGUCAAAACUACGCCAUAUACGAUAAGGGCUCGUUGACCAGCGU | |
| | CGACGGCACCUCGUGCUCGGCACCCGCCUUCGCCGGUGUCAUCGC | |
| | CCUCCUCAACGACGCCCGCCUCAAGGCCAACAAACCACCCAUGGGC | |
| | UUCCUCAAUCCCUGGCUGUACUCGACAGGCCGUGACGGCCUGAAC | |
| | GACAUUGUUCAUGGCGGCAGCACUGGCUGUGAUGGCAACGCCCGC | |
| | UUCGGCGGCCCCGGUAACGGCAGUCCGAGGGUUCCAGGUGCCAGC | |
| | UGGAACGCUACUAAGGGCUGGGACCCUGUUUCUGGUCUUGGAUCA | |
| | CCCAACUUUGCUACCAUGCGCAAGCUCGCGAACGGUGAGUAG | |
| 79 | AUGCUGUCCUCGACUCUCUACGCAGGGUUGCUCUGCUCCCUCGCA | Neosartorya |
| | GCCCCAGCCCUUGGUGUGGUGCACGAGAAGCUCUCAGCUGUUCCU | fischeri |
| | AGUGGCUGGACACUCGUCGAGGAUGCAUCGGAGAGCGACACGACC | NRRL 181 |
| | ACUCUCUCAAUUGCCCUUGCUCGGCAGAACCUCGACCAGCUCGAG | |
| | UCCAAGUUGACCACACUGGCGACCCCAGGGAACGCGGAGUACGGC | |
| | AAGUGGCUGGACCAGUCCGACAUUGAGUCCCUAUUUCCUACUGCA | |
| | AGCGAUGACGCUGUUAUCCAAUGGCUCAAGGAUGCCGGGGUCACC | |
| | CAAGUGUCUCGUCAGGGCAGCUUGGUGAACUUUGCCACCACUGUG | |
| | GGAACGGCGAACAAGCUCUUUGACACCAAGUUCUCCUACUACCGCA | |
| | AUGGUGCUUCCCAGAAACUGCGUACCACGCAGUACUCCAUUCCCGA | |
| | UAGCCUGACAGAGUCGAUCGAUCUGAUUGCCCCCACUGUCUUCUU | |
| | UGGCAAGGAGCAAGACAGCGCACUGCCACCUCACGCAGUGAAGCU | |
| | UCCAGCCCUUCCCAGGAGGGCAGCCACCAACAGUUCUUGCGCCAA | |
| | CCUGAUCACUCCCGACUGCCUAGUGGAGAUGUACAACCUCGGCGA | |
| | CUACAAGCCUGAUGCAUCUUCGGGCAGUCGAGUCGGCUUUGGUAG | |
| | CUUCUUGAAUCAGUCAGCCAACUAUGCAGAUCUGGCUGCUUAUGA | |
| | GCAACUGUUCAACAUCCCACCCCAGAAUUUCUCAGUCGAAUUGAUU | |
| | AACGGAGGCGCCAAUGAUCAGAAUUGGGCCACUGCUUCCCUCGGC | |
| | GAGGCCAAUCUGGACGUGGAGUUGAUUGUAGCCGUCAGCCACGCC | |
| | CUGCCAGUAGUGGAGUUUAUCACUGGCGGUUCACCUCCGUUUGUU | |
| | CCCAAUGUCGACGAGCCAACCGCUGCGGACAACCAGAAUGAGCCCU | |
| | ACCUCCAGUACUACGAGUACUUGCUCUCCAAACCCAACUCCCAUCU | |
| | UCCUCAGGUGAUUUCCAACUCGUAUGGUGACGAUGAACAGACUGU | |
| | UCCCGAGUACUACGCCAGGAGAGUUUGCAACUUGAUCGGCUUGAU | |
| | GGGUCUUCGUGGUAUCACUGUGCUCGAGUCCUCUGGUGAUACCGG | |
| | AAUCGGCUCGGCGUGCAUGUCCAAUGACGGCACCAACACGCCUCA | |
| | GUUCACUCCUACAUUCCCUGGCACCUGCCCCUUCAUCACCGCAGU | |
| | UGGUGGUACACAGUCCUAUGCUCCUGAAGUUGCCUGGGACGCCAG | |
| | CUCGGGUGGAUUCAGCAACUACUUCAGCCGUCCCUGGUACCAGUA | |
| | UUUCGCGGUGGAGAACUACCUCAAUAAUCACAUUACCAAGGACACC | |
| | AAGAAGUACUAUUCGCAGUACACCAACUUCAAGGGCCGUGGAUUCC | |
| | CUGAUGUUUCUGCCCAUAGCUUGACCCCUGACUACGAGGUCGUCC | |
| | UAACUGGCAAACAUUACAAGUCCGGUGGCACAUCGGCCGCCUGCC | |
| | CCGUCUUUGCUGGUAUCGUCGGCCUGUUGAAUGACGCCCGUCUGC | |
| | GCGCCGGCAAGUCCACCCUUGGCUUCCUGAACCCAUUGCUGUAUA | |
| | GCAUACUCGCGGAAGGAUUCACCGAUAUCACUGCCGGAAGUUCUA | |
| | UCGGUUGUAAUGGUAUCAACCCACAGACCGGAAAGCCAGUCCCCG | |
| | GUGGUGGUAUCAUCCCCUACGCUCACUGGAACGCUACUGCCGGCU | |
| | GGGAUCCUGUUACAGGUCUUGGGGUUCCUGAUUUCAUGAAGUUGA | |
| | AGGAGUUGGUUUUGUCGUUGUAA | |
| 80 | AUGCUGUCCUCGACUCUCUACGCAGGGUGGCUCCUCUCCCUCGCA | Aspergillus |
| | GCCCCAGCCCUUUGUGUGGUGCAGGAGAAGCUCUCAGCUGUUCCU | fumigatus |
| | AGUGGCUGGACACUCAUCGAGGAUGCAUCGGAGAGCGACACGAUC | CAE17675 |
| | ACUCUCUCAAUUGCCCUUGCUCGGCAGAACCUCGACCAGCUUGAG | |
| | UCCAAGCUGACCACGCUGGCGACCCCAGGGAACCCGGAGUACGGC | |
| | AAGUGGCUGGACCAGUCCGACAUUGAGUCCCUAUUUCCUACUGCA | |
| | AGCGAUGAUGCUGUUCUCCAAUGGCUCAAGGCGGCCGGGAUUACC | |
| | CAAGUGUCUCGUCAGGGCAGCUUGGUGAACUUCGCCACCACUGUG | |
| | GGAACAGCGAACAAGCUCUUUGACACCAAGUUCUCUUACUACCGCA | |
| | AUGGUGCUUCCCAGAAACUGCGUACCACGCAGUACUCCAUCCCCGA | |
| | UCACCUGACAGAGUCGAUCGAUCUGAUUGCCCCCACUGUCUUCUU | |
| | UGGCAAGGAGCAGAACAGCGCACUGUCAUCUCACGCAGUGAAGCU | |
| | UCCAGCUCUUCCUAGGAGGGCAGCCACCAACAGUUCUUGCGCCAA | |
| | CCUGAUCACCCCCGACUGCCUAGUGGAGAUGUACAACCUCGGCGA | |
| | CUACAAACCUGAUGCAUCUUCGGGAAGUCGAGUCGGCUUCGGUAG | |
| | CUUCUUGAAUGAGUCGGCCAACUAUGCAGAUUUGGCUGCGUAUGA | |
| | GCAACUCUUCAACAUCCCACCCCAGAAUUUCUCAGUCGAAUUGAUC | |
| | AACAGAGGCGUCAAUGAUCAGAAUUGGGCCACUGCUUCCCUCGGC | |
| | GAGGCCAAUCUGGACGUGGAGUUGAUUGUAGCCGUCAGCCACCCC | |
| | CUGCCAGUAGUGGAGUUUAUCACUGGCGCCCUACCUCCAGUACUA | |
| | CGAGUACUUGCUCUCCAAACCCAACUCCCAUCUUCCUCAGGUGAUU | |
| | UCCAACUCACUGUUCCCGAGUACUACGCCAGGAGAGUUUGCAACUU | |
| | GAUCGGCUUGAUGGGUCUUCGUGGCAUCACGGUGCUCGAGUCCUC | |
| | UGGUGAUACCGGAAUCGGCUCGGCAUGCAUGUCCAAUGACGGCAC | |
| | CAACAAGCCCCAAUUCACUCCUACAUUCCCUGGCACCUGCCCCUUC | |
| | AUCACCGCAGUUGGUGGUACUCAGUCCUAUGCUCCUGAAGUUGCU | |
| | UGGGACGGCAGUUCCGGCGGAUUCAGCAACUACUUCAGCCGUCCC | |
| | UGGUACCAGUCUUUCGCGGUGGACAACUACCUCAACAACCACAUUA | |
| | CCAAGGAUACCAAGAAGUACUAUUCGCAGUACACCAACUUCAAGGG | |
| | CCGUGGAUUCCCUGAUGUUUCCGCCCAUAGUUUGACCCCUUACUA | |
| | CGAGGUCGUCUUGACUGGCAAACACUACAAGUCUGGCGGCACAUC | |
| | CGCCGCCAGCCCCGUCUUUGCCGGUAUUGUCGGUCUGCUGAACGA | |
| | CGCCCGUCUGCGCGCCGGCAAGUCCACUCUUGGCUUCCUGAACCC | |
| | AUUGCUGUAUAGCAUCCUGGCCGAAGGAUUCACCGAUAUCACUGC | |
| | CGGAAGUUCAAUCGGUUGUAAUGGUAUCAACCCACAGACCGGAAAG | |
| | CCAGUUCCUGGUGGUGGUAUUAUCCCCUACGCUCACUGGAACGCU | |
| | ACUGCCGGCUGGGAUCCUGUUACUGGCCUUGGGGUUCCUGAUUUC | |
| | AUGAAAUUGAAGGAGUUGGUUCUGUCGUUGUAA | |
| 81 | ATGCAGACCTTCGGTGCTTTTCTCGTTTCCTTCCTCGCCGCCAGCGG | Phaeosphaer |
| | CCTGGCCGCGGCCGAGCCCTTCGAGAAGCTCTTTAGCACCCCCGAG | ianodorum |
| | GGCTGGAAGATGCAGGGCCTCGCCACCAACGAGCAGATCGTCAAGC | SN15 |
| | TCCAGATCGCCCTCCAGCAGGGCGACGTGGCCGGCTTTGAGCAGCA | |
| | CGTCATCGACATCAGCACCCCCAGCCACCCCAGCTACGGCGCTCAC | |
| | TACGGCAGCCACGAAGAGATGAAGCGCATGATCCAGCCCAGCAGCG | |
| | AGACTGTCGCCAGCGTCAGCGCCTGGCTCAAGGCCGCTGGCATCAA | |
| | CGACGCCGAGATCGACAGCGACTGGGTCACCTTCAAGACCACCGTC | |
| | GGCGTCGCCAACAAGATGCTCGACACCAAGTTCGCCTGGTACGTCA | |
| | GCGAGGAAGCCAAGCCCCGCAAGGTCCTCCGCACCCTTGAGTACAG | |
| | CGTCCCCGACGACGTCGCCGAGCACATCAACCTCATCCAGCCCACC | |
| | ACCCGCTTCGCCGCCATCCGCCAGAACCACGAGGTCGCCCACGAGA | |
| | TCGTCGGCCTCCAGTTTGCCGCCCTCGCCAACAACACCGTCAACTGC | |
| | GACGCCACCATCACCCCCCAGTGCCTCAAGACCCTCTACAAGATCGA | |
| | CTACAAGGCCGACCCCAAGAGCGGCAGCAAGGTCGCCTTCGCCAGC | |
| | TACCTTGAGCAGTACGCCCGCTACAACGACCTCGCCCTCTTCGAGAA | |
| | GGCCTTCCTGCCTGAGGCCGTCGGCCAGAACTTCAGCGTCGTCCAG | |
| | TTCTCTGGCGGCCTCAACGACCAGAACACCACCCAGGATAGCGGCG | |
| | AGGCCAACCTCGACCTCCAGTACATCGTCGGCGTCAGCGCCCCTCT | |
| | GCCCGTCACCGAGTTTAGCACTGGCGGCCGAGGCCCTTGGGTCGCC | |
| | GATCTCGATCAGCCTGACGAGGCCGACAGCGCCAACGAGCCCTACC | |
| | TTGAGTTCCTCCAGGGCGTCCTCAAGCTCCCCCAGAGCGAGCTGCC | |
| | CCAGGTCATCAGCACCTCGTACGGCGAGAACGAGCAGAGCGTCCCC | |
| | AAGAGCTACGCCCTCAGCGTCTGCAACCTCTTCGCCCAGCTTGGCTC | |
| | TCGCGGCGTCAGCGTCATCTTCAGCAGCGGCGATAGCGGCCCTGGC | |
| | AGCGCCTGCCAGTCTAACGACGGCAAGAACACCACCAAGTTCCAGC | |
| | CCCAGTACCCTGCCGCCTGCCCCTTCGTCACTAGCGTCGGCTCTACC | |
| | CGCTACCTCAACGAGACTGCCACCGGCTTCAGCTCCGGCGGCTTCA | |
| | GCGACTACTGGAAGCGCCCCAGCTACCAGGACGACGCCGTCAAGGC | |
| | CTACTTCCACCACCTCGGCGAGAAGTTCAAGCCCTACTTCAACCGCC | |
| | ACGGCCGAGGCTTCCCTGACGTCGCCACTCAGGGCTACGGCTTCCG | |
| | CGTCTACGACCAGGGCAAGCTCAAGGGCCTCCAGGGCACTTCTGCC | |
| | AGCGCCCCTGCCTTCGCCGGCGTCATTGGCCTGCTCAACGACGCCC | |
| | GCCTCAAGGCCAAGAAGCCCACCCTCGGCTTTCTCAACCCCCTGCTC | |
| | TACAGCAACAGCGACGCCCTCAACGACATCGTCCTCGGCGGCTCCA | |
| | AGGGCTGCGACGGCCACGCTAGGTTTAACGGCCCTCCCAACGGCAG | |
| | CCCCGTCATCCCTTACGCCGGCTGGAACGCCACTGCCGGCTGGGAC | |
| | CCTGTTACCGGCCTCGGCACCCCCAACTTCCCCAAGCTCCTCAAGGC | |
| | CGCCGTCCCCTCTCGATACCGCGCTTAA | |
| 82 | ATGCAGACCTTCGGTGCTTTTCTCGTTTCCTTCCTCGCCGCCAGCGG | Trichoderma |
| | CCTGGCCGCGGCCAACGCTGCTGTCCTCCTCGACAGCCTCGACAAG | atroviride IMI |
| | GTCCCCGTCGGCTGGCAGGCTGCTTCTGCCCCTGCTCCCAGCAGCA | 206040 |
| | AGATCACCCTCCAGGTCGCCCTCACCCAGCAGAACATCGACCAGCTT | |
| | GAGAGCAAGCTCGCCGCCGTCAGCACCCCCAACAGCAGCAACTACG | |
| | GCAAGTACCTCGACGTCGACGAGATCAACCAGATCTTCGCCCCCAGC | |
| | AGCGCCAGCACTGCCGCTGTCGAGAGCTGGCTCAAGAGCTACGGCG | |
| | TCGACTACAAGGTCCAGGGCAGCAGCATCTGGTTCCAGACCGACGT | |
| | CAGCACGGCCAACAAGATGCTCAGCACCAACTTCCACACCTACACCG | |
| | ACAGCGTCGGCGCCAAGAAGGTCCGCACCCTCCAGTACAGCGTCCC | |
| | CGAGACTCTCGCCGACCACATCGACCTCATCAGCCCCACCACCTACT | |
| | TCGGCACCAGCAAGGCCATGCGAGCCCTCAAGATCCAGAACGCCGC | |
| | CAGCGCCGTCAGCCCTCTCGCTGCTCGACAAGAGCCCAGCAGCTGC | |
| | AAGGGCACCATCGAGTTCGAGAACCGCACCTTCAACGTCTTTCAGCC | |
| | CGACTGCCTCCGCACCGAGTACAGCGTCAACGGCTACAAGCCCAGC | |
| | GCCAAGAGCGGCAGCCGAATCGGCTTCGGCAGCTTCCTCAACCAGA | |
| | GCGCCAGCAGCAGCGACCTCGCCCTCTTCGAGAAGCACTTCGGCTT | |
| | CGCCAGCCAGGGCTTCAGCGTCGAGCTGATCAACGGCGGCAGCAAC | |
| | CCCCAGCCTCCCACCGATGCTAACGACGGCGAGGCCAACCTCGACG | |
| | CCCAGAACATCGTCAGCTTCGTCCAGCCCCTGCCCATCACCGAGTTT | |
| | ATCGCTGGCGGCACCGCCCCCTACTTCCCCGATCCTGTTGAGCCTG | |
| | CCGGCACCCCCGACGAGAACGAGCCCTACCTTGAGTACTACGAGTA | |
| | CCTCCTCAGCAAGAGCAACAAGGAACTCCCCCAGGTCATCACCAACA | |
| | GCTACGGCGACGAGGAACAGACCGTCCCCCAGGCCTACGCCGTCCG | |
| | CGTCTGCAACCTCATCGGCCTCATGGGCCTCCGCGGCATCAGCATC | |
| | CTTGAGAGCAGCGGCGACGAGGGCGTCGGCGCTTCTTGCCTCGCCA | |
| | CCAACAGCACCACCACCCCCCAGTTCAACCCCATCTTCCCCGCCACG | |
| | TGCCCCTACGTCACTAGCGTCGGCGGCACCGTCAGCTTCAACCCCG | |
| | AGGTCGCTTGGGACGGCAGCAGCGGCGGCTTCAGCTACTACTTCAG | |
| | CCGCCCCTGGTATCAAGAGGCCGCCGTCGGCACCTACCTCAACAAG | |
| | TACGTCAGCGAGGAAACGAAGGAATATTACAAGAGCTACGTCGACTT | |
| | CAGCGGCCGAGGCTTCCCTGACGTCGCCGCTCACTCTGTCAGCCCC | |
| | GACTACCCCGTCTTTCAGGGCGGCGAGCTGACTCCTTCTGGCGGCA | |
| | CTTCTGCCGCCAGCCCCATCGTCGCCAGCGTCATTGCCCTGCTCAAC | |
| | GACGCCCGACTCCGAGCCGGCAAGCCTGCCCTCGGCTTTCTCAACC | |
| | CCCTCATCTACGGCTACGCCTACAAGGGCTTCACCGACATCACCTCC | |
| | GGCCAGGCCGTTGGCTGCAACGGCAACAACACCCAGACCGGCGGAC | |
| | CCCTTCCTGGCGCTGGCGTTATCCCTGGCGCCTTCTGGAACGCCAC | |
| | CAAGGGCTGGGACCCCACCACCGGCTTTGGCGTCCCCAACTTCAAG | |
| | AAGCTCCTTGAGCTGGTCCGCTACATC | |
| 83 | ATGCAGACCTTCGGTGCTTTTCTCGTTTCCTTCCTCGCCGCCAGCGG | Arthroderma |
| | CCTGGCCGCGGCCAAGCCTACTCCTGGCGCTTCCCACAAGGTCATC | benhamiae |
| | GAGCACCTCGACTTCGTCCCCGAGGGCTGGCAGATGGTCGGCGCTG | CBS 112371 |
| | CTGACCCTGCCGCCATCATCGACTTTTGGCTCGCCATCGAGCGCGAG | |
| | AACCCCGAGAAGCTCTACGACACCATCTACGACGTCAGCACCCCCG | |
| | GACGCGCCCAGTACGGCAAGCACCTCAAGCGCGAGGAACTCGACGA | |
| | CCTCCTCCGCCCTCGCGCCGAGACTAGCGAGAGCATCATCAACTGG | |
| | CTCACCAACGGCGGCGTCAACCCCCAGCACATTCGCGACGAGGGCG | |
| | ACTGGGTCCGCTTCAGCACCAACGTCAAGACCGCCGAGACTCTCATG | |
| | AACACCCGCTTCAACGTCTTTAAGGACAACCTCAACAGCGTCAGCAA | |
| | GATCCGCACCCTTGAGTACAGCGTCCCCGTCGCCATCAGCGCCCAC | |
| | GTCCAGATGATCCAGCCCACCACCCTCTTCGGCCGCCAGAAGCCCC | |
| | AGAACAGCCTCATCCTCAACCCCCTCACCAAGGACCTTGAGAGCATG | |
| | AGCGTCGAAGAGTTCGCCGCCAGCCAGTGCCGCAGCCTCGTCACTA | |
| | CTGCCTGCCTCCGCGAGCTGTACGGCCTCGGCGATCGAGTCACCCA | |
| | GGCCCGCGACGACAACCGAATTGGCGTCAGCGGCTTCCTCGAAGAG | |
| | TACGCCCAGTACCGCGACCTTGAGCTGTTCCTCAGCCGCTTCGAGCC | |
| | CAGCGCCAAGGGCTTCAACTTCAGCGAGGGCCTGATCGCTGGCGGC | |
| | AAGAACACCCAGGGTGGCCCTGGCTCTAGCACCGAGGCCAACCTCG | |
| | ACATGCAGTACGTCGTCGGCCTCAGCCACAAGGCCAAGGTCACCTAC | |
| | TACAGCACTGCCGGCCGAGGCCCCCTCATCCCTGATCTCTCACAGCC | |
| | CAGCCAGGCCAGCAACAACAACGAGCCCTACCTTGAGCAGCTCCGC | |
| | TACCTCGTCAAGCTCCCCAAGAACCAGCTCCCCAGCGTCCTCACCAC | |
| | CAGCTACGGCGACACCGAGCAGAGCCTCCCCGCCAGCTACACCAAG | |
| | GCCACGTGCGACCTCTTCGCCCAGCTCGGCACTATGGGCGTCAGCG | |
| | TCATCTTCAGCAGCGGCGACACTGGCCCTGGCAGCTCGTGCCAGAC | |
| | CAACGACGGCAAGAACGCCACGCGCTTCAACCCCATCTACCCCGCC | |
| | AGCTGCCCCTTCGTCACCAGCATTGGCGGCACCGTCGGCACCGGCC | |
| | CTGAGCGAGCTGTCAGCTTTAGCAGCGGCGGCTTCAGCGACCGCTT | |
| | CCCTCGCCCTCAGTACCAGGACAACGCCGTCAAGGACTACCTCAAGA | |
| | TCCTCGGCAACCAGTGGTCCGGCCTCTTCGACCCTAACGGCCGAGC | |
| | CTTCCCCGACATTGCCGCCCAGGGCAGCAACTACGCCGTCTACGAC | |
| | AAGGGCCGCATGACCGGCGTTAGCGGCACTTCTGCTTCCGCCCCTG | |
| | CTATGGCCGCCATCATTGCCCAGCTCAACGACTTCCGCCTCGCCAAG | |
| | GGCAGCCCCGTCCTCGGCTTTCTCAACCCCTGGATCTACAGCAAGG | |
| | GCTTCAGCGGCTTCACCGACATCGTCGACGGCGGCTCTAGGGGCTG | |
| | CACCGGCTACGACATCTACAGCGGCCTCAAGGCCAAGAAGGTCCCC | |
| | TACGCCAGCTGGAACGCCACCAAGGGCTGGGACCCCGTCACCGGCT | |
| | TTGGCACCCCCAACTTCCAGGCCCTGACCAAGGTCCTGCCCTAA | |
| 84 | ATGCAGACCTTCGGTGCTTTTCTCGTTTCCTTCCTCGCCGCCAGCGG | Fusarium |
| | CCTGGCCGCGGCCAAGAGCTACTCTCACCACGCCGAGGCCCCCAAG | graminearum |
| | GGCTGGAAGGTCGACGATACTGCCCGCGTCGCCAGCACCGGCAAGC | PH-1 |
| | AGCAGGTCTTTTCGATCGCCCTGACCATGCAGAACGTCGACCAGCTT | |
| | GAGAGCAAGCTCCTCGACCTCAGCAGCCCCGACAGCAAGAACTACG | |
| | GCCAGTGGATGAGCCAGAAGGACGTCACCACCGCCTTCTACCCCAG | |
| | CAAGGAAGCCGTCAGCAGCGTCACCAAGTGGCTCAAGAGCAAGGGC | |
| | GTCAAGCACTACAACGTCAACGGCGGCTTCATCGACTTCGCCCTCGA | |
| | CGTGAAGGGCGCCAACGCCCTCCTCGACAGCGACTACCAGTACTAC | |
| | ACCAAGGAAGGCCAGACCAAGCTCCGCACCCTCAGCTACAGCATCC | |
| | CCGACGACGTCGCCGAGCACGTCCAGTTCGTCGACCCCAGCACCAA | |
| | CTTCGGCGGCACCCTCGCCTTTGCCCCCGTCACTCACCCTAGCCGC | |
| | ACCCTCACCGAGCGCAAGAACAAGCCCACCAAGAGCACCGTCGACG | |
| | CCAGCTGCCAGACCAGCATCACCCCCAGCTGCCTCAAGCAGATGTAC | |
| | AACATCGGCGACTACACCCCCAAGGTCGAGAGCGGCAGCACGATCG | |
| | GCTTCAGCAGCTTCCTCGGCGAGAGCGCTATCTACAGCGACGTCTTT | |
| | CTGTTCGAGGAAAAGTTCGGCATCCCCACCCAGAACTTCACCACCGT | |
| | CCTCATCAACAACGGCACCGACGACCAGAACACCGCCCACAAGAACT | |
| | TCGGCGAGGCCGACCTCGACGCCGAGAACATCGTCGGCATTGCCCA | |
| | CCCCCTGCCCTTCACCCAGTACATCACTGGCGGCAGCCCCCCCTTCC | |
| | TGCCCAACATCGATCAGCCCACTGCCGCCGACAACCAGAACGAGCC | |
| | CTACGTCCCCTTCTTCCGCTACCTCCTCAGCCAGAAGGAAGTCCCCG | |
| | CCGTCGTCAGCACCAGCTACGGCGACGAAGAGGACAGCGTCCCCCG | |
| | CGAGTACGCCACCATGACCTGCAACCTCATCGGCCTGCTCGGCCTC | |
| | CGCGGCATCAGCGTCATCTTCAGCAGCGGCGACATCGGCGTCGGCG | |
| | CTGGCTGTCTTGGCCCCGACCACAAGACCGTCGAGTTCAACGCCATC | |
| | TTCCCCGCCACGTGCCCCTACCTCACTAGCGTCGGCGGCACGGTCG | |
| | ACGTCACCCCCGAGATTGCTTGGGAGGGCAGCAGCGGCGGCTTCAG | |
| | CAAGTACTTCCCTCGCCCCAGCTACCAGGACAAGGCCGTCAAGACCT | |
| | ACATGAAGACCGTCAGCAAGCAGACCAAGAAGTACTACGGCCCCTAC | |
| | ACCAACTGGGAGGGCCGAGGCTTTCCTGACGTCGCCGGCCACAGCG | |
| | TCAGCCCCAACTACGAGGTCATCTACGCCGGCAAGCAGAGCGCCTC | |
| | TGGCGGCACTTCTGCTGCCGCCCCTGTCTGGGCTGCCATCGTCGGC | |
| | CTGCTCAACGACGCCCGATTCCGAGCCGGCAAGCCTAGCCTCGGCT | |
| | GGCTCAACCCCCTCGTCTACAAGTACGGCCCCAAGGTCCTCACCGAC | |
| | ATCACCGGCGGCTACGCCATTGGCTGCGACGGCAACAACACCCAGA | |
| | GCGGCAAGCCCGAGCCTGCCGGCTCTGGCATTGTCCCTGGCGCCCG | |
| | ATGGAACGCCACTGCCGGATGGGACCCTGTCACCGGCTACGGCACC | |
| | CCCGACTTCGGCAAGCTCAAGGACCTCGTCCTCAGCTTCTAA | |
| 85 | ATGCAGACCTTCGGTGCTTTTCTCGTTTCCTTCCTCGCCGCCAGCGG | Acremonium |
| | CCTGGCCGCGGCCGCCGTCGTCATTCGCGCCGCCGTCCTCCCCGAC | alcalophilum |
| | GCCGTCAAGCTGATGGGCAAGGCCATGCCCGACGACATTATTTCCCT | |
| | CCAGTTTTCCCTGAAGCAGCAGAACATCGACCAGCTGGAGACCCGCC | |
| | TCCGCGCCGTCTCGGACCCCAGCTCCCCCGAGTACGGCCAGTACAT | |
| | GAGCGAGTCCGAGGTCAACGAGTTCTTTAAGCCCCGCGACGACTCG | |
| | TTCGCCGAGGTCATTGACTGGGTCGCCGCCAGCGGCTTTCAGGACA | |
| | TCCACCTGACGCCCCAGGCTGCCGCCATTAACCTCGCCGCCACCGT | |
| | CGAGACGGCCGACCAGCTCCTGGGCGCCAACTTCAGCTGGTTTGAC | |
| | GTCGACGGCACCCGCAAGCTCCGCACCCTGGAGTACACGATCCCCG | |
| | ACCGCCTCGCCGACCACGTCGACCTGATTTCCCCCACCACGTACTTC | |
| | GGCCGCGCCCGACTGGACGGCCCCCGCGAGACCCCCACGCGCCTC | |
| | GACAAGCGCCAGCGCGACCCCGTCGCCGACAAGGCCTACTTCCACC | |
| | TCAAGTGGGACCGCGGCACCAGCAACTGCGACCTGGTCATCACGCC | |
| | CCCCTGCCTGGAGGCCGCCTACAACTACAAGAACTACATGCCCGACC | |
| | CCAACTCGGGCAGCCGCGTCTCGTTCACCAGCTTTCTGGAGCAGGC | |
| | CGCCCAGCAGAGCGACCTCACCAAGTTCCTCTCCCTGACGGGCCTC | |
| | GACCGCCTGCGCCCCCCCAGCAGCAAGCCCGCCAGCTTCGACACGG | |
| | TCCTGATCAACGGCGGCGAGACCCACCAGGGCACGCCCCCCAACAA | |
| | GACCTCCGAGGCCAACCTCGACGTCCAGTGGCTGGCCGCCGTCATT | |
| | AAGGCCCGACTCCCCATCACCCAGTGGATTACGGGCGGCCGCCCCC | |
| | CCTTCGTCCCCAACCTCCGCCTGCGCCACGAGAAGGACAACACGAA | |
| | CGAGCCCTACCTGGAGTTCTTTGAGTACCTCGTCCGCCTGCCCGCCC | |
| | GCGACCTCCCCCAGGTCATCTCCAACTCGTACGCCGAGGACGAGCA | |
| | GACCGTCCCCGAGGCCTACGCCCGACGCGTCTGCAACCTCATCGGC | |
| | ATTATGGGCCTGCGCGGCGTCACCGTCCTCACGGCCTCCGGCGACT | |
| | CGGGCGTCGGCGCCCCCTGCCGCGCCAACGACGGCAGCGACCGCC | |
| | TGGAGTTCTCCCCCCAGTTTCCCACCTCGTGCCCCTACATCACCGCC | |
| | GTCGGCGGCACGGAGGGCTGGGACCCCGAGGTCGCCTGGGAGGCC | |
| | TCCTCGGGCGGCTTCAGCCACTACTTTCTCCGCCCCTGGTACCAGGC | |
| | CAACGCCGTCGAGAAGTACCTCGACGAGGAGCTGGACCCCGCCACC | |
| | CGCGCCTACTACGACGGCAACGGCTTCGTCCAGTTTGCCGGCCGAG | |
| | CCTACCCCGACCTGTCCGCCCACAGCTCCTCGCCCCGCTACGCCTA | |
| | CATCGACAAGCTCGCCCCCGGCCTGACCGGCGGCACGAGCGCCTCC | |
| | TGCCCCGTCGTCGCCGGCATCGTCGGCCTCCTGAACGACGCCCGAC | |
| | TCCGCCGCGGCCTGCCCACGATGGGCTTCATTAACCCCTGGCTGTA | |
| | CACGCGCGGCTTTGAGGCCCTCCAGGACGTCACCGGCGGCCGCGC | |
| | CTCGGGCTGCCAGGGCATCGACCTCCAGCGCGGCACCCGCGTCCC | |
| | CGGCGCCGGCATCATTCCCTGGGCCTCCTGGAACGCCACCCCCGGC | |
| | TGGGACCCCGCCACGGGCCTCGGCCTGCCCGACTTCTGGGCCATGC | |
| | GCGGCCTCGCCCTGGGCCGCGGCACCTAA | |
| 86 | ATGCAGACCTTCGGTGCTTTTCTCGTTTCCTTCCTCGCCGCCAGCGG | Sodiomyces |
| | CCTGGCCGCGGCCGCCGTCGTCATTCGCGCCGCCCCCCTCCCCGA | alkalinus |
| | GAGCGTCAAGCTCGTCCGCAAGGCCGCCGCCGAGGACGGCATTAAC | |
| | CTCCAGCTCTCCCTGAAGCGCCAGAACATGGACCAGCTGGAGAAGTT | |
| | CCTCCGCGCCGTCAGCGACCCCTTTTCCCCCAAGTACGGCCAGTACA | |
| | TGTCGGACGCCGAGGTCCACGAGATCTTCCGCCCCACCGAGGACTC | |
| | CTTTGACCAGGTCATTGACTGGCTCACCAAGTCGGGCTTCGGCAACC | |
| | TGCACATCACGCCCCAGGCTGCCGCCATTAACGTCGCCACCACGGT | |
| | CGAGACCGCCGACCAGCTGTTTGGCGCCAACTTCTCCTGGTTTGACG | |
| | TCGACGGCACGCCCAAGCTCCGCACCGGCGAGTACACGATCCCCGA | |
| | CCGCCTCGTCGAGCACGTCGACCTGGTCAGCCCCACCACGTACTTC | |
| | GGCCGCATGCGCCCCCCCCCTCGCGGCGACGGCGTCAACGACTGG | |
| | ATCACCGAGAACTCGCCCGAGCAGCCCGCCCCCCTGAACAAGCGCG | |
| | ACACCAAGACGGAGAGCGACCAGGCCCGCGACCACCCCTCCTGGGA | |
| | CTCGCGCACCCCCGACTGCGCCACCATCATTACGCCCCCCTGCCTG | |
| | GAGACGGCCTACAACTACAAGGGCTACATCCCCGACCCCAAGTCCG | |
| | GCTCGCGCGTCAGCTTCACCAGCTTCCTGGAGCAGGCCGCCCAGCA | |
| | GGCCGACCTGACCAAGTTCCTCAGCCTGACGCGCCTGGAGGGCTTT | |
| | CGCACCCCCGCCAGCAAGAAGAAGACCTTCAAGACGGTCCTGATCAA | |
| | CGGCGGCGAGTCCCACGAGGGCGTCCACAAGAAGTCGAAGACCAGC | |
| | GAGGCCAACCTCGACGTCCAGTGGCTGGCCGCCGTCACCCAGACGA | |
| | AGCTGCCCATCACCCAGTGGATTACGGGCGGCCGCCCCCCCTTCGT | |
| | CCCCAACCTCCGCATCCCCACCCCCGAGGCCAACACGAACGAGCCC | |
| | TACCTGGAGTTCCTGGAGTACCTCTTTCGCCTGCCCGACAAGGACCT | |
| | CCCCCAGGTCATCAGCAACTCCTACGCCGAGGACGAGCAGAGCGTC | |
| | CCCGAGGCCTACGCCCGACGCGTCTGCGGCCTCCTGGGCATTATGG | |
| | GCCTCCGCGGCGTCACCGTCCTGACGGCCTCCGGCGACTCGGGCG | |
| | TCGGCGCCCCCTGCCGCGCCAACGACGGCTCGGGCCGCGAGGAGT | |
| | TCAGCCCCCAGTTTCCCAGCTCCTGCCCCTACATCACCACGGTCGGC | |
| | GGCACCCAGGCCTGGGACCCCGAGGTCGCCTGGAAGGGCAGCAGC | |
| | GGCGGCTTCTCCAACTACTTTCCCCGCCCCTGGTACCAGGTCGCCG | |
| | CCGTCGAGAAGTACCTGGAGGAGCAGCTGGACCCCGCCGCCCGCG | |
| | AGTACTACGAGGAGAACGGCTTCGTCCGCTTTGCCGGCCGAGCCTT | |
| | CCCCGACCTGAGCGCCCACAGCAGCAGCCCCAAGTACGCCTACGTC | |
| | GACAAGCGCGTCCCCGGCCTCACCGGCGGCACGTCGGCCAGCTGC | |
| | CCCGTCGTCGCCGGCATCGTCGGCCTCCTGAACGACGCCCGACTCC | |
| | GCCGCGGCCTGCCCACGATGGGCTTCATTAACCCCTGGCTCTACGC | |
| | CAAGGGCTACCAGGCCCTGGAGGACGTCACCGGCGGCGCCGCCGT | |
| | CGGCTGCCAGGGCATCGACATTCAGACGGGCAAGCGCGTCCCCGGC | |
| | GCCGGCATCATTCCCGGCGCCAGCTGGAACGCCACCCCCGACTGGG | |
| | ACCCCGCCACGGGCCTCGGCCTGCCCAACTTCTGGGCCATGCGCGA | |
| | GCTCGCCCTGGAGGACTAA | |
| 87 | ATGCAGACCTTCGGTGCTTTTCTCGTTTCCTTCCTCGCCGCCAGCGG | Aspergillus |
| | CCTGGCCGCGGCCGTCGTCCATGAGAAGCTCGCTGCTGTCCCCAGC | kawachii IFO |
| | GGCTGGCACCACCTTGAGGATGCCGGCAGCGACCACCAGATCAGCC | 4308 |
| | TCTCGATTGCCCTCGCCCGCAAGAACCTCGACCAGCTTGAGAGCAAG | |
| | CTCAAGGACCTCAGCACCCCTGGCGAGAGCCAGTACGGCCAGTGGC | |
| | TCGACCAAGAGGAAGTCGACACCCTGTTCCCCGTCGCCAGCGACAA | |
| | GGCCGTCATCAGCTGGCTCCGCAGCGCCAACATCACCCACATTGCC | |
| | CGCCAGGGCAGCCTCGTCAACTTCGCCACCACCGTCGACAAGGTCA | |
| | ACAAGCTCCTCAACACCACCTTCGCCTACTACCAGCGCGGCAGCTCT | |
| | CAGCGCCTCCGCACCACCGAGTACAGCATCCCCGACGACCTCGTCG | |
| | ACAGCATCGACCTGATCAGCCCCACCACGTTCTTCGGCAAGGAAAAG | |
| | ACCTCTGCCGGCCTCACCCAGCGCAGCCAGAAGGTCGATAACCACG | |
| | TCGCCAAGCGCAGCAACAGCAGCAGCTGCGCCGACACCATCACCCT | |
| | CAGCTGCCTCAAGGAAATGTACAACTTCGGCAACTACACCCCCAGCG | |
| | CCAGCAGCGGCAGCAAGCTCGGCTTCGCCAGCTTCCTCAACGAGAG | |
| | CGCCAGCTACAGCGACCTCGCCAAGTTCGAGCGCCTCTTCAACCTCC | |
| | CCAGCCAGAACTTCAGCGTCGAGCTGATCAACGGCGGCGTCAACGA | |
| | CCAGAACCAGAGCACCGCCAGCCTCACCGAGGCCGACCTCGATGTC | |
| | GAGCTGCTTGTCGGCGTCGGCCACCCCCTGCCCGTCACCGAGTTTA | |
| | TCACCAGCGGCGAGCCCCCCTTCATCCCCGACCCTGATGAGCCTTCT | |
| | GCCGCCGACAACGAGAACGAGCCCTACCTCCAGTACTACGAGTACCT | |
| | CCTCAGCAAGCCCAACAGCGCCCTGCCCCAGGTCATCAGCAACAGC | |
| | TACGGCGACGACGAGCAGACCGTCCCCGAGTACTACGCCAAGCGCG | |
| | TCTGCAACCTCATCGGCCTCGTCGGCCTCCGCGGCATCAGCGTCCTT | |
| | GAGTCTAGCGGCGACGAGGGCATCGGCTCTGGCTGCCGAACCACCG | |
| | ACGGCACCAACAGCACCCAGTTCAACCCCATCTTCCCCGCCACGTGC | |
| | CCCTACGTCACTGCCGTCGGCGGCACCATGAGCTACGCCCCCGAGA | |
| | TTGCTTGGGAGGCCAGCTCCGGCGGCTTCAGCAACTACTTCGAGCG | |
| | AGCCTGGTTCCAGAAGGAAGCCGTCCAGAACTACCTCGCCAACCACA | |
| | TCACCAACGAGACTAAGCAGTACTACAGCCAGTTCGCCAACTTCAGC | |
| | GGCCGAGGCTTCCCCGACGTCAGCGCCCACAGCTTCGAGCCCAGCT | |
| | ACGAGGTCATCTTCTACGGCGCTCGCTACGGCAGCGGCGGCACTTC | |
| | TGCTGCCTGCCCCCTGTTTTCTGCCCTCGTCGGCATGCTCAACGACG | |
| | CCCGACTCCGAGCCGGCAAGTCGACCCTCGGCTTCCTCAACCCCCT | |
| | GCTCTACAGCAAGGGCTACAAGGCCCTCACCGACGTCACCGCTGGC | |
| | CAGAGCATTGGCTGCAACGGCATCGACCCCCAGAGCGACGAGGCTG | |
| | TCGCTGGCGCTGGCATCATTCCCTGGGCCCACTGGAACGCCACCGT | |
| | CGGCTGGGACCCTGTCACTGGCCTTGGCCTCCCCGACTTCGAGAAG | |
| | CTCCGCCAGCTCGTCCTCAGCCTCTAA | |
| 88 | ATGCAGACCTTCGGTGCTTTTCTCGTTTCCTTCCTCGCCGCCAGCGG | Talaromyces |
| | CCTGGCCGCGGCCGCTGCTGCTCTTGTTGGCCACGAGTCTCTCGCC | stipitatus |
| | GCCCTCCCTGTCGGCTGGGACAAGGTCAGCACTCCTGCCGCTGGCA | ATCC 10500 |
| | CCAACATCCAGCTCAGCGTCGCCCTCGCCCTCCAGAACATCGAGCA | |
| | GCTTGAGGACCACCTCAAGAGCGTCAGCACCCCCGGCTCTGCCAGC | |
| | TACGGCCAGTACCTCGACAGCGACGGCATTGCCGCCCAGTACGGCC | |
| | CTTCTGACGCCAGCGTCGAGGCCGTCACCAACTGGCTCAAGGAAGC | |
| | CGGCGTCACCGACATCTACAACAACGGCCAGAGCATCCACTTCGCCA | |
| | CCAGCGTCAGCAAGGCCAACAGCCTCCTCGGCGCCGACTTCAACTA | |
| | CTACAGCGACGGCTCCGCCACCAAGCTCCGCACCCTCGCTTACAGC | |
| | GTCCCCAGCGACCTGAAGGAAGCCATCGACCTCGTCAGCCCCACCA | |
| | CCTACTTCGGCAAGACCACCGCCAGCCGCAGCATCCAGGCCTACAA | |
| | GAACAAGCGAGCCAGCACCACCAGCAAGAGCGGCAGCAGCAGCGTC | |
| | CAGGTCAGCGCCTCTTGCCAGACCAGCATCACCCCCGCCTGCCTCA | |
| | AGCAGATGTACAACGTCGGCAACTACACCCCCAGCGTCGCCCACGG | |
| | CTCTCGCGTTGGCTTCGGCAGCTTCCTCAACCAGAGCGCCATCTTCG | |
| | ACGACCTCTTCACCTACGAGAAGGTCAACGACATCCCCAGCCAGAAC | |
| | TTCACCAAGGTCATCATTGCCAACGCCAGCAACAGCCAGGACGCCAG | |
| | CGACGGCAACTACGGCGAGGCCAACCTCGACGTCCAGAACATTGTC | |
| | GGCATCAGCCACCCCCTGCCCGTCACCGAGTTTCTCACTGGCGGCA | |
| | GCCCACCCTTCGTCGCCAGCCTCGACACCCCCACCAACCAGAACGA | |
| | GCCCTACATCCCCTACTACGAGTACCTCCTCAGCCAGAAGAACGAGG | |
| | ACCTCCCCCAGGTCATCAGCAACAGCTACGGCGACGACGAGCAGAG | |
| | CGTCCCCTACAAGTACGCCATCCGCGCCTGCAACCTCATCGGCCTCA | |
| | CTGGCCTCCGCGGCATCAGCGTCCTTGAGAGCAGCGGCGATCTCGG | |
| | CGTTGGCGCTGGCTGCCGATCCAACGACGGCAAGAACAAGACCCAG | |
| | TTCGACCCCATCTTCCCCGCCACGTGCCCCTACGTCACTAGCGTCGG | |
| | CGGCACCCAGAGCGTCACCCCCGAGATTGCTTGGGTCGCTTCCAGC | |
| | GGCGGCTTCAGCAACTACTTCCCCCGCACCTGGTATCAAGAGCCCG | |
| | CCATCCAGACCTACCTCGGCCTCCTCGACGACGAGACTAAGACCTAC | |
| | TACAGCCAGTACACCAACTTCGAGGGCCGAGGCTTCCCCGACGTCA | |
| | GCGCCCATTCTCTCACCCCCGACTACCAGGTCGTCGGCGGAGGCTA | |
| | CCTTCAGCCTTCTGGCGGCACTTCTGCCGCCAGCCCTGTCTTTGCCG | |
| | GCATCATTGCCCTGCTCAACGACGCCCGACTCGCCGCTGGCAAGCC | |
| | CACCCTCGGCTTTCTCAACCCCTTCTTCTACCTCTACGGCTACAAGG | |
| | GCCTCAACGACATCACTGGCGGCCAGAGCGTCGGCTGCAACGGCAT | |
| | CAACGGCCAGACTGGCGCCCCTGTTCCCGGCGGAGGAATTGTCCCT | |
| | GGCGCCGCTTGGAACAGCACCACCGGATGGGACCCTGCCACCGGC | |
| | CTTGGCACCCCCGACTTTCAGAAGCTCAAGGAACTCGTCCTCAGCTT | |
| | CTAA | |
| 89 | ATGCAGACCTTCGGTGCTTTTCTCGTTTCCTTCCTCGCCGCCAGCGG | Fusarium |
| | CCTGGCCGCGGCCAAGTCGTTTTCCCACCACGCCGAGGCCCCCCAG | oxysporum f. |
| | GGCTGGCAGGTCCAGAAGACCGCCAAGGTCGCCTCCAACACGCAGC | sp. cubense |
| | ACGTCTTTAGCCTCGCCCTGACCATGCAGAACGTCGACCAGCTGGAG | race 4 |
| | TCGAAGCTCCTGGACCTGAGCTCCCCCGACAGCGCCAACTACGGCA | |
| | ACTGGCTCAGCCACGACGAGCTGACCTCCACGTTCTCGCCCAGCAA | |
| | GGAGGCCGTCGCCTCGGTCACCAAGTGGCTGAAGAGCAAGGGCATC | |
| | AAGCACTACAAGGTCAACGGCGCCTTCATTGACTTTGCCGCCGACGT | |
| | CGAGAAGGCCAACACCCTCCTGGGCGGCGACTACCAGTACTACACG | |
| | AAGGACGGCCAGACCAAGCTGCGCACGCTCTCCTACTCGATCCCCG | |
| | ACGACGTCGCCGGCCACGTCCAGTTCGTCGACCCCAGCACCAACTT | |
| | CGGCGGCACGGTCGCCTTTAACCCCGTCCCCCACCCCTCCCGCACC | |
| | CTCCAGGAGCGCAAGGTCTCCCCCTCCAAGTCGACGGTCGACGCCT | |
| | CCTGCCAGACCTCGATCACGCCCAGCTGCCTGAAGCAGATGTACAAC | |
| | ATTGGCGACTACACCCCCGACGCCAAGAGCGGCTCCGAGATCGGCT | |
| | TCAGCAGCTTCCTCGGCCAGGCCGCCATTTACAGCGACGTCTTCAAG | |
| | TTTGAGGAGCTCTTCGGCATCCCCAAGCAGAACTACACCACGATCCT | |
| | GATTAACAACGGCACCGACGACCAGAACACGGCCCACGGCAACTTT | |
| | GGCGAGGCCAACCTCGACGCCGAGAACATCGTCGGCATTGCCCACC | |
| | CCCTGCCCTTCAAGCAGTACATCACCGGCGGCAGCCCCCCCTTTGTC | |
| | CCCAACATTGACCAGCCCACGGAGAAGGACAACCAGAACGAGCCCT | |
| | ACGTCCCCTTCTTTCGCTACCTCCTGGGCCAGAAGGACCTGCCCGCC | |
| | GTCATCTCGACCAGCTACGGCGACGAGGAGGACTCCGTCCCCCGCG | |
| | AGTACGCCACCCTCACGTGCAACATGATCGGCCTCCTGGGCCTGCG | |
| | CGGCATCTCCGTCATTTTCTCCTCGGGCGACATTGGCGTCGGCTCGG | |
| | GCTGCCTCGCCCCCGACTACAAGACCGTCGAGTTCAACGCCATCTTT | |
| | CCCGCCACCTGCCCCTACCTGACGTCCGTCGGCGGCACCGTCGACG | |
| | TCACGCCCGAGATTGCCTGGGAGGGCAGCTCCGGCGGCTTCTCCAA | |
| | GTACTTTCCCCGCCCCTCGTACCAGGACAAGGCCATCAAGAAGTACA | |
| | TGAAGACCGTCTCGAAGGAGACGAAGAAGTACTACGGCCCCTACACC | |
| | AACTGGGAGGGCCGCGGCTTCCCCGACGTCGCCGGCCACTCCGTC | |
| | GCCCCCGACTACGAGGTCATCTACAACGGCAAGCAGGCCCGATCCG | |
| | GCGGCACCAGCGCCGCCGCCCCCGTCTGGGCCGCCATCGTCGGCC | |
| | TCCTGAACGACGCCCGATTCAAGGCCGGCAAGAAGAGCCTGGGCTG | |
| | GCTCAACCCCCTGATCTACAAGCACGGCCCCAAGGTCCTCACCGACA | |
| | TCACGGGCGGCTACGCCATTGGCTGCGACGGCAACAACACCCAGAG | |
| | CGGCAAGCCCGAGCCCGCCGGCTCCGGCCTGGTCCCCGGCGCCCG | |
| | ATGGAACGCCACCGCCGGCTGGGACCCCACCACGGGCTACGGCAC | |
| | GCCCAACTTCCAGAAGCTCAAGGACCTCGTCCTGTCCCTCTAA | |
| 90 | ATGCAGACCTTCGGTGCTTTTCTCGTTTCCTTCCTCGCCGCCAGCGG | Trichoderma |
| | CCTGGCCGCGGCCGTCGTCCATGAGAAGCTCGCTGCTGTCCCCAGC | virens Gv29- |
| | GGCTGGCACCACCTTGAGGATGCCGGCAGCGACCACCAGATCAGCC | 8 |
| | TCTCGATTGCCCTCGCCCGCAAGAACCTCGACCAGCTTGAGAGCAAG | |
| | CTCAAGGACCTCAGCACCCCTGGCGAGAGCCAGTACGGCCAGTGGC | |
| | TCGACCAAGAGGAAGTCGACACCCTGTTCCCCGTCGCCAGCGACAA | |
| | GGCCGTCATCAGCTGGCTCCGCAGCGCCAACATCACCCACATTGCC | |
| | CGCCAGGGCAGCCTCGTCAACTTCGCCACCACCGTCGACAAGGTCA | |
| | ACAAGCTCCTCAACACCACCTTCGCCTACTACCAGCGCGGCAGCTCT | |
| | CAGCGCCTCCGCACCACCGAGTACAGCATCCCCGACGACCTCGTCG | |
| | ACAGCATCGACCTGATCAGCCCCACCACGTTCTTCGGCAAGGAAAAG | |
| | ACCTCTGCCGGCCTCACCCAGCGCAGCCAGAAGGTCGATAACCACG | |
| | TCGCCAAGCGCAGCAACAGCAGCAGCTGCGCCGACACCATCACCCT | |
| | CAGCTGCCTCAAGGAAATGTACAACTTCGGCAACTACACCCCCAGCG | |
| | CCAGCAGCGGCAGCAAGCTCGGCTTCGCCAGCTTCCTCAACGAGAG | |
| | CGCCAGCTACAGCGACCTCGCCAAGTTCGAGCGCCTCTTCAACCTCC | |
| | CCAGCCAGAACTTCAGCGTCGAGCTGATCAACGGCGGCGTCAACGA | |
| | CCAGAACCAGAGCACCGCCAGCCTCACCGAGGCCGACCTCGATGTC | |
| | GAGCTGCTTGTCGGCGTCGGCCACCCCCTGCCCGTCACCGAGTTTA | |
| | TCACCAGCGGCGAGCCCCCCTTCATCCCCGACCCTGATGAGCCTTCT | |
| | GCCGCCGACAACGAGAACGAGCCCTACCTCCAGTACTACGAGTACCT | |
| | CCTCAGCAAGCCCAACAGCGCCCTGCCCCAGGTCATCAGCAACAGC | |
| | TACGGCGACGACGAGCAGACCGTCCCCGAGTACTACGCCAAGCGCG | |
| | TCTGCAACCTCATCGGCCTCGTCGGCCTCCGCGGCATCAGCGTCCTT | |
| | GAGTCTAGCGGCGACGAGGGCATCGGCTCTGGCTGCCGAACCACCG | |
| | ACGGCACCAACAGCACCCAGTTCAACCCCATCTTCCCCGCCACGTGC | |
| | CCCTACGTCACTGCCGTCGGCGGCACCATGAGCTACGCCCCCGAGA | |
| | TTGCTTGGGAGGCCAGCTCCGGCGGCTTCAGCAACTACTTCGAGCG | |
| | AGCCTGGTTCCAGAAGGAAGCCGTCCAGAACTACCTCGCCAACCACA | |
| | TCACCAACGAGACTAAGCAGTACTACAGCCAGTTCGCCAACTTCAGC | |
| | GGCCGAGGCTTCCCCGACGTCAGCGCCCACAGCTTCGAGCCCAGCT | |
| | ACGAGGTCATCTTCTACGGCGCTCGCTACGGCAGCGGCGGCACTTC | |
| | TGCTGCCTGCCCCCTGTTTTCTGCCCTCGTCGGCATGCTCAACGACG | |
| | CCCGACTCCGAGCCGGCAAGTCGACCCTCGGCTTCCTCAACCCCCT | |
| | GCTCTACAGCAAGGGCTACAAGGCCCTCACCGACGTCACCGCTGGC | |
| | CAGAGCATTGGCTGCAACGGCATCGACCCCCAGAGCGACGAGGCTG | |
| | TCGCTGGCGCTGGCATCATTCCCTGGGCCCACTGGAACGCCACCGT | |
| | CGGCTGGGACCCTGTCACTGGCCTTGGCCTCCCCGACTTCGAGAAG | |
| | CTCCGCCAGCTCGTCCTCAGCCTCTAA | |
| 91 | ATGCAGACCTTCGGTGCTTTTCTCGTTTCCTTCCTCGCCGCCAGCGG | Trichoderma |
| | CCTGGCCGCGGCCGCTGTCCTTGTCGAGTCTCTCAAGCAGGTCCCC | atroviride IMI |
| | AACGGCTGGAACGCCGTCAGCACCCCTGACCCCAGCACCAGCATCG | 206040 |
| | TCCTCCAGATCGCCCTCGCCCAGCAGAACATCGACGAGCTTGAGTG | |
| | GCGCCTCGCCGCCGTGTCTACCCCCAACTCTGGCAACTACGGCAAG | |
| | TACCTCGACATCGGCGAGATCGAGGGCATCTTCGCCCCCAGCAACG | |
| | CCAGCTACAAGGCCGTCGCTTCCTGGCTCCAGAGCCACGGCGTCAA | |
| | GAACTTCGTCAAGCAGGCCGGCAGCATCTGGTTCTACACCACCGTCA | |
| | GCACCGCCAACAAGATGCTCAGCACCGACTTCAAGCACTACAGCGAC | |
| | CCCGTCGGCATCGAGAAGCTCCGCACCCTCCAGTACAGCATCCCCG | |
| | AGGAACTCGTCGGCCACGTCGACCTCATCAGCCCCACCACCTACTTC | |
| | GGCAACAACCACCCTGCCACCGCCCGCACCCCCAACATGAAGGCCA | |
| | TCAACGTCACCTACCAGATCTTCCACCCCGACTGCCTCAAGACCAAG | |
| | TACGGCGTCGACGGCTACGCCCCCTCACCTCGATGCGGCAGCCGAA | |
| | TCGGCTTCGGCAGCTTCCTCAACGAGACTGCCAGCTACAGCGACCTC | |
| | GCCCAGTTCGAGAAGTACTTCGACCTCCCCAACCAGAACCTCAGCAC | |
| | CCTCCTCATCAACGGCGCCATCGACGTCCAGCCCCCCAGCAACAAG | |
| | AACGACAGCGAGGCCAACATGGACGTCCAGACCATCCTCACCTTCGT | |
| | CCAGCCCCTGCCCATCACCGAGTTCGTCGTCGCCGGCATCCCCCCC | |
| | TACATTCCCGATGCCGCCCTCCCCATTGGCGACCCCGTTCAGAACGA | |
| | GCCCTGGCTTGAGTACTTCGAGTTCCTCATGAGCCGCACCAACGCCG | |
| | AGCTGCCCCAGGTCATTGCCAACAGCTACGGCGACGAGGAACAGAC | |
| | CGTCCCCCAGGCCTACGCCGTCCGCGTCTGCAACCAGATTGGCCTC | |
| | CTCGGCCTCCGCGGCATCAGCGTCATTGCCTCTAGCGGCGACACCG | |
| | GCGTCGGCATGTCTTGCATGGCCAGCAACAGCACCACCCCCCAGTT | |
| | CAACCCCATGTTCCCCGCCAGCTGCCCCTACATCACCACCGTCGGC | |
| | GGCACCCAGCACCTCGACAACGAGATCGCCTGGGAGCTGAGCAGCG | |
| | GCGGCTTCAGCAACTACTTCACCCGCCCCTGGTATCAAGAGGACGCC | |
| | GCCAAGACCTACCTTGAGCGCCACGTCAGCACCGAGACTAAGGCCT | |
| | ACTACGAGCGCTACGCCAACTTCCTGGGCCGAGGCTTTCCTGACGTC | |
| | GCCGCCCTCAGCCTCAACCCCGACTACCCCGTCATCATCGGCGGCG | |
| | AGCTTGGCCCTAACGGCGGCACTTCTGCTGCCGCCCCTGTCGTCGC | |
| | CAGCATCATTGCCCTGCTCAACGACGCCCGCCTCTGCCTCGGCAAG | |
| | CCTGCCCTCGGCTTTCTCAACCCCCTCATCTACCAGTACGCCGACAA | |
| | GGGCGGCTTCACCGACATCACCAGCGGCCAGTCTTGGGGCTGCGCC | |
| | GGCAACACCACTCAGACTGGACCTCCCCCTCCTGGCGCTGGCGTCA | |
| | TTCCTGGCGCTCACTGGAACGCCACCAAGGGCTGGGACCCCGTCAC | |
| | CGGCTTTGGCACCCCCAACTTCAAGAAGCTCCTCAGCCTCGCCCTCA | |
| | GCGTCTAA | |
| 92 | ATGCAGACCTTCGGTGCTTTTCTCGTTTCCTTCCTCGCCGCCAGCGG | Agaricus |
| | CCTGGCCGCGGCCTCTCCTCTTGCTCGACGCTGGGACGACTTCGCC | bisporus var. |
| | GAGAAGCACGCCTGGGTCGAGGTTCCTCGCGGCTGGGAGATGGTCA | burnettii |
| | GCGAGGCCCCTAGCGACCACACCTTCGACCTCCGCATCGGCGTCAA | JB137-S8 |
| | GAGCAGCGGCATGGAACAGCTCATCGAGAACCTCATGCAGACCAGC | |
| | GACCCCACCCACAGCCGCTACGGCCAGCACCTCAGCAAGGAAGAAC | |
| | TCCACGACTTCGTCCAGCCCCACCCCGACTCTACTGGCGCCGTCGA | |
| | GGCCTGGCTTGAGGACTTCGGCATCAGCGACGACTTCATCGACCGC | |
| | ACCGGCAGCGGCAACTGGGTCACCGTCCGAGTCTCTGTCGCCCAGG | |
| | CCGAGCGAATGCTCGGCACCAAGTACAACGTCTACCGCCACAGCGA | |
| | GAGCGGCGAGTCCGTCGTCCGCACCATGAGCTACAGCCTCCCCAGC | |
| | GAGCTGCACAGCCACATCGACGTCGTCGCCCCCACCACCTACTTCG | |
| | GCACCATGAAGTCGATGCGCGTCACCTCGTTCCTCCAGCCCGAGATC | |
| | GAGCCCGTCGACCCCTCTGCCAAGCCTTCTGCTGCTCCCGCCAGCT | |
| | GCCTCAGCACCACCGTCATTACCCCCGACTGCCTCCGCGACCTCTAC | |
| | AACACCGCCGACTACGTCCCCAGCGCCACCAGCCGCAACGCCATTG | |
| | GCATTGCCGGCTACCTCGACCGCAGCAACCGAGCCGACCTCCAGAC | |
| | CTTCTTCCGCCGCTTTCGCCCTGACGCCGTCGGCTTCAACTACACCA | |
| | CCGTCCAGCTCAACGGCGGAGGCGACGACCAGAACGACCCTGGCGT | |
| | CGAGGCCAACCTCGACATCCAGTACGCCGCTGGCATTGCCTTCCCCA | |
| | CCCCCGCCACCTACTGGTCTACTGGCGGCAGCCCCCCCTTCATCCC | |
| | CGACACCCAGACCCCCACCAACACCAACGAGCCCTACCTCGACTGG | |
| | ATCAACTTCGTCCTCGGCCAGGATGAGATCCCCCAGGTCATCAGCAC | |
| | CAGCTACGGCGACGACGAGCAGACCGTCCCCGAGGACTACGCCACC | |
| | AGCGTCTGCAACCTCTTCGCCCAGCTTGGCTCTCGCGGCGTCACCGT | |
| | CTTTTTCAGCAGCGGCGACTTCGGCGTCGGCGGTGGCGACTGCCTC | |
| | ACTAACGACGGCAGCAACCAGGTCCTCTTCCAGCCCGCCTTCCCTGC | |
| | CAGCTGCCCCTTTGTCACTGCCGTCGGCGGCACCGTCCGACTCGAC | |
| | CCTGAGATCGCCGTCAGCTTCAGCGGCGGTGGCTTCAGCCGCTACT | |
| | TCAGCCGCCCCAGCTACCAGAACCAGACCGTCGCCCAGTTCGTCAG | |
| | CAACCTCGGCAACACCTTCAACGGCCTCTACAACAAGAACGGCCGAG | |
| | CCTACCCCGACCTCGCCGCTCAGGGCAACGGCTTCCAGGTCGTCAT | |
| | CGACGGCATCGTCCGATCGGTCGGCGGCACTTCTGCCAGCAGCCCT | |
| | ACCGTCGCCGGCATCTTCGCCCTGCTCAACGACTTCAAGCTCTCTCG | |
| | CGGCCAGAGCACCCTCGGCTTCATCAACCCCCTCATCTACAGCAGCG | |
| | CCACCTCCGGCTTCAACGACATCCGAGCCGGCACCAACCCTGGCTG | |
| | TGGCACCCGAGGCTTTACCGCCGGCACTGGCTGGGACCCTGTCACC | |
| | GGACTCGGCACCCCTGACTTTCTCCGCCTCCAGGGCCTCATCTAA | |
| 93 | ATGCAGACCTTCGGTGCTTTTCTCGTTTCCTTCCTCGCCGCCAGCGG | Magnaporthe |
| | CCTGGCCGCGGCCCGCGTCTTTGATTCTCTCCCTCACCCCCCTCGC | oryzae 70-15 |
| | GGCTGGTCCTACTCTCACGCCGCTGAGAGCACCGAGCCCCTCACCC | |
| | TCCGAATTGCCCTCCGCCAGCAGAACGCCGCTGCCCTTGAGCAGGT | |
| | CGTCCTCCAGGTCAGCAACCCCCGCCACGCCAACTACGGCCAGCAC | |
| | CTCACCCGAGATGAGCTGCGCTCTTACACCGCCCCTACCCCTCGCG | |
| | CTGTCCGCTCTGTCACTAGCTGGCTCGTCGACAACGGCGTCGACGA | |
| | CTACACCGTCGAGCACGACTGGGTCACCCTCCGCACCACTGTCGGC | |
| | GCTGCCGATCGACTCCTCGGCGCCGACTTTGCCTGGTACGCTGGCC | |
| | CTGGCGAGACTCTCCAGCTCCGCACTCTCAGCTACGGCGTGGACGA | |
| | CAGCGTCGCCCCTCACGTCGATCTCGTCCAGCCCACCACCCGCTTTG | |
| | GCGGCCCTGTTGGCCAGGCCAGCCACATCTTCAAGCAGGACGACTT | |
| | CGACGAGCAGCAGCTCAAGACCCTCAGCGTCGGCTTCCAGGTCATG | |
| | GCCGACCTCCCTGCTAACGGCCCTGGCAGCATTAAGGCCGCCTGCA | |
| | ACGAGAGCGGCGTCACCCCTCTCTGCCTCCGCACCCTCTACCGCGT | |
| | CAACTACAAGCCCGCCACCACCGGCAACCTCGTCGCCTTCGCCAGC | |
| | TTCCTTGAGCAGTACGCCCGCTACAGCGACCAGCAGGCCTTCACCCA | |
| | GCGAGTCCTTGGCCCTGGCGTCCCGCTCCAGAACTTCAGCGTCGAG | |
| | ACTGTCAACGGCGGAGCCAACGACCAGCAGAGCAAGCTCGATAGCG | |
| | GCGAGGCCAACCTCGACCTCCAGTACGTCATGGCCATGTCCCACCC | |
| | CATCCCCATCCTTGAGTACAGCACTGGCGGCCGAGGCCCCCTCGTC | |
| | CCTACTCTCGATCAGCCCAACGCCAACAACAGCAGCAACGAGCCCTA | |
| | CCTTGAGTTCCTCACCTACCTGCTCGCCCAGCCCGACAGCGCCATTC | |
| | CCCAGACTCTCAGCGTGAGCTACGGCGAGGAAGAACAGAGCGTCCC | |
| | CCGCGACTACGCCATCAAGGTCTGCAACATGTTCATGCAGCTCGGCG | |
| | CTCGCGGCGTCAGCGTCATGTTTAGCAGCGGCGATAGCGGCCCTGG | |
| | CAACGACTGCGTCCGAGCCTCTGACAACGCCACCTTCTTCGGCAGCA | |
| | CCTTCCCTGCCGGCTGCCCCTACGTCACTAGCGTCGGCAGCACCGT | |
| | CGGCTTCGAGCCTGAGCGAGCCGTCAGCTTTAGCTCCGGCGGCTTC | |
| | AGCATCTACCACGCCCGACCCGACTACCAGAACGAGGTCGTCCCCA | |
| | AGTACATCGAGAGCATCAAGGCCAGCGGCTACGAGAAGTTCTTCGAC | |
| | GGCAACGGCCGAGGCATCCCCGATGTCGCTGCTCAGGGCGCTCGCT | |
| | TCGTCGTCATCGACAAGGGCCGCGTCAGCCTCATCAGCGGCACTAG | |
| | CGCTTCCAGCCCCGCCTTCGCTGGCATGGTCGCCCTCGTCAACGCC | |
| | GCTCGCAAGAGCAAGGATATGCCCGCCCTCGGCTTCCTCAACCCCAT | |
| | GCTCTACCAGAACGCTGCCGCCATGACCGACATCGTCAACGGCGCT | |
| | GGCATCGGCTGCCGCAAGCAGCGCACCGAGTTTCCCAACGGTGCCC | |
| | GCTTCAACGCCACCGCCGGATGGGACCCTGTCACTGGCCTTGGCAC | |
| | CCCCCTGTTCGACAAGCTCCTCGCCGTTGGCGCTCCCGGCGTCCCT | |
| | AACGCCTAA | |
| 94 | ATGCAGACCTTCGGTGCTTTTCTCGTTTCCTTCCTCGCCGCCAGCGG | Togninia |
| | CCTGGCCGCGGCCTCCGATGTCGTCCTTGAGTCTCTCCGCGAGGTC | minima |
| | CCCCAGGGCTGGAAGCGACTCCGAGATGCCGACCCCGAGCAGAGCA | UCRPA7 |
| | TCAAGCTCCGCATTGCCCTTGAGCAGCCCAACCTCGACCTCTTCGAG | |
| | CAGACCCTCTACGACATCAGCAGCCCCGACCACCCCAAGTACGGCC | |
| | AGCACCTCAAGAGCCACGAGCTGCGCGACATCATGGCCCCTCGCGA | |
| | GGAATCCACTGCCGCCGTCATTGCCTGGCTCCAGGATGCTGGCCTC | |
| | AGCGGCAGCCAGATCGAGGACGACAGCGACTGGATCAACATCCAGA | |
| | CCACCGTCGCCCAGGCCAACGACATGCTCAACACCACCTTCGGCCT | |
| | CTTCGCCCAAGAGGGCACCGAGGTCAACCGCATTCGCGCCCTCGCC | |
| | TACAGCGTCCCCGAGGAAATTGTCCCCCACGTCAAGATGATCGCCCC | |
| | CATCATCCGCTTCGGCCAGCTCCGCCCTCAGATGAGCCACATCTTCA | |
| | GCCACGAGAAGGTCGAGGAAACCCCCAGCATCGGCACCATCAAGGC | |
| | CGCTGCCATCCCCAGCGTCGACCTCAACGTCACCGCCTGCAACGCC | |
| | AGCATCACCCCCGAGTGCCTCCGCGCCCTCTACAACGTCGGCGACT | |
| | ACGAGGCCGACCCCAGCAAGAAGTCCCTCTTCGGCGTCTGCGGCTA | |
| | CCTTGAGCAGTACGCCAAGCACGACCAGCTCGCCAAGTTCGAGCAG | |
| | ACGTACGCCCCCTACGCCATCGGCGCCGACTTCAGCGTCGTCACCA | |
| | TCAACGGCGGAGGCGACAACCAGACCAGCACCATCGACGACGGCGA | |
| | GGCCAACCTCGACATGCAGTACGCCGTCAGCATGGCCTACAAGACC | |
| | CCCATCACCTACTACAGCACTGGCGGCCGAGGCCCCCTCGTCCCTG | |
| | ATCTCGATCAGCCCGACCCCAACGACGTCAGCAACGAGCCCTACCTC | |
| | GACTTCGTCAGCTACCTCCTCAAGCTCCCCGACAGCAAGCTCCCCCA | |
| | GACCATCACCACCAGCTACGGCGAGGACGAGCAGAGCGTCCCCCGC | |
| | AGCTACGTCGAGAAGGTCTGCACCATGTTCGGCGCCCTTGGCGCCC | |
| | GAGGCGTCAGCGTCATTTTCAGCTCTGGCGACACCGGCGTCGGCAG | |
| | CGCCTGCCAGACTAACGACGGCAAGAACACCACCCGCTTTCTGCCCA | |
| | TCTTCCCTGCCGCCTGCCCCTACGTCACTAGCGTCGGCGGCACCCG | |
| | CTACGTCGATCCTGAGGTCGCCGTCAGCTTCAGCAGCGGCGGCTTC | |
| | AGCGACATCTTCCCCACCCCCCTGTACCAGAAGGGCGCCGTCAGCG | |
| | GCTACCTCAAGATCCTCGGCGACCGCTGGAAGGGCCTCTACAACCCT | |
| | CACGGCCGAGGCTTCCCTGACGTCAGCGGCCAGTCTGTCCGCTACC | |
| | ACGTCTTTGACTACGGCAAGGACGTCATGTACAGCGGCACCAGCGC | |
| | CAGCGCCCCCATGTTTGCTGCTCTCGTCAGCCTCCTCAACAACGCCC | |
| | GCCTCGCCAAGAAGCTCCCCCCTATGGGCTTCCTCAACCCCTGGCTC | |
| | TACACCGTCGGCTTCAACGGCCTCACCGACATCGTCCACGGCGGCT | |
| | CTACTGGCTGCACCGGCACCGATGTCTACAGCGGCCTGCCTACCCC | |
| | CTTCGTCCCCTACGCCTCTTGGAACGCCACCGTCGGCTGGGACCCT | |
| | GTCACTGGCCTTGGCACCCCCCTGTTCGACAAGCTCCTCAACCTCAG | |
| | CACCCCCAACTTCCACCTCCCCCACATCGGCGGCCACTAA | |
| 95 | ATGCAGACCTTCGGTGCTTTTCTCGTTTCCTTCCTCGCCGCCAGCGG | Bipolaris |
| | CCTGGCCGCGGCCTCTACCACTTCTCACGTCGAGGGCGAGGTCGTC | maydis C5 |
| | GAGCGCCTTCATGGCGTCCCTGAGGGCTGGTCACAGGTCGGCGCTC | |
| | CCAACCCCGACCAGAAGCTCCGCTTCCGCATTGCCGTCCGCAGCGC | |
| | CGACAGCGAGCTGTTCGAGCGCACCCTCATGGAAGTCAGCAGCCCC | |
| | AGCCACCCCCGCTACGGCCAGCACCTCAAGCGCCACGAGCTGAAGG | |
| | ACCTCATCAAGCCTCGCGCCAAGAGCACCAGCAACATCCTCAACTGG | |
| | CTCCAAGAGAGCGGCATCGAGGCCCGCGACATCCAGAACGACGGCG | |
| | AGTGGATCAGCTTCTACGCCCCCGTCAAGCGAGCCGAGCAGATGAT | |
| | GAGCACCACCTTCAAGACCTACCAGAACGAGGCCCGAGCCAACATCA | |
| | AGAAGATCCGCAGCCTCGACTACAGCGTCCCCAAGCACATCCGCGA | |
| | CGACATCGACATCATCCAGCCCACCACGCGCTTCGGCCAGATCCAG | |
| | CCTGAGCGCAGCCAGGTCTTTAGCCAAGAGGAAGTCCCCTTCAGCG | |
| | CCCTCGTCGTCAACGCCACGTGCAACAAGAAGATCACCCCCGACTGC | |
| | CTCGCCAACCTCTACAACTTCAAGGACTACGACGCCAGCGACGCCAA | |
| | CGTCACGATCGGCGTCAGCGGCTTCCTTGAGCAGTACGCCCGCTTC | |
| | GACGACCTCAAGCAGTTCATCAGCACCTTCCAGCCCAAGGCCGCTG | |
| | GCTCCACCTTCCAGGTCACCAGCGTCAACGCTGGCCCCTTCGACCA | |
| | GAACAGCACCGCCTCTAGCGTCGAGGCCAACCTCGACATCCAGTACA | |
| | CCACCGGCCTCGTCGCCCCCGACATCGAGACTCGCTACTTCACCGT | |
| | CCCCGGACGCGGCATCCTCATCCCCGACCTCGACCAGCCTACCGAG | |
| | AGCGACAACGCCAACGAGCCCTACCTCGACTACTTCACCTACCTCAA | |
| | CAACCTTGAGGACGAGGAACTCCCCGACGTCCTCACCACCAGCTAC | |
| | GGCGAGAGCGAGCAGAGCGTCCCTGCCGAGTACGCCAAGAAGGTCT | |
| | GCAACCTCATCGGCCAGCTCGGCGCTCGCGGCGTCAGCGTCATTTT | |
| | CAGCAGCGGCGACACCGGCCCTGGCAGCGCCTGCCAGACTAACGAC | |
| | GGCAAGAACACCACCCGCTTTCTGCCCATCTTCCCCGCCAGCTGCCC | |
| | CTACGTCACTAGCGTCGGCGGCACTGTCGGCGTCGAGCCTGAGAAG | |
| | GCCGTCAGCTTTAGCAGCGGCGGCTTCAGCGACCTCTGGCCCCGAC | |
| | CTGCCTACCAAGAGAAGGCCGTGAGCGAGTACCTTGAGAAGCTCGG | |
| | CGACCGCTGGAACGGCCTCTACAACCCTCAGGGCCGAGGCTTCCCT | |
| | GACGTCGCTGCTCAGGGCCAGGGCTTCCAGGTCTTTGACAAGGGCC | |
| | GCCTCATCTCGGTCGGCGGCACATCTGCTTCCGCCCCTGTCTTTGCC | |
| | AGCGTCGTCGCCCTCCTCAACAACGCCCGAAAGGCTGCCGGAATGA | |
| | GCAGCCTCGGCTTCCTCAACCCCTGGATCTACGAGCAGGGCTACAA | |
| | GGGCCTCACCGACATCGTCGCTGGCGGCTCTACTGGCTGCACCGGC | |
| | CGCTCTATCTACAGCGGCCTCCCTGCCCCCCTGGTCCCTTACGCTTC | |
| | TTGGAACGCCACCGAGGGCTGGGACCCCGTCACTGGCTATGGCACC | |
| | CCCGACTTCAAGCAGCTCCTCACCCTCGCCACCGCCCCCAAGTCTG | |
| | GCGAGCGACGAGTTCGACGAGGCGGCCTTGGAGGCCAGGCTTAA | |

| SEQ | | | |
|---|---|---|---|
| ID | | | |
| No.: | Description | Sequence | Origin |
| 96 | TRI045 | ATGCGTCTTCTCAAATTTGTGTGCCTGTTGGCATC | Human skin |
| | Genomic | AGTTGCCGCCGCAAAGCCTACTCCAGGGGCGTC | fungus |
| | sequence | ACACAAGGTCATTGAACATCTTGACTTTGTTCCAG | Arthroderma |
| | CDS | AAGGATGGCAGATGGTTGGTGCCGCGGACCCTG | benhamiae |
| | | CTGCTATCATTGATTTCTGGCTTGCCATCGAGCGC | |
| | | GAAAACCCAGAAAAGCTCTACGACACCATCTATG | |
| | | ACGTCTCCACCCCTGGACGCGCACAATATGGCAA | |
| | | ACATTTGAAGCGTGAGGAATTGGATGACTTACTAC | |
| | | GCCCAAGGGCAGAGACGAGTGAGAGCATCATCA | |
| | | ACTGGCTCACCAATGGTGGAGTCAACCCACAACA | |
| | | TATTCGGGATGAAGGGGACTGGGTCAGATTCTCT | |
| | | ACCAATGTCAAGACTGCCGAAACGTTGATGAATA | |
| | | CCCGCTTCAACGTCTTCAAGGACAACCTAAATTCC | |
| | | GTTTCAAAAATTCGAACTTTGGAGTATTCCGTCCC | |
| | | TGTAGCTATATCAGCTCATGTCCAAATGATCCAGC | |
| | | CAACTACCTTATTTGGACGACAGAAGCCACAGAA | |
| | | CAGTTTGATCCTAAACCCCTTGACCAAGGATCTAG | |
| | | AATCCATGTCCGTTGAAGAATTTGCTGCTTCTCAG | |
| | | TGCAGGTCCTTAGTGACTACTGCCTGCCTTCGAG | |
| | | AATTGTACGGACTTGGTGACCGTGTCACTCAGGC | |
| | | TAGGGATGACAACCGTATTGGAGTATCCGGCTTT | |
| | | TTGGAGGAGTACGCCCAATACCGCGATCTTGAGC | |
| | | TCTTCCTCTCTCGCTTTGAGCCATCCGCCAAAGG | |
| | | ATTTAATTTCAGTGAAGGCCTTATTGCCGGAGGAA | |
| | | AGAACACTCAGGGTGGTCCTGGAAGCTCTACTGA | |
| | | GGCCAACCTTGATATGCAATATGTCGTCGGTCTG | |
| | | TCCCACAAGGCAAAGGTCACCTATTACTCCACCG | |
| | | CTGGCCGTGGCCCATTAATTCCCGATCTATCTCA | |
| | | GCCAAGCCAAGCTTCAAACAACAACGAACCATAC | |
| | | CTTGAACAGCTGCGGTACCTCGTAAAGCTCCCCA | |
| | | AGAACCAGCTTCCATCTGTATTGACAACTTCCTAT | |
| | | GGAGACACAGAACAGAGCTTGCCCGCCAGCTATA | |
| | | CCAAAGCCACTTGCGACCTCTTTGCTCAGCTAGG | |
| | | AACTATGGGTGTGTCTGTTATCTTCAGCAGTGGTG | |
| | | ATACCGGGCCCGGAAGCTCATGCCAGACCAACG | |
| | | ATGGCAAGAATGCGACTCGCTTCAACCCTATCTA | |
| | | CCCAGCTTCTTGCCCGTTTGTGACCTCCATCGGT | |
| | | GGAACCGTTGGTACCGGTCCTGAGCGTGCAGTTT | |
| | | CATTCTCCTCTGGTGGCTTCTCAGACAGGTTCCC | |
| | | CCGCCCACAATATCAGGATAACGCTGTTAAAGAC | |
| | | TACCTGAAAATTTTGGGCAACCAGTGGAGCGGAT | |
| | | TGTTTGACCCCAACGGCCGTGCTTTCCCAGATAT | |
| | | CGCAGCTCAGGGATCAAATTATGCTGTCTATGAC | |
| | | AAGGGAAGGATGACTGGAGTCTCCGGCACCAGT | |
| | | GCATCCGCCCCTGCCATGGCTGCCATCATTGCCC | |
| | | AGCTTAACGATTTCCGACTGGCAAAGGGCTCTCC | |
| | | TGTGCTGGGATTCTTGAACCCATGGATATATTCCA | |
| | | AGGGTTTCTCTGGCTTTACAGATATTGTTGATGGC | |
| | | GGTTCCAGGGGTTGCACTGGTTACGATATATACA | |
| | | GCGGCTTGAAAGCGAAGAAGGTTCCCTACGCAAG | |
| | | CTGGAATGCAACTAAGGGATGGGACCCAGTAACG | |
| | | GGATTTGGTACTCCCAACTTCCAAGCTCTCACTAA | |
| | | AGTGCTGCCCTAA | |
| 97 | TRI045 | ATGCAGACCTTCGGTGCTTTTCTCGTTTCCTTCCT | Human skin |
| | Synthetic | CGCCGCCAGCGGCCTGGCCGCGGCCAAGCCTAC | fungus |
| | Gene | TCCTGGCGCTTCCCACAAGGTCATCGAGCACCTC | Arthroderma |
| | optimized for | GACTTCGTCCCCGAGGGCTGGCAGATGGTCGGC | benhamiae |
| | expression in | GCTGCTGACCCTGCCGCCATCATCGACTTTTGGC | |
| | Trichoderma | TCGCCATCGAGCGCGAGAACCCCGAGAAGCTCTA | |
| | with | CGACACCATCTACGACGTCAGCACCCCCGGACG | |
| | Trichoderma | CGCCCAGTACGGCAAGCACCTCAAGCGCGAGGA | |
| | sequence | AGCGAGAGCATCATCAACTGGCTCACCAACGGCG | |
| | underlined | GCGTCAACCCCCAGCACATTCGCGACGAGGGCG | |
| | | ACTGGGTCCGCTTCAGCACCAACGTCAAGACCGC | |
| | | CGAGACTCTCATGAACACCCGCTTCAACGTCTTTA | |
| | | AGGACAACCTCAACAGCGTCAGCAAGATCCGCAC | |
| | | CCTTGAGTACAGCGTCCCCGTCGCCATCAGCGCC | |
| | | CACGTCCAGATGATCCAGCCCACCACCCTCTTCG | |
| | | GCCGCCAGAAGCCCCAGAACAGCCTCATCCTCAA | |
| | | CCCCCTCACCAAGGACCTTGAGAGCATGAGCGTC | |
| | | GAAGAGTTCGCCGCCAGCCAGTGCCGCAGCCTC | |
| | | GTCACTACTGCCTGCCTCCGCGAGCTGTACGGCC | |
| | | TCGGCGATCGAGTCACCCAGGCCCGCGACGACA | |
| | | ACCGAATTGGCGTCAGCGGCTTCCTCGAAGAGTA | |
| | | CGCCCAGTACCGCGACCTTGAGCTGTTCCTCAGC | |
| | | CGCTTCGAGCCCAGCGCCAAGGGCTTCAACTTCA | |
| | | GCGAGGGCCTGATCGCTGGCGGCAAGAACACCC | |
| | | AGGGTGGCCCTGGCTCTAGCACCGAGGCCAACC | |
| | | TCGACATGCAGTACGTCGTCGGCCTCAGCCACAA | |
| | | GGCCAAGGTCACCTACTACAGCACTGCCGGCCG | |
| | | AGGCCCCCTCATCCCTGATCTCTCACAGCCCAGC | |
| | | CAGGCCAGCAACAACAACGAGCCCTACCTTGAGC | |
| | | AGCTCCGCTACCTCGTCAAGCTCCCCAAGAACCA | |
| | | GCTCCCCAGCGTCCTCACCACCAGCTACGGCGA | |
| | | CACCGAGCAGAGCCTCCCCGCCAGCTACACCAA | |
| | | GGCCACGTGCGACCTCTTCGCCCAGCTCGGCAC | |
| | | TATGGGCGTCAGCGTCATCTTCAGCAGCGGCGAC | |
| | | ACTGGCCCTGGCAGCTCGTGCCAGACCAACGAC | |
| | | GGCAAGAACGCCACGCGCTTCAACCCCATCTACC | |
| | | CCGCCAGCTGCCCCTTCGTCACCAGCATTGGCG | |
| | | GCACCGTCGGCACCGGCCCTGAGCGAGCTGTCA | |
| | | GCTTTAGCAGCGGCGGCTTCAGCGACCGCTTCCC | |
| | | TCGCCCTCAGTACCAGGACAACGCCGTCAAGGAC | |
| | | TACCTCAAGATCCTCGGCAACCAGTGGTCCGGCC | |
| | | TCTTCGACCCTAACGGCCGAGCCTTCCCCGACAT | |
| | | TGCCGCCCAGGGCAGCAACTACGCCGTCTACGA | |
| | | CAAGGGCCGCATGACCGGCGTTAGCGGCACTTC | |
| | | TGCTTCCGCCCCTGCTATGGCCGCCATCATTGCC | |
| | | CAGCTCAACGACTTCCGCCTCGCCAAGGGCAGC | |
| | | CCCGTCCTCGGCTTTCTCAACCCCTGGATCTACA | |
| | | GCAAGGGCTTCAGCGGCTTCACCGACATCGTCGA | |
| | | CGGCGGCTCTAGGGGCTGCACCGGCTACGACAT | |
| | | CTACAGCGGCCTCAAGGCCAAGAAGGTCCCCTAC | |
| | | GCCAGCTGGAACGCCACCAAGGGCTGGGACCCC | |
| | | GTCACCGGCTTTGGCACCCCCAACTTCCAGGCCC | |
| | | TGACCAAGGTCCTGCCCTAA | |
| 98 | TRI045 | KPTPGASHKVIEHLDFVPEGWQMVGAADPAAIIDFW | Human skin |
| | pre_pro | LAIERENPEKLYDTIYDVSTPGRAQYGKHLKREELD | fungus |
| | amino acid | DLLRPRAETSESIINWLTNGGVNPQHIRDEGDVVVRF | Arthroderma |
| | sesquence | STNVKTAETLMNTRFNVFKDNLNSVSKIRTLEYSVP | benhamiae |
| | | VAISAHVQMIQPTTLFGRQKPQNSLILNPLTKDLESM | |
| | | SVEEFAASQCRSLVTTACLRELYGLGDRVTQARDD | |
| | | NRIGVSGFLEEYAQYRDLELFLSRFEPSAKGFNFSE | |
| | | GLIAGGKNTQGGPGSSTEANLDMQYVVGLSHKAKV | |
| | | TYYSTAGRGPLIPDLSQPSQASNNNEPYLEQLRYLV | |
| | | KLPKNQLPSVLTTSYGDTEQSLPASYTKATCDLFAQ | |
| | | LGTMGVSVIFSSGDTGPGSSCQTNDGKNATRFNPIY | |
| | | PASCPFVTSIGGTVGTGPERAVSFSSGGFSDRFPR | |
| | | PQYQDNAVKDYLKILGNQWSGLFDPNGRAFPDIAA | |
| | | QGSNYAVYDKGRMTGVSGTSASAPAMAAIIAQLND | |
| | | FRLAKGSPVLGFLNPWIYSKGFSGFTDIVDGGSRGC | |
| | | TGYDIYSGLKAKKVPYASWNATKGWDPVTGFGTPN | |
| | | FQALTKVLP | |
| 99 | TRI045 | CRSLVTTACLRELYGLGDRVTQARDDNRIGVSGFLE | Human skin |
| | mature | EYAQYRDLELFLSRFEPSAKGFNFSEGLIAGGKNTQ | fungus |
| | Interpro | GGPGSSTEANLDMQYVVGLSHKAKVTYYSTAGRGP | Arthroderma |
| | domain | LIPDLSQPSQASNNNEPYLEQLRYLVKLPKNQLPSV | benhamiae |
| | IPR000209 | LTTSYGDTEQSLPASYTKATCDLFAQLGTMGVSVIF | |
| | Peptidase | SSGDTGPGSSCQTNDGKNATRFNPIYPASCPFVTSI | |
| | S8/S53 dom | GGTVGTGPERAVSFSSGGFSDRFPRPQYQDNAVK | |
| | | DYLKILGNQWSGLFDPNGRAFPDIAAQGSNYAVYD | |
| | | KGRMTGVSGTSASAPAMAAIIAQLNDFRLAKGSPVL | |
| | | GFLNPWIYSKGFSGFTDIVDGGSRGCTGYDIYSGLK | |
| | | AKKVPYASWNATKGWDPVTGFGTPNFQALTKVLP | |

The at least one proline tolerant tripeptidyl peptidase may:
(a) comprise the amino acid sequence SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof;
(b) comprise an amino acid having at least 70% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof;
(c) be encoded by a nucleotide sequence comprising the sequence SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97;
(d) be encoded by a nucleotide sequence comprising at least about 70% sequence identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97;
(e) be encoded by a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 under medium stringency conditions; or
(f) be encoded by a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 due to degeneracy of the genetic code.

The proline tolerant tripeptidyl peptidase may be expressed as a polypeptide sequence which undergoes further post-transcriptional and/or post-translational modification.

In one embodiment the proline tolerant tripeptidyl peptidase may comprise the amino acid sequence SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 98 or a functional fragment thereof.

In another embodiment the proline tolerant tripeptidyl peptidase comprise an amino acid having at least 70% identity to SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 98 or a functional fragment thereof.

In one embodiment the proline tolerant tripeptidyl peptidase may comprise the amino acid sequence SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 98 or a functional fragment thereof.

In another embodiment the proline tolerant tripeptidyl peptidase comprise an amino acid having at least 70% identity to SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 98 or a functional fragment thereof;

In another embodiment the proline tolerant tripeptidyl peptidase may be a “mature” proline tolerant tripeptidyl peptidase which has undergone post-transcriptional and/or post-translational modification (e.g. post-translational cleavage). Suitably such modification may lead to an activation of the enzyme.

Suitably the proline tolerant tripeptidyl peptidase may comprise the amino acid sequence SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 99 or a functional fragment thereof.

In another embodiment the proline tolerant tripeptidyl peptidase comprise an amino acid having at least 70% identity to SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 99 or a functional fragment thereof.

In a yet further embodiment the proline tolerant tripeptidyl peptidase may comprise the amino acid sequence SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 99 or a functional fragment thereof.

In another embodiment the proline tolerant tripeptidyl peptidase comprise an amino acid having at least 70% identity to SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 99 or a functional fragment thereof.

The term “functional fragment” is a portion of an amino acid sequence that retains its peptidase enzyme activity. Therefore, a functional fragment of a proline tolerant tripeptidyl peptidase is a portion of a proline tolerant tripeptidyl peptidase predominantly having exopeptidase activity wherein said proline tolerant tripeptidyl peptidase is capable of cleaving tri-peptides from the N-terminus of peptides having
(i) (A) Proline at P1; and
   (B) An amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; and/or
(ii) (a′) Proline at P1′; and
   (b′) An amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′. Alternatively or additionally a functional fragment of a proline tolerant tripeptidyl peptidase is a portion of a proline tolerant tripeptidyl peptidase predominantly having exopeptidase activity and capable of cleaving tri-peptides from the N-terminus of peptides having proline at P1 and P1′.

The “portion” is any portion that still has the activity as defined above; suitably a portion may be at least 50 amino acids in length, more suitably at least 100. In other embodiments the portion may be about 150 or about 200 amino acids in length.

In one embodiment the functional fragment may be portion of a proline tolerant tripeptidyl peptidase following post transcriptional and/or post-translational modification (e.g. cleavage). Suitably the functional fragment may comprise a sequence shown as: SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55 or SEQ ID No. 99.

The proline tolerant tripeptidyl peptidase may comprise one or more amino acid sequence selected from SEQ ID No. 1, or a functional fragment thereof.

The proline tolerant tripeptidyl peptidase may comprise an amino acid having at least 70% identity to SEQ ID No. 1 or a functional fragment thereof.

The proline tolerant tripeptidyl peptidase may comprise one or more amino acid sequence selected from SEQ ID No. 2, or a functional fragment thereof.

The proline tolerant tripeptidyl peptidase may comprise an amino acid having at least 70% identity to SEQ ID No. 2 or a functional fragment thereof.

The proline tolerant tripeptidyl peptidase may comprise one or more amino acid sequence selected from SEQ ID No. 3 or a functional fragment thereof.

The proline tolerant tripeptidyl peptidase may comprise an amino acid having at least 70% identity to SEQ ID No. 3 or a functional fragment thereof.

The proline tolerant tripeptidyl peptidase may comprise one or more amino acid sequence selected from SEQ ID No. 4 or a functional fragment thereof.

The proline tolerant tripeptidyl peptidase may comprise an amino acid having at least 70% identity to SEQ ID No. 4 or a functional fragment thereof.

The proline tolerant tripeptidyl peptidase may comprise one or more amino acid sequence selected from SEQ ID No. 5 or a functional fragment thereof.

The proline tolerant tripeptidyl peptidase may comprise an amino acid having at least 70% identity to SEQ ID No. 5 or a functional fragment thereof.

The proline tolerant tripeptidyl peptidase may comprise one or more amino acid sequence selected from SEQ ID No. 6 or a functional fragment thereof.

The proline tolerant tripeptidyl peptidase may comprise an amino acid having at least 70% identity to SEQ ID No. 6 or a functional fragment thereof.

The proline tolerant tripeptidyl peptidase may comprise one or more amino acid sequence selected from SEQ ID No. 7 or a functional fragment thereof.

The proline tolerant tripeptidyl peptidase may comprise an amino acid having at least 70% identity to SEQ ID No. 7 or a functional fragment thereof.

The proline tolerant tripeptidyl peptidase may comprise one or more amino acid sequence selected from SEQ ID No. 8 or a functional fragment thereof.

The proline tolerant tripeptidyl peptidase may comprise an amino acid having at least 70% identity to SEQ ID No. 8 or a functional fragment thereof.

The proline tolerant tripeptidyl peptidase may comprise one or more amino acid sequence selected from SEQ ID No. 9 or a functional fragment thereof.

The proline tolerant tripeptidyl peptidase may comprise an amino acid having at least 70% identity to SEQ ID No. 9 or a functional fragment thereof.

The proline tolerant tripeptidyl peptidase may comprise one or more amino acid sequence selected from SEQ ID No. 10 or a functional fragment thereof.

The proline tolerant tripeptidyl peptidase may comprise an amino acid having at least 70% identity to SEQ ID No. 10 or a functional fragment thereof.

The proline tolerant tripeptidyl peptidase may comprise one or more amino acid sequence selected from SEQ ID No. 11 or a functional fragment thereof.

The proline tolerant tripeptidyl peptidase may comprise an amino acid having at least 70% identity to SEQ ID No. 11 or a functional fragment thereof.

The proline tolerant tripeptidyl peptidase may comprise one or more amino acid sequence selected from SEQ ID No. 12 or a functional fragment thereof.

The proline tolerant tripeptidyl peptidase may comprise an amino acid having at least 70% identity to SEQ ID No. 12 or a functional fragment thereof.

The proline tolerant tripeptidyl peptidase may comprise one or more amino acid sequence selected from SEQ ID No. 13 or a functional fragment thereof.

The proline tolerant tripeptidyl peptidase may comprise an amino acid having at least 70% identity to SEQ ID No. 13 or a functional fragment thereof.

The proline tolerant tripeptidyl peptidase may comprise one or more amino acid sequence selected from SEQ ID No. 14 or a functional fragment thereof.

The proline tolerant tripeptidyl peptidase may comprise an amino acid having at least 70% identity to SEQ ID No. 14 or a functional fragment thereof.

The proline tolerant tripeptidyl peptidase may comprise one or more amino acid sequence selected from SEQ ID No. 15 or a functional fragment thereof.

The proline tolerant tripeptidyl peptidase may comprise an amino acid having at least 70% identity to SEQ ID No. 15 or a functional fragment thereof.

The proline tolerant tripeptidyl peptidase may comprise one or more amino acid sequence selected from SEQ ID No. 16 or a functional fragment thereof.

The proline tolerant tripeptidyl peptidase may comprise an amino acid having at least 70% identity to SEQ ID No. 16 or a functional fragment thereof.

The proline tolerant tripeptidyl peptidase may comprise one or more amino acid sequence selected from SEQ ID No. 17 or a functional fragment thereof.

The proline tolerant tripeptidyl peptidase may comprise an amino acid having at least 70% identity to SEQ ID No. 17 or a functional fragment thereof.

The proline tolerant tripeptidyl peptidase may comprise one or more amino acid sequence selected from SEQ ID No. 18 or a functional fragment thereof.

The proline tolerant tripeptidyl peptidase may comprise an amino acid having at least 70% identity to SEQ ID No. 18 or a functional fragment thereof.

The proline tolerant tripeptidyl peptidase may comprise one or more amino acid sequence selected from SEQ ID No. 19 or a functional fragment thereof.

The proline tolerant tripeptidyl peptidase may comprise an amino acid having at least 70% identity to SEQ ID No. 19 or a functional fragment thereof.

The proline tolerant tripeptidyl peptidase may comprise one or more amino acid sequence selected from SEQ ID No. 20 or a functional fragment thereof.

The proline tolerant tripeptidyl peptidase may comprise an amino acid having at least 70% identity to SEQ ID No. 20 or a functional fragment thereof.

The proline tolerant tripeptidyl peptidase may comprise one or more amino acid sequence selected from SEQ ID No. 21 or a functional fragment thereof.

The proline tolerant tripeptidyl peptidase may comprise an amino acid having at least 70% identity to SEQ ID No. 21 or a functional fragment thereof.

The proline tolerant tripeptidyl peptidase may comprise one or more amino acid sequence selected from SEQ ID No. 22 or a functional fragment thereof.

The proline tolerant tripeptidyl peptidase may comprise an amino acid having at least 70% identity to SEQ ID No. 22 or a functional fragment thereof.

The proline tolerant tripeptidyl peptidase may comprise one or more amino acid sequence selected from SEQ ID No. 23 or a functional fragment thereof.

The proline tolerant tripeptidyl peptidase may comprise an amino acid having at least 70% identity to SEQ ID No. 23 or a functional fragment thereof.

The proline tolerant tripeptidyl peptidase may comprise one or more amino acid sequence selected from SEQ ID No. 24 or a functional fragment thereof.

The proline tolerant tripeptidyl peptidase may comprise an amino acid having at least 70% identity to SEQ ID No. 24 or a functional fragment thereof.

The proline tolerant tripeptidyl peptidase may comprise one or more amino acid sequence selected from SEQ ID No. 25 or a functional fragment thereof.

The proline tolerant tripeptidyl peptidase may comprise an amino acid having at least 70% identity to SEQ ID No. 25 or a functional fragment thereof.

The proline tolerant tripeptidyl peptidase may comprise one or more amino acid sequence selected from SEQ ID No. 26 or a functional fragment thereof.

The proline tolerant tripeptidyl peptidase may comprise an amino acid having at least 70% identity to SEQ ID No. 26 or a functional fragment thereof.

The proline tolerant tripeptidyl peptidase may comprise one or more amino acid sequence selected from SEQ ID No. 27 or a functional fragment thereof.

The proline tolerant tripeptidyl peptidase may comprise an amino acid having at least 70% identity to SEQ ID No. 27 or a functional fragment thereof.

The proline tolerant tripeptidyl peptidase may comprise one or more amino acid sequence selected from SEQ ID No. 28, or a functional fragment thereof.

The proline tolerant tripeptidyl peptidase may comprise an amino acid having at least 70% identity to SEQ ID No. 28 or a functional fragment thereof.

The proline tolerant tripeptidyl peptidase may comprise one or more amino acid sequence selected from SEQ ID No. 29, or a functional fragment thereof.

The proline tolerant tripeptidyl peptidase may comprise an amino acid having at least 70% identity to SEQ ID No. 29 or a functional fragment thereof.

The proline tolerant tripeptidyl peptidase may comprise one or more amino acid sequence selected from SEQ ID No. 30 or a functional fragment thereof.

The proline tolerant tripeptidyl peptidase may comprise an amino acid having at least 70% identity to SEQ ID No. 30 or a functional fragment thereof.

The proline tolerant tripeptidyl peptidase may comprise one or more amino acid sequence selected from SEQ ID No. 31 or a functional fragment thereof.

The proline tolerant tripeptidyl peptidase may comprise an amino acid having at least 70% identity to SEQ ID No. 31 or a functional fragment thereof.

The proline tolerant tripeptidyl peptidase may comprise one or more amino acid sequence selected from SEQ ID No. 32 or a functional fragment thereof.

The proline tolerant tripeptidyl peptidase may comprise an amino acid having at least 70% identity to SEQ ID No. 32 or a functional fragment thereof.

The proline tolerant tripeptidyl peptidase may comprise one or more amino acid sequence selected from SEQ ID No. 33 or a functional fragment thereof.

The proline tolerant tripeptidyl peptidase may comprise an amino acid having at least 70% identity to SEQ ID No. 33 or a functional fragment thereof.

The proline tolerant tripeptidyl peptidase may comprise one or more amino acid sequence selected from SEQ ID No. 34 or a functional fragment thereof.

The proline tolerant tripeptidyl peptidase may comprise an amino acid having at least 70% identity to SEQ ID No. 34 or a functional fragment thereof.

The proline tolerant tripeptidyl peptidase may comprise one or more amino acid sequence selected from SEQ ID No. 35 or a functional fragment thereof.

The proline tolerant tripeptidyl peptidase may comprise an amino acid having at least 70% identity to SEQ ID No. 35 or a functional fragment thereof.

The proline tolerant tripeptidyl peptidase may comprise one or more amino acid sequence selected from SEQ ID No. 36 or a functional fragment thereof.

The proline tolerant tripeptidyl peptidase may comprise an amino acid having at least 70% identity to SEQ ID No. 36 or a functional fragment thereof.

The proline tolerant tripeptidyl peptidase may comprise one or more amino acid sequence selected from SEQ ID No. 37 or a functional fragment thereof.

The proline tolerant tripeptidyl peptidase may comprise an amino acid having at least 70% identity to SEQ ID No. 37 or a functional fragment thereof.

The proline tolerant tripeptidyl peptidase may comprise one or more amino acid sequence selected from SEQ ID No. 38 or a functional fragment thereof.

The proline tolerant tripeptidyl peptidase may comprise an amino acid having at least 70% identity to SEQ ID No. 38 or a functional fragment thereof.

The proline tolerant tripeptidyl peptidase may comprise one or more amino acid sequence selected from SEQ ID No. 39 or a functional fragment thereof.

The proline tolerant tripeptidyl peptidase may comprise an amino acid having at least 70% identity to SEQ ID No. 39 or a functional fragment thereof.

The proline tolerant tripeptidyl peptidase may comprise one or more amino acid sequence selected from SEQ ID No. 40 or a functional fragment thereof.

The proline tolerant tripeptidyl peptidase may comprise an amino acid having at least 70% identity to SEQ ID No. 40 or a functional fragment thereof.

The proline tolerant tripeptidyl peptidase may comprise one or more amino acid sequence selected from SEQ ID No. 41 or a functional fragment thereof.

The proline tolerant tripeptidyl peptidase may comprise an amino acid having at least 70% identity to SEQ ID No. 41 or a functional fragment thereof.

The proline tolerant tripeptidyl peptidase may comprise one or more amino acid sequence selected from SEQ ID No. 42 or a functional fragment thereof.

The proline tolerant tripeptidyl peptidase may comprise an amino acid having at least 70% identity to SEQ ID No. 42 or a functional fragment thereof.

The proline tolerant tripeptidyl peptidase may comprise one or more amino acid sequence selected from SEQ ID No. 43 or a functional fragment thereof.

The proline tolerant tripeptidyl peptidase may comprise an amino acid having at least 70% identity to SEQ ID No. 43 or a functional fragment thereof.

The proline tolerant tripeptidyl peptidase may comprise one or more amino acid sequence selected from SEQ ID No. 44 or a functional fragment thereof.

The proline tolerant tripeptidyl peptidase may comprise an amino acid having at least 70% identity to SEQ ID No. 44 or a functional fragment thereof.

The proline tolerant tripeptidyl peptidase may comprise one or more amino acid sequence selected from SEQ ID No. 45 or a functional fragment thereof.

The proline tolerant tripeptidyl peptidase may comprise an amino acid having at least 70% identity to SEQ ID No. 45 or a functional fragment thereof.

The proline tolerant tripeptidyl peptidase may comprise one or more amino acid sequence selected from SEQ ID No. 46 or a functional fragment thereof.

The proline tolerant tripeptidyl peptidase may comprise an amino acid having at least 70% identity to SEQ ID No. 46 or a functional fragment thereof.

The proline tolerant tripeptidyl peptidase may comprise one or more amino acid sequence selected from SEQ ID No. 47 or a functional fragment thereof.

The proline tolerant tripeptidyl peptidase may comprise an amino acid having at least 70% identity to SEQ ID No. 47 or a functional fragment thereof.

The proline tolerant tripeptidyl peptidase may comprise one or more amino acid sequence selected from SEQ ID No. 48 or a functional fragment thereof.

The proline tolerant tripeptidyl peptidase may comprise an amino acid having at least 70% identity to SEQ ID No. 48 or a functional fragment thereof.

The proline tolerant tripeptidyl peptidase may comprise one or more amino acid sequence selected from SEQ ID No. 49 or a functional fragment thereof.

The proline tolerant tripeptidyl peptidase may comprise an amino acid having at least 70% identity to SEQ ID No. 49 or a functional fragment thereof.

The proline tolerant tripeptidyl peptidase may comprise one or more amino acid sequence selected from SEQ ID No. 50 or a functional fragment thereof.

The proline tolerant tripeptidyl peptidase may comprise an amino acid having at least 70% identity to SEQ ID No. 50 or a functional fragment thereof.

The proline tolerant tripeptidyl peptidase may comprise one or more amino acid sequence selected from SEQ ID No. 51 or a functional fragment thereof.

The proline tolerant tripeptidyl peptidase may comprise an amino acid having at least 70% identity to SEQ ID No. 51 or a functional fragment thereof.

The proline tolerant tripeptidyl peptidase may comprise one or more amino acid sequence selected from SEQ ID No. 52 or a functional fragment thereof.

The proline tolerant tripeptidyl peptidase may comprise an amino acid having at least 70% identity to SEQ ID No. 52 or a functional fragment thereof.

The proline tolerant tripeptidyl peptidase may comprise one or more amino acid sequence selected from SEQ ID No. 53 or a functional fragment thereof.

The proline tolerant tripeptidyl peptidase may comprise an amino acid having at least 70% identity to SEQ ID No. 53 or a functional fragment thereof.

The proline tolerant tripeptidyl peptidase may comprise one or more amino acid sequence selected from SEQ ID No. 54 or a functional fragment thereof.

The proline tolerant tripeptidyl peptidase may comprise an amino acid having at least 70% identity to SEQ ID No. 54 or a functional fragment thereof.

The proline tolerant tripeptidyl peptidase may comprise one or more amino acid sequence selected from SEQ ID No. 55 or a functional fragment thereof.

The proline tolerant tripeptidyl peptidase may comprise an amino acid having at least 70% identity to SEQ ID No. 55 or a functional fragment thereof.

The proline tolerant tripeptidyl peptidase may comprise one or more amino acid sequence selected from SEQ ID No. 98 or a functional fragment thereof.

The proline tolerant tripeptidyl peptidase may comprise an amino acid sequence having at least 70% identity to SEQ ID No. 98 or a functional fragment thereof.

The proline tolerant tripeptidyl peptidase may comprise one or more amino acid sequence selected from SEQ ID No. 99 or a functional fragment thereof.

The proline tolerant tripeptidyl peptidase may comprise an amino acid sequence having at least 70% identity to SEQ ID No. 99 or a functional fragment thereof.

Suitably the proline tolerant tripeptidyl peptidase may comprise an amino acid sequence having at least 80% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof.

Suitably the proline tolerant tripeptidyl peptidase may comprise an amino acid sequence having at least 85% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof.

Suitably the proline tolerant tripeptidyl peptidase may comprise an amino acid sequence having at least 90% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof.

Suitably the proline tolerant tripeptidyl peptidase may comprise an amino acid sequence having at least 95% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof.

Suitably the proline tolerant tripeptidyl peptidase may comprise an amino acid sequence having at least 97% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof.

Suitably the proline tolerant tripeptidyl peptidase may comprise an amino acid sequence having at least 99% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof.

In one embodiment the proline tolerant tripeptidyl peptidase may comprise an amino acid sequence selected from one more of the group consisting of: SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98 and SEQ ID No. 99.

When used for food applications, the proline tolerant tripeptidyl peptidase may comprise an amino acid sequence selected from one more of the group consisting of: SEQ ID No. 1, SEQ ID No. 2, SEQ ID No. 3, SEQ ID No. 29 and SEQ ID No. 30, or a sequence having at least 70% identity thereto; more suitably a sequence having at least 80% thereto or at least 90% thereto.

In some embodiments it may be suitable that the proline tolerant tripeptidyl peptidase may comprise an amino acid sequence selected from the group consisting of SEQ ID No. 3, SEQ ID No. 4, SEQ ID No. 30 and SEQ ID No. 31, or a sequence having at least 70% identity thereto. Suitably a sequence having at least 80% thereto or at least 90% thereto.

Advantageously these particular amino acid sequences may be particularly suited to cleaving peptide and/or protein substrates enriched in lysine, arginine and/or glycine. Particularly where lysine, arginine and/or glycine are present at the P1 position.

When used for feed applications, suitably, the proline tolerant tripeptidyl peptidase may comprise an amino acid sequence selected from one more of the group consisting of: SEQ ID No. 1, SEQ ID No. 2, SEQ ID No. 5, SEQ ID No. 6, SEQ ID No. 7, SEQ ID No. 29, SEQ ID No. 32, SEQ ID No. 33, SEQ ID No. 34, SEQ ID No. 98 and SEQ ID No. 99, or a sequence having at least 70% identity thereto. Suitably a sequence having at least 80% thereto or at least 90% thereto.

Suitably the proline tolerant tripeptidyl peptidase may have the sequence SEQ ID No. 1, SEQ ID No. 2 or SEQ ID No. 29.

The proline tolerant tripeptidyl peptidase may comprise one or more of the sequence motifs selected from the group consisting of: xEANLD, y′Tzx′G and QNFSV.

Suitably, the proline tolerant tripeptidyl peptidase may comprise xEANLD.

x may be one or more amino acid selected from the group consisting of: G, T, S and V.

In another embodiment the proline tolerant tripeptidyl peptidase may comprise y′Tzx′G.

y′ may be one or more amino acid selected from the group consisting of: I, L and V.

z may be one or more amino acid selected from the group consisting of: S and T.

x′ may be one or more amino acid selected from the group consisting of: I and V.

In another embodiment the proline tolerant tripeptidyl peptidase may comprise the sequence motif QNFSV.

In a further embodiment the proline tolerant tripeptidyl peptidase may comprise the sequence motifs xEANLD and y′Tzx′G or xEANLD and QNFSV.

In a yet further embodiment the proline tolerant tripeptidyl peptidase may comprise the sequence motifs y′Tzx′G and QNFSV.

Suitably the proline tolerant tripeptidyl peptidase may comprise the sequence motifs xEANLD, y′Tzx′G and QNFSV.

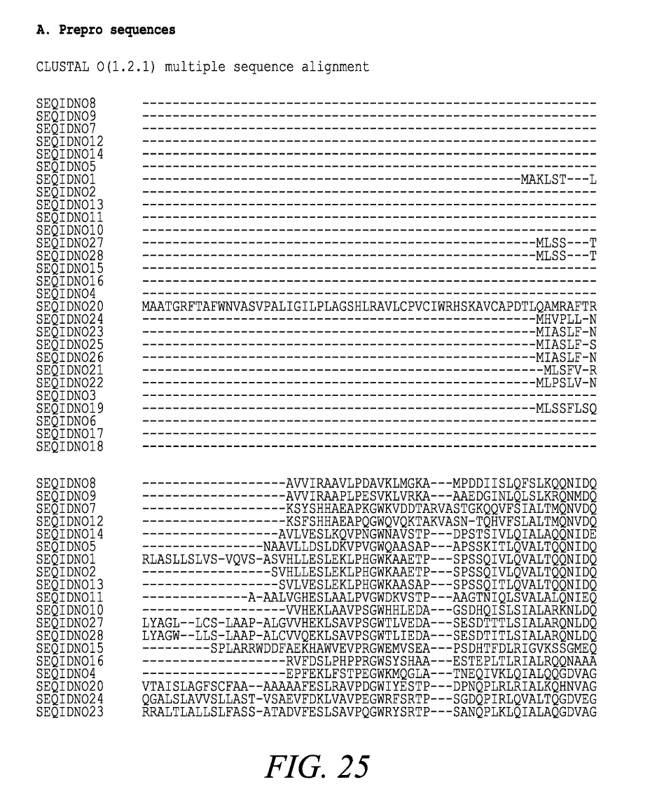

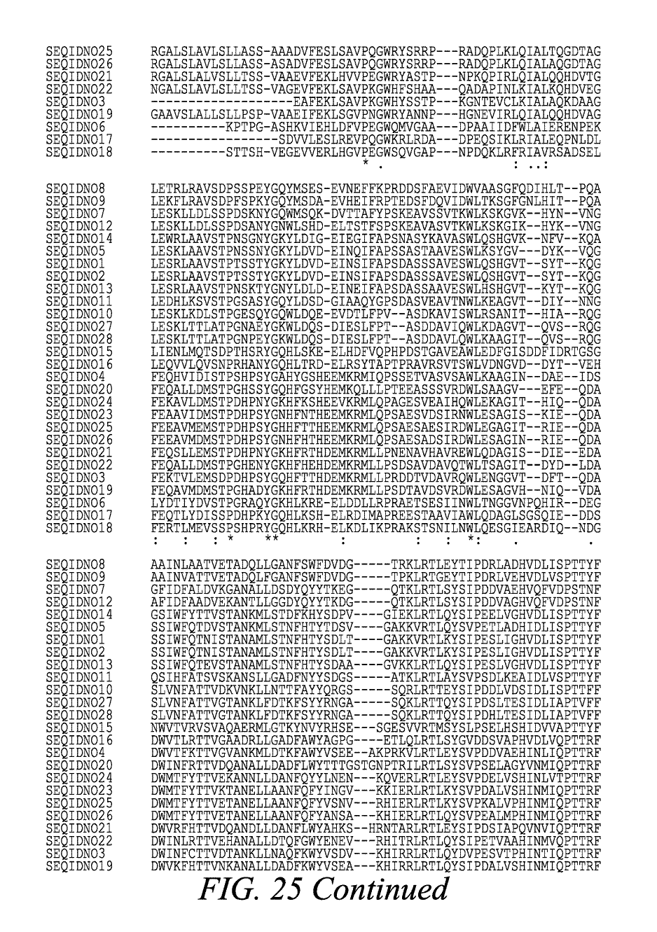

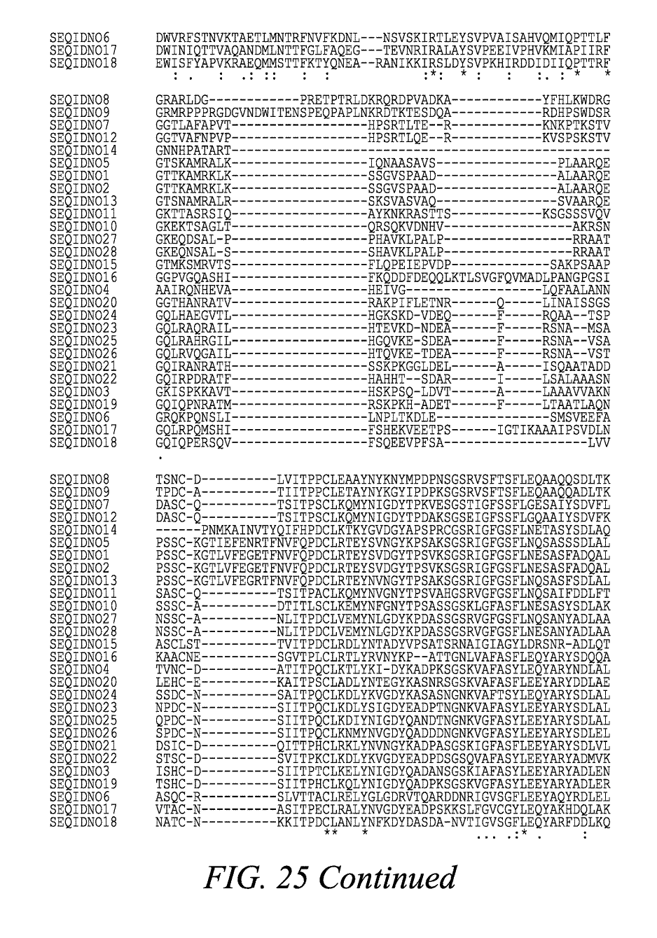

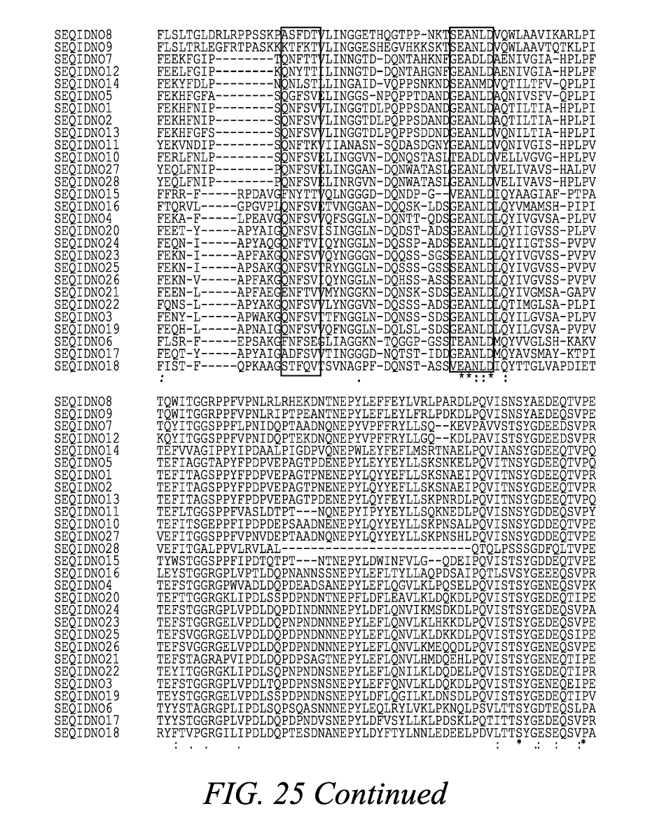

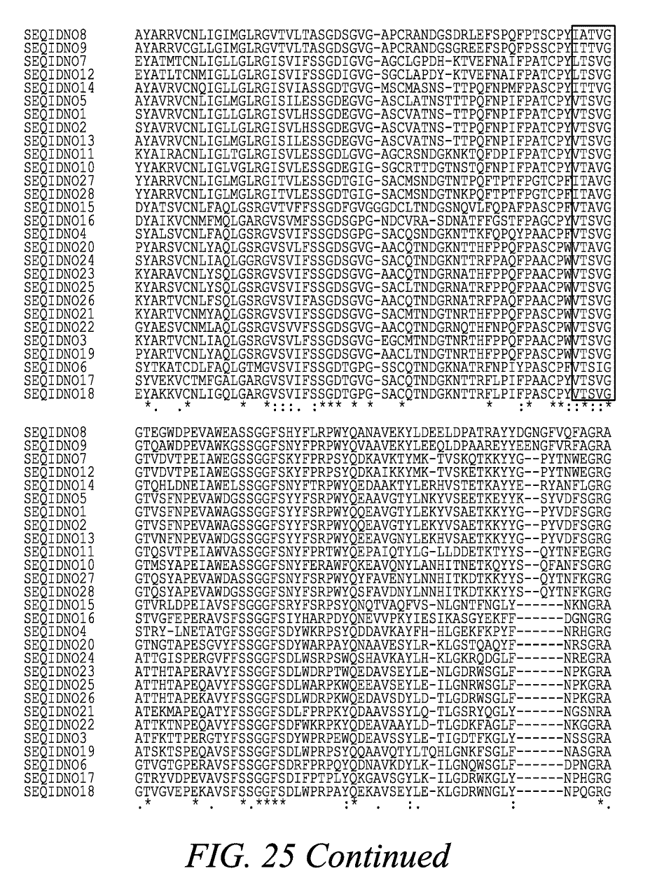

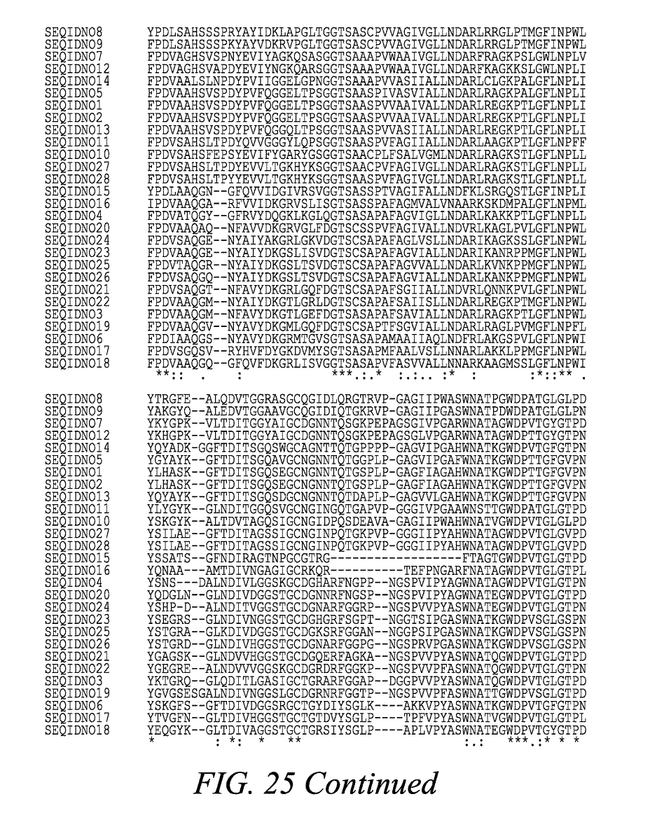

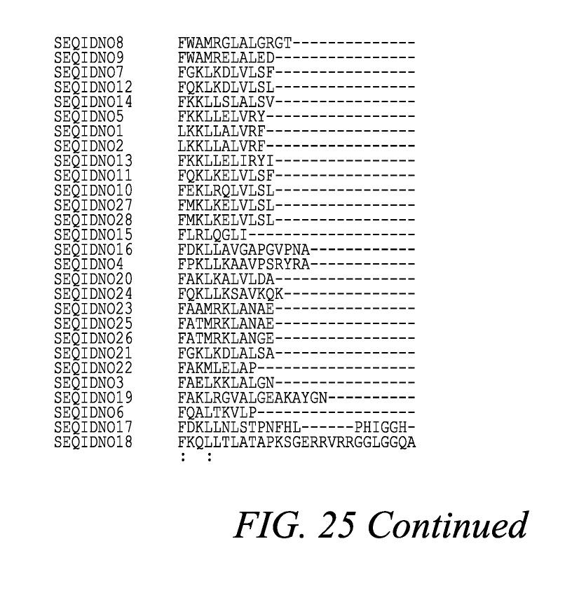

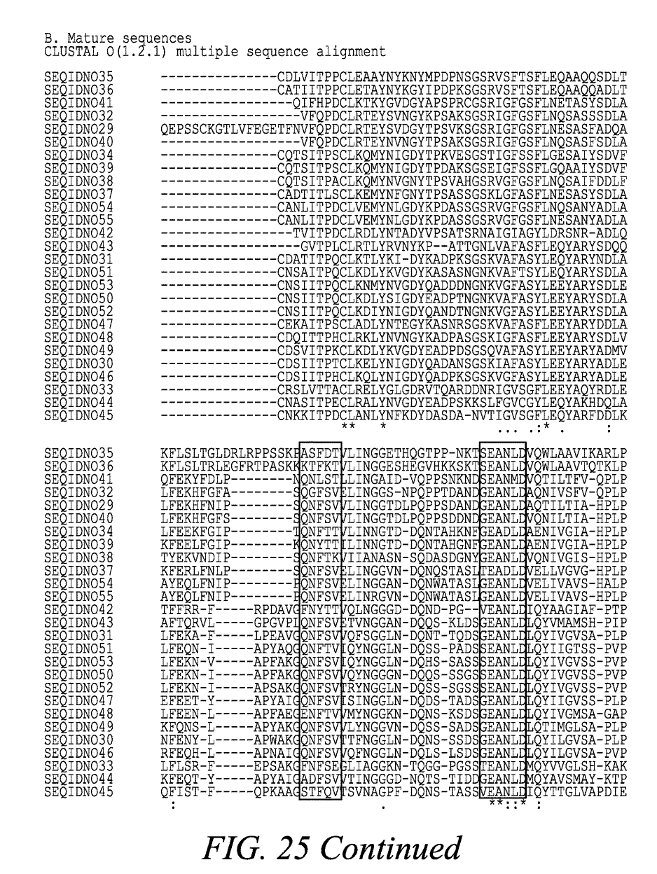

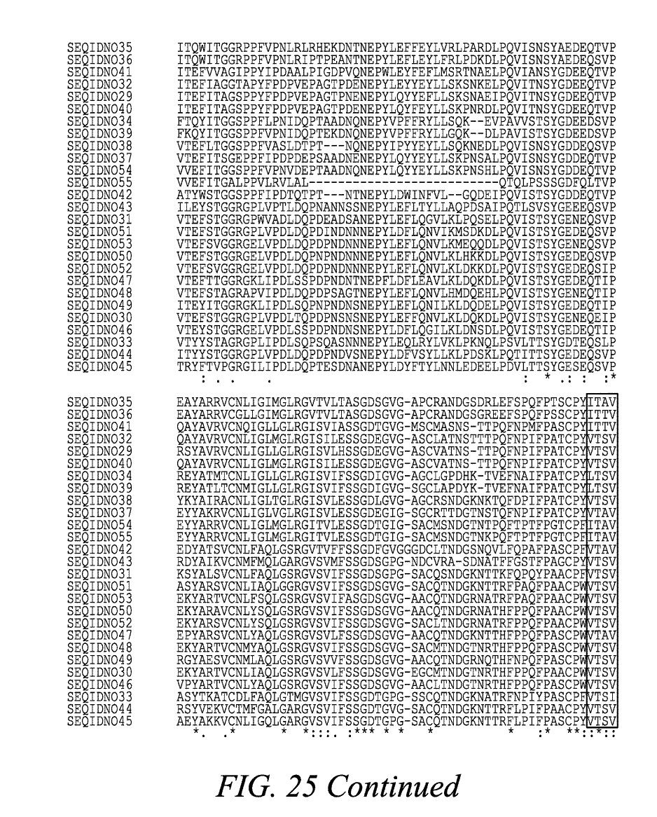

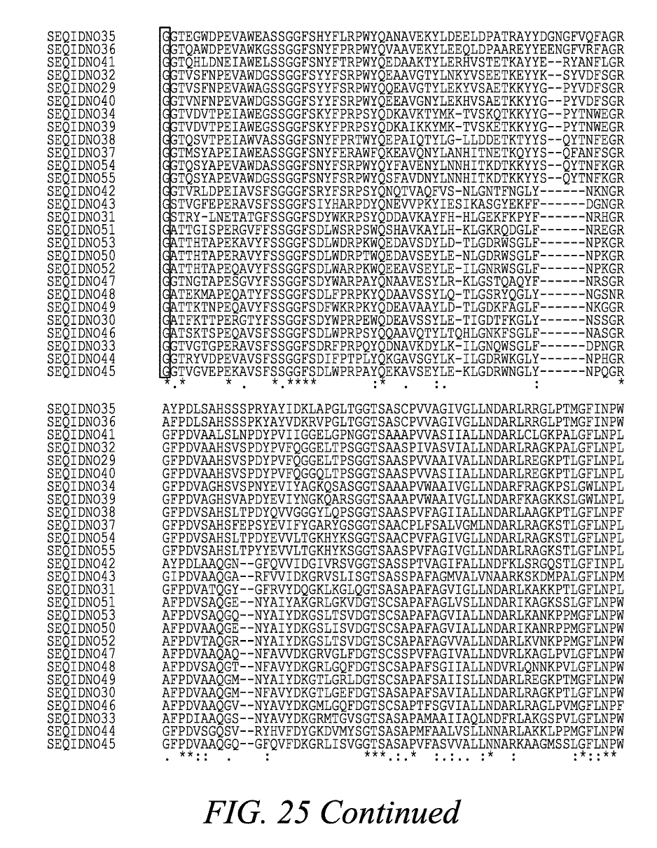

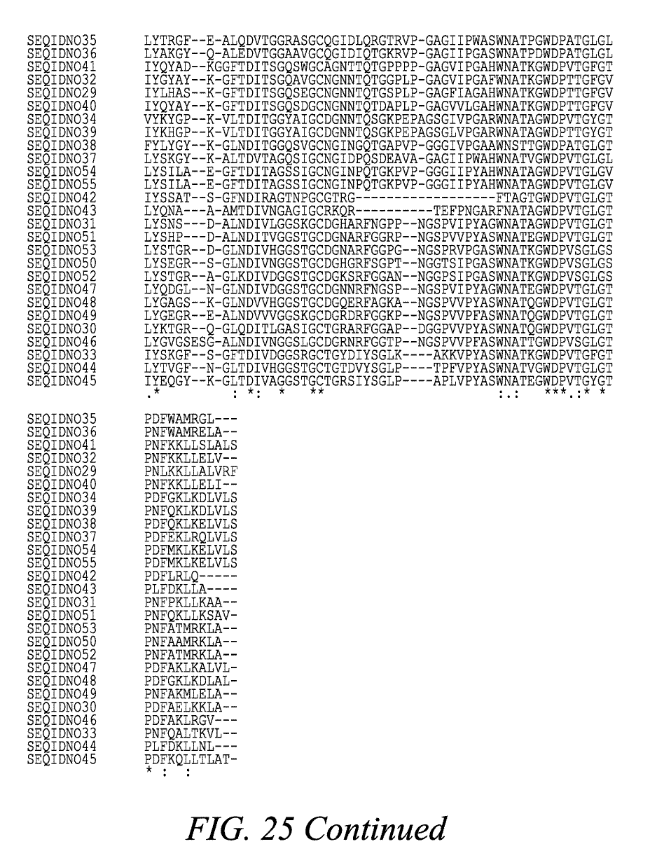

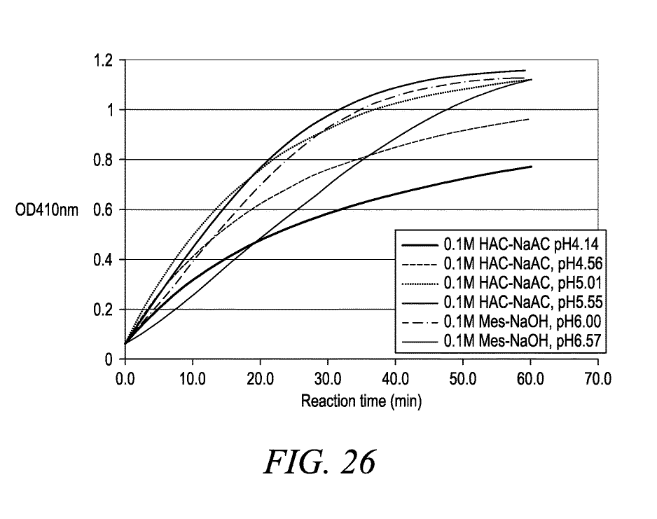

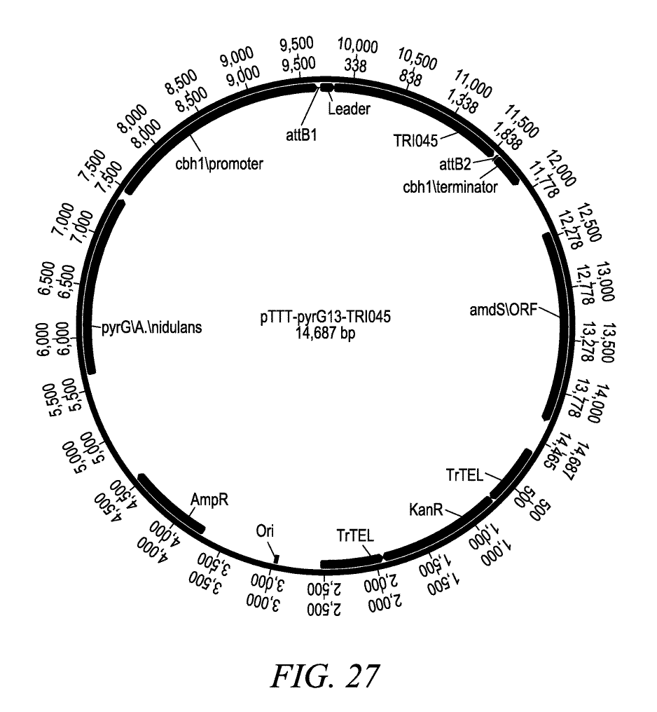

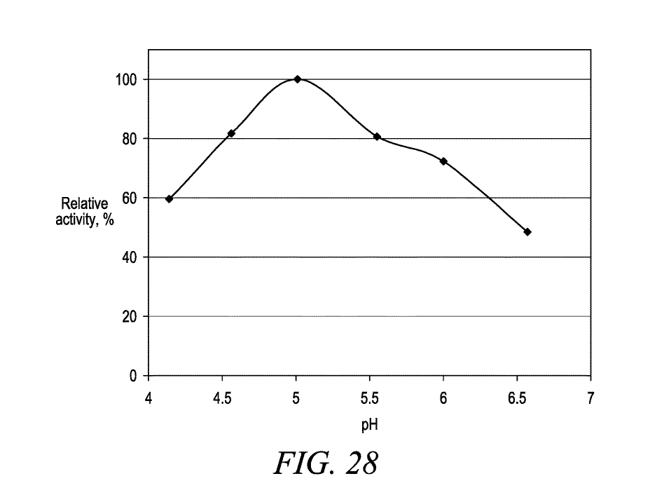

One or more of the motifs are present in the proline tolerant tripeptidyl peptidases for use in the present invention. FIG. 25 indicates the positioning of these motifs.

In one embodiment the proline tolerant tripeptidyl peptidase may be encoded by a nucleotide sequence shown as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 96, SEQ ID No. 97 or a nucleotide sequence having at least 70% identity thereto. Suitably a sequence having at least 80% thereto or at least 90% thereto.

Preferably the proline tolerant tripeptidyl peptidase may be encoded by a nucleotide sequence having at least 95% sequence identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79 or SEQ ID No. 80, more preferably at least 99% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 96 or SEQ ID No. 97.

In another embodiment the proline tolerant tripeptidyl peptidase may be encoded by a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 96 or SEQ ID No. 97 under medium stringency conditions. Suitably, a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 96 or SEQ ID No. 97 under high stringency conditions.

In a further embodiment, the proline tolerant tripeptidyl peptidase may be encoded by a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 96 or SEQ ID No. 97 due to degeneracy of the genetic code.

The present invention also provides for an isolated nucleic acid comprising:

(a) a nucleotide sequence as shown herein as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97;

(b) a nucleotide sequence which has at least about 70% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97; or

(c) a sequence that hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 under medium stringency conditions; or

(d) a nucleotide sequence which differs from

SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 due to degeneracy of the genetic code.

Suitably the nucleotide sequence may be a nucleotide sequence having at least about 80% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97, more suitably at least about 90% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97.

The nucleotide sequence may be a nucleotide sequence having at least about 95% identity, suitably at least about 99% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97.

In one embodiment the isolated nucleic acid may comprise a nucleotide sequence that hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 under high stringency conditions.

In one embodiment the isolated nucleotide sequence comprising a nucleotide sequence shown as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 may be a DNA, cDNA, synthetic DNA and/or RNA sequence.

Preferably the sequence is a DNA sequence, more preferably a cDNA sequence coding for the proline tolerant tripeptidyl peptidase of the present invention.

Suitably the present invention also provides for an isolated nucleic acid comprising:

(a) a nucleotide sequence as shown herein as SEQ ID No. 56

(b) a nucleotide sequence which has at least about 70% identity to SEQ ID No. 56; or

(c) a sequence that hybridises to SEQ ID No. 56 under medium stringency conditions; or

(c) a nucleotide sequence which differs from SEQ ID No. 56 due to degeneracy of the genetic code.

Suitably the nucleotide sequence may be a nucleotide sequence having at least about 80% identity to SEQ ID No. 56, more suitably at least about 90% identity to SEQ ID No. 56.

The nucleotide sequence may be a nucleotide sequence having at least bout 95% identity, suitably at least about 99% identity to SEQ ID No. 56.

In one embodiment the isolated nucleic acid may comprise a nucleotide sequence that hybridises to SEQ ID No. 56 under high stringency conditions.

Suitably the isolated nucleic acid may be comprised in a vector (e.g. a plasmid).

The present invention also provides for a host cell comprising an isolated nucleic acid sequence or vector according to the invention.

Suitably, the host cell may be a Trichoderma host cell, preferably a Trichoderma reesei host cell.

In one aspect, preferably the amino acid and/or nucleotide sequence for use in the present invention is in an isolated form. The term “isolated” means that the sequence is at least substantially free from at least one other component with which the sequence is naturally associated in nature and as found in nature. The amino acid and/or nucleotide sequence for use in the present invention may be provided in a form that is substantially free of one or more contaminants with which the substance might otherwise be associated. Thus, for example it may be substantially free of one or more potentially contaminating polypeptides and/or nucleic acid molecules.

In one aspect, preferably the amino acid and/or nucleotide sequence for use in the present invention is in a purified form. The term “purified” means that a given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

Enzymes

In one embodiment the tripeptidyl peptidase or exo-tripeptidyl peptidase of the S53 family for use in accordance with the present invention may have be selected from any of the enzyme sequences selected below.

In one embodiment the enzyme for use in the present invention may be a tripeptidyl peptidase comprising SEQ ID No. 1, a functional fragment thereof or a sequence having at least 70% identity to SEQ ID No. 1. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 2, a functional fragment thereof or a sequence having at least 70% identity to SEQ ID No. 2. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 3, a functional fragment thereof or a sequence having at least 70% identity to SEQ ID No. 3. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 4, a functional fragment thereof or a sequence having at least 70% identity to SEQ ID No. 4. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 5, a functional fragment thereof or a sequence having at least 70% identity to SEQ ID No. 5. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 6, a functional fragment thereof or a sequence having at least 70% identity to SEQ ID No. 6. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 7, a functional fragment thereof or a sequence having at least 70% identity to SEQ ID No. 7. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 8, a functional fragment thereof or a sequence having at least 70% identity to SEQ ID No. 8. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 9, a functional fragment thereof or a sequence having at least 70% identity to SEQ ID No. 9. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 10, a functional fragment thereof or a sequence having at least 70% identity to SEQ ID No. 10. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 11, a functional fragment thereof or a sequence having at least 70% identity to SEQ ID No. 11. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 12, a functional fragment thereof or a sequence having at least 70% identity to SEQ ID No. 12. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 13, a functional fragment thereof or a sequence having at least 70% identity to SEQ ID No. 13. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 14, a functional fragment thereof or a sequence having at least 70% identity to SEQ ID No. 14. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 15, a functional fragment thereof or a sequence having at least 70% identity to SEQ ID No. 15. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 16, a functional fragment thereof or a sequence having at least 70% identity to SEQ ID No. 16. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 17, a functional fragment thereof or a sequence having at least 70% identity to SEQ ID No. 17. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 18, a functional fragment thereof or a sequence having at least 70% identity to SEQ ID No. 18. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 19, a functional fragment thereof or a sequence having at least 70% identity to SEQ ID No. 19. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 20, a functional fragment thereof or a sequence having at least 70% identity to SEQ ID No. 20. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 21, a functional fragment thereof or a sequence having at least 70% identity to SEQ ID No. 21. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 22, a functional fragment thereof or a sequence having at least 70% identity to SEQ ID No. 22. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 23, a functional fragment thereof or a sequence having at least 70% identity to SEQ ID No. 23. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 24, a functional fragment thereof or a sequence having at least 70% identity to SEQ ID No. 24. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 25, a functional fragment thereof or a sequence having at least 70% identity to SEQ ID No. 25. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 26, a functional fragment thereof or a sequence having at least 70% identity to SEQ ID No. 26. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 27, a functional fragment thereof or a sequence having at least 70% identity to SEQ ID No. 27. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 28, a functional fragment thereof or a sequence having at least 70% identity to SEQ ID No. 28. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 29, a functional fragment thereof or a sequence having at least 70% identity to SEQ ID No. 29. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 30, a functional fragment thereof or a sequence having at least 70% identity to SEQ ID No. 30. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 31, a functional fragment thereof or a sequence having at least 70% identity to SEQ ID No. 31. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 32, a functional fragment thereof or a sequence having at least 70% identity to SEQ ID No. 32. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 33, a functional fragment thereof or a sequence having at least 70% identity to SEQ ID No. 33. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 34, a functional fragment thereof or a sequence having at least 70% identity to SEQ ID No. 34. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 35, a functional fragment thereof or a sequence having at least 70% identity to SEQ ID No. 35. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 36, a functional fragment thereof or a sequence having at least 70% identity to SEQ ID No. 36. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 37, a functional fragment thereof or a sequence having at least 70% identity to SEQ ID No. 37. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 38, a functional fragment thereof or a sequence having at least 70% identity to SEQ ID No. 38. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 39, a functional fragment thereof or a sequence having at least 70% identity to SEQ ID No. 39. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 40, a functional fragment thereof or a sequence having at least 70% identity to SEQ ID No. 40. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 41, a functional fragment thereof or a sequence having at least 70% identity to SEQ ID No. 41. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 42, a functional fragment thereof or a sequence having at least 70% identity to SEQ ID No. 42. Suitably the enzyme may have at least 80% or 85% identity thereto. Preferably at least 90% or 95% identity thereto.

In one embodiment the enzyme for use in the present invention may be a tripeptidyl peptidase comprising SEQ ID No. 43, a functional fragment thereof or a sequence having at least 70% identity to SEQ ID No. 43. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 44, a functional fragment thereof or a sequence having at least 70% identity to SEQ ID No. 44. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 45, a functional fragment thereof or a sequence having at least 70% identity to SEQ ID No. 45. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 46, a functional fragment thereof or a sequence having at least 70% identity to SEQ ID No. 46. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 47, a functional fragment thereof or a sequence having at least 70% identity to SEQ ID No. 47. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 48, a functional fragment thereof or a sequence having at least 70% identity to SEQ ID No. 48. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 49, a functional fragment thereof or a sequence having at least 70% identity to SEQ ID No. 49. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 50, a functional fragment thereof or a sequence having at least 70% identity to SEQ ID No. 50. Suitably the enzyme may have at least 80% or 85% identity thereto. Preferably at least 90% or 95% identity thereto.

In one embodiment the enzyme for use in the present invention may be a tripeptidyl peptidase comprising SEQ ID No. 51, a functional fragment thereof or a sequence having at least 70% identity to SEQ ID No. 51. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 52, a functional fragment thereof or a sequence having at least 70% identity to SEQ ID No. 52. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 53, a functional fragment thereof or a sequence having at least 70% identity to SEQ ID No. 53. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 54, a functional fragment thereof or a sequence having at least 70% identity to SEQ ID No. 54. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 55, a functional fragment thereof or a sequence having at least 70% identity to SEQ ID No. 55. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 98, a functional fragment thereof or a sequence having at least 70% identity to SEQ ID No. 98. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase comprising SEQ ID No. 99, a functional fragment thereof or a sequence having at least 70% identity to SEQ ID No. 99. Suitably the enzyme may have at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 56 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 57 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 58 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 59 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 60 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 61 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 62 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 63 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 64 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 65 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 66 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 67 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 68 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 69 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 70 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 71 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 72 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 73 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 74 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 75 or a sequence having at least 70% identity thereto.

Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 76 or a sequence having at least 70% identity thereto.

Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 77 or a sequence having at least 70% identity thereto.

Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 78 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 79 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 80 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 81 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 82 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 83 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 84 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 85 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 86 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 87 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 88 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 89 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 90 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 91 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 92 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 93 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 94 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 95 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 96 or a sequence having at least 70% identity thereto. Suitably by a sequence having at least 80% or 85% identity thereto. Preferably at least 90% or 95% identity thereto. More preferably at least 97% or 99% identity thereto.

In one embodiment the enzyme for use in the present invention may be a tripeptidyl peptidase encoded by a nucleotide sequence comprising the sequence shown as SEQ ID No. 97 or a sequence having at least 70% identity thereto. Suitably by a sequence having at least 80% or 85% identity thereto. Preferably at least 90% or 95% identity thereto. More preferably at least 97% or 99% identity thereto.

Nucleotide Sequence

The scope of the present invention encompasses nucleotide sequences encoding proteins having the specific properties as defined herein.

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

Preparation of the Nucleotide Sequence

A nucleotide sequence encoding either a protein which has the specific properties as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used. Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

Amino Acid Sequences

The scope of the present invention also encompasses amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

The protein encompassed in the present invention may be used in conjunction with other proteins, particularly enzymes. Thus the present invention also covers a combination of proteins wherein the combination comprises the protein/enzyme of the present invention and another protein/enzyme, which may be another protein/enzyme according to the present invention. This aspect is discussed in a later section.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

Isolated

In one aspect, preferably the amino acid sequence, or nucleic acid, or enzyme according to the present invention is in an isolated form. The term “isolated” means that the sequence or enzyme or nucleic acid is at least substantially free from at least one other component with which the sequence, enzyme or nucleic acid is naturally associated in nature and as found in nature. The sequence, enzyme or nucleic acid of the present invention may be provided in a form that is substantially free of one or more contaminants with which the substance might otherwise be associated. Thus, for example it may be substantially free of one or more potentially contaminating polypeptides and/or nucleic acid molecules.

Purified

In one aspect, preferably the sequence, enzyme or nucleic acid according to the present invention is in a purified form. The term “purified” means that the given component is present at a high level. The component is desirably the predominant component present in a composition. Preferably, it is present at a level of at least about 80% said level being determined on a dry weight/dry weight basis with respect to the total composition under consideration. Suitably it may be present at a level of at least about 90%, or at least about 95, or at least about 98% said level being determined on a dry weight/dry weight basis with respect to the total composition under consideration.

Sequence Identity or Sequence Homology

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, a homologous sequence is taken to include an amino acid or a nucleotide sequence which may be at least 75, 85 or 90% identical, preferably at least 95 or 98% identical to the subject sequence. Typically, the homologues will comprise the same active sites etc. as the subject amino acid sequence for instance. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids, preferably over at least 100 contiguous amino acids, preferably over at least 200 contiguous amino acids.

In one embodiment the present invention relates to a nucleic acid sequence (or gene) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In the present context, a homologous sequence is taken to include a nucleotide sequence which may be at least 75, 85 or 90% identical, preferably at least 95 or 98% identical to a nucleotide sequence encoding a polypeptide of the present invention (the subject sequence). Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences. % homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. Calculation of maximum % homology or % identity therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60), such as for example in the GenomeQuest search tool (www.genomequest.com).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate % homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 9 |
| GAP EXTENSION | 2 |

| FOR CLUSTAL | DNA | PROTEIN |
|---|---|---|
| Weight Matrix | IUB | Gonnet 250 |
| GAP OPENING | 15 | 10 |
| GAP EXTEND | 6.66 | 0.1 |

In one embodiment, CLUSTAL may be used with the gap penalty and gap extension set as defined above.

Suitably, the degree of identity with regard to a nucleotide sequence is determined over at least 20 contiguous nucleotides, preferably over at least 30 contiguous nucleotides, preferably over at least 40 contiguous nucleotides, preferably over at least 50 contiguous nucleotides, preferably over at least 60 contiguous nucleotides, preferably over at least 100 contiguous nucleotides.

Suitably, the degree of identity with regard to a nucleotide sequence is determined over at least 100 contiguous nucleotides, preferably over at least 200 contiguous nucleotides, preferably over at least 300 contiguous nucleotides, preferably over at least 400 contiguous nucleotides, preferably over at least 500 contiguous nucleotides, preferably over at least 600 contiguous nucleotides, preferably over at least 700 contiguous nucleotides, preferably over at least 800 contiguous nucleotides.

Suitably, the degree of identity with regard to a nucleotide sequence may be determined over the whole sequence.

Suitably, the degree of identity with regard to a protein (amino acid) sequence is determined over at least 100 contiguous amino acids, preferably over at least 200 contiguous amino acids, preferably over at least 300 contiguous amino acids.

Suitably, the degree of identity with regard to an amino acid or protein sequence may be determined over the whole sequence taught herein.

In the present context, the term “query sequence” means a homologous sequence or a foreign sequence, which is aligned with a subject sequence in order to see if it falls within the scope of the present invention. Accordingly, such query sequence can for example be a prior art sequence or a third party sequence.

In one preferred embodiment, the sequences are aligned by a global alignment program and the sequence identity is calculated by identifying the number of exact matches identified by the program divided by the length of the subject sequence.

In one embodiment, the degree of sequence identity between a query sequence and a subject sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the subject sequence.

In yet a further preferred embodiment, the global alignment program is selected from the group consisting of CLUSTAL and BLAST (preferably BLAST) and the sequence identity is calculated by identifying the number of exact matches identified by the program divided by the length of the subject sequence.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Replacements may also be made by synthetic amino acids (e.g. unnatural amino acids) include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-c-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid ^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention. Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any derivative, fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under medium stringency conditions (e.g. 50° C. and 0.2×SSC {1×SSC=0.15 M NaCl, 0.015 M Na₃ citrate pH 7.0}) to the nucleotide sequences presented herein.

More preferably, the term “variant” encompasses sequences that are complementary to sequences that are capable of hybridising under high stringency conditions (e.g. 65° C. and 0.1×SSC {1×SSC=0.15 M NaCl, 0.015 M Na₃ citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Also included within the scope of the present invention are polynucleotide sequences that are capable of hybridising to the nucleotide sequences presented herein under conditions of intermediate to maximal stringency.

In a preferred aspect, the present invention covers nucleotide sequences that can hybridise to the nucleotide sequence of the present invention, or the complement thereof, under medium stringency conditions (e.g. 50° C. and 0.2×SSC {1×SSC=0.15 M NaCl, 0.015 M Na₃ citrate pH 7.0}).

In a more preferred aspect, the present invention covers nucleotide sequences that can hybridise to the nucleotide sequence of the present invention, or the complement thereof, under high stringent conditions (e.g. 65° C. and 0.1×SSC {1×SSC=0.15 M NaCl, 0.015 M Na₃ citrate pH 7.0}).

Preferably hybridisation is analysed over the whole of the sequences taught herein.

Molecular Evolution

As a non-limiting example, it is possible to produce numerous site directed or random mutations into a nucleotide sequence, either in vivo or in vitro, and to subsequently screen for improved functionality of the encoded polypeptide by various means.

In addition, mutations or natural variants of a polynucleotide sequence can be recombined with either the wildtype or other mutations or natural variants to produce new variants. Such new variants can also be screened for improved functionality of the encoded polypeptide. The production of new preferred variants can be achieved by various methods well established in the art, for example the Error Threshold Mutagenesis (WO 92/18645), oligonucleotide mediated random mutagenesis (U.S. Pat. No. 5,723,323), DNA shuffling (U.S. Pat. No. 5,605,793), exo-mediated gene assembly WO00/58517. The application of these and similar random directed molecular evolution methods allows the identification and selection of variants of the enzymes of the present invention which have preferred characteristics without any prior knowledge of protein structure or function, and allows the production of non-predictable but beneficial mutations or variants. There are numerous examples of the application of molecular evolution in the art for the optimisation or alteration of enzyme activity, such examples include, but are not limited to one or more of the following: optimised expression and/or activity in a host cell or in vitro, increased enzymatic activity, altered substrate and/or product specificity, increased or decreased enzymatic or structural stability, altered enzymatic activity/specificity in preferred environmental conditions, e.g. temperature, pH, substrate.

Site-Directed Mutagenesis

Once a protein-encoding nucleotide sequence has been isolated, or a putative protein-encoding nucleotide sequence has been identified, it may be desirable to mutate the sequence in order to prepare a protein of the present invention.

Mutations may be introduced using synthetic oligonucleotides. These oligonucleotides contain nucleotide sequences flanking the desired mutation sites.

A suitable method is disclosed in Morinaga et al., (Biotechnology (1984) 2, p 646-649). Another method of introducing mutations into enzyme-encoding nucleotide sequences is described in Nelson and Long (Analytical Biochemistry (1989), 180, p 147-151).

Recombinant

In one aspect the sequence for use in the present invention is a recombinant sequence—i.e. a sequence that has been prepared using recombinant DNA techniques.

These recombinant DNA techniques are within the capabilities of a person of ordinary skill in the art. Such techniques are explained in the literature, for example, J. Sambrook, E. F. Fritsch, and T. Maniatis, 1989, Molecular Cloning: A Laboratory Manual, Second Edition, Books 1-3, Cold Spring Harbor Laboratory Press.

Synthetic

In one aspect the sequence for use in the present invention is a synthetic sequence—i.e. a sequence that has been prepared by in vitro chemical or enzymatic synthesis. It includes, but is not limited to, sequences made with optimal codon usage for host organisms—such as the methylotrophic yeasts Pichia and Hansenula.

Proteins and/or peptides for use in the present invention may also be of a synthetic origin.

Expression of Enzymes

The present invention also provides a method for the expression of a proline tolerant tripeptidyl peptidase predominantly having exopeptidase activity wherein said proline tolerant tripeptidyl peptidase is capable of cleaving tri-peptides from the N-terminus of peptides having the consensus sequence cleavage sites:

(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or

(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′:

said method comprising:
(a) transforming a host cell with a nucleic acid or a vector comprising a nucleotide sequence encoding a proline tolerant tripeptidyl peptidase;
(b) expressing the nucleic acid sequence or vector of step (a); and
(c) obtaining the proline tolerant tripeptidyl peptidase or a fermentate comprising said proline tolerant tripeptidyl peptidase.

Suitably the method may further comprise isolating and/or purifying and/or packaging the proline tolerant tripeptidyl peptidase.

The nucleic acid may be any nucleic acid encoding a proline tolerant tripeptidyl peptidase having the activity detailed herein. Suitably, the nucleic acid may be any one of the nucleic acids detailed herein. Suitably, the nucleic acid may be an isolated nucleic acid of the invention.

As used herein the term “fermentate” refers to the mixture of constituents present following (e.g. at the end of) the culturing of the host cell which includes the tripeptidyl peptidase (e.g. proline tolerant tripeptidyl peptidase) and/or exo-tripeptidyl peptidase of the S53 family. The fermentate may comprises as well as the tripeptidyl peptidase in accordance with the present invention other components such as particulate matter, solids, substrates not utilised during culturing, debris, media, cell waste, etc. In one aspect, bacterial cells (and, preferably, spores) are removed from the fermentate and/or inactivated to provide a cell-free fermentate.

The nucleotide sequence for use in the present invention may be incorporated into a recombinant replicable vector. The vector may be used to replicate and express the nucleotide sequence, in protein/enzyme form, in and/or from a compatible host cell. Expression may be controlled using control sequences e.g. regulatory sequences.

The protein produced by a host recombinant cell by expression of the nucleotide sequence may be secreted or may be contained intracellularly depending on the sequence and/or the vector used. The coding sequences may be designed with signal sequences which direct secretion of the substance coding sequences through a particular prokaryotic or eukaryotic cell membrane.

The term “expression vector” means a construct capable of in vivo or in vitro expression.

In one embodiment the proline tolerant tripeptidyl peptidase and/or endoprotease for use in the present invention may be encoded by a vector. In other words the vector may comprise a nucleotide sequence encoding the proline tolerant tripeptidyl peptidase.

Preferably, the expression vector is incorporated into the genome of a suitable host organism. The term “incorporated” preferably covers stable incorporation into the genome.

The nucleotide sequence of the present invention may be present in a vector in which the nucleotide sequence is operably linked to regulatory sequences capable of providing for the expression of the nucleotide sequence by a suitable host organism.

The vectors for use in the present invention may be transformed into a suitable host cell as described below to provide for expression of a polypeptide of the present invention.

The choice of vector e.g. a plasmid, cosmid, or phage vector will often depend on the host cell into which it is to be introduced.

The vectors for use in the present invention may contain one or more selectable marker genes—such as a gene, which confers antibiotic resistance e.g. ampicillin, kanamycin, chloramphenicol or tetracyclin resistance. Alternatively, the selection may be accomplished by co-transformation (as described in WO91/17243).

Vectors may be used in vitro, for example for the production of RNA or used to transfect, transform, transduce or infect a host cell.

Thus, in a further embodiment, the invention provides a method of making nucleotide sequences of the present invention by introducing a nucleotide sequence of the present invention into a replicable vector, introducing the vector into a compatible host cell, and growing the host cell under conditions which bring about replication of the vector.

The vector may further comprise a nucleotide sequence enabling the vector to replicate in the host cell in question. Examples of such sequences are the origins of replication of plasmids pUC19, pACYC177, pUB110, pE194, pAMB1 and pIJ702.

The nucleotide sequence and/or vector encoding the proline tolerant tripeptidyl peptidase and/or the endoprotease may be codon optimised for expression in a particular host organism.

The nucleotide sequence and/or vector encoding the proline tolerant tripeptidyl peptidase and/or the endoprotease may be codon optimised for expression in a prokaryotic or eukaryotic cell. Suitably, the nucleotide sequence and/or vector encoding the proline tolerant tripeptidyl peptidase and/or the endoprotease may be codon optimised for expression in a fungal host organism (e.g. Trichoderma, preferably Trichoderma reesei).

Codon optimisation refers to a process of modifying a nucleic acid sequence for enhanced expression in a host cell of interest by replacing at least one codon (e.g. at least about more than 1, 2, 3, 4, 5, 10, 15, 20, 25, 50, 60, 70, 80 or 100 codons) of the native sequence with codons that are more frequently used in the genes of the host cell, whilst maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, amongst other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimisation. A nucleotide sequence and/vector that has undergone this tailoring can be referred to therefore as a “codon optimised” nucleotide sequence and/or vector. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimising a particular sequence for expression in a particular host cell are also available, such as Gene Forge (Aptagen; Jacobus, Pa.). In some embodiments, one or more codons (e.g. 1, 2, 3, 4, 5, 10, 15, 20, 25, 50, or more, or all codons) in a sequence encoding a proline tolerant tripeptidyl peptidase and/or endoprotease for use in the present invention correspond to the most frequently used codon for a particular amino acid.

In one embodiment the nucleotide sequence encoding the proline tolerant tripeptidyl peptidase may be a nucleotide sequence which has been codon optimised for expression in Trichoderma reesei.

In one embodiment the codon optimised sequence may comprise a nucleotide sequence shown as SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 97 or a nucleotide sequence having at least 70% identity thereto. Suitably a sequence having at least 80% thereto or at least 90% thereto.

Preferably the codon optimised sequence may comprise a nucleotide sequence having at least 95% sequence identity to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, more preferably at least 99% identity to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95 or SEQ ID No. 97.

In one embodiment the proline tolerant tripeptidyl peptidase may be encoded by a nucleotide sequence which hybridises to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 97 under medium stringency conditions. Suitably, a nucleotide sequence which hybridises to SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95 or SEQ ID No. 97 under high stringency conditions.

In a further embodiment, the proline tolerant tripeptidyl peptidase may be encoded by a nucleotide sequence which differs from SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95 or SEQ ID No. 97 due to degeneracy of the genetic code.

There is also provided a vector (e.g. plasmid) comprising one or more of the sequences selected from the group consisting of: SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95 or SEQ ID No. 97.

Regulatory Sequences

In some applications, the nucleotide sequence for use in the present invention is operably linked to a regulatory sequence which is capable of providing for the expression of the nucleotide sequence, such as by the chosen host cell. By way of example, the present invention covers a vector comprising the nucleotide sequence of the present invention operably linked to such a regulatory sequence, i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under condition compatible with the control sequences.

The term “regulatory sequences” includes promoters and enhancers and other expression regulation signals.

The term “promoter” is used in the normal sense of the art, e.g. an RNA polymerase binding site.

Enhanced expression of the nucleotide sequence encoding the enzyme of the present invention may also be achieved by the selection of heterologous regulatory regions, e.g. promoter, secretion leader and terminator regions.

Preferably, the nucleotide sequence according to the present invention is operably linked to at least a promoter.

Other promoters may even be used to direct expression of the polypeptide of the present invention.

Examples of suitable promoters for directing the transcription of the nucleotide sequence in a bacterial, fungal or yeast host are well known in the art.

The promoter can additionally include features to ensure or to increase expression in a suitable host. For example, the features can be conserved regions such as a Pribnow Box or a TATA box.

Constructs

The term “construct”—which is synonymous with terms such as “conjugate”, “cassette” and “hybrid”—includes a nucleotide sequence for use according to the present invention directly or indirectly attached to a promoter.

An example of an indirect attachment is the provision of a suitable spacer group such as an intron sequence, such as the Sh1-intron or the ADH intron, intermediate the promoter and the nucleotide sequence of the present invention. The same is true for the term “fused” in relation to the present invention which includes direct or indirect attachment. In some cases, the terms do not cover the natural combination of the nucleotide sequence coding for the protein ordinarily associated with the wild type gene promoter and when they are both in their natural environment.

The construct may even contain or express a marker, which allows for the selection of the genetic construct.

For some applications, preferably the construct of the present invention comprises at least the nucleotide sequence of the present invention operably linked to a promoter.

Host Cells

The term “host cell”—in relation to the present invention includes any cell that comprises either the nucleotide sequence or an expression vector as described above and which is used in the recombinant production of a protein having the specific properties as defined herein.

Thus, a further embodiment of the present invention provides host cells transformed or transfected with a nucleotide sequence that expresses the protein of the present invention.

The cells will be chosen to be compatible with the said vector and may for example be prokaryotic (for example bacterial), fungal, yeast or plant cells.

Examples of suitable bacterial host organisms are gram positive or gram negative bacterial species.

Depending on the nature of the nucleotide sequence encoding the polypeptide of the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

The use of suitable host cells—such as yeast, fungal and plant host cells—may provide for post-translational modifications (e.g. myristoylation, glycosylation, truncation, lipidation and tyrosine, serine or threonine phosphorylation) as may be needed to confer optimal biological activity on recombinant expression products of the present invention.

The host cell may be a protease deficient or protease minus strain. This may for example be the protease deficient strain Aspergillus oryzae JaL 125 having the alkaline protease gene named “alp” deleted. This strain is described in WO97/35956.

Organism

The term “organism” in relation to the present invention includes any organism that could comprise the nucleotide sequence coding for the polypeptide according to the present invention and/or products obtained therefrom, and/or wherein a promoter can allow expression of the nucleotide sequence according to the present invention when present in the organism.

Suitable organisms may include a prokaryote, fungus, yeast or a plant.

The term “transgenic organism” in relation to the present invention includes any organism that comprises the nucleotide sequence coding for the polypeptide according to the present invention and/or the products obtained therefrom, and/or wherein a promoter can allow expression of the nucleotide sequence according to the present invention within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism of the present invention includes an organism comprising any one of, or combinations of, the nucleotide sequence coding for the polypeptide according to the present invention, constructs according to the present invention, vectors according to the present invention, plasmids according to the present invention, cells according to the present invention, tissues according to the present invention, or the products thereof. For example the transgenic organism may also comprise the nucleotide sequence coding for the polypeptide of the present invention under the control of a heterologous promoter.

Transformation of Host Cells/Organism

As indicated earlier, the host organism can be a prokaryotic or a eukaryotic organism. Examples of suitable prokaryotic hosts include E. coli and Bacillus subtilis.

Teachings on the transformation of prokaryotic hosts are well documented in the art, for example see Sambrook et al (Molecular Cloning: A Laboratory Manual, 2nd edition, 1989, Cold Spring Harbor Laboratory Press). If a prokaryotic host is used then the nucleotide sequence may need to be suitably modified before transformation—such as by removal of introns.

Filamentous fungi cells may be transformed using various methods known in the art—such as a process involving protoplast formation and transformation of the protoplasts followed by regeneration of the cell wall in a manner known. The use of Aspergillus as a host microorganism is described in EP 0 238 023.

Another host organism can be a plant. A review of the general techniques used for transforming plants may be found in articles by Potrykus (Annu Rev Plant Physiol Plant Mol Biol [1991] 42:205-225) and Christou (Agro-Food-Industry Hi-Tech March/April 1994 17-27).

Further teachings on plant transformation may be found in EP-A-0449375.

General teachings on the transformation of fungi, yeasts and plants are presented in following sections.

Transformed Fungus

A host organism may be a fungus—such as a mould. Examples of suitable such hosts include any member belonging to the genera Thermomyces, Acremonium, Aspergillus, Penicillium, Mucor, Neurospora, Trichoderma and the like.

In one embodiment, the host organism may be a filamentous fungus.

Transforming filamentous fungi is discussed in U.S. Pat. No. 5,741,665 which states that standard techniques for transformation of filamentous fungi and culturing the fungi are well known in the art. An extensive review of techniques as applied to N. crassa is found, for example in Davis and de Serres, Methods Enzymol (1971) 17A: 79-143.

Further teachings which may also be utilised in transforming filamentous fungi are reviewed in U.S. Pat. No. 5,674,707.

In addition, gene expression in filamentous fungi is taught in in Punt et al. (2002) Trends Biotechnol 2002 May; 20(5):200-6, Archer & Peberdy Crit Rev Biotechnol (1997) 17(4):273-306.

The present invention encompasses the production of transgenic filamentous fungi according to the present invention prepared by use of these standard techniques.

Suitably the host organism is a Trichoderma host organism, e.g. a Trichoderma reesei host organism.

In another embodiment, the host organism can be of the genus Aspergillus, such as Aspergillus niger.

A transgenic Aspergillus according to the present invention can also be prepared by following, for example, the teachings of Turner G. 1994 (Vectors for genetic manipulation. In: Martinelli, S. D., Kinghorn J. R. (Editors) Aspergillus: 50 years on. Progress in industrial microbiology vol 29. Elsevier Amsterdam 1994. pp. 641-666).

Transformed Yeast

In another embodiment, the transgenic organism can be a yeast.

A review of the principles of heterologous gene expression in yeast are provided in, for example, Methods Mol Biol (1995), 49:341-54, and Curr Opin Biotechnol (1997) October; 8(5):554-60.

In this regard, yeast—such as the species Saccharomyces cerevisiae or Pichia pastoris (see FEMS Microbiol Rev (2000 24(1):45-66), may be used as a vehicle for heterologous gene expression.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

For the transformation of yeast, several transformation protocols have been developed. For example, a transgenic Saccharomyces according to the present invention can be prepared by following the teachings of Hinnen et al., (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells may be selected using various selective markers—such as auxotrophic markers dominant antibiotic resistance markers.

Culturing and Production

Host cells transformed with the nucleotide sequence of the present invention may be cultured under conditions conducive to the production of the encoded polypeptide and which facilitate recovery of the polypeptide from the cells and/or culture medium.

The medium used to cultivate the cells may be any conventional medium suitable for growing the host cell in questions and obtaining expression of the polypeptide.

The protein produced by a recombinant cell may be displayed on the surface of the cell.

The protein may be secreted from the host cells and may conveniently be recovered from the culture medium using well-known procedures.

Secretion

Often, it is desirable for the protein to be secreted from the expression host into the culture medium from where the protein may be more easily recovered. According to the present invention, the secretion leader sequence may be selected on the basis of the desired expression host. Hybrid signal sequences may also be used with the context of the present invention.

Typical examples of heterologous secretion leader sequences are those originating from the fungal amyloglucosidase (AG) gene (glaA—both 18 and 24 amino acid versions e.g. from Aspergillus), the α-factor gene (yeasts e.g. Saccharomyces, Kluyveromyces and Hansenula) or the α-amylase gene (Bacillus).

By way of example, the secretion of heterologous proteins in E. coli is reviewed in Methods Enzymol (1990) 182:132-43.

Post-Transcription and Post-Translational Modifications

Suitably the proline tolerant tripeptidyl peptidase and/or the endoprotease for use in the present invention may be encoded by any one of the nucleotide sequences taught herein. Depending upon the host cell used post-transcriptional and/or post-translational modifications may be made. It is envisaged that the enzymes (e.g. the proline tolerant tripeptidyl peptidase and/or the endoprotease) for use in the present methods and/or uses encompasses enzymes (e.g. the proline tolerant tripeptidyl peptidase and/or the endoprotease) which have undergone post-transcriptional and/or post-translational modification.

One non-limiting example of a post-transcriptional and/or post-translational modifications is “clipping” or “cleavage” of a polypeptide (e.g. of the proline tolerant tripeptidyl peptidase and/or the endoprotease).

In some embodiments the polypeptide (e.g. the tripeptidyl peptidase of the present invention e.g. proline tolerant tripeptidyl peptidase and/or the endoprotease) may be clipped or cleaved. This may result in the conversion of the proline tolerant tripeptidyl peptidase and/or the endoprotease from an inactive or substantially inactive state to an active state (i.e. capable of performing the activity described herein).

The proline tolerant tripeptidyl peptidase may be a pro-peptide which undergoes further post-translational modification to a mature peptide, i.e. a polypeptide which has the proline tolerant tripeptidyl peptidase activity.

By way of example only SEQ ID No. 1 is the same as SEQ ID No. 29 except that SEQ ID No. 1 has undergone post-translational and/or post-transcriptional modification to remove some amino acids, more specifically 197 amino acids from the N-terminus. Therefore the polypeptide shown herein as SEQ ID No. 1 could be considered in some circumstances (i.e. in some host cells) as a pro-peptide—which is further processed to a mature peptide (SEQ ID No. 29) by post-translational and/or post-transcriptional modification. The precise modifications, e.g. cleavage site(s), in respect of the post-translational and/or post-transcriptional modification may vary slightly depending on host species. In some host species there may be no post translational and/or post-transcriptional modification, hence the pro-peptide would then be equivalent to the mature peptide (i.e. a polypeptide which has the tripeptidyl peptidase activity of the present invention). Without wishing to be bound by theory, the cleavage site(s) may be shifted by a few residues (e.g. 1, 2 or 3 residues) in either direction compared with the cleavage site shown by reference to SEQ ID No. 29 compared with SEQ ID No. 1. In other words, rather than cleavage at position 197 (R) for example, the cleavage may be at position 196-A, 195-A, 194-A, 198Q, 199E, 200P for example. In addition or alternatively, the cleavage may result in the removal of about 197 amino acids, in some embodiments the cleavage may result in the removal of between 194 and 200 residues.

Other examples of post-transcriptional and/or post-translational modifications include but are not limited to myristoylation, glycosylation, truncation, lipidation and tyrosine, serine or threonine phosphorylation. The skilled person will appreciate that the type of post-transcriptional and/or post-translational modifications that may occur to a protein (e.g. the proline tolerant tripeptidyl peptidase and/or the endoprotease) may depend on the host organism in which the protein (e.g. the proline tolerant tripeptidyl peptidase and/or the endoprotease) is expressed.

Detection

A variety of protocols for detecting and measuring the expression of the amino acid sequence are known in the art. Examples include enzyme-linked immunosorbent assay (ELISA), radioimmunoassay (RIA) and fluorescent activated cell sorting (FACS).

A wide variety of labels and conjugation techniques are known by those skilled in the art and can be used in various nucleic and amino acid assays.

A number of companies such as Pharmacia Biotech (Piscataway, N.J.), Promega (Madison, Wis.), and US Biochemical Corp (Cleveland, Ohio) supply commercial kits and protocols for these procedures.

Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Also, recombinant immunoglobulins may be produced as shown in U.S. Pat. No. 4,816,567.

Fusion Proteins

The amino acid sequence for use according to the present invention may be produced as a fusion protein, for example to aid in extraction and purification. Examples of fusion protein partners include glutathione-S-transferase (GST), 6×His, GAL4 (DNA binding and/or transcriptional activation domains) and (β-galactosidase). It may also be convenient to include a proteolytic cleavage site between the fusion protein partner and the protein sequence of interest to allow removal of fusion protein sequences.

Preferably, the fusion protein will not hinder the activity of the protein sequence.

Gene fusion expression systems in E. coli have been reviewed in Curr Opin Biotechnol (1995) 6(5):501-6.

In another embodiment of the invention, the amino acid sequence may be ligated to a heterologous sequence to encode a fusion protein. For example, for screening of peptide libraries for agents capable of affecting the substance activity, it may be useful to encode a chimeric substance expressing a heterologous epitope that is recognised by a commercially available antibody.

General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press.

Dosages

The proline tolerant tripeptidyl peptidase and/or the endoprotease and/or exo-tripeptidyl peptidase of the S53 family for use in the methods and/or uses of the present invention may be dosed in any suitable amount.

In one embodiment the proline tolerant tripeptidyl peptidase and/or exo-tripeptidyl peptidase of the S53 family may be dosed in an amount of about 5 mg to 3 g of enzyme per kg of protein substrate and/or food and/or feed additive composition.

In the preparation of a hydrolyzate suitably the enzyme proline tolerant tripeptidyl peptidase and/or exo-tripeptidyl peptidase of the S53 family may be dosed in an amount of 5 mg to 3 g of enzyme per kg of protein substrate.

In one embodiment suitably the enzyme proline tolerant tripeptidyl peptidase and/or exo-tripeptidyl peptidase of the S53 family may be dosed in an amount of 25 mg to 1000 mg of enzyme per kg of protein substrate.

In one embodiment the proline tolerant tripeptidyl peptidase may be dosed in an amount of about 0.01 mg-100 mg; 0.5 mg-100 mg; 1 mg-50 mg; 5 mg-100 mg; 5 mg-20 mg, 10 mg-100 mg; 0.05 mg-50 mg; or 0.10 mg-10 mg of enzyme per kg of feed additive composition. In certain embodiments the proline tolerant tripeptidyl peptidase may be dosed in an amount of about 0.01 g to 1000 g of enzyme per kg of feed additive composition, such as 0.1 g to 500 g, such as 0.5 g to 700 g, such as in an amount of about 0.01 g-200 g, 0.01 g-100 g;

0.5 g-100 g; 1 g-50 g; 5 g-100 g; 5 g-20 g, 5 g-15 g, 10 g-100 g; 0.05 g-50 g; or 0.10 g-10 g of enzyme per kg of feed additive composition.

In one preferred embodiment, the proline tolerant tripeptidyl peptidase may be dosed in an amount of about 5 mg-20 mg of enzyme per kg of feed additive composition,

The exact amount will depend on the particular type of composition employed and on the specific protease activity per mg of protein.

In another embodiment the proline tolerant tripeptidyl peptidase and/or exo-tripeptidyl peptidase of the S53 family may be dosed in an amount of about 1 mg to about 1 kg of enzyme per kg of food and/or feed and/or feedstuff and/or premix. Suitably the proline tolerant tripeptidyl peptidase and/or exo-tripeptidyl peptidase of the S53 family may be dosed at about 1 mg to about 250 g per kg of food and/or feed and/or feedstuff and/or premix. Preferably at about 1 mg to about 100 g (more preferably at about 1 mg to about 1 g) per kg of food and/or feed and/or feedstuff and/or premix.

The endoprotease may be dosed in an amount of about 50 to about 3000 mg of enzyme per kg of protein substrate, e.g. 0.05 to 3 g of enzyme per metric ton (MT) of protein substrate. Suitably, the endoprotease may be dosed in an amount of less than about 4.0 g of enzyme per MT of protein substrate.

In another embodiment the endoprotease may be dosed at between about 0.5 g and about 5.0 g of enzyme per MT of protein substrate. Suitably the endoprotease may be dosed at between about 0.5 g and about 3.0 g of enzyme per MT of protein substrate. More suitably, the endoprotease may be dosed at about 1.0 g to about 2.0 g of enzyme per MT of protein substrate.

In one embodiment the aminopeptidase may be dosed in an amount of between about 0.5 mg to about 2 g of enzyme per kg of protein substrate and/or food and/or feed additive composition. Suitably the aminopeptidase may be dosed in an amount of between about 1 mg to about 2 g of enzyme per kg of protein substrate and/or food and/or feed additive composition. More suitably in an amount of between about 5 mg to about 1.5 g of enzyme per kg of protein substrate and/or food and/or feed additive composition.

In the preparation of a hydrolysate the aminopeptidase may be dosed in an amount of between about 0.5 mg to about 2 g of enzyme per kg of protein substrate. Suitably the aminopeptidase may be dosed in an amount of between about 1 mg to about 2 g of enzyme per kg of protein substrate. More suitably in an amount of between about 5 mg to about 1.5 g of enzyme per kg of protein substrate.

In one embodiment the aminopeptidase may be dosed in an amount of between about 5 mg to about 500 mg of enzyme per kg of protein substrate. Suitably the aminopeptidase may be dosed in an amount of between about 50 mg to about 500 mg of enzyme per kg of protein substrate. Suitably the aminopeptidase may be dosed in an amount of between about 100 mg to about 450 mg of enzyme per kg of protein substrate.

Subject

The term “subject” may be used to refer to an “animal” or a “human”.

Suitably the subject may be a “sensitive individual” predisposed to having an immune reaction to an untreated hydrolysate comprising one or more particular proteins or portions thereof. For example, the subject may be a sensitive individual having: a gluten (e.g. gliadin) allergy, a milk protein allergy and/or a soy protein allergy.

The term “animal”, as used herein, means an animal that is to be or has been administered with a feed additive composition according to the present invention or a feedstuff comprising said feed additive composition according to the present invention.

Preferably, the animal is a mammal, a ruminant animal, monogastric animal, fish or crustacean including for example livestock or a domesticated animal (e.g. a pet).

In one embodiment the “animal” is livestock.

The term “livestock”, as used herein refers to any farmed animal. Preferably, livestock is one or more of cows or bulls (including calves), pigs (including piglets, swine, growing pigs, sows), poultry (including broilers, chickens, egg layers and turkeys), birds, fish (including freshwater fish, such as salmon, cod, trout and carp, e.g. koi carp, and marine fish, such as sea bass), crustaceans (such as shrimps, mussels and scallops), horses (including race horses), sheep (including lambs).

In another embodiment the “animal” is a domesticated animal or pet or an animal maintained in a zoological environment.

The term “domesticated animal or pet or animal maintained in a zoological environment” as used herein refers to any relevant animal including canines (e.g. dogs), felines (e.g. cats), rodents (e.g. guinea pigs, rats, mice), birds, fish (including freshwater fish and marine fish), and horses.

In one embodiment the animal is a monogastric animal. In a preferred embodiment the monogastric animal may be poultry or pig (or a combination thereof).

In another embodiment the animal is a ruminant animal.

The term animal is not intended to refer to a human being.

Compositions

The present invention provides a general composition comprising at least one proline tolerant tripeptidyl peptidase predominantly having exopeptidase wherein said proline tolerant tripeptidyl peptidase is capable of cleaving tri-peptides from the N-terminus of peptides having:
(i) Proline at P1; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
   an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

In one embodiment there is provided a food additive composition comprising at least one proline tolerant tripeptidyl peptidase wherein said proline tolerant tripeptidyl peptidase predominantly has exopeptidase activity and is capable of cleaving tri-peptides from the N-terminus of peptides having:

(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or

(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

There is also provided a feed additive composition comprising at least one proline tolerant tripeptidyl peptidase wherein said proline tolerant tripeptidyl peptidase predominantly has exopeptidase activity and is capable of cleaving tri-peptides from the N-terminus of peptides having:

(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or

(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

Alternatively or additionally the composition and/or food additive composition and/or feed additive composition may comprise a proline tolerant tripeptidyl peptidase obtainable by any one of the methods of the invention.

In another embodiment there is provided a composition and/or food additive and/or feed additive composition comprising a hydrolysate of the invention. Suitably, such a food and/or feed additive composition may further comprise a proline tolerant tripeptidyl peptidase (optionally in combination with an endoprotease).

The enzymes of the invention such as the proline tolerant tripeptidyl peptidase for use in the methods and/or uses of the present invention and/or the exo-tripeptidyl peptidase and/or the composition and/or food additive and/or feed additive composition may be formulated in any appropriate manner known in the art.

In some embodiments further ingredients may be admixed with the tripeptidyl peptidase (e.g. proline tolerant tripeptidyl peptidase) such as salts such as Na₂SO₄, maltodextrin, limestone (calcium carbonate), cyclodextrin, wheat or a wheat component, starch, Talc, PVA, polyols such as sorbitol and glycerol, benzoate, sorbiate, sugars such as sucrose and glucose, propylene glycol, 1,3-propane diol, parabens, sodium chloride, citrate, acetate, sodium acetate, phosphate, calcium, metabisulfite, formate or mixtures thereof.

In a preferred embodiment the food additive composition or feed additive composition according to the present invention comprises the tripeptidyl peptidase (e.g. proline tolerant tripeptidyl peptidase) according to the present invention or fermentate according to the present invention and further comprises one or more ingredients selected from the group consisting of: a salt, polyol including sorbitol and glycerol, wheat or a wheat component, sodium acetate, sodium acetate trihydrate, potassium sorbate Talc, PVA, benzoate, sorbiate, 1,3-propane diol, glucose, parabens, sodium chloride, citrate, metabisulfite, formate or a combination thereof.

In one embodiment the salt may be selected from the group consisting of: Na₂SO₄, NaH₂PO₄, Na₂HPO₄, Na₃PO₄, (NH₄)H₂PO₄, K₂HPO₄, KH₂PO₄, K₂SO₄, KHSO₄, ZnSO₄, MgSO₄, CuSO₄, Mg(NO₃)₂, (NH₄)₂SO₄, sodium borate, magnesium acetate, sodium citrate or combinations thereof.

Typical liquid formulations of food grade enzymes may include the following components (% is in w/w): enzyme of interest 0.2%-30%, preferably 2%-20%.

The stability of the enzyme formulation might also be increased by using salts like NaCl, KCl, CaCl2, Na2SO4 or other food grade salts in concentrations from about 0.1% to about 20% (suitably from about 0.1% to about 5%). Without wishing to be bound by theory, it is believed that the high salt concentrations might again be a way of achieving microbial stability either alone or in combination with further ingredients. The mechanism of action may be due to lower water activity or a specific action between a certain enzyme and a salt. Therefore in some embodiments the proline tolerant tripeptidyl peptidase may be admixed with at least one salt.

Suitably the preservative may be sodium benzoate and/or potassium sorbate. These preservatives can be typically used in a combined concentration of about 0.1-1%, suitably about 0.2-0.5%. Sodium benzoate is most efficient at pH <5.5 and sodium sorbate at pH <6.

Suitably the sugar is sorbitol.

Suitably the salt is sodium sulphate.

In one embodiment the one or more ingredients (e.g. used for the formulation of the composition and/or food additive composition and/or feed additive composition) may be selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other food grade salts; a preservative, e.g. sodium benzoate and/or potassium sorbate; or combinations thereof.

Suitably the one or more ingredients (e.g. used for the formulation of the composition and/or food additive composition and/or feed additive composition) may be selected from the group consisting of: a wheat carrier, sorbitol and sodium sulphate.

Suitably, the proline tolerant tripeptidyl peptidase and/or exo-tripeptidyl peptidase of the S53 family and/or the composition and/or food additive and/or feed additive composition may be admixed with a wheat carrier.

Suitably, the proline tolerant tripeptidyl peptidase and/or exo-tripeptidyl peptidase of the S53 family and/or the composition and/or food additive and/or feed additive composition may be admixed with sorbitol.

Suitably the proline tolerant tripeptidyl peptidase and/or exo-tripeptidyl peptidase of the S53 family and/or the composition and/or food additive and/or feed additive composition may be admixed with sodium sulphate.

In a preferred embodiment the composition and/or food additive and/or feed additive composition may further comprise any endoprotease detailed herein.

Forms

The feed additive composition of the present invention and other components and/or the feedstuff comprising same may be used in any suitable form.

The feed additive composition of the present invention may be used in the form of solid or liquid preparations or alternatives thereof. Examples of solid preparations include powders, pastes, boluses, capsules, pellets, tablets, dusts, and granules which may be wettable, spray-dried or freeze-dried. Examples of liquid preparations include, but are not limited to, aqueous, organic or aqueous-organic solutions, suspensions and emulsions.

In some applications, feed additive composition of the present invention may be mixed with feed or administered in the drinking water.

Suitable examples of forms include one or more of: powders, pastes, boluses, pellets, tablets, pills, granules, capsules, ovules, solutions or suspensions, which may contain flavouring or colouring agents, for immediate-, delayed-, modified-, sustained-, pulsed- or controlled-release applications.

By way of example, if the composition of the present invention is used in a solid, e.g. pelleted form, it may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such as polyvinylpyrrolidone, hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

Preferred excipients for the forms include lactose, starch, a cellulose, milk sugar or high molecular weight polyethylene glycols.

For aqueous suspensions and/or elixirs, the composition of the present invention may be combined with various sweetening or flavouring agents, colouring matter or dyes, with emulsifying and/or suspending agents and with diluents such as water, propylene glycol and glycerin, and combinations thereof.

Combination with Other Components

The proline tolerant tripeptidyl peptidase and endoprotease and/or exo-tripeptidyl peptidase of the S53 family and/or three enzyme composition and/or the composition and/or food and/or feed additive composition and/or hydrolysate of the present invention may be used in combination with other components.

The proline tolerant tripeptidyl peptidase and endoprotease and/or exo-tripeptidyl peptidase of the S53 family and/or three enzyme composition and/or the composition and/or food and/or feed additive composition and/or hydrolysate of the present invention and another component which is suitable for animal consumption and is capable of providing a medical or physiological benefit to the consumer.

In one embodiment the “another component” may be one or more enzymes.

Suitable additional enzymes for use in the present invention may be one or more of the enzymes selected from the group consisting of: endoglucanases (E.C. 3.2.1.4); celliobiohydrolases (E.C. 3.2.1.91), β-glucosidases (E.C. 3.2.1.21), cellulases (E.C. 3.2.1.74), lichenases (E.C. 3.1.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), alpha-amylases (E.C. 3.2.1.1), xylanases (E.C. 3.2.1.8, E.C. 3.2.1.32, E.C. 3.2.1.37, E.C. 3.1.1.72, E.C. 3.1.1.73), glucoamylases (E.C. 3.2.1.3), proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or mannanases (e.g. a β-mannanase (E.C. 3.2.1.78)).

Suitably the other component may be a phytase (e.g. a 6-phytase (E.C. 3.1.3.26) or a 3-phytase (E.C. 3.1.3.8)).

In one embodiment (particularly for feed applications) the other component may be one or more of the enzymes selected from the group consisting of xylanases (E.C. 3.2.1.8, E.C. 3.2.1.32, E.C. 3.2.1.37, E.C. 3.1.1.72, E.C. 3.1.1.73), an amylase (including α-amylases (E.C. 3.2.1.1), G4-forming amylases (E.C. 3.2.1.60), β-amylases (E.C. 3.2.1.2) and γ-amylases (E.C. 3.2.1.3); and/or a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)).

In one embodiment (particularly for feed applications) the other component may be a combination of an amylase (e.g. α-amylases (E.C. 3.2.1.1)) and a protease (e.g. subtilisin (E.C. 3.4.21.62)).

In one embodiment (particularly for feed applications) the other component may be a β-glucanase, e.g. an endo-1,3(4)-β-glucanases (E.C. 3.2.1.6).

In one embodiment (particularly for feed applications) the other component may be a mannanases (e.g. a β-mannanase (E.C. 3.2.1.78)).

In one embodiment (particularly for feed applications) the other component may be a lipase (E.C. 3.1.1.3), a lipid acyltransferase (generally classified as E.C. 2.3.1.x), or a phospholipase (E.C. 3.1.1.4, E.C. 3.1.1.32 or E.C. 3.1.1.5), suitably a lipase (E.C. 3.1.1.3).

In one embodiment (particularly for feed applications) the other component may be a protease (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)).

In another embodiment the other component for combining with a proline tolerant tripeptidyl peptidase (optionally in combination with an endoprotease) and/or for combining with an endoprotease in combination with an exo-tripeptidyl peptidase of the S53 family and aminopeptidase may be a further protease. Suitably, the further protease may be selected from the group consisting of: an aminopeptidase and a carboxypeptidase.

The term “carboxypeptidase” as used herein has its usual meaning in the art and refers to an exopeptidase that is capable of cleaving n amino acids from the C-terminus of a peptide and/or protein substrate. In one embodiment n may be at least 1, suitably n may be at least 2. In other embodiments n may be at least 3, suitably at least 4.

In other embodiments, the proline tolerant tripeptidyl peptidase (optionally in combination with an endoprotease) and/or three enzyme composition may be used with one or more further exopeptidase.

In one embodiment the proline tolerant tripeptidyl peptidase (optionally in combination with an endoprotease) and/or three enzyme composition is not combined with a proline-specific exopeptidase.

In a particularly preferred embodiment the proline tolerant tripeptidyl peptidase and/or three enzyme composition may not be combined with an enzyme having the following polypeptide sequence:

MRTAAASLTLAATCLFELASALMPRAPLIPAMKAKVALPSGNATFEQYID
HNNPGLGTFPQRYWYNPEFWAGPGSPVLLFTPGESDAADYDGFLTNKTIV
GRFAEEIGGAVILLEHRYWGASSPYPELTTETLQYLTLEQSIADLVHFAK
TVNLPFDEIHSSNADNAPWVMTGGSYSGALAAWTASIAPGTFWAYHASSA
PVQAIYDFWQYFVPVVEGMPKNCSKDLNRVVEYIDHVYESGDIERQQEIK
EMFGLGALKHFDDFAAAITNGPWLWQDMNFVSGYSRFYKFCDAVENVTPG
AKSVPGPEGVGLEKALQGYASWFNSTYLPGSCAEYKYWTDKDAVDCYDSY
ETNSPIYTDKAVNNTSNKQWTWFLCNEPLFYWQDGAPKDESTIVSRIVSA
EYWQRQCHAYFPEVNGYTFGSANGKTAEDVNKWTKGWDLTNTTRLIWANG
QFDPWRDASVSSKTRPGGPLQSTEQAPVHVIPGGFHCSDQWLVYGEANAG
VQKVIDEEVAQIKAWVAEYPKYRKP

In one embodiment the additional component may be a stabiliser or an emulsifier or a binder or carrier or an excipient or a diluent or a disintegrant.

The term “stabiliser” as used here is defined as an ingredient or combination of ingredients that keeps a product (e.g. a feed product) from changing over time.

The term “emulsifier” as used herein refers to an ingredient (e.g. a feed ingredient) that prevents the separation of emulsions. Emulsions are two immiscible substances, one present in droplet form, contained within the other. Emulsions can consist of oil-in-water, where the droplet or dispersed phase is oil and the continuous phase is water; or water-in-oil, where the water becomes the dispersed phase and the continuous phase is oil. Foams, which are gas-in-liquid, and suspensions, which are solid-in-liquid, can also be stabilised through the use of emulsifiers.

As used herein the term “binder” refers to an ingredient (e.g. a feed ingredient) that binds the product together through a physical or chemical reaction. During “gelation” for instance, water is absorbed, providing a binding effect. However, binders can absorb other liquids, such as oils, holding them within the product. In the context of the present invention binders would typically be used in solid or low-moisture products for instance baking products: pastries, doughnuts, bread and others. Examples of granulation binders include one or more of: polyvinylpyrrolidone, hydroxypropylmethylcellulose (HPMC), hydroxypropylcellulose (HPC), sucrose, maltose, gelatin and acacia.

“Carriers” mean materials suitable for administration of the enzyme and include any such material known in the art such as, for example, any liquid, gel, solvent, liquid diluent, solubilizer, or the like, which is non-toxic and which does not interact with any components of the composition in a deleterious manner.

The present invention provides a method for preparing a composition (e.g. a feed additive composition) comprising admixing feed additive of the present invention (and preferably corn or a corn by-product) with at least one physiologically acceptable carrier selected from at least one of maltodextrin, limestone (calcium carbonate), cyclodextrin, wheat or a wheat component, sucrose, starch, Na₂SO₄, Talc, PVA, sorbitol, benzoate, sorbiate, glycerol, sucrose, propylene glycol, 1,3-propane diol, glucose, parabens, sodium chloride, citrate, acetate, phosphate, calcium, metabisulfite, formate and mixtures thereof.

Examples of “excipients” include one or more of: microcrystalline cellulose and other celluloses, lactose, sodium citrate, calcium carbonate, dibasic calcium phosphate, glycine, starch, milk sugar and high molecular weight polyethylene glycols.

Examples of “disintegrants” include one or more of: starch (preferably corn, potato or tapioca starch), sodium starch glycollate, croscarmellose sodium and certain complex silicates.

Examples of “diluents” include one or more of: water, ethanol, propylene glycol and glycerin, and combinations thereof.

The other components may be used simultaneously (e.g. when they are in admixture together or even when they are delivered by different routes) or sequentially (e.g. they may be delivered by different routes) to the feed additive composition or feed additive composition or hydrolysate of the present invention.

In one embodiment preferably the feed additive composition, or feed ingredient, or feed or feedstuff or premix according to the present invention does not comprise chromium or organic chromium.

In one embodiment preferably the feed additive composition, or feed ingredient, or feed or feedstuff or premix according to the present invention does not contain sorbic acid.

Packaging

In one embodiment the proline tolerant tripeptidyl peptidase and endoprotease and/or the exo-tripeptidyl peptidase of the S53 family and/or three enzyme composition and/or the composition and/or food and/or feed additive composition and/or hydrolysate and/or foodstuff and/or feedstuff according to the present invention is packaged.

In one preferred embodiment the proline tolerant tripeptidyl peptidase and endoprotease and/or the exo-tripeptidyl peptidase of the S53 family and/or three enzyme composition and/or the composition and/or food and/or feed additive composition and/or hydrolysate and/or foodstuff and/or feedstuff is packaged in a bag, such as a paper bag.

In an alternative embodiment the proline tolerant tripeptidyl peptidase and endoprotease and/or the exo-tripeptidyl peptidase of the S53 family and/or three enzyme composition and/or the composition and/or food and/or feed additive composition and/or hydrolysate and/or foodstuff and/or feedstuff may be sealed in a container. Any suitable container may be used.

Foodstuff

The term “foodstuff” is used synonymously herein with “food”.

Here, the term “foodstuff” is used to refer to food for humans.

The food may be in the form of a solution or as a solid—depending on the use and/or the mode of application and/or the mode of administration.

When used as—or in the preparation of—a food—such as functional food—the hydrolysate and/or composition and/or food additive composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

In one embodiment the present invention provides a foodstuff comprising a hydrolysate according to the invention. The foodstuff may additionally comprise a proline tolerant tripeptidyl peptidase (such as one obtainable by any of the methods herein), optionally in combination with an endoprotease.

Optionally, the foodstuff may further comprise at least one food ingredient.

Suitably the foodstuff may comprise at least one proline tolerant tripeptidyl peptidase comprising an amino acid sequence selected from:

SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith.

Suitably the proline tolerant tripeptidyl peptidase may comprise one or more amino acid sequence selected from SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28 or a functional fragment thereof or an amino acid sequence having at least 70% identity therewith.

Suitably the proline tolerant tripeptidyl peptidase may comprise one or more amino acid sequence selected from SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith.

The present invention also relates to a method for the production of a foodstuff comprising contacting a food component with a hydrolysate of the invention or a composition and/or food additive composition of the invention.

Where a food component is contacted with a composition and/or food additive composition, suitably the food component may also be contacted with an endoprotease.

The composition of the present invention can be used in the preparation of food products such as one or more of: jams, marmalades, jellies, dairy products (such as milk or cheese), meat products, poultry products, fish products and bakery products.

In one embodiment, the composition of the present invention can be used in the preparation of protein rich waste streams. The term “protein rich waste streams” as used herein may include substrates which are byproducts from plant or animal processing, e.g. soybean processing, byproducts from rendering facilities (e.g. waste from abattoirs and/or fish processing facilities).

By way of example, the composition of the present invention can be used as ingredients to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, breakfast cereals, instant noodles and cup noodles, instant soups and cup soups, balanced foods and drinks, sweeteners, texture improved snack bars, fibre bars, bake stable fruit fillings, care glaze, chocolate bakery filling, cheese cake flavoured filling, fruit flavoured cake filling, cake and doughnut icing, heat stable bakery filling, instant bakery filling creams, filing for cookies, ready-to-use bakery filling, reduced calorie filling, adult nutritional beverage, acidified soy/juice beverage, aseptic/retorted chocolate drink, bar mixes, beverage powders, calcium fortified soy/plaim and chocolate milk, calcium fortified coffee beverage, powdered shakes (e.g. protein supplement shakes or health shakes or sports shakes or elderly person shakes)), a sports nutrition product, a performance food, a baby food, a food for elderly, a food for people in medical care, satiety supplements.

A composition according to the present invention can further be used as an ingredient in food products such as American cheese sauce, anti-caking agent for grated and shredded cheese, chip dip, cream cheese, dry blended whip topping fat free sour cream, freeze/thaw dairy whipping cream, freeze/thaw stable whipped tipping, low fat and lite natural cheddar cheese, low fat Swiss style yoghurt, aerated frozen desserts, and novelty bars, hard pack ice cream, label friendly, improved economics and indulgence of hard pack ice cream, low fat ice cream: soft serve, barbecue sauce, cheese dip sauce, cottage cheese dressing, dry mix Alfredo sauce, mix cheese sauce, dry mix tomato sauce and others.

For certain aspects, preferably the foodstuff is a beverage.

Preferably the foodstuff may be a bakery product—such as bread, Danish pastry, biscuits or cookies.

The present invention also provides a method of preparing a food or a food ingredient, the method comprising admixing 5-KGA produced by the process of the present invention or the composition according to the present invention with another food ingredient. The method for preparing or a food ingredient is also another aspect of the present invention.

The foodstuff according to the invention may be a dairy product, a whey-protein product, a bakery product, a fermentation product, a performance food, a baby food, a beverage, a shake, a casing, a sports nutrition product, a performance food, a baby food, a food for elderly, or a food for people in medical care.

Suitably the dairy product may be a milk-based product. Such milk-based products may comprise one or more milk proteins or fragments thereof.

Preferably the dairy (e.g. milk-based product) may be an infant formula.

Suitably the bakery product may be a bread product.

Suitably a fermentation product may be a soy-based fermentation product.

Suitably a casing may be a beverage (preferably a casing for beer) or dairy casing.

Food Ingredient

The composition and/or food additive composition of the present invention may be used as a food ingredient.

As used herein the term “food ingredient” includes a formulation which is or can be added to functional foods or foodstuffs as a nutritional supplement and/or fiber supplement. The term food ingredient as used here also refers to formulations which can be used at low levels in a wide variety of products that require gelling, texturising, stabilising, suspending, film-forming and structuring, retention of juiciness, improved mouthfeel, improvements in peptide content, improvements in nutritional components, without adding viscosity.

The food ingredient may be in the form of a solution or as a solid—depending on the use and/or the mode of application and/or the mode of administration.

Food Supplements

The hydrolysate and/or composition and/or food additive composition of the present invention may be—or may be added to—food supplements.

Functional Foods

The composition of the present invention may be—or may be added to—functional foods.

As used herein, the term “functional food” means food which is capable of providing not only a nutritional effect and/or a taste satisfaction, but is also capable of delivering a further beneficial effect to consumer.

Accordingly, functional foods are ordinary foods that have components or ingredients (such as those described herein) incorporated into them that impart to the food a specific functional—e.g. medical or physiological benefit—other than a purely nutritional effect.

Although there is no legal definition of a functional food, most of the parties with an interest in this area agree that they are foods marketed as having specific health effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

Surveys have suggested that consumers place the most emphasis on functional food claims relating to heart disease. Preventing cancer is another aspect of nutrition which interests consumers a great deal, but interestingly this is the area that consumers feel they can exert least control over. In fact, according to the World Health Organization, at least 35% of cancer cases are diet-related. Furthermore claims relating to osteoporosis, gut health and obesity effects are also key factors that are likely to incite functional food purchase and drive market development.

Feed

The feed additive composition of the present invention may be used as—or in the preparation of—a feed.

In one embodiment the present invention provides a feedstuff comprising a hydrolysate according to the invention. The feedstuff may additionally comprise a proline tolerant tripeptidyl peptidase (such as one obtainable by any of the methods herein), optionally in combination with an endoprotease.

Optionally, the feedstuff may further comprise at least one feed ingredient.

Suitably the feedstuff may comprise at least one proline tolerant tripeptidyl peptidase comprising an amino acid sequence selected from:

SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith.

Suitably the proline tolerant tripeptidyl peptidase may comprise one or more amino acid sequence selected from SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28 or a functional fragment thereof or an amino acid sequence having at least 70% identity therewith.

Suitably the proline tolerant tripeptidyl peptidase may comprise one or more amino acid sequence selected from SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity therewith.

The present invention also relates to a method for the production of a feedstuff comprising contacting a feed component with a hydrolysate of the invention or a composition and/or feed additive composition of the invention.

Where a feed component is contacted with a composition and/or feed additive composition, suitably the feed component may also be contacted with an endoprotease.

The term “feed” is used synonymously herein with “feedstuff”.

The feed may be in the form of a solution or as a solid—depending on the use and/or the mode of application and/or the mode of administration.

When used as—or in the preparation of—a feed—such as functional feed—the composition of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

In a preferred embodiment the feed additive composition of the present invention is admixed with a feed component to form a feedstuff.

The term “feed component” as used herein means all or part of the feedstuff. Part of the feedstuff may mean one constituent of the feedstuff or more than one constituent of the feedstuff, e.g. 2 or 3 or 4. In one embodiment the term “feed component” encompasses a premix or premix constituents.

In one embodiment a feed additive composition comprising a proline tolerant tripeptidyl peptidase and one or more ingredients selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other food grade salts; a preservative, e.g. sodium benzoate and/or potassium sorbate; or combinations thereof (optionally in combination with an endoprotease) may be admixed with at least one protein or portion thereof is an animal protein or a vegetable protein (e.g. selected from one or more of a gliadin, a beta-casein, a beta-lactoglobulin or an immunogenic fragment of a gliadin, a beta-casein, a beta-lactoglobulin, glycinin, beta-conglycinin, cruciferin, napin, collagen, whey protein, fish protein, meat protein, egg protein, soy protein a hordein or grain protein), preferably comprised in corn, soybean meal, corn dried distillers grains with solubles (DDGS), wheat, wheat proteins including gluten, wheat by products, wheat bran, corn by products including corn gluten meal, barley, oat, rye, triticale, full fat soy, animal by-product meals, an alcohol-soluble protein (preferably a zein (e.g. a maize zein maize) and/or a kafirin (e.g. from sorghum)), a protein from oil seeds (preferably from soybean seed proteins, sun flower seed proteins, rapeseed proteins, canola (rapeseed) seed proteins or combinations thereof) or a combination thereof.

Preferably the feed may be a fodder, or a premix thereof, a compound feed, or a premix thereof. In one embodiment the feed additive composition according to the present invention may be admixed with a compound feed, a compound feed component or to a premix of a compound feed or to a fodder, a fodder component, or a premix of a fodder.

The term fodder as used herein means any food which is provided to an animal (rather than the animal having to forage for it themselves). Fodder encompasses plants that have been cut.

The term fodder includes hay, straw, silage, compressed and pelleted feeds, oils and mixed rations, and also sprouted grains and legumes.

Fodder may be obtained from one or more of the plants selected from: alfalfa (Lucerne), barley, birdsfoot trefoil, brassicas, Chau moellier, kale, rapeseed (canola), rutabaga (swede), turnip, clover, alsike clover, red clover, subterranean clover, white clover, grass, false oat grass, fescue, Bermuda grass, brome, heath grass, meadow grasses (from naturally mixed grassland swards, orchard grass, rye grass, Timothy-grass, corn (maize), millet, oats, sorghum, soybeans, trees (pollard tree shoots for tree-hay), wheat, and legumes.

The term “compound feed” means a commercial feed in the form of a meal, a pellet, nuts, cake or a crumble. Compound feeds may be blended from various raw materials and additives. These blends are formulated according to the specific requirements of the target animal.

Compound feeds can be complete feeds that provide all the daily required nutrients, concentrates that provide a part of the ration (protein, energy) or supplements that only provide additional micronutrients, such as minerals and vitamins.

The main ingredients used in compound feed are the feed grains, which include corn, wheat, rye, maize, soybeans, sorghum, oats, and barley.

Suitably a premix as referred to herein may be a composition composed of microingredients such as vitamins, minerals, chemical preservatives, antibiotics, fermentation products, and other essential ingredients. Premixes are usually compositions suitable for blending into commercial rations.

Any feedstuff of the present invention may comprise one or more feed materials selected from the group comprising a) cereals, such as small grains (e.g., wheat, barley, rye, oats and combinations thereof) and/or large grains such as maize or sorghum; b) by products from plants, such as Distillers Dried Grain Solubles (DDGS), wheat bran, wheat middlings, wheat shorts, rice bran, rice hulls, oat hulls, palm kernel, citrus pulp, corn fibre, corn germ meal, corn bran, Hominy feed, corn gluten feed, gluten meal, wheat shorts, wheat middlings or combinations thereof; c) protein obtained from sources such as soya, sunflower, peanut, lupin, peas, fava beans, cotton, canola (rapeseed), fish meal, dried plasma protein, meat and bone meal, potato protein, whey, copra, sesame; d) oils and fats obtained from vegetable and animal sources; e) minerals and vitamins.

A feedstuff of the present invention may contain at least 30%, at least 40%, at least 50% or at least 60% by weight corn and soybean meal or corn and full fat soy, or wheat meal or sunflower meal.

In addition or in the alternative, a feedstuff of the present invention may comprise at least one high fibre feed material and/or at least one by-product of the at least one high fibre feed material to provide a high fibre feedstuff. Examples of high fibre feed materials include: wheat, barley, rye, oats, by products from plants (e.g. cereals), such as Distillers Dried Grain Solubles (DDGS), wheat bran, wheat middlings, wheat shorts, rice bran, rice hulls, oat hulls, palm kernel, citrus pulp, corn fibre, corn germ meal, corn bran, Hominy feed, corn gluten feed, gluten meal, wheat shorts, wheat middlings or combinations thereof. Some protein sources may also be regarded as high fibre: protein obtained from sources such as sunflower, lupin, fava beans and cotton.

In the present invention the feed may be one or more of the following: a compound feed and premix, including pellets, nuts or (cattle) cake; a crop or crop residue: corn, soybeans, sorghum, oats, barley, corn stover, copra, straw, chaff, sugar beet waste; fish meal; freshly cut grass and other forage plants; meat and bone meal; molasses; oil cake and press cake; oligosaccharides; conserved forage plants: hay and silage; seaweed; seeds and grains, either whole or prepared by crushing, milling etc.; sprouted grains and legumes; yeast extract.

The term “feed” in the present invention also encompasses in some embodiments pet food. A pet food is plant or animal material intended for consumption by pets, such as dog food or cat food. Pet food, such as dog and cat food, may be either in a dry form, such as kibble for dogs, or wet canned form. Cat food may contain the amino acid taurine.

The term “feed” in the present invention also encompasses in some embodiments fish food.

A fish food normally contains macro nutrients, trace elements and vitamins necessary to keep captive fish in good health. Fish food may be in the form of a flake, pellet or tablet. Pelleted forms, some of which sink rapidly, are often used for larger fish or bottom feeding species. Some fish foods also contain additives, such as beta carotene or sex hormones, to artificially enhance the colour of ornamental fish.

The term “feed” in the present invention also encompasses in some embodiment bird food. Bird food includes food that is used both in birdfeeders and to feed pet birds. Typically bird food comprises of a variety of seeds, but may also encompass suet (beef or mutton fat). As used herein the term “contacting” refers to the indirect or direct application of the composition of the present invention to the product (e.g. the feed). Examples of the application methods which may be used, include, but are not limited to, treating the product in a material comprising the feed additive composition, direct application by mixing the feed additive composition with the product, spraying the feed additive composition onto the product surface or dipping the product into a preparation of the feed additive composition.

In one embodiment the feed additive composition of the present invention is preferably admixed with the product (e.g. feedstuff). Alternatively, the feed additive composition may be included in the emulsion or raw ingredients of a feedstuff.

For some applications, it is important that the composition is made available on or to the surface of a product to be affected/treated. This allows the composition to impart one or more of the following favourable characteristics: biophysical characteristic is selected from the group consisting of one or more of the following: performance of the animal, growth performance of an animal, feed conversion ratio (FCR), ability to digest a raw material (e.g. nutrient digestibility, including starch, fat, protein, fibre digestibility), nitrogen digestibility (e.g. ileal nitrogen digestibility) and digestible energy (e.g. ileal digestible energy) nitrogen retention, carcass yield, growth rate, weight gain, body weight, mass, feed efficiency, body fat percentage, body fat distribution, growth, egg size, egg weight, egg mass, egg laying rate, lean gain, bone ash %, bone ash mg, back fat %, milk output, milk fat %, reproductive outputs such as litter size, litter survivability, hatchability % and environmental impact, e.g. manure output and/or nitrogen excretion.

The feed additive compositions of the present invention may be applied to intersperse, coat and/or impregnate a product (e.g. feedstuff or raw ingredients of a feedstuff) with a controlled amount of enzyme(s).

Preferably, the feed additive composition of the present invention will be thermally stable to heat treatment up to about 70° C.; up to about 85° C.; or up to about 95° C. The heat treatment may be performed for up to about 1 minute; up to about 5 minutes; up to about 10 minutes; up to about 30 minutes; up to about 60 minutes. The term thermally stable means that at least about 75% of the enzyme components that were present/active in the additive before heating to the specified temperature are still present/active after it cools to room temperature. Preferably, at least about 80% of the enzyme components that were present and active in the additive before heating to the specified temperature are still present and active after it cools to room temperature.

In a particularly preferred embodiment the feed additive composition is homogenized to produce a powder.

In an alternative preferred embodiment, the feed additive composition is formulated to granules as described in WO2007/044968 (referred to as TPT granules).

In another preferred embodiment when the feed additive composition is formulated into granules the granules comprise a hydrated barrier salt coated over the protein core. The advantage of such salt coating is improved thermo-tolerance, improved storage stability and protection against other feed additives otherwise having adverse effect on the enzyme.

Preferably, the salt used for the salt coating has a water activity greater than 0.25 or constant humidity greater than 60% at 20° C.

Preferably, the salt coating comprises a Na₂SO₄.

The method of preparing a feed additive composition may also comprise the further step of pelleting the powder. The powder may be mixed with other components known in the art. The powder, or mixture comprising the powder, may be forced through a die and the resulting strands are cut into suitable pellets of variable length.

Optionally, the pelleting step may include a steam treatment, or conditioning stage, prior to formation of the pellets. The mixture comprising the powder may be placed in a conditioner, e.g. a mixer with steam injection. The mixture is heated in the conditioner up to a specified temperature, such as from 60-100° C., typical temperatures would be 70° C., 80° C., 85° C., 90° C. or 95° C. The residence time can be variable from seconds to minutes and even hours. Such as 5 seconds, 10 seconds, 15 seconds, 30 seconds, 1 minutes 2 minutes., 5 minutes, 10 minutes, 15 minutes, 30 minutes and 1 hour.

It will be understood that the feed additive composition of the present invention is suitable for addition to any appropriate feed material.

As used herein, the term feed material refers to the basic feed material to be consumed by an animal. It will be further understood that this may comprise, for example, at least one or more unprocessed grains, and/or processed plant and/or animal material such as soybean meal or bone meal.

As used herein, the term “feedstuff” refers to a feed material to which one or more feed additive compositions have been added.

It will be understood by the skilled person that different animals require different feedstuffs, and even the same animal may require different feedstuffs, depending upon the purpose for which the animal is reared.

Preferably, the feedstuff may comprise feed materials comprising maize or corn, wheat, barley, triticale, rye, rice, tapioca, sorghum, and/or any of the by-products, as well as protein rich components like soybean mean, rape seed meal, canola meal, cotton seed meal, sunflower seed mean, animal-by-product meals and mixtures thereof. More preferably, the feedstuff may comprise animal fats and/or vegetable oils.

Optionally, the feedstuff may also contain additional minerals such as, for example, calcium and/or additional vitamins.

Preferably, the feedstuff is a corn soybean meal mix.

Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting—in particular by suitable techniques that may include at least the use of steam.

The feedstuff may be a feedstuff for a monogastric animal, such as poultry (for example, broiler, layer, broiler breeders, turkey, duck, geese, water fowl), swine (all age categories), a pet (for example dogs, cats) or fish, preferably the feedstuff is for poultry.

By way of example only a feedstuff for chickens, e.g. broiler chickens may be comprises of one or more of the ingredients listed in the table below, for example in the % ages given in the table below:

| Ingredients | Starter (%) | Finisher (%) |
|---|---|---|
| Maize | 46.2 | 46.7 |
| Wheat Middlings | 6.7 | 10.0 |
| Maize DDGS | 7.0 | 7.0 |
| Soyabean Meal 48% CP | 32.8 | 26.2 |
| An/Veg Fat blend | 3.0 | 5.8 |
| L-Lysine HCl | 0.3 | 0.3 |
| DL-methionine | 0.3 | 0.3 |
| L-threonine | 0.1 | 0.1 |
| Salt | 0.3 | 0.4 |
| Limestone | 1.1 | 1.1 |
| Dicalcium Phosphate | 1.2 | 1.2 |
| Poultry Vitamins and Micro-minerals | 0.3 | 0.3 |

By way of example only the diet specification for chickens, such as broiler chickens, may be as set out in the Table below:

Diet specification

| | | |
|---|---|---|
| Crude Protein (%) | 23.00 | 20.40 |
| Metabolizable Energy Poultry | 2950 | 3100 |
| (kcal/kg) | | |
| Calcium (%) | 0.85 | 0.85 |
| Available Phosphorus (%) | 0.38 | 0.38 |
| Sodium (%) | 0.18 | 0.19 |
| Dig. Lysine (%) | 1.21 | 1.07 |
| Dig. Methionine (%) | 0.62 | 0.57 |
| Dig. Methionine + Cysteine (%) | 0.86 | 0.78 |
| Dig. Threonine (%) | 0.76 | 0.68 |

By way of example only a feedstuff laying hens may be comprises of one or more of the ingredients listed in the table below, for example in the % ages given in the table below:

| Ingredient | Laying phase (%) |
|---|---|
| Maize | 10.0 |
| Wheat | 53.6 |
| Maize DDGS | 5.0 |
| Soybean Meal 48% CP | 14.9 |
| Wheat Middlings | 3.0 |
| Soybean Oil | 1.8 |
| L-Lysine HCl | 0.2 |
| DL-methionine | 0.2 |
| L-threonine | 0.1 |
| Salt | 0.3 |
| Dicalcium Phosphate | 1.6 |
| Limestone | 8.9 |
| Poultry Vitamins and Micro-minerals | 0.6 |

By way of example only the diet specification for laying hens may be as set out in the Table below:

Diet specification

| | |
|---|---|
| Crude Protein (%) | 16.10 |
| Metabolizable Energy Poultry | 2700 |
| (kcal/kg) | |
| Lysine (%) | 0.85 |
| Methionine (%) | 0.42 |
| Methionine + Cysteine (%) | 0.71 |
| Threonine (%) | 0.60 |
| Calcium (%) | 3.85 |
| Available Phosphorus (%) | 0.42 |
| Sodium (%) | 0.16 |

By way of example only a feedstuff for turkeys may be comprises of one or more of the ingredients listed in the table below, for example in the % ages given in the table below:

| | Phase 1 | Phase 2 | Phase 3 | Phase 4 |
|---|---|---|---|---|
| Ingredient | (%) | (%) | (%) | (%) |
| Wheat | 33.6 | 42.3 | 52.4 | 61.6 |
| Maize DDGS | 7.0 | 7.0 | 7.0 | 7.0 |
| Soyabean Meal 48% CP | 44.6 | 36.6 | 27.2 | 19.2 |
| Rapeseed Meal | 4.0 | 4.0 | 4.0 | 4.0 |
| Soyabean Oil | 4.4 | 4.2 | 3.9 | 3.6 |
| L-Lysine HCl | 0.5 | 0.5 | 0.4 | 0.4 |
| DL-methionine | 0.4 | 0.4 | 0.3 | 0.2 |
| L-threonine | 0.2 | 0.2 | 0.1 | 0.1 |
| Salt | 0.3 | 0.3 | 0.3 | 0.3 |
| Limestone | 1.0 | 1.1 | 1.1 | 1.0 |
| Dicalcium Phosphate | 3.5 | 3.0 | 2.7 | 2.0 |
| Poultry Vitamins and | 0.4 | 0.4 | 0.4 | 0.4 |
| Micro-minerals | | | | |

By way of example only the diet specification for turkeys may be as set out in the Table below:

Diet specification

| | | | | |
|---|---|---|---|---|
| Crude Protein (%) | 29.35 | 26.37 | 22.93 | 20.00 |
| Metabolizable Energy Poultry | 2.850 | 2.900 | 2.950 | 3.001 |
| (kcal/kg) | | | | |
| Calcium (%) | 1.43 | 1.33 | 1.22 | 1.02 |
| Available Phosphorus (%) | 0.80 | 0.71 | 0.65 | 0.53 |
| Sodium (%) | 0.16 | 0.17 | 0.17 | 0.17 |
| Dig. Lysine (%) | 1.77 | 1.53 | 1.27 | 1.04 |
| Dig. Methionine (%) | 0.79 | 0.71 | 0.62 | 0.48 |
| Dig. Methionine + Cysteine (%) | 1.12 | 1.02 | 0.90 | 0.74 |
| Dig. Threonine (%) | 1.03 | 0.89 | 0.73 | 0.59 |

By way of example only a feedstuff for piglets may be comprises of one or more of the ingredients listed in the table below, for example in the % ages given in the table below:

| Ingredient | Phase 1 (%) | Phase 2 (%) |
|---|---|---|
| Maize | 20.0 | 7.0 |
| Wheat | 25.9 | 46.6 |
| Rye | 4.0 | 10.0 |
| Wheat middlings | 4.0 | 4.0 |
| Maize DDGS | 6.0 | 8.0 |
| Soyabean Meal 48% CP | 25.7 | 19.9 |
| Dried Whey | 10.0 | 0.0 |
| Soyabean Oil | 1.0 | 0.7 |
| L-Lysine HCl | 0.4 | 0.5 |
| DL-methionine | 0.2 | 0.2 |
| L-threonine | 0.1 | 0.2 |
| L-tryptophan | 0.03 | 0.04 |
| Limestone | 0.6 | 0.7 |
| Dicalcium Phosphate | 1.6 | 1.6 |
| Swine Vitamins and Micro- | 0.2 | 0.2 |
| minerals | | |
| Salt | 0.2 | 0.4 |

By way of example only the diet specification for piglets may be as set out in the Table below:

Diet specification

| | | |
|---|---|---|
| Crude Protein (%) | 21.50 | 20.00 |
| Swine Digestible Energy | 3380 | 3320 |
| (kcal/kg) | | |
| Swine Net Energy (kcal/kg) | 2270 | 2230 |
| Calcium (%) | 0.80 | 0.75 |
| Digestible Phosphorus (%) | 0.40 | 0.35 |
| Sodium (%) | 0.20 | 0.20 |
| Dig. Lysine (%) | 1.23 | 1.14 |
| Dig. Methionine (%) | 0.49 | 0.44 |
| Dig. Methionine + Cysteine (%) | 0.74 | 0.68 |
| Dig. Threonine (%) | 0.80 | 0.74 |

By way of example only a feedstuff for grower/finisher pigs may be comprises of one or more of the ingredients listed in the table below, for example in the % ages given in the table below:

| Ingredient | Grower/Finisher (%) |
|---|---|
| Maize | 27.5 |
| Soyabean Meal 48% CP | 15.4 |
| Maize DDGS | 20.0 |
| Wheat bran | 11.1 |
| Rice bran | 12.0 |
| Canola seed meal | 10.0 |
| Limestone | 1.6 |
| Dicalcium phosphate | 0.01 |
| Salt | 0.4 |
| Swine Vitamins and Micro-minerals | 0.3 |
| Lysine-HCl | 0.2 |
| Vegetable oil | 0.5 |

By way of example only the diet specification for grower/finisher pigs may be as set out in the Table below:

Diet specification

| | |
|---|---|
| Crude Protein (%) | 22.60 |
| Swine Metabolizable Energy | 3030 |
| (kcal/kg) | |
| Calcium (%) | 0.75 |
| Available Phosphorus (%) | 0.29 |
| Digestible Lysine (%) | 1.01 |
| Dig. Methionine + Cysteine (%) | 0.73 |
| Digestible Threonine (%) | 0.66 |

Meat Based Food/Feed Product

The proline tolerant tripeptidyl peptidase and endoprotease and/or composition and/or food/feed composition and/or hydrolysate may be used in the manufacture of a meat based food/feed product.

A “meat based food product” and “meat based feed product” according to the present invention is any product based on meat.

The meat based food product is suitable for human and/or animal consumption as a food and/or a feed. In one embodiment of the invention the meat based food product is a feed product for feeding animals, such as for example a pet food product. In another embodiment of the invention the meat based food product is a food product for humans.

A meat based food/feed product may comprise non-meat ingredients such as for example water, salt, flour, milk protein, vegetable protein, starch, hydrolysed protein, phosphate, acid, spices, colouring agents and/or texturising agents.

A meat based food/feed product in accordance with the present invention preferably comprises between 5-90% (weight/weight) meat. In some embodiments the meat based food product may comprise at least 30% (weight/weight) meat, such as at least 50%, at least 60% or at least 70% meat.

In some embodiments the meat based food/feed product is a cooked meat, such as ham, loin, picnic shoulder, bacon and/or pork belly for example.

The meat based food/feed product may be one or more of the following:

Dry or semi-dry cured meats—such as fermented products, dry-cured and fermented with starter cultures, for example dry sausages, salami, pepperoni and dry ham; Emulsified meat products (e.g. for cold or hot consumption), such as mortadella, frankfurter, luncheon meat and pâté;

Fish and seafood, such as shrimps, salmon, reformulated fish products, frozen cold-packed fish;

Fresh meat muscle, such as whole injected meat muscle, for example loin, shoulder ham, marinated meat;

Ground and/or restructured fresh meat—or reformulated meat, such as upgraded cut-away meat by cold setting gel or binding, for example raw, uncooked loin chops, steaks, roasts, fresh sausages, beef burgers, meat balls, pelmeni;

Poultry products—such as chicken or turkey breasts or reformulated poultry, e.g. chicken nuggets and/or chicken sausages;

Retorted products—autoclaved meat products, for example picnic ham, luncheon meat, emulsified products.

In one embodiment of the present invention the meat based food/feed product is a processed meat product, such as for example a sausage, bologna, meat loaf, comminuted meat product, ground meat, bacon, polony, salami or pate.

A processed meat product may be for example an emulsified meat product, manufactured from a meat based emulsion, such as for example mortadella, bologna, pepperoni, liver sausage, chicken sausage, wiener, frankfurter, luncheon meat, meat pate.

The meat based emulsion may be cooked, sterilised or baked, e.g. in a baking form or after being filled into a casing of for example plastic, collagen, cellulose or a natural casing. A processed meat product may also be a restructured meat product, such a for example restructured ham. A meat product of the invention may undergo processing steps such as for example salting, e.g. dry salting; curing, e.g. brine curing; drying; smoking; fermentation; cooking; canning; retorting; slicing and/or shredding.

In another embodiment the food/feed product may be an emulsified meat product.

Meat

The term “meat” as used herein means any kind of tissue derived from any kind of animal.

The term meat as used herein may be tissue comprising muscle fibres derived from an animal. The meat may be an animal muscle, for example a whole animal muscle or pieces cut from an animal muscle.

In another embodiment the meat may comprise inner organs of an animal, such as heart, liver, kidney, spleen, thymus and brain for example.

The term meat encompasses meat which is ground, minced or cut into smaller pieces by any other appropriate method known in the art.

The meat may be derived from any kind of animal, such as from cow, pig, lamb, sheep, goat, chicken, turkey, ostrich, pheasant, deer, elk, reindeer, buffalo, bison, antelope, camel, kangaroo; any kind of fish e.g. sprat, cod, haddock, tuna, sea eel, salmon, herring, sardine, mackerel, horse mackerel, saury, round herring, Pollack, flatfish, anchovy, pilchard, blue whiting, pacific whiting, trout, catfish, bass, capelin, marlin, red snapper, Norway pout and/or hake; any kind of shellfish, e.g. clam, mussel, scallop, cockle, periwinkle, snail, oyster, shrimp, lobster, langoustine, crab, crayfish, cuttlefish, squid, and/or octopus.

In one embodiment the meat is beef, pork, chicken, lamb and/or turkey.

Biophysical Characteristic

Feeding an animal a tripeptidyl peptidase (optionally in combination with an endoprotease) and/or a feedstuff and/or feed additive composition and/or composition of the invention may improve a biophysical characteristic of animal so fed.

Suitably the method and/or use may further comprising administering to an animal at least one feed component, at least one mineral, at least one vitamin or combinations thereof.

Alternatively or additionally the method and/or use may further comprise administering to an animal at least one endoprotease.

As used herein, “biophysical characteristic” means any biophysical property of an animal which improves its health and/or performance and/or output.

By way of example, the biophysical characteristic may be one or more selected from the group consisting of one or more of the following: performance of the animal, growth performance of an animal, feed conversion ratio (FCR), ability to digest a raw material (e.g. nutrient digestibility, including starch, fat, protein, fibre digestibility), nitrogen digestibility (e.g. ileal nitrogen digestibility) and digestible energy (e.g. ileal digestible energy), nitrogen retention, carcass yield, growth rate, weight gain, body weight, mass, feed efficiency, body fat percentage, body fat distribution, growth, egg size, egg weight, egg mass, egg laying rate, lean gain, bone ash %, bone ash mg, back fat %, milk output, milk fat %, reproductive outputs such as litter size, litter survivability, hatchability % and environmental impact, e.g. manure output and/or nitrogen excretion.

Suitably the biophysical characteristic may be one or more selected from the group consisting of: feed conversion ratio, nitrogen digestibility (e.g. ileal nitrogen digestibility) and digestible energy (e.g. ileal digestible energy).

In a preferred embodiment the biophysical characteristic may be the ability to digest a protein.

In one embodiment the biophysical characteristic of the animal means the performance of the animal.

Suitably, administering to an animal a feed additive composition and/or feed and/or feedstuff and/or feed ingredient and/or premix of the invention may not substantially increase the incidence of necrotic enteritis in the animal when compared to an animal not fed with the feed additive composition and/or feed and/or feedstuff and/or feed ingredient and/or premix of the invention.

The term “substantially increase the incidence of necrotic enteritis” as used herein means that the incidence is not increased by more than about 20%, suitably not increased by more than about 10%. Preferably it is meant that the incidence of necrotic enteritis is not increased by more than about 5%, more preferably more than about 1%.

Performance

As used herein, “performance of the animal” may be determined by the feed efficiency and/or weight gain of the animal and/or by the feed conversion ratio and/or by the digestibility of a nutrient in a feed (e.g. amino acid digestibility) and/or digestible energy or metabolizable energy in a feed and/or by nitrogen retention.

Preferably “performance of the animal” is determined by feed efficiency and/or weight gain of the animal and/or by the feed conversion ratio.

By “improved performance of the animal” it is meant that there is increased feed efficiency, and/or increased weight gain and/or reduced feed conversion ratio and/or improved digestibility of nutrients or energy in a feed and/or by improved nitrogen retention in the subject resulting from the use of a hydrolysate of the present invention or a composition (e.g. food or feed additive composition) of the present invention in a feed compared with feeding the animal a feed not comprising a hydrolysate or a composition prepared in accordance with the present invention.

Preferably, by “improved animal performance” it is meant that there is increased feed efficiency and/or increased weight gain and/or reduced feed conversion ratio.

As used herein, the term “feed efficiency” refers to the amount of weight gain in an animal that occurs when the animal is fed ad-libitum or a specified amount of food during a period of time.

By “increased feed efficiency” it is meant that the use of a hydrolysate of the present invention or a composition (e.g. food or feed additive composition) according the present invention in feed results in an increased weight gain per unit of feed intake compared with an animal not fed with a hydrolysate or a composition prepared in accordance with the present invention.

Feed Conversion Ratio (FCR)

As used herein, the term “feed conversion ratio” refers to the amount of feed fed to an animal to increase the weight of the animal by a specified amount.

An improved feed conversion ratio means a lower feed conversion ratio.

By “lower feed conversion ratio” or “improved feed conversion ratio” it is meant that the use of a hydrolysate of the present invention or a composition (e.g. food or feed additive composition) in feed results in a lower amount of feed being required to be fed to an animal to increase the weight of the animal by a specified amount compared to the amount of feed required to increase the weight of the animal by the same amount without said hydrolysate or a composition prepared in accordance with the present invention.

Nutrient Digestibility

Nutrient digestibility as used herein means the fraction of a nutrient that disappears from the gastro-intestinal tract or a specified segment of the gastrointestinal tract, e.g. the small intestine. Nutrient digestibility may be measured as the difference between what is administered to the subject and what comes out in the faeces of the subject, or between what is administered to the subject and what remains in the digesta on a specified segment of the gastro intestinal tract, e.g. the ileum.

Nutrient digestibility as used herein may be measured by the difference between the intake of a nutrient and the excreted nutrient by means of the total collection of excreta during a period of time; or with the use of an inert marker that is not absorbed by the animal, and allows the researcher calculating the amount of nutrient that disappeared in the entire gastro-intestinal tract or a segment of the gastro-intestinal tract. Such an inert marker may be titanium dioxide, chromic oxide or acid insoluble ash. Digestibility may be expressed as a percentage of the nutrient in the feed, or as mass units of digestible nutrient per mass units of nutrient in the feed.

Nutrient digestibility as used herein encompasses starch digestibility, fat digestibility, protein digestibility, and amino acid digestibility.

Suitably use of a proline tolerant tripeptidyl peptidase according to the methods and/or uses or any of the aspects of the present invention (optionally in combination with at least one endoprotease) increases protein and/or amino acid digestibility in an animal fed with the feed additive composition and/or feed ingredient and/or feed and/or feedstuff and/or premix and/or hydrolysate of the invention.

Energy digestibility as used herein means the gross energy of the feed consumed minus the gross energy of the faeces or the gross energy of the feed consumed minus the gross energy of the remaining digesta on a specified segment of the gastro-intestinal tract of the animal, e.g. the ileum. Metabolizable energy as used herein refers to apparent metabolizable energy and means the gross energy of the feed consumed minus the gross energy contained in the faeces, urine, and gaseous products of digestion. Energy digestibility and metabolizable energy may be measured as the difference between the intake of gross energy and the gross energy excreted in the faeces or the digesta present in specified segment of the gastro-intestinal tract using the same methods to measure the digestibility of nutrients, with appropriate corrections for nitrogen excretion to calculate metabolizable energy of feed.

Nitrogen Retention

Nitrogen retention as used herein means as subject's ability to retain nitrogen from the diet as body mass. A negative nitrogen balance occurs when the excretion of nitrogen exceeds the daily intake and is often seen when the muscle is being lost. A positive nitrogen balance is often associated with muscle growth, particularly in growing animals.

Nitrogen retention may be measured as the difference between the intake of nitrogen and the excreted nitrogen by means of the total collection of excreta and urine during a period of time. It is understood that excreted nitrogen includes undigested protein from the feed, endogenous proteinaceous secretions, microbial protein, and urinary nitrogen.

Carcass Yield and Meat Yield

The term carcass yield as used herein means the amount of carcass as a proportion of the live body weight, after a commercial or experimental process of slaughter. The term carcass means the body of an animal that has been slaughtered for food, with the head, entrails, part of the limbs, and feathers or skin removed. The term meat yield as used herein means the amount of edible meat as a proportion of the live body weight, or the amount of a specified meat cut as a proportion of the live body weight.

Weight Gain

The present invention further provides a method of increasing weight gain in a subject, e.g. poultry or swine, comprising feeding said subject a feedstuff comprising a feed additive composition according to the present invention.

An “increased weight gain” refers to an animal having increased body weight on being fed feed comprising a hydrolysate of the present invention or a composition (e.g. food or feed additive composition) according to the present invention compared with an animal being fed a feed not comprising a hydrolysate or a composition prepared in accordance with the present invention.

Nonfood Products

The present invention provides a nonfood product comprising the hydrolysate of the invention.

The hydrolysate obtainable (e.g. obtained) may be used in the manufacture of a topically applied product, such as a lotion, cream, ointment, rub, cleanser, or the like. Accordingly, such products comprising the hydrolyzed protein compositions described herein are herein contemplated. Such products are useful for example for therapeutic purposes, for example, to provide relief from dry skin, itching, discomfort, and the like.

These products preferably comprise, in addition to the hydrolyzed protein component, a lipid, wax, oil, water in oil emulsion, oil-in-water emulsion, or the like, as a base. Typically, they may further comprise one or more fragrance components, as well as other ingredients such as surfactants or emulsifiers.

Cosmetic products and other appearance aids or beauty aids comprising the milk or whey protein hydrolyzates described herein are also provided.

In one embodiment, the cosmetic product may be applied to the face, cheeks, lips, or eyes of a person. In another embodiment the product may be used anywhere on the body to help improve the cosmetic appearance of the skin or, for example, to diminish the appearance of wrinkles moles, freckles, scars, blemishes, and the like.

Advantages

The inventors have shown for the first time that a proline tolerant tripeptidyl peptidase according to the present invention is highly advantageous for use in the preparation of hydrolysates and confers advantages to subjects fed the hydrolysate or a food and/or feed comprising the hydrolysate and/or a food or feed additive composition comprising the proline tolerant tripeptidyl peptidase (optionally in combination with at least one endoprotease).

Alternatively or additionally, the hydrolysate produced using an endoprotease and tripeptidyl peptidase may also have reduced bitterness when compared to an untreated hydrolysate. Advantageously, a proline tolerant tripeptidyl peptidase taught for use in the present invention is capable of acting on a wide range of peptide and/or protein substrates and due to having such a broad substrate-specificity is not readily inhibited from cleaving substrates enriched in certain amino acids (e.g. proline and/or lysine and/or arginine and/or glycine). The use of such a proline tolerant tripeptidyl peptidase therefore may efficiently and/or rapidly breakdown protein substrates (e.g. present in a substrate for preparation of a hydrolysate). The proline tolerant tripeptidyl peptidase may have a preferential activity on peptides and/or proteins having one or more of lysine, arginine or glycine in the P1 position. Without wishing to be bound by theory peptide and/or protein substrates comprising these amino acids at the P1 position may be difficult to digest for many tripeptidyl peptidases and/or proteases in generally and upon encountering such residues cleavage of the peptide and/or protein substrate by a tripeptidyl peptidase and/or protease may halt or slow. Advantageously, by using a proline tolerant tripeptidyl peptidase of the invention it is possible to digest protein and/or peptide substrates comprising lysine, arginine and/or glycine at P1 efficiently and/or without significantly slowing the cleavage reaction.

The present invention also provides for proline tolerant tripeptidyl peptidase that, in addition to having the activities described above, may be tolerant of proline at position P2, P2′, P3 and P3′. This is advantageous as it allows the efficient cleavage of peptide and/or protein substrates having stretches of proline and allows cleavage of a wide range of peptide and/or protein substrates.

Advantageously the proline tolerant tripeptidyl peptidase may have a preferential activity on peptides and/or proteins having lysine at the P1 position, this allows the efficient cleavage of substrates having high lysine content, such as whey protein.

The present invention also provides for thermostable proline tolerant tripeptidyl peptidases which are less prone to being denatured and/or will therefore retain activity for a longer period of time when compared to a non-thermostable variant.

Advantageously the proline tolerant tripeptidyl peptidase may have activity in a pH range of about pH 7 and can therefore be used with an alkaline endoprotease. This means that changing the pH of the reaction medium comprising the protein and/or peptide substrate for hydrolysate production is not necessary between enzyme treatments. In other words it allows the proline tolerant tripeptidyl peptidase and the endoprotease to be added to a reaction simultaneously, which may make the process for producing the hydrolysate quicker and/or more efficient and/or more cost-effective. Moreover, this allows for a more efficient reaction as at lower pH values the substrate may precipitate out of solution and therefore not be cleaved.

A proline tolerant tripeptidyl peptidase having activity at an acidic pH can be used in combination with an acid endoprotease and advantageously does not require the pH of the reaction medium comprising the protein and/or peptide substrate for hydrolysate production to be changed between enzyme treatments. In other words it allows the proline tolerant tripeptidyl peptidase and the endoprotease to be added to a reaction simultaneously, which may make the process for producing the hydrolysate quicker and/or more efficient and/or more cost-effective.

Advantageously, the endoprotease in combination with a proline tolerant tripeptidyl peptidase is capable of cleaving protein substrates associated with causing an immune response in sensitive individuals suffering from a disease, such as a milk protein allergy and/or a soy protein allergy.

Advantageously, the use of an endoprotease in combination with a proline tolerant tripeptidyl peptidase can increase the efficiency of substrate cleavage. Without wishing to be bound by theory, it is believed that an endoprotease is able to cleave a peptide and/or protein substrate at multiple regions away from the C or N-terminus, thereby producing more N-terminal ends for the proline tolerant tripeptidyl peptidase to use as a substrate, thereby advantageously increasing reaction efficiency and/or reducing reaction times.

Many current feeding practices involve administering an alkaline protease active at a high pH (e.g. pH 8) to animals. Alkaline proteases are therefore only active lower down (e.g. later) in the gastrointestinal tract of an animal where the gastrointestinal tract becomes more alkaline, such as in the later part of the small intestine and the large intestine and caecum. Without wishing to be bound by theory, it is believed that producing oligopeptides in the later parts of the gastrointestinal tract increases populations of microbes which utilise the oligopeptides which in turn can lead to enteric disease challenges and/or reduced nutrients available for uptake by the animal. Additionally, later in the gastrointestinal tract (i.e. lower down) the mucosa is less well-protected than in the upper portions (e.g. the gizzard, proventriculus or stomach) and so is more easily damaged leading to inflammation. Advantageously, the use of a proline tolerant tripeptidyl peptidase having activity at an acid pH alleviates this problem as it is capable of digesting its substrate in the upper gastrointestinal tract thereby not substantially increasing populations of microbes and/or increasing the amount of nutrient (e.g. amino acids/peptides) available for uptake by an animal and/or reducing inflammation.

Advantageously, the use of an endoprotease in combination with a proline tolerant tripeptidyl peptidase can increase the efficiency of substrate cleavage. Without wishing to be bound by theory, it is believed that an endoprotease is able to cleave a peptide and/or protein substrate at multiple regions away from the C or N-terminus, thereby producing more N-terminal ends for the proline tolerant tripeptidyl peptidase to use as a substrate, thereby advantageously increasing reaction efficiency and/or reducing reaction times.

The use of an acid endoprotease and a proline tolerant tripeptidyl peptidase having activity at an acid pH is highly advantageous as the two enzymes can co-operate to digest a peptide and/or protein substrate in the upper gastrointestinal tract (e.g. gizzard, proventriculus or stomach) of an animal and can be active in combination with other endogenous proteases (e.g. pepsin, trypsin or chymotrypsin) present in the animal.

Advantageously feeding a proline tolerant tripeptidyl peptidase to an animal results in increased body weight gain and/or a reduction in feed conversion ratio and/or increased nitrogen digestibility (e.g. ileal nitrogen digestibility) and/or increased energy digestion (e.g. ileal energy digestion).

Use of an endoprotease, an exo-tripeptidyl peptidase of the S53 family (e.g. such as a proline tolerant tripeptidyl peptidase) and an aminopeptidase optionally in combination with one or more further components has many advantages:
it allows for the efficient production of single amino acids and/or dipeptides and/or tripeptides which can efficiently be absorbed by a subject (e.g. due to having a better osmotic potential for uptake);
a protein and/or peptide substrate may be more efficiently and/or more quickly digested;
reduced end-point inhibition (i.e. inhibition by its reaction products) of a the proline tolerant tripeptidyl peptidase, particularly when used in vitro, such as in the manufacture of a hydrolysate by digesting the tripeptides into single amino acids and/or dipeptides; and/or
synergistic and/or additive activity on substrates containing high levels of proline, lysine, arginine and/or glycine.

Additional Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure which can be had by reference to the specification as a whole. Accordingly, the terms defined immediately below are more fully defined by reference to the specification as a whole.

Amino acids are referred to herein using the name of the amino acid, the three letter abbreviation or the single letter abbreviation.

The term “protein”, as used herein, includes proteins, polypeptides, and peptides.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a proline tolerant tripeptidyl peptidase”, “an endoprotease”, “exo-tripeptidyl peptidase of the S53 family”, “aminopeptidase” or “an enzyme” includes a plurality of such candidate agents and reference to “the feed”, “the feedstuff”, “the premix” or “the feed additive composition” includes reference to one or more feeds, feedstuffs, premixes and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Figures and Examples.

### EXAMPLES

### Example 1

Cloning and Expression of Proline Tolerant Tripeptidyl Peptidases in Trichoderma reesei.

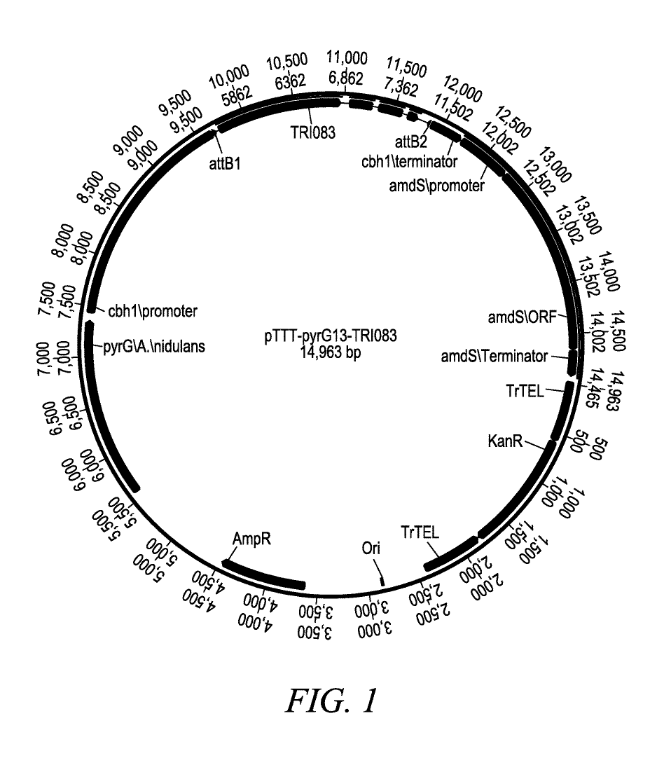

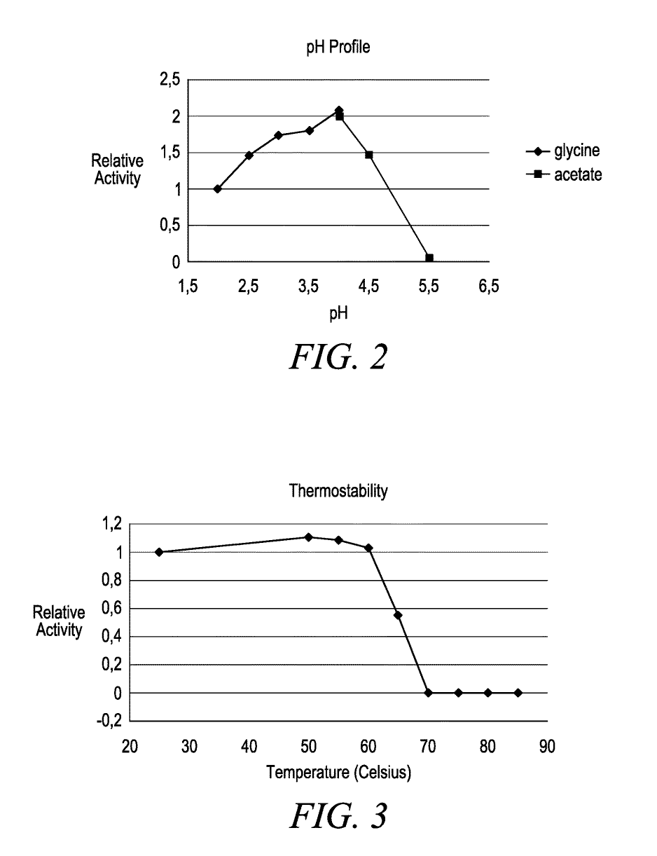

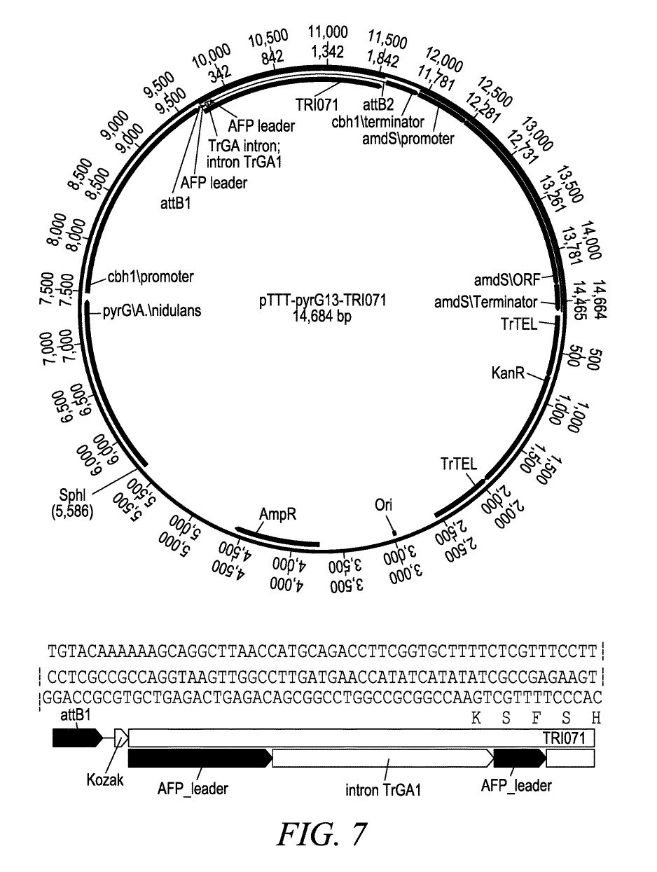

Synthetic genes encoding proline tolerant tripeptidyl peptidases were generated using preferred codons for expression in Trichoderma reesei except for TRI079 (SEQ ID No. 57) and TRI083 (SEQ ID No. 56) that were generated as genomic sequences. The predicted secretion signal sequences (SignalP 4.0: Discriminating signal peptides from transmembrane regions. Thomas Nordahl Petersen, Søren Brunak, Gunnar von Heijne & Henrik Nielsen. Nature Methods, 8:785-786, 2011) were replaced (except for TRI079 and TRI083) by the secretion signal sequence from the Trichoderma reesei acidic fungal protease (AFP) and an intron from a Trichoderma reesei glucoamylase gene (TrGA1) (see FIG. 7 lower panel). Synthetic genes were introduced into the destination vector pTTT-pyrG13 (as described in U.S. Pat. No. 8,592,194 B2, the teaching of which is incorporated herein by reference) using LR Clonase™ enzyme mix (Life Technologies) resulting in the construction of expression vectors pTTT-pyrG13 for the proline tolerant tripeptidyl peptidases herein. Expression vectors encoding SEQ ID No's 1, 2 and 29 are shown in FIG. 1 and encoding SEQ ID No's 12 and 39 are shown in FIG. 7. Expression vectors encoding SEQ ID No's 96 or 97 (TRI045) are shown in FIGS. 2.5-10 μg of the expression vectors were transformed individually into a suitable Trichoderma reesei strain using PEG mediated protoplast transformation essentially as described in (U.S. Pat. No. 8,592,194 B2). Germinating spores were harvested by centrifugation, washed and treated with 45 mg/ml of lysing enzyme solution (Trichoderma harzianum, Sigma L1412) to lyse the fungal cell walls. Further preparation of protoplasts was performed by a standard method, as described by Penttilä et al. (Gene 61(1987) 155-164).

Spores were harvested using a solution of 0.85% NaCl, 0.015% Tween 80. Spore suspensions were used to inoculate liquid cultures

Cultures were grown for 7 days at 28° C. and 80% humidity with shaking at 180 rpm. Culture supernatants were harvested by vacuum filtration and used to measure expression and enzyme performance.

### Example 2

Purification and Characterization

A. Purification of Proline Tolerant Tripeptidyl Peptidase

Desalting of samples was performed on PD10 column (GE Life Sciences, USA) equilibrated with 20 mM Na-acetate, pH 4.5 (buffer A). For ion exchange chromatography on Source S15 HR25/5 (GE Life Sciences, USA) the column was equilibrated with buffer A. The desalted sample (7 ml) was applied to the column at a flow rate of 6 ml/min and the column was washed with buffer A. The bound proteins were eluted with a linier gradient of 0-0.35 M NaCl in 20 mM Na-acetate, pH 4.5 (35 min). During the entire run 10 ml fractions were collected. The collected samples were assay for tripeptidyl amino-activity as described below. Protein concentration was calculated based on the absorbance measure at 280 nm and the theoretical absorbance of the protein calculated using the ExPASy ProtParam tool (http://web.expasy.org/cgi-bin/protparam/protparam).

B. Determination of Proline Tolerant Tripeptidyl Peptidase and Endopeptidase Activity

The chromogenic peptide H-Ala-Ala-Ala-pNA (MW=387.82; Bachem, Switzerland) was used to determine the activity of proline tolerant tripeptidyl peptidase in the samples produced as described above. The assay was conducted as follows, 10 μL of the chromogenic peptide solution (10 mM dissolved in dimethly sulfoxide; DMSO) were added to 130 μl Na-acetate (20 mM, adjusted to pH 4.0 with acetic acid) in a microtiter plate and heated for 5 minutes at 40° C. 10 μL of appropriately diluted enzyme was added and the absorption was measured in an MTP reader (Versa max, Molecular Devices, Denmark) at 405 nm. One katal of proteolytic activity was defined as the amount of enzyme required to release 1 mole of p-nitroaniline per second.

Azoscasein Assay for Endoprotease Activity.

A modified version of the endoprotease assay described by Iversen and Jørgensen, 1995 is used. An enzyme sample of 50 μl is added to 250 μl of azocasein (0.25% w/v; from Sigma) in 4 times diluted Mcllvaine buffer, pH 5 and incubated for 15 min at 40° C. with shaking (800 rpm). The reaction is terminated by adding 50 μl of 2 M TCA and centrifugation for 5 min at 20,000 g. To a 195 μl sample of the supernatant 65 μl of 1 M NaOH is added and absorbance at 450 nm is measured. One unit of endoprotease activity is defined as the amount which yields an increase in absorbance of 0.1 in 15 min at 40° C.

The proline tolerant tripeptidyl peptidase samples produced as described in Example 1 were found to be essentially free of endopeptidase side-activity. Upon purification as described in Example 2A, substantially no endopeptidase side activity was detected.

C. pH Profile

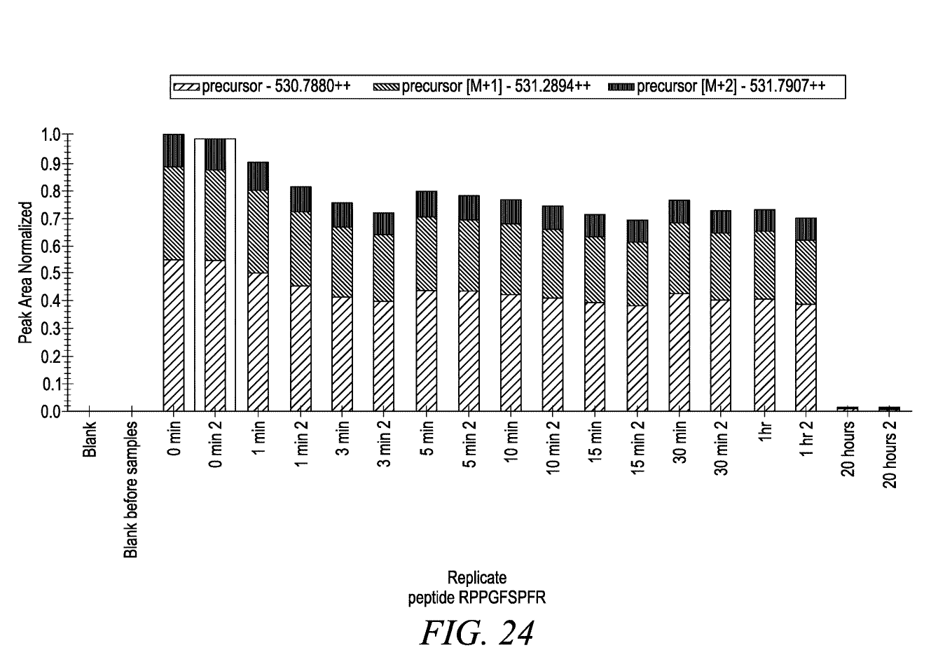

The proline tolerant tripeptidyl peptidase assay described with H-Ala-Ala-Ala-pNA substrate above with modification of using the buffers 20 mM Na-glycine (pH 2.0, 2.5, 3.0, 3.5 and 4.0) or 20 mM Na-acetate buffer (pH 4.0, 4.5 and 5.5) was used to determine the pH profile of proline tolerant tripeptidyl peptidase TRI083 (FIG. 2) and TRI045 (FIG. 24). Optimum pH of TRI083 and TRI045 was observed to be 4.0.

D. Temperature Profile

The proline tolerant tripeptidyl peptidase assay described above was used at temperatures 25, 50, 55, 60, 65, 70, 75, 80 and 85° C. The optimum temperature of proline tolerant tripeptidyl peptidase TRI083 was found to be 50° C., whereas no activity was found at 70° C. and higher temperatures (FIG. 3).

Protein Hydrolysis Using a Proline Tolerant Tripeptidyl Peptidase in Combination with an Endoprotease (Alphalase® AFP)

The enzymes: Alphalase® AFP and the proline tolerant tripeptidyl peptidase TRI083 expressed as described in Example 1.

Assay buffer: 50 mM NaOAc, pH 4.0, 3% dimethylhemoglobin (Sigma Aldrich), 37° C., 1 h incubation (100 μl reaction mixture per MTP well).

Stop/colour reagent: 0.05% trinitrobenzenesulfonic acid in 125 mM Na borate pH 8.6 (200 μl per well).

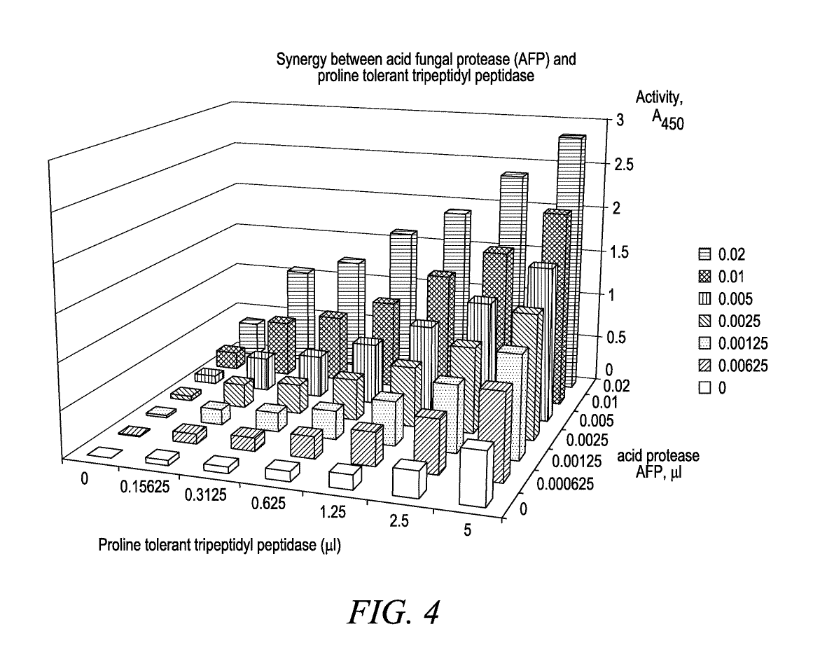

The plate was read at 450 nm using a Versa max microplate reader (Molecular Devices) after about 20 min incubation with stop/colour reagent. The results of the assay (FIG. 4) show a synergistic effect when an endoprotease (Alphalase® AFP) is used in combination with the proline tolerant tripeptidyl peptidase.

Hydrolysis of Whey Protein (WPI) Employing Proline Tolerant Tripeptidyl Peptidase in Combination with an Endopeptidase

For whey protein hydrolysis, Lacprodan-9224 (Arla Food Ingredients, Denmark) was employed. A 20% (w/w) WPI suspension was prepared in H₂Od and adjusted to pH 4.0 with acetic acid (Sigma-Aldrich, Denmark). To prevent microbial growth, 0.0285% (w/w) NaN₃ was added. A volume of 200 μl of the WPI suspension was transferred into each of the 96 wells of a microtiter plate (MTP; VWR, Denmark). Subsequently, 5 μl of each consecutively diluted peptidase, Alphalase® FP2 and proline tolerant tripeptidyl peptidase (3PP=TRI083; 0-8750 nkat H-Ala-Ala-Ala-pNA/mL activity in stock solution added), were added to each well, resulting in dosages as shown in Table 1. The MTP was sealed and placed in an iEMS incubator/shaker (Thermo Scientific, Denmark) at 40° C. After 24 h of incubation and shaking at 400 rpm, the hydrolyses were stopped by addition of 20 μl of 2 M trichloroacetic acid (TCA; Sigma-Aldrich, Denmark). As a reference, a plain 20% WPI (w/w) suspension was used which was treated in the same manner as the WPI hydrolyses. Unhydrolysed, precipitated WPI was removed by filtration (0.22 μm). On the filtered WPI hydrolysate o-phthaldehyde (OPA) derivatization was conducted according to Nielsen et al. (2001) with minor modifications. A sample volume of 25 μl was transferred to a well and 175 μl of OPA-reagent, dissolved in phosphate buffer pH 11, was added subsequently. The measured absorptions at 340 nm in an MTP reader (Molecular Devices, Denmark) were transformed into the relative degree of hydrolysis (DH) in percent.

A 20% (w/w) WPI suspension hydrolysed in 6 M HCl (Sigma-Aldrich, Denmark) at 100° C. for 24 h served as reference (100% DH).

As shown in table 1, the combination of proline tolerant tripeptidyl peptidase and endopeptidase Alphalase® FP2 gave the best degree of hydrolysis. The supplementation of endopeptidase with the proline tolerant tripeptidyl peptidase resulted in generally higher DH than without the addition of TRI083 (SEQ ID No. 1, SEQ ID No. 2 or SEQ ID No. 29). For example, the addition of 5 ul 2,188 nkat/ml TRI083 to 0.6% Alphalase® FP2 resulted in an increased degree of hydrolyses of 25.0%, whereas the supplementation of 1.2% (w/w) Alphalase® FP2 with 5 ul 8,375 nkat/ml TRI083 resulted in an increase of 21.4% in DH.

TABLE 1
Degree of hydrolysis of 20% (w/w) WPI hydrolysed with an
endopeptidase in combination with a proline tolerant tripeptidyl
peptidase (3PP)

| | |
|---|---|
| Alphalase ® | |
| FP2 | TRI083 dosage |
| dosage_{applied WPI} | [nkat/mL stock solution] |

| [%] | 0.00 | 2,188 | 4,375 | 8,375 |
|---|---|---|---|---|
| 0.0 | 0.0 | 0.5 | −0.9 | 1.4 |
| 0.6 | 8.8 | 11.1 | 11.0 | 11.5 |
| 1.2 | 11.2 | 12.2 | 12.9 | 13.6 |
| 4.6 | 17.8 | 19.4 | 20.2 | 20.2 |

Hydrolysis of 33-Mer Gliadin Peptide

The proline tolerant tripeptidyl peptidase was examined for its ability to hydrolyse a synthetic substrate from alpha gliadin (alpha-2-gliadin) by LC-MS and label-free quantification. It was found to cleave the substrate into tri-peptides irrespective of high proline content in the substrate.

Experimental Set-Up

The 33-mer of gliadin (alpha-2-gliadin) (aa56-88) H-LQLQPFPQPQLPYPQPQLPYPQPQLPYPQPQPF-OH (Zedira GmbH; D-64293 Darmstadt, Germany) C190H273N43O47 (MW=3911.46) (1 mg/ml; 0.26 mM) was incubated at 24° C. in the presence of proline tolerant tripeptidyl peptidase (0.01 mg/ml TRI083) in a total volume of 1000 ul buffer (pH=4.5) (the ratio substrate/enzyme 100:1, w/w). Aliquots (100 ul) of enzyme reaction were stopped with 20 ul 5% trifluoroacetic acid (TFA) after 0, 1, 3, 5, 10, 15 and 30 minutes, respectively. The samples were then transferred to new vials and analyzed on the LTQ Orbitrap mass spectrometer.

Data Acquisition, Label Free Quantification and MS/MS Data Analysis

Nano LC-MS/MS analyses were performed using an Easy LC system (Thermo Scientific, Odense, DK) interfaced to a LTQ Orbitrap Classic hybrid mass spectrometer (Thermo Scientific, Bremen, Germany). Samples were loaded onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle. Separation was performed at a flow rate of 300 nL/min using a 10 min gradient of 0-34% Solvent B (H2O/CH3CN/TFE/HCOOH (100/800//100/1 v/v/v/v)) into the nanoelectrospray ion source (Thermo Scientific, Odense, DK). The LTQ Orbitrap Classic instrument was operated in a data-dependent MS/MS mode. The peptide masses were measured by the Orbitrap (MS scans were obtained with a resolution of 60 000 at m/z 400), and up to 2 of the most intense peptide m/z were selected and subjected to fragmentation using CID in the linear ion trap (LTQ). Dynamic exclusion was enabled with a list size of 500 masses, duration of 40 s, and an exclusion mass width of ±10 ppm relative to masses on the list.

The RAW files were accessed with the open source program Skyline 1.4.0.4421 (available from MacCoss Lab Software, University of Washington, Department of Genome Sciences, 3720 15^{th} Ave NE Seattle, Wash., US) which can use the MS1 intensities to build chromatograms. The precursor isotopic import filter was set to a count of three, (M, M+1, and M+2) at a resolution of 60,000 and the most intense charge state was used. Peptide sequences of the two substrates as well as their cleavage products were typed into Skyline and intensities were calculated in each sample.

The LC-MS/MS data was manually annotated using GPMAW to calculate theoretical values of fragmentation.

The triple charged mass of the intact alpha-2-gliadin peptide was isolated and followed over time. The intact peptide was not detectable after 3 min and was very quickly hydrolyzed (Table 2). Full hydrolysis of the 33-mer alpha-2-gliadin peptide would give the following tri-peptides: LQL″ QPF″ PQP″ QLP″ YPQ″ PQL″ PYP″ QPQ″ LPY″ PQP″ QPF. The intermediate product YPQPQLPYPQPQLPYPQPQPF resulting from cleaving off the four tri-peptides LQL, QPF, PQP and QLP from the alpha-2-gliadin substrate was found to accumulate and then to decrease (Table 2).

TABLE 2
Relative MS peak intensities of alpha-
2-gliadin and derived peptides

| | 0 | 1 | 3 | 5 | 10 | 15 | 30 |
|---|---|---|---|---|---|---|---|
| | min | min | min | min | min | min | min |
| Alpha-2 gliadin | 100 | 10 | 0 | 0 | 0 | 0 | 0 |
| YPQPQLPYPQP | 79 | 100 | 52 | 23 | 23 | 23 | 20 |
| QLPYPQPQPF | | | | | | | |
| LQL | 3 | 91 | 88 | 94 | 94 | 100 | 100 |
| QPF | 0 | 4 | 55 | 7 | 129 | 26 | 100 |
| PQL | 8 | 21 | 30 | 32 | 42 | 58 | 100 |
| PYP | 4 | 7 | 12 | 15 | 28 | 48 | 101 |
| LPY | 5 | 21 | 46 | 62 | 90 | 100 | 94 |

The accumulation of most of the expected tri-peptides was detected. The underlined tri-peptides were found and confirmed based their MS/MS fragmentation: LQL″ QPF″ PQP″ QLP″ YPQ″ PQL″ PYP″ QPQ″ LPY″ PQP″ QPF, whereas the QPF tri-peptides were found only based on their mass.

In conclusion, proline tolerant tripeptidyl peptidase was found to cleave the substrate alpha-2-gliadin consecutively into tri-peptides irrespective of a high proline content. During the hydrolysis proline was present in P3,P2,P1,P1′,P2″ and P3″ positions, respectively. This is in contrast to that previously found tripeptidyl peptidase do not cleave proline in P1 or P1″ positions (U.S. Pat. No. 7,972,808B2, U.S. Pat. No. 5,821,104, Reichard et al. 2006, Applied and Environmental Microbiology 72, 1739-1748).

Cleavage of AAPPA Peptide

Proline tolerant tripeptidyl peptidase was examined for its ability to hydrolyse a synthetic substrate AAPPA by LC-MS and label free quantification. The peptide H-AAPPA-NH2 (MW=424.49, from Schafer-N, Copenhagen) was dissolved in 20 mM MES buffer, pH=4.0 (1 mg/ml). 1000 ul of the H-AAPPA-NH2 solution was incubated with 200 ul proline tolerant tripeptidyl peptidase (TRI083) solution (40 ug/ml) (substrate/enzyme 100:0.8) at room temperature. At seven time points (0, 5, 15, 60, 180, 720 and 1440 min) 100 ul samples were withdrawn, diluted with 50 ul 5% TFA, heat inactivated (10 min at 80° C.) and kept at −20° C. until LC-MS analysis.

Nano LC-MS/MS analyses were performed using an Easy LC system (Thermo Scientific, Odense, DK) interfaced to a LTQ Orbitrap Classic hybrid mass spectrometer (Thermo Scientific, Bremen, Germany). Samples were loaded onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle. Separation was performed at a flow rate of 300 nL/min using a 10 min gradient of 0-34% Solvent B (H2O/CH3CN/TFE/HCOOH (100/800//100/1 v/v/v/v)) into the nanoelectrospray ion source (Thermo Scientific, Odense, DK). The LTQ Orbitrap Classic instrument was operated in a data-dependent MS/MS mode. The peptide masses were measured by the Orbitrap (MS scans were obtained with a resolution of 60 000 at m/z 400), and up to 2 of the most intense peptide m/z were selected and subjected to fragmentation using CID in the linear ion trap (LTQ). Dynamic exclusion was enabled with a list size of 500 masses, duration of 40 s, and an exclusion mass width of ±10 ppm relative to masses on the list.

The RAW files were accessed with the open source program Skyline 1.4.0.4421 which can use the MS1 intensities to build chromatograms. The precursor isotopic import filter was set to a count of three, (M, M+1, and M+2) at a resolution of 60,000 and the most intense charge state was used. Peptide sequences of the substrate as well as a cleavage product was typed into Skyline and intensities were calculated in each sample.

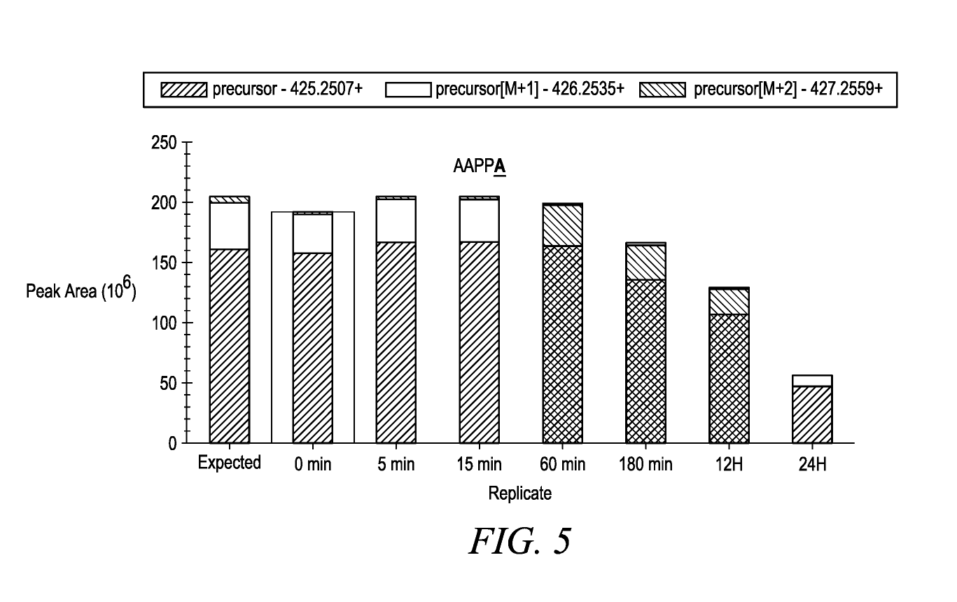

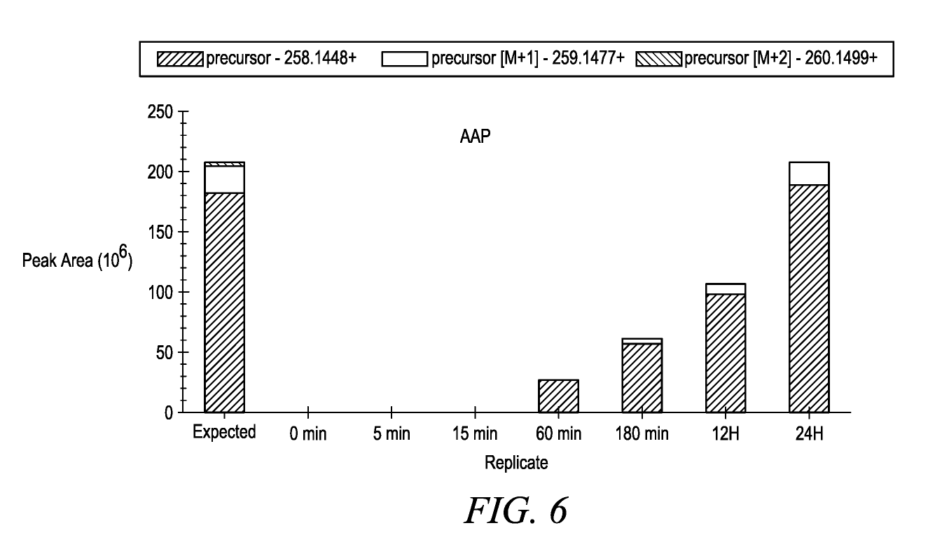

The analysis showed that proline tolerant tripeptidyl peptidase over time is able to degrade the peptide AAPPA (FIG. 5) and generate the product AAP (FIG. 6), indicating that the PP peptide bond in AAPPA is hydrolysed by the proline tolerant tripeptidyl peptidase.

Use of Proline Tolerant Tripeptidyl Peptidase to Reduce β-Lactoglobulin Allergenicity

The experiment was carried out using the kit: “Milk Protein ELISA Kit (β-lactoglobulin)”, supplied by MIoBS (Morianga Institute of Biological Sciences, Inc., Yokohama, Japan. Catalog No. 171LG).

Reduction in potential allergenicity was investigated by treating a β-lactoglobulin solution with proline tolerant tripeptidyl peptidase, endoprotease and a combination of proline tolerant tripeptidyl peptidase and endoprotease.

Buffer: 50 mM acetate, pH 4.5

β-Lactoglobulin solution: 100 μg/ml in buffer

Enzymes: proline tolerant tripeptidyl peptidase TRI083 (40000 nkat/ml in buffer) and Alphalase® AFP (100 SAPU/ml in buffer)

β-Lactoglobulin solution (1000 μL) was placed in an Eppendorf tube and heated to 40° C. 20 μL of buffer and 50 μL of proline tolerant tripeptidyl peptidase and was added (t=0), and the solution mixed by pumping with the pipette. Immediately after addition of enzyme an aliquot of 50 μL was transferred to 950 μL ice cold sample extraction solution (supplied by the kit). Subsequent aliquots were taken and treated in the same way after 1 h, 3 h, 6 h and 24 h. All samples were stored on ice until further analysis. The experiment was repeated using 20 μl Alphalase® AFP+50 μl buffer; 20 μl Alphalase® AFP+50 μl proline tolerant tripeptidyl peptidase or 70 μl buffer (no enzyme). All samples were analyzed using the protocol supplied in the kit (including incubating all samples in extraction solution at room temperature for 24 h as the initial step of the procedure).

Using the Milk protein ELISA Kit (β-lactoglobulin) it was found that proline tolerant tripeptidyl peptidase in combination with endoprotease is able to reduce assay response and thereby the potential allergenicity significantly more than Alphalase® AFP does alone. Proline tolerant tripeptidyl peptidase alone only had a minor effect on assay response.

### Example 3

Materials and Methods

1. Enzyme Treatment of Cornsoy Feed.

Feed flour was sifted to a particle size less than 212 μm and suspended in water to 10% (w/w) slurry and pH adjusted to pH3.5. Then, 138 μL of the 10% slurry was added to each well in 96 MTP well-plate using Agilent 0.5 mL instruments with Biomeck NX robot. Wide bore tips were used. Then 20 μl of enzyme solution containing proteases to be tested in 20 mM acetate pH 3.5 was added, after that 10 μL (1.14 U/μL) pepsin dissolved in water was added. The plate was incubated at 40° C. for 45 minutes in iEMS incubator/shaker at 1150 rpm. Then 34 μL pancreatin 1.126 mg/mL in 1M Na-bicarbonate was added and the plate was incubated at 40° C. for 60 minutes in iEMS at 1150 rpm. Afterwards, the plate was centrifuged at 5° C., 4000 rpm for 15 min, 10 μL supernatant was transferred to new plates (corning plate #3641 nonbinding) containing 190 μL water in each well to a 20× dilution. The obtained plates (master plates) were stored in the freezer −20° C.

2. Degree of Hydrolysis Measurements.

The method of analysis of degree of hydrolysis (DH) of soluble protein is based on the reaction of primary amino groups with o-phthaldialdehyde (OPA—assay). Reference: P. M. Nielsen, D. Petersen and C. Dambmann. Improved Method for Determining Food Protein Degree of Hydrolysis. Journal of Food Science. 66 (2001) 642-646.

For OPA assay the following procedure was carried on. 10-25 μl feed sample treated by enzyme from master plate was transferred to the new plate, then 175 μl of OPA reagent containing sodium borate, dodecyl sulfate and dithiothreitol, were added to the plate. The end point measurements of optical density at 340 nm were performed right after 2 min and 5 second mixing.

The Effect of Alphalase® AFP (an Acid Protease) on Corn Soy Feed in the Presence of Pepsin and Pancreatin. In Vitro Studies.

The composition of cornsoy feed is presented in the Table below (Interactions of phytate and myo-inositol phosphate esters (IP₁₋₅) including IP₅ isomers with dietary protein and iron and inhibition of pepsin. S. Yu, A. Cowieson, C. Gilbert, P. Plumstead and S. Dalsgaard J. Anim. Sci. 90 (2012) 1824-1832. Supplementary Information).

| Ingredient | Amount, % |
|---|---|
| Corn | 60.01 |
| Soybean meal | 31.52 |
| Soy oil | 4.00 |
| Salt | 0.40 |
| DL-Methionine | 0.20 |
| Limestone | 1.16 |
| Dicalcium Phosphate | 1.46 |
| Vitamin and mineral mixture | 1.25 |

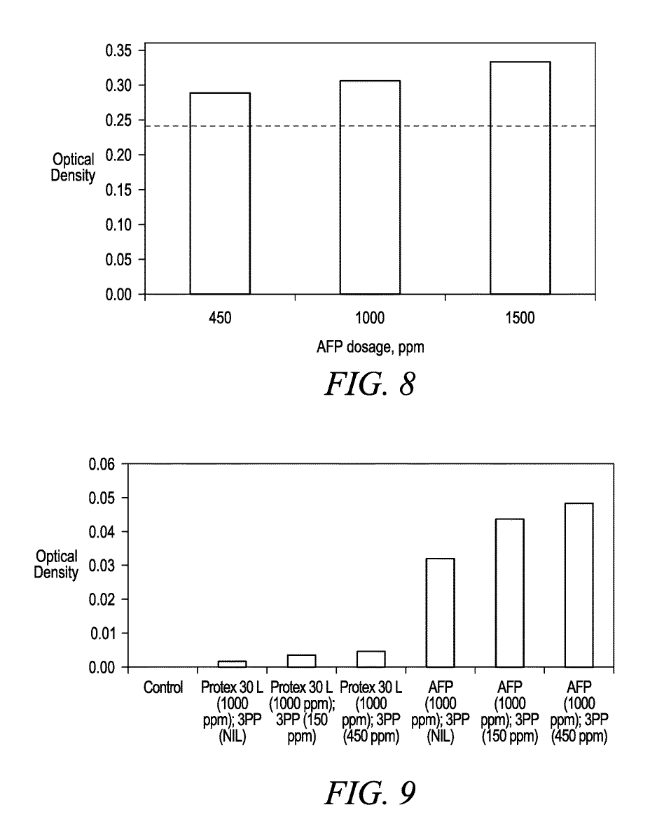

Cornsoy feed were treated with Alphalase® AFP (NSP24, available at Genencor Division, Food Enzymes) (herein referred to as “AFP”) at different concentrations (450, 1000 and 1500 ppm in relation to the cornsoy feed) in the presence of pepsin and pancreatin (FIG. 8). The results are presented below; the level of improvement of cornsoy feed DH is 4.5, 6.3 and 9.0%, respectively.

### Example 4

The Effect of a Proline Tolerant Tripeptidyl Peptidase on Cornsoy Feed in the Presence of Pepsin and Pancreatin and in the Presence and in the Absence of Alphalase® AFP. In Vitro Studies.

In these studies Alphalase® AFP and proline tolerant tripeptidyl peptidase were used only at the dosages of 1000 and 450 ppm, respectively. The results are presented in Table 3. In the control experiment only pepsin and pancreatin were used. Improvement of hydrolysis is the ratio between the treatment and control.

TABLE 3
The effect of proline tolerant tripeptidyl peptidase and Alphalase ® AFP
on hydrolysis of cornsoy feed.

| | Control | 3PP | AFP | AFP + 3PP |
|---|---|---|---|---|
| Degree of | 24.5 ± 2.0 | 25.5 ± 1.6 | 28.6 ± 3.6 | 32.3 ± 2.6 |
| hydrolysis, % | | | | |
| Improvement of | 100 ± 8.2 | 104 ± 6.5 | 117 ± 14.7 | 132 ± 10.7 |
| hydrolysis, %. | | | | |

As can be seen from Table 3 proline tolerant tripeptidyl peptidase on its own does not give sufficient benefit in cornsoy protein hydrolysis. The performance of Alphalase® AFP is similar to the results presented in the Example 3. However, the combination of proline tolerant tripeptidyl peptidase and Alphalase® AFP gives the maximal results and can be associated with the synergetic action of endo- and exo-proteases.

### Example 5

Validation of Dose Response of Proline Tolerant Tripeptidyl Peptidase in Combination with Endo-Proteases on Cornsoy Feed Hydrolysis in the Absence of Pepsin and Pancreatin.

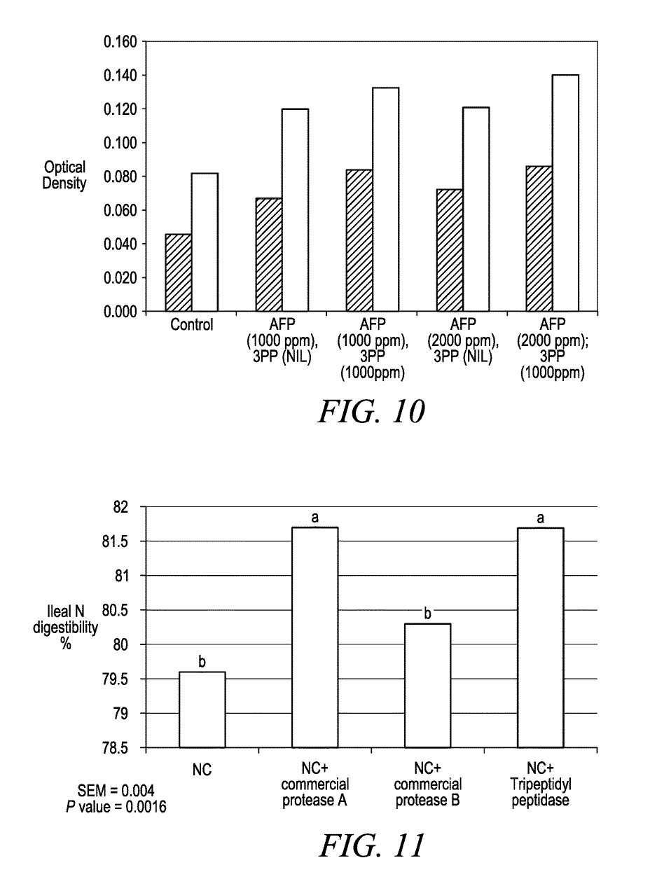

The aim of the work was to identify the origin of the enzymes performance and truly monitor possible additive or synergetic performance of endo- and exo-proteases. For this reason, the experiments were performed in the absence of pepsin and pancreatin and the results are summarized in FIG. 9.

As can be seen from FIG. 9, the degree of hydrolysis when proline tolerant tripeptidyl peptidase is used with endoproteases is very pronounced. It is clear that this phenomenon is synergetic, since the dose response depends on the level of proline tolerant tripeptidyl peptidase and the nature of endoprotease. In the case of Alphalase® AFP the effect is much more pronounced.

### Example 6

Validation of Dose Response of Proline Tolerant Tripeptidyl Peptidase in Combination with Different Dosages of Alphalase® AFP Endo-Proteases on Cornsoy Feed Hydrolysis in the Presence of Pepsin and Pancreatin.

To estimate the level of synergism between Alphalase® AFP and proline tolerant tripeptidyl peptidase, the experiment with different dosages of the enzymes and in the presence of pepsin and pancreatin were performed.

As can be seen from FIG. 10 the level of degree of hydrolysis is not dependent on concentration of proline tolerant tripeptidyl peptidase and Alphalase® AFP in the composition. It likely that for both enzymes the concentrations 1000 ppm and 2000 ppm correspond to the saturation level. However the degree of hydrolysis is dramatically increased when the treatment time increased from 100 min to 200 min.

### Example 7

Analysis of 20 Tripeptidyl Peptidases for their Low pH Stability at pH 2.5 40° C. 60 Min

As requirements for utilization of the enzymes in feed for monogastric animals, the enzymes should be active at lower pH. For this reason the number of tripeptidyl peptidases has been tested with the reaction on synthetic substrate AAF (H-Ala-Ala-Phe-pNA. BACHEM, L-1095), at pH 2.5.

The tripeptide substrate AAF was prepared at 2.5 mg/ml in DMSO. Tripeptidyl peptidases were used as broth. The reaction mixture containing 2.5 μl enzyme and 90 μL glycine acetic acid-Tris buffer (each buffer components at 50 mM, pH 2.5) was prepared in 96 well incubation plates.

The incubation plate was mixed and incubated at 40° C. for 60 min. Then 50 μl 0.2M Mes-NaOH pH6.0 was added to each well, mixed at 600 rpm for 2 min. After that, 5 ul of the incubation mixture were taken to 96 well assay plate already filled with 85 μl 0.1M acetic acid buffer, pH4.0 and 5 μl 2.5 mg/ml AAF substrate solution.

The reaction mixture was stirred at 600 rpm for 2 min and read directly in a microplate reader (Molecular Devices, Denmark) at 410 nm at 30° C. every 30 second for 15 min.

To measure initial enzyme activity, similar procedure was performed except the step of enzyme incubation at 40° C. for 60 min.

The results of measuring initial and final activity with activity recovery are presented in Table 4.

TABLE 4
Initial, final activity and its recovery for tripeptidyl peptidases.

| | OD₄₁₀ nm | OD₄₁₀ nm | Activity recovery |
|---|---|---|---|
| Protein name | (Initial) | (Final) | (%) |
| TRI050.3 (SEQ ID No. | 0.603 | 0.501 | 83 |
| 7/SEQ ID No. 34) | | | |
| TRI053.1 (SEQ ID No. | 0.345 | 0.356 | 103 |
| 10/SEQ ID No. 37) | | | |
| 29.9 Induction control | | | |
| Morph 1.1 Background | | | |
| TRI050 (SEQ ID No. | 0.895 | 0.814 | 91 |
| 7/SEQ ID No. 34) | | | |
| TRI071 (pool1) 2% | 0.757 | 0.589 | 78 |
| glu/soph (SEQ ID No. | | | |
| 12/SEQ ID No. 39) | | | |
| TRI071 (pool1) 2% | 0.815 | 0.672 | 82 |
| glu/soph (SEQ ID No. | | | |
| 12/SEQ ID No. 39) | | | |
| TRI071 (pool1) 4% | 0.466 | 0.453 | 97 |
| glu/soph (SEQ ID No. | | | |
| 12/SEQ ID No. 39) | | | |
| TRI071 (pool1) 4% | 0.67 | 0.6 | 90 |
| glu/soph (SEQ ID No. | | | |
| 12/SEQ ID No. 39) | | | |

From Table 4 it can been seen that 11 out of 20 samples of tripeptidyl peptidases are considerably stable at pH2.5 and 40° C. for 60 min since over 70% activities were retained. Keeping in mind that the final four samples are the same molecule (TRI071), and 2 samples are negative controls. From these results it can be concluded that proline tolerant tripeptidyl peptidases are stable at low pH.

Conclusions

This work demonstrated the synergies of the serine exopeptidase proline tolerant tripeptidyl peptidase with the serine endoprotease FoodPro® 30L and also the aspartic acid protease Alphalase® AFP in the hydrolysis of corn soy based feed under conditions mimicking the monogastric digestion system. It was also shown that many tripeptidyl peptidases are stable at low pH and can be considered for feed applications.

### Example 8

Proline Tolerant Tripeptidyl Peptidase in Animal Feed

A total of 288 one day old Ross 308 male broiler chicks were purchased form a commercial hatchery. At study initiation, 8 birds were randomly allocated to battery cages according to respective treatments by blocks. Only healthy birds were selected for the experiment, and no birds were replaced throughout the course of the study. The study consisted of the following treatments (Table 5):

TABLE 5
Experimental design

| | Protease | Phytase level |
|---|---|---|
| Dietary treatment | inclusion | (FTU/kg) |
| 1. Negative control (NC) | — | 500 |
| 2. NC + commercial product A | 0.2 g/kg | 500 |
| (trypsin family protease 75000 | | |
| PROT/g activity) | | |
| 3. NC + commercial product B | 4000 U/kg | 500 |
| (subtilisin family protease 2750-3500 GSU/g | | |
| activity) | | |
| 4. NC + tripeptidyl peptidase | 0.01 g/kg | 500 |

Bird weights were recorded at study initiation (d0) on day 14, and at study termination (d21). The cage is the experimental unit. Diets were fed in mash form and were formulated to meet or exceed NRC standards, except for Ca and AvP (Table 5). All feed was mixed using a

Davis S-20 mixer, the mixer was flushed between each treatment to prevent cross contamination between rations. Samples were collected from each treatment diet from the beginning, middle and end of each batch and were minced together for analysis of enzyme activity in feed.

All birds were fed a corn soy base ration until day 14; from day 14 the treatment rations were fed. Phytase was added to all treatment rations. At the feed change, feeders were removed from the cages, weighed back, emptied, and refilled with the appropriate treatment diet. On the final day of the study (d21), feed and birds were weighed, to determine feed intake (FI) and body weight gain (BWG) for the experimental period. Pens were checked daily for mortality. When a bird was culled or found dead, the date and removal weight (kg) were recorded. A gross necropsy was performed on all dead or culled birds to determine the possible cause of death. Feed conversion ratio (FCR) corrected for mortality was determined.

On d 21, all birds per cage were euthanised by intracardial injection of sodium pentobarbitone and contents of the lower ileum were expressed by gentle flushing with distilled water. Digesta from birds within a cage were pooled, resulting in eight samples per dietary treatment. The digesta samples were frozen immediately after collection, lyophilised and processed. Diets and digesta samples were analysed for the marker, nitrogen (N) and gross energy to enable calculation of digestibility co-efficients.

TABLE 6
Diet formulations

| Ingredient % | 0-14 days | 14-21 days |
|---|---|---|
| Maize | 48.78 | 57.09 |
| Soybean Meal 48% CP | 40.06 | 34.7 |
| Canola meal | 4 | 4 |
| Soybean Oil | 3 | 1.35 |
| L-Lysine HCl | 0.13 | 0.07 |
| DL-methionine | 0.28 | 0.22 |
| L-threonine | 0.03 | 0 |
| Salt | 0.33 | 0.33 |
| Limestone | 1 | 0.98 |
| Dicalcium Phosphate | 2.09 | 0.97 |
| Poultry Vits/TE's | 0.3 | 0.3 |
| Calculated Analyses | | |
| PROTEIN % | 24.98 | 22.97 |
| MEP MJ/KG | 12.4 | 12.34 |
| CALCIUM % | 1.05 | 0.76 |
| AV PHOS % | 0.5 | 0.3 |
| ALYS % | 1.27 | 1.1 |
| AM + C % | 0.94 | 0.84 |
| ATHRE % | 0.83 | 0.73 |
| ATRYP % | 0.26 | 0.23 |

Statistical Analysis

Data were analyzed using ANOVA, and means separation conducted to test differences between the different enzymes and enzyme dosages. Cage was used as the experimental unit.

Results

TABLE 7
Performance and digestibility results

| | BWG | | FCR | Ileal N | Ileal digestible |
|---|---|---|---|---|---|
| | (g) | FI (g) | (kg/kg) | Digestibility % | energy (MJ/kg) |
| NC | 386.3^{b} | 576.3^{a} | 1.496^{a} | 79.6^{b} | 12.45b |
| NC + commercial protease | 414.1^{ab} | 582^{a} | 1.429^{b} | 81.7^{a} | 12.98a |
| A (trypsin family protease | | | | | |
| 75000 PROT/g activity) | | | | | |
| NC + commercial protease | 386.2^{b} | 572.2^{a} | 1.486^{a} | 80.3^{b} | 12.89a |
| B (subtilisin family protease | | | | | |
| 2750-3500 GSU/g activity) | | | | | |
| NC + tripeptidyl peptidase | 420.8^{a} | 597.7^{a} | 1.422^{b} | 81.7^{a} | 13.12a |
| SEM | 11.1 | 9.4 | 0.018 | 0.4 | 0.112 |
| Effect tests | | | | | |
| Treatment | 0.0627 | 0.2563 | 0.0092 | 0.0016 | 0.0011 |

Each value represents the mean of 9 replicates (8 birds per replicate).
^{ab}Means in a column not sharing a common superscript are different (P < 0.05).

Supplementation of the proline tolerant tripeptidyl peptidase resulted in a significant reduction in FCR compared to the negative control and commercial protease B and a numerical reduction in comparison to commercial protease A.

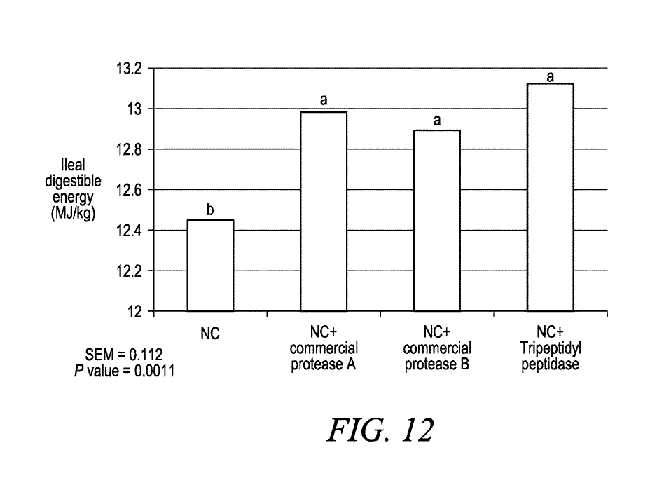

Proline tolerant tripeptidyl peptidase supplementation significantly increased BWG compared to the NC, this was not the case for either commercial protease A or commercial protease B. Proline tolerant tripeptidyl peptidase significantly increased ileal N digestibility % compared to the control and commercial protease A (FIG. 11). Proline tolerant tripeptidyl peptidase also significantly increased the energy digested to a level significantly greater than the negative control and numerically greater than the two commercial proteases (FIG. 12).

Conclusions

In conclusion, supplementation of proline tolerant tripeptidyl peptidase resulted in significantly better bird performance than the NC and commercial protease B in terms of FCR and BWG. There was a numerical increase in BWG and reduction in FCR when proline tolerant tripeptidyl peptidase was supplemented compared to commercial protease A.

The improvements in bird performance we driven by improved energy and protein (N) digestibility compared to the commercial proteases.

### Example 9

Determination of Proline Tolerant Tripeptidyl Peptidase, Aminopeptidase and Endopeptidase Activities

For proline tolerant tripeptidyl peptidase the chromogenic peptide H-Ala-Ala-Ala-pNA was used. The assay was conducted as follows, 27.5 μL of appropriately diluted enzyme was added to 200 μl Mcllvaine buffer (20 mM, pH 4.0). After incubation at 50° C. for 5 min, 12.5 μL of the chromogenic peptide solution (5 mg/mL dissolved in dimethly sulfoxide (DMSO); Sigma-Aldrich, Denmark) were added to the reaction mixture. The reaction was terminated by adding 50 μL of 0.5 M trichloroacetic acid (TCA; Sigma-Aldrich, Denmark). After centrifugation (15,000×g, 5 min), 240 μL of the solution were transferred to a microtiter plate (MTP; Kisker-biotech, Germany) and the absorption was measured in an MTP reader (Molecular Devices, Denmark) at 405 nm. One katal of proteolytic activity was defined as the amount of enzyme required to release 1 mol of p-nitroaniline per second.

Aminopeptidase activity was measured as described in Stressler, Eisele et al. 2013. Endopeptidase side-activity was measured with the azocasein as described in Example 2.

### Example 10

Whey Protein Hydrolysis Employing an Endopeptidase in Combination with a Proline Tolerant Tripeptidyl Peptidase and a General Aminopeptidase

A recombinantly in E. coli expressed and His-tag purified general aminopeptidase (PepN) from Lactobacillus helveticus ATCC 12046 was employed for whey protein isolate (WPI) hydrolyses (Stressler, Eisele et al. 2013). The applied PepN had biochemical parameters such as pH-optimum, temperature-range and sequence specificity comparable to other PepNs in the scientific literature (Table 8) and is therefore representative for general aminopeptidases from pro- and eukaryotic species.

TABLE 8
Biochemical parameters and sequence specificity of general aminopeptidases
(PepN) from different origin

| | pH- | temp.- | | |
|---|---|---|---|---|
| Origin | optimum [—] | range [C.] | Substrate Specificity | Literature |
| Streptomyces sp. | 7.0 | 30-55 | Gly, Ile, Val, Asp, Ala, Glu, His, | 1 |
| TH-4 | | | Pro, Lys, Phe, Leu, Arg, Tyr | |
| Aspergillus oryzae | 8.0 | 20-80 | Leu, Gly, Pro, Tyr, Lys, Ala, Phe, | 2 |
| | | | Glu, Arg, Met | |
| Geobacillus | 8.0 | 20-90 | Leu, Met, Phe, Val, Gly, Asp | 3 |
| thermodenitrificans | | | | |
| NG80-2 | | | | |
| Lactobacillus | 6.5 | 10-50 | Ala, Lys, Arg, Leu, Phe, Val, Ile, | 4 |
| helveticus ATCC | | | Pro | |
| 12046 | | | | |

1: Hatanaka, 2007; Appl Microbiol Biotechnol 74: 347-356;
2: (Nakadai, Nasuno et al. 1973);
3: (Wang, Guo et al. 2012);
4: (Stressler, Eisele et al. 2013)

The applied PepN in WPI hydrolysis was free of endopeptidase and X-prolyl-dipeptidyl (PepX) side-activity, which has been assayed by either the azocasein assay or with H-Ala-Pro-pNA as a substrate for PepX, respectively.

For whey protein hydrolysis, Lacprodan-9224 (Arla Food Ingredients, Denmark) was employed, a 15% (w/w) WPI suspension was prepared in H₂O_{d} and adjusted to pH 4.5 with acetic acid (Sigma-Aldrich, Denmark). To prevent microbial growth, 0.0285% (w/w) NaN₃ was added. Prior to the exopeptidase hydrolysis, 1% (w/w_{applied WPI}) Alphalase® FP2 was added to initiate the hydrolysis of the WPI in order to generate substrates for the exopeptidases (tripeptidyl peptidase and general aminopeptidase). A volume of 200 μl of the WPI suspension was transferred into each of the 96 wells of a microtiter plate (MTP; VWR, Denmark). Subsequently, 5 μl of consecutively diluted PepN (0-1572 nkat_{H-Ala-pNA}/mL) and proline tolerant tripeptidyl peptidase (0-70,000 nkat_{H-Ala-Ala-Ala-pNA}/mL) were added to each well. Then, the MTP was sealed and placed in an incubator (iEMS incubator/shaker HT, Thermo scientific, Denmark) which was incubated at 40° C. After 24 h of incubation at 400 rpm, the hydrolyses were stopped by addition of 20 μl of 2 M trichloroacetic acid (TCA; Sigma-aldrich, Denmark), except for the reference (0 h), whereas the TCA was added prior to endo- and exopeptidase addition. Unhydrolysed, precipitated WPI was removed by filtration (0.22 μm; Corning 3504 filter plate, Corning incorporated, USA). The filtered WPI hydrolysate was employed for o-phthaldehyde (OPA) derivatization (Nielsen, Petersen et al. 2001). The OPA derivatization was conducted according to Nielsen et al., (2001) with minor modifications. A sample volume of 25 μl was transferred to a well and 175 μl of OPA-reagent, containing trisodium phosphate-dodecahydrate, was added. The measured absorptions at 340 nm in a MTP reader (VersaMax, Molecular Devices, Denmark) were optionally transformed in the relative degree of hydrolysis (DH) in percent, whereas a 15% (w/w) WPI suspension was prepared in 6 M HCl (Sigma-Aldrich, Denmark) and held at 100° C. for 24 h. The acid hydrolysate of the 15% (w/w) WPI served as a reference (100% DH) for the calculation of the degree of hydrolysis.

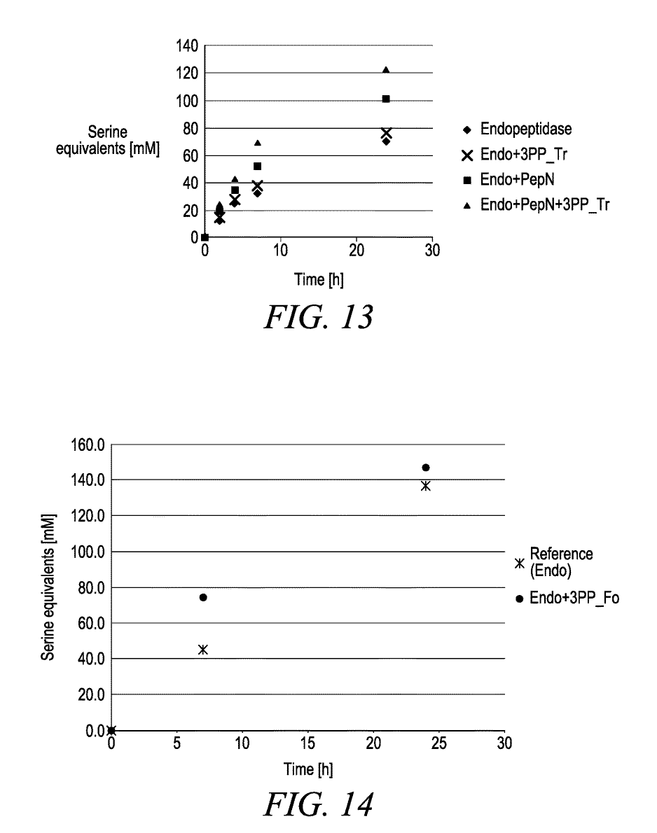

FIG. 13 shows the increase of free alpha amino groups, expressed as serine equivalents (SE), over 24 h employing different endo- and exopeptidase combinations. The lowest increase in SE was determined with the sole addition of endopeptidases, followed by the combination of endopeptidase and proline tolerant tripeptidyl peptidase (TRI083), which releases tripeptides from the N-terminal end of peptides. Due to the fact that the proline tolerant tripeptidyl peptidase TRI083 is product inhibited by its liberated tripeptides, the increase in SE is not very high. The supplementation of the endopeptidase with a recombinantly expressed and His-tag purified general aminopeptidase (PepN) from Lactobacillus helveticus ATCC12046 resulted in a significant increase in SE due to the liberation of free amino acids (FIG. 13). The combined application of endopeptidase, TRI083 and PepN resulted in a significantly higher release of free alpha amino groups compared to the addition of endopeptidase and TRI083 as well as endopeptidase and PepN of 43.5% (FIG. 13).

A summary of the additional effect of the endopeptidase, the proline tolerant tripeptidyl peptidase and the general aminopeptidase (PepN) is shown in table 9.

TABLE 9
Summary of the additional effect of an endopeptidase combined with a proline tolerant tripeptidyl
peptidase (TRI083) and a general aminopeptidase (PepN) in a 15% WPI hydrolysis after 24 h.

| | | | | | | | | | | | | |
|---|---|---|---|---|---|---|---|---|---|---|---|---|
| PepN [nkat/mL] | 0 | 197 | 393 | 0 | 0 | 0 | 197 | 197 | 197 | 393 | 393 | 393 |
| 3PP_Tr [nkat/ml] | 0 | 0 | 0 | 2,188 | 4,375 | 8,750 | 2,188 | 4,375 | 8,750 | 2,188 | 4,375 | 8,750 |
| Increase of DH [%] | | | | | | | 32.8 | 43.5 | 38.8 | 62.4 | 41.7 | 50.1 |

### Example 11

Casein Hydrolysis Employing an Endopeptidase in Combination with a Proline Tolerant Tripeptidyl Peptidase (TRI083) and a General Aminopeptidase (PepN)

The hydrolysis of a 5% (w/w) sodium caseinate solution was conducted in the same set-up as described for the whey protein hydrolysis in Example 2, except that 5% (w/w) sodium caseinate (Arla Foods, Denmark) was employed. As shown in Table 10, independent of the applied activity of PepN or TRI083, the determined serine equivalents (SE) could be increased either by the addition of TRI083 to the endopeptidase or by addition of PepN to the endopeptidase.

In addition, a synergistic effect was observed for high PepN and high TRI083 dosages (Table 11). The doubling of the PepN activity from 786 to 1572 nkat/mL resulted in a rather small increase of serine equivalents of 10 mM, whereas the application of 17,500 nkat/mL TRI083 increased the SE by 18 mM. The application of both TRI083 and PepN at 17,500 and 1,572 nkat/mL, resulted in an increase of 97 mM SE. This corresponds to a 21% increased SE value compared to the sum of the sole additions of TRI083 and PepN on top of the endopeptidase.

TABLE 10
Dose response of a 5% (w/w) sodium caseinate hydrolysis with an
endopeptidase in combination with different activities of TRI083 and
PepN after 24 h of hydrolysis, which is quantified in serine equivalents
(SE)

| | TRI083 [nkat/mL] | | | | |
|---|---|---|---|---|---|
| PepN [nkat/mL] | 0 | 1,094 | 2,188 | 4,375 | 8,750 |
| 0 | 0 | 4 | 4 | 11 | 11 |
| 25 | 8 | 16 | 15 | 16 | 17 |
| 49 | 15 | 26 | 17 | 27 | 35 |
| 98 | 27 | 33 | 34 | 44 | 39 |
| 197 | 36 | 41 | 37 | 45 | 47 |
| 393 | 49 | 58 | 51 | 56 | 62 |
| 786 | 52 | 59 | 62 | 67 | 67 |

### Example 12

Casein Hydrolysis Employing an Endopeptidase in Combination with a Proline Tolerant Tripeptidyl Peptidase and Partly Inactivated Flavourzyme 500

The sodium caseinate hydrolysis was conducted as describe in Example 10 except that partly inactivated Flavourzyme 500 (FZ500; Sigma-Aldrich, Denmark) instead of a purified PepN was applied. In order to inactivate the endopeptidases, FZ 500 was heat treated (55° C., 24 h). The endopeptidase activity was significantly reduced (96% inactivated, 4% residual activity). After heat treatment of the FZ 500, about 97% residual PepN activity was determined.

TABLE 11
Dose response of a 5% (w/w) sodium caseinate hydrolysis with an
endopeptidase in combination with a proline tolerant tripeptidyl peptidase
(TRI083) and partly inactivated FZ 500 after 24 h of hydrolysis, which is
quantified in serine equivalents (SE).

| | TRI083 [nkat/mL] | | | | |
|---|---|---|---|---|---|
| Flavourzyme 500 | 0 | 1,094 | 2,188 | 4,375 | 8,750 |
| Without FZ 500 addition | 0 | 8 | 10 | 17 | 21 |
| 64-fold diluted | 2 | 7 | 12 | 20 | 23 |
| 32-fold diluted | 0 | 18 | 13 | 27 | 39 |
| 16-fold diluted | 4 | 19 | 9 | 26 | 30 |
| 8-fold diluted | 17 | 25 | 25 | 26 | 35 |
| 4-fold diluted | 25 | 43 | 33 | 37 | 69 |
| 2-fold diluted | 54 | 65 | 56 | 67 | 86 |
| undiluted | 92 | 107 | 101 | 107 | 94 |

As shown in Table 11, the addition of TRI083 to either an endopeptidase, or endopeptidase which has been supplemented with several exopeptidases (Flavourzyme 500 (FZ500; Sigma-Aldrich, Denmark)) resulted for both in increased serine equivalents values, suggesting that the degree of hydrolysis of a hydrolysate produced with a complex mixture of exopeptidases, such as FZ500, can be increased by applying a tripeptidyl peptidase (e.g. a proline tolerant tripeptidyl peptidase).

### Example 13

Whey Protein Hydrolysis (WPI) Employing an Endopeptidase in Combination with Proline Tolerant Tripeptidyl Peptidase Homologues.

For WPI hydrolysis, Lacprodan-9224 (Arla Food Ingredients, Denmark) was employed, a 15% (w/w) WPI suspension was prepared in H₂O_{d} and adjusted to pH 4.5 with acetic acid (Sigma-Aldrich, Denmark). To prevent microbial growth, 0.0285% (w/w) NaN₃ was added. Subsequently, 1% (w/w) Alphalase® FP2 was added and a volume of 200 μl of the WPI suspension was transferred into each of the 96 wells of a microtiter plate (MTP; VWR, Denmark). Following this, 5 μl of each proline tolerant tripeptidyl peptidase homologue containing either 0 or 4375 _{nkatH-Ala-Ala-Ala-pNA}/mL were added to the particular wells of the MTP. Then, the MTP was sealed and placed in an incubator at 40° C. (iEMS incubator/shaker HT, Thermo Scientific, Denmark).

After 24 h of incubation and shaking at 400 rpm, the hydrolyses were stopped by addition of 20 μl of 2 M trichloroacetic acid (TCA; Sigma-Aldrich, Denmark), except for the reference (0 h), to which the TCA was added prior to endo- and exopeptidase addition. Unhydrolyzed, precipitated WPI was removed by filtration (0.22 μm; Corning 3504 filter plate, Corning Incorporated, USA). The filtered WPI hydrolysate was employed for o-phthaldehyde (OPA) derivatization (Nielsen, Petersen et al. 2001). The OPA derivatization was conducted according to Nielsen et al., (2001) with minor modifications. A sample volume of 25 μl was transferred to a well and 175 μl of OPA-reagent, dissolved in trisodium phosphate-dodecahydrate, was added subsequently. The measured absorptions at 340 nm in a MTP reader (VersaMax, Molecular Devices, Denmark) were transformed into serine equivalents employing a serine calibration curve (0-2 mM).

Proline tolerant tripeptidyl peptidase TRI071 (SEQ ID No. 12 or SEQ ID No. 39) increased the measured serine equivalents significantly over time compared to the reference (endopeptidase). The relative increase of SE was about 64% for TRI071 after 7 h of hydrolysis, respectively (FIG. 14).

### Example 14

Product Inhibition of a Proline Tolerant Tripeptidyl Peptidase (TRI083) with a Native Tripeptide (H-Ala-Ala-Ala-OH)

Inhibition experiments were conducted with TRI083 and Ala-Ala-Ala-pNA (0-2000 nM) as substrate in the assay described in Example 8. The assay was run without inhibitor, with 3.6 mM tripeptide (H-Ala-Ala-Ala-OH; Bachem, Switzerland), 18 mM dipeptide (H-Ala-Ala-OH; Bachem, Switzerland) or 18 mM alanine (Sigma-Aldrich, Denmark). Curve fitting and inhibition analysis was done by treating H-Ala-Ala-Ala-OH, H-Ala-Ala-OH and alanine as competitive inhibitors using GraphPad Prism version 6.02 for Windows, GraphPad Software, La Jolla Calif. USA, www.graphpad.com.

For the tripeptide H-Ala-Ala-Ala-OH strong inhibition with a Ki of 0.34 mM was found, whereas the dipeptide H-Ala-Ala-OH only showed very weak inhibition with a Ki of 40 mM. Alanine showed no inhibition at all.

### Example 15

Decreased Product Inhibition of a Proline Tolerant Tripeptidyl Peptidase (TRI083) with a Native Tripeptide (H-Ala-Ala-Ala-OH) Pre-Hydrolysed with a General Aminopeptidase (PepN)

The inhibition experiments were set up as described in Example 14. Furthermore, 1 mg/mL H-Ala-Ala-Ala-OH (Bachem, Switzerland) was subjected to general aminopeptidase (PepN) hydrolysis for 24 h at 40° C. in 12.5 times diluted Mcllvaine buffer (pH 4.0). For this, 1.2 mg/mL H-Ala-Ala-Ala-OH was dissolved in 8 mL 10 times diluted Mcllvaine buffer (pH 4.0) and filled up to 10 mL with purified PepN (1,572 nkat/mL [pH 6.5]). After 24 h of hydrolysis, the PepN hydrolysis was terminated by heat inactivation of the PepN (90° C.; 10 min). The degree of pre-hydrolysis of H-Ala-Ala-Ala-OH was determined by gas chromatography (Husek, 1991; FEBS-Letters 280, 354-356). About 0.18 mg/mL alanine was measured, which corresponds to about 54% hydrolyses of the initially applied H-Ala-Ala-Ala-OH.

Subsequently, TRI083 was analyzed for product inhibition without inhibitor, with 1 mg/mL H-Ala-Ala-Ala-OH as well with the PepN pre-hydrolyzed H-Ala-Ala-Ala-OH (1 mg/mL).

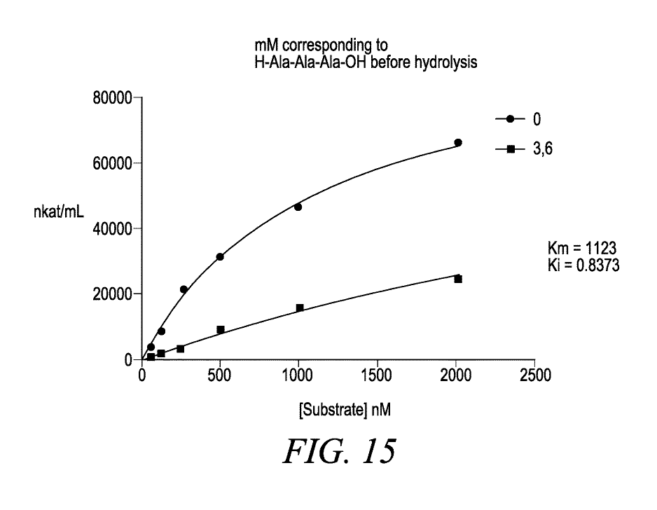

As shown in FIG. 15 the pre-hydrolyzed H-Ala-Ala-Ala-OH was found to have Ki of 0.8 mM. This is in good agreement with the previously determined H-Ala-Ala-Ala-OH Ki of 0.34 mM, since slightly more than half of the H-Ala-Ala-Ala-OH had been pre-hydrolysed to H-Ala-Ala-OH and alanine. This indicates that a general aminopeptidase can reduce the product inhibition of a proline tolerant tripeptidyl peptidase by cleaving tripeptides into dipeptides and free amino acids.

### Example 16

Proline Tolerant Tripeptidyl Peptidase in Reduction of Immunogenicity of β-Lactoglobulin Peptides in Cow Milk Allergy (CMA)

Description of Hydrolysis Procedure

β-lactoglobulin (1 mg/ml), in 10 mM Tris-HCl, pH 8.0, was hydrolysed at 25° C. for 24 h with trypsin (10 ug/ml). After 24 h trypsin was inactivated by warming up to 80° C. for 15 minutes.

The β-lactoglobulin used has the amino acid sequence:

>sp|P02754|LACB_BOVIN Beta-lactoglobulin
OS = Bos taurus GN = LGB PE = 1 SV = 3
MKCLLLALALTCGAQALIVTQTMKGLDIQKVAGTWYSLAMAASDISLLDA
QSAPLRVYVEELKPTPEGDLEILLQKWENGECAQKKIIAEKTKIPAVFKI
DALNENKVLVLDTDYKKYLLFCMENSAEPEQSLACQCLVRTPEVDDEALE
KFDKALKALPMHIRLSFNPTQLEEQCHI

The tryptic β-lactoglobulin hydrolysate (1 mg/ml) in 10 mM TRIS HCL pH 4.0 was diluted five-fold in 20 mM MES buffer pH 4.0. The hydrolysis was initiated by adding 100 ul of proline tolerant tripeptidyl peptidase TRI083 solution (80 ug/ml) in 20 mM MES buffer, pH 4.0, to 1 ml of the 5-fold diluted β-lactoglobulin solution (200 ug/ml) and incubated at RT.

The proteolysis by proline tolerant tripeptidyl peptidase was terminated after 1, 5, 15, 30, 60, 120, and 180 min as well as 24 h by addition of 5% TFA (50 ul) to 100 ul of the β-lactoglobulin solution. The proline tolerant tripeptidyl peptidase was heat inactivated at 80° C. for 10 minutes. The solution was stored at −20° C. until further LC-MS/MS analysis.

Nano LC-MS/MS analyses were performed using an Easy LC system (Thermo Scientific, Odense, DK) interfaced to a LTQ Orbitrap Classic hybrid mass spectrometer (Thermo Scientific, Bremen, Germany). Samples were loaded onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle. Separation was performed at a flow rate of 300 nL/min using a 10 min gradient of 0-34% Solvent B (H2O/CH3CN/TFE/HCOOH (100/800//100/1 v/v/v/v)) into the nanoelectrospray ion source (Thermo Scientific, Odense, DK). The LTQ Orbitrap Classic instrument was operated in a data-dependent MS/MS mode. The peptide masses were measured by the Orbitrap (MS scans were obtained with a resolution of 60 000 at m/z 400), and up to 2 of the most intense peptide m/z were selected and subjected to fragmentation using CID in the linear ion trap (LTQ). Dynamic exclusion was enabled with a list size of 500 masses, duration of 40 s, and an exclusion mass width of ±10 ppm relative to masses on the list.

The RAW files were accessed with the open source program Skyline 1.4.0.4421 which can use the MS1 intensities to build chromatograms. The precursor isotopic import filter was set to a count of three, (M, M+1, and M+2) at a resolution of 60,000 and the most intense charge state was used. Peptide sequences of the substrate as well as a cleavage product was typed into Skyline and intensities were calculated in each sample.

Results

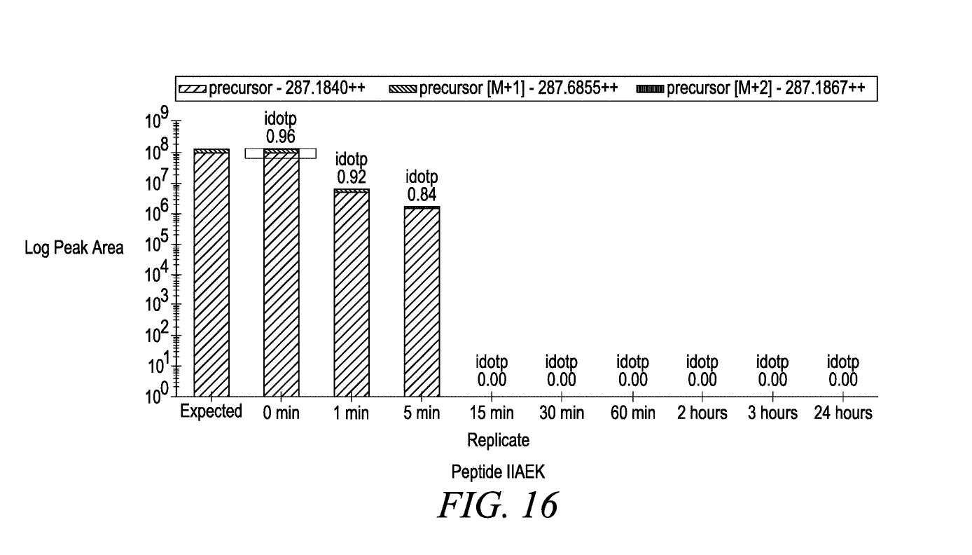

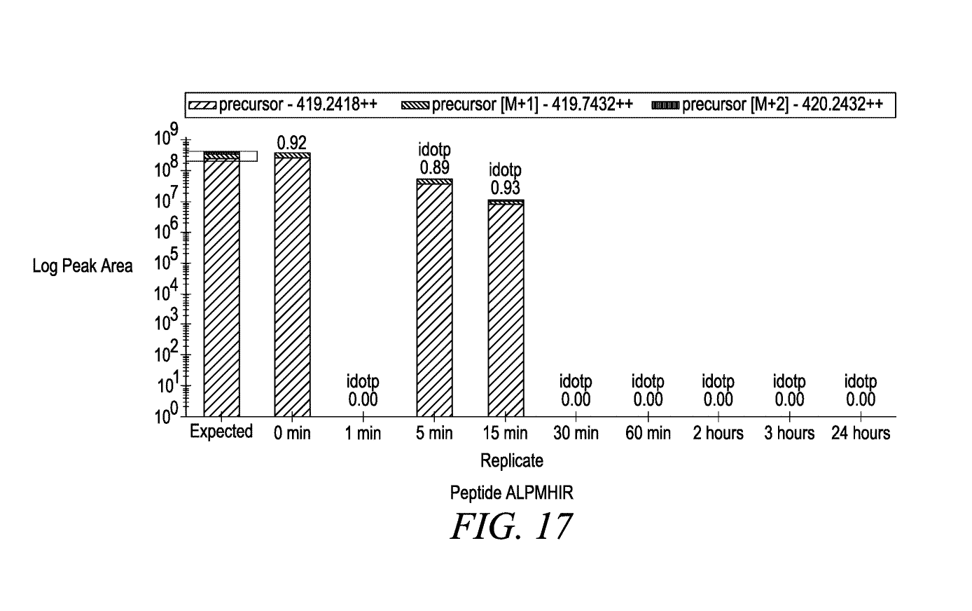

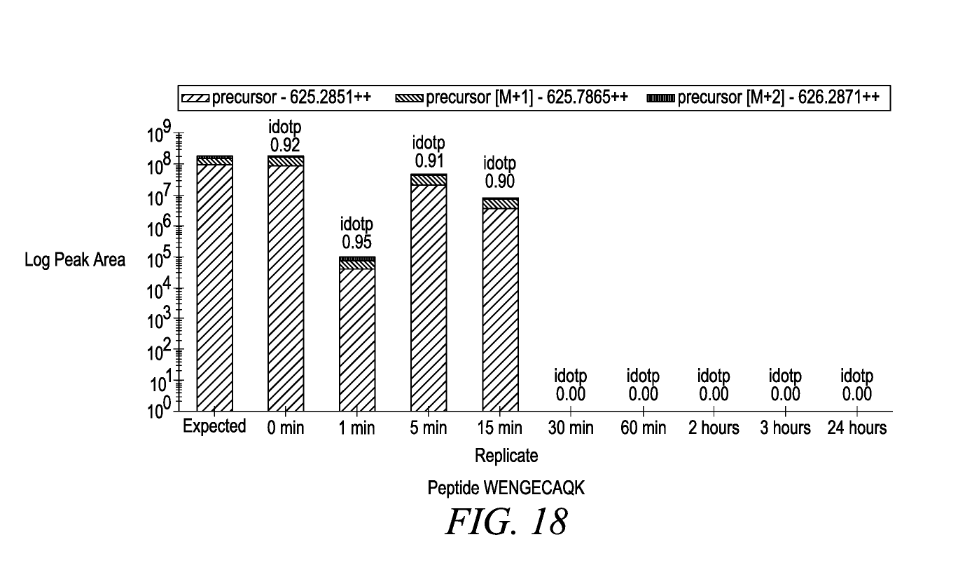

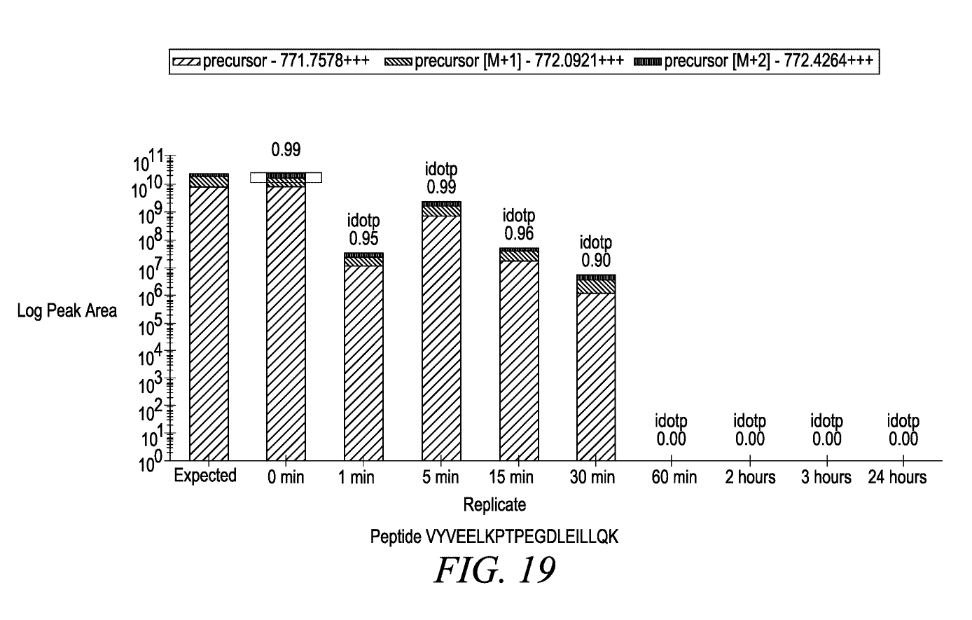

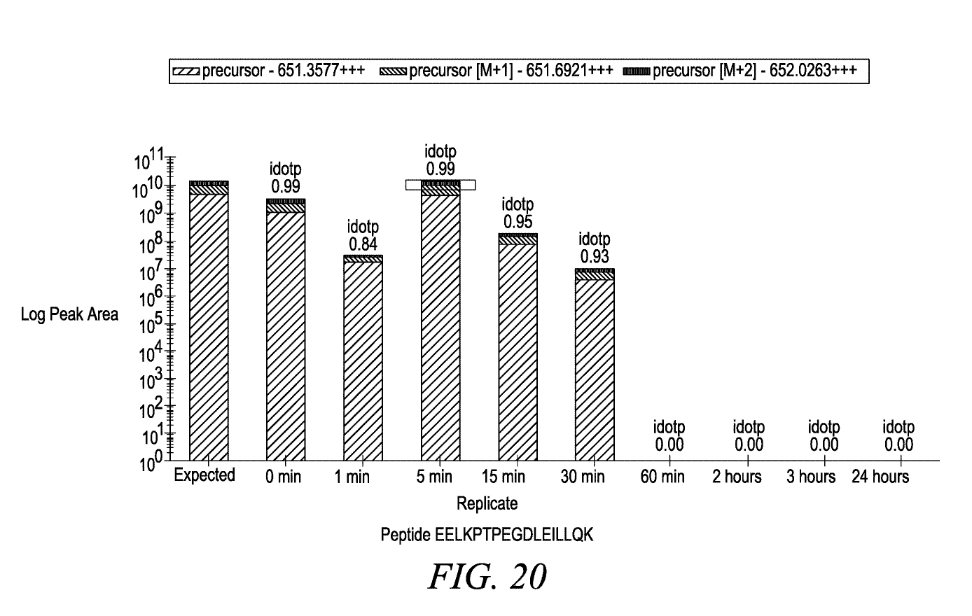

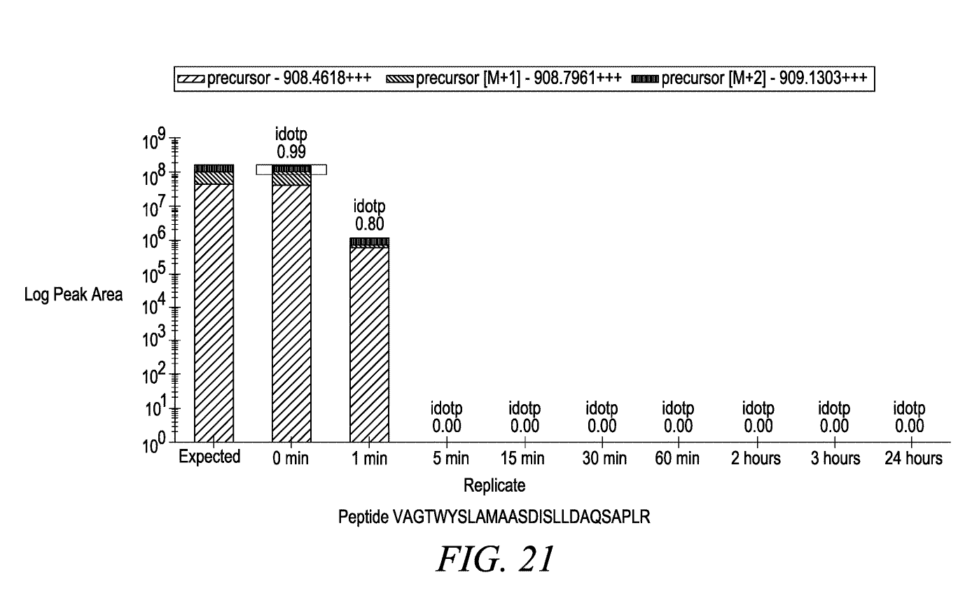

Trypsin treated β-lactoglobulin was efficiently hydrolysed by the proline tolerant tripeptidyl peptidase as shown in FIGS. 16-21. Notably, the proline tolerant tripeptidyl peptidase had a very broad substrate specificity and was able to cleave peptides having proline at P1 (FIG. 17) and glycine at P1 (FIG. 21).

### Example 17

Hydrolysis of Beta-Casein by a Proline Tolerant Tripeptidyl Peptidase

β-casein was first hydrolysed with trypsin before being subjected to cleavage with a proline tolerant tripeptidyl peptidase. β-casein (1 mg/ml) in 10 mM Tris-HCl pH=8.0, was hydrolysed at RT for 24 h with trypsin (10 ug/ml) and then inactivated by warming up to 80° C. for 15 minutes.

The tryptic β-casein hydrolysate (1 mg/ml) in 10 mM TRIS HCL pH 4.0 was diluted five-fold in 20 mM MES buffer pH 4.0. The hydrolysis was initiated by adding 100 ul of proline tolerant tripeptidyl peptidase TRI083 solution (80 ug/ml) in 20 mM MES buffer, pH 4.0, to 1 ml of the 5-fold diluted β-casein solution (200 ug/ml) and incubated at RT.

The proteolysis by proline tolerant tripeptidyl peptidase was terminated after 1, 5, 15, 30, 60, 120, and 180 min as well as 24 h by addition of 5% TFA (50 ul) to 100 ul of the β-casein solution. The proline tolerant tripeptidyl peptidase was heat inactivated at 80° C. for 10 minutes. The solution was stored at −20° C. until further LC-MS/MS analysis performed as described in Example 16.

The beta-casein used had the following amino acid sequence:

>sp|P02666|CASB_BOVIN Beta-casein OS = Bos taurus
GN = CSN2 PE = 1 SV = 2
MKVLILACLVALALARELEELNVPGEIVESLSSSEESITRINKKIEKFQS
EEQQQTEDELQDKIHPFAQTQSLVYPFPGPIPNSLPQNIPPLTQTPVVVP
PFLQPEVMGVSKVKEAMAPKHKEMPFPKYPVEPFTESQSLTLTDVENLHL
PLPLLQSWMHQPHQPLPPTVMFPPQSVLSLSQSKVLPVPQKAVPYPQRDM
PIQAFLLYQEPVLGPVRGPFPIIV

Results

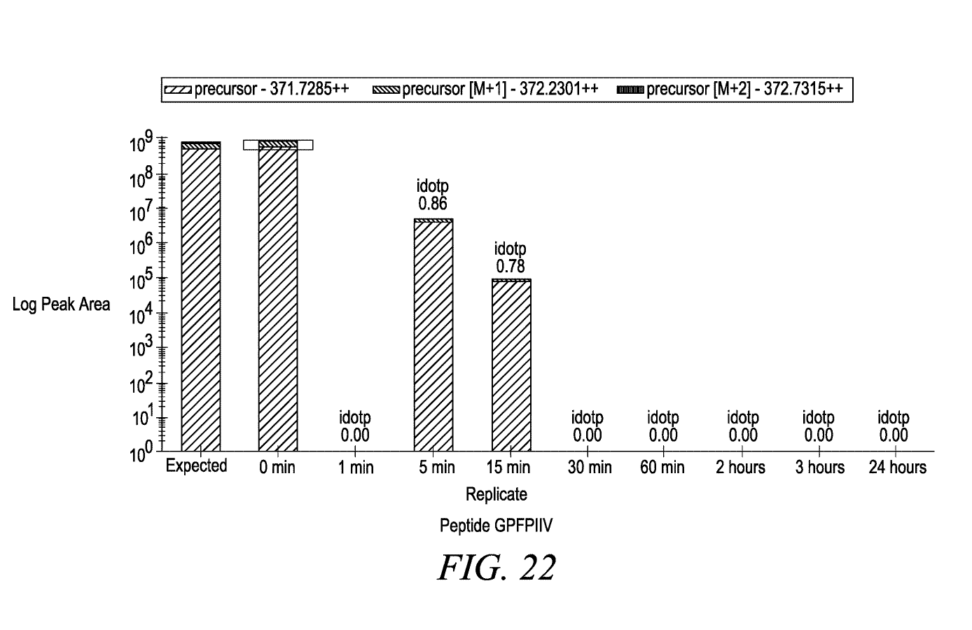

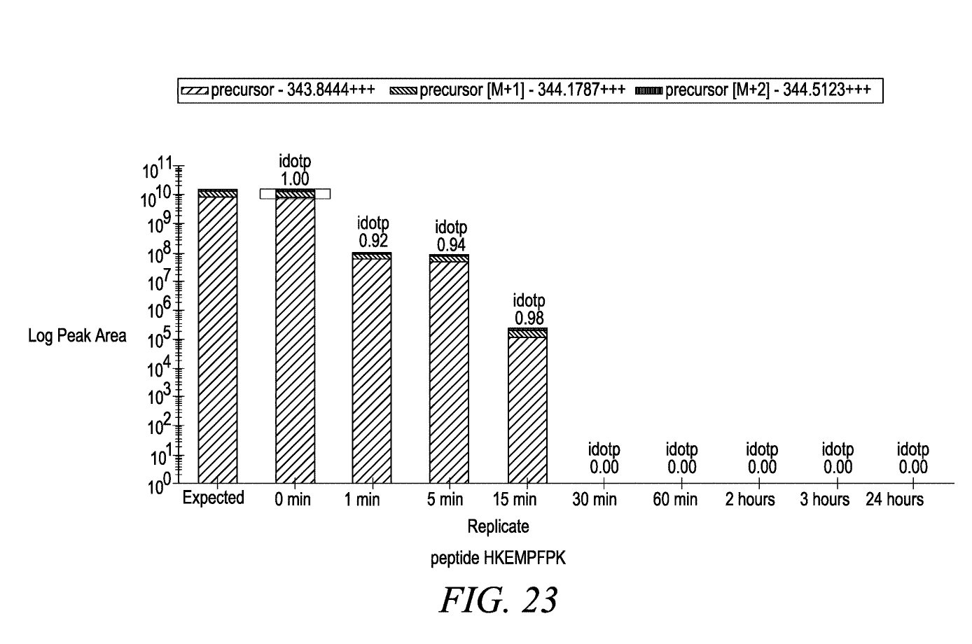

The peptide GPFPIIV resulting from a tryptic digest of β-casein is very hydrophobic and has a very bitter taste. FIG. 22 shows that the proline tolerant tripeptidyl peptidase is able to efficiently digest this peptide with proline in the P1′ position and in the P2 position. FIG. 23 shows that the peptide HKEMPFPK is also efficiently digested by the proline tolerant tripeptidyl peptidase.

### Example 18

Degradation of a Bradykinin Peptide by a Proline Tolerant Tripeptidyl Peptidase

Bradykinin (RPPGFSPFR; MW 1660,21; B2259 from Sigma Aldrich) was dissolved at 1 mg/ml in 20 mM Mcllvaine buffer (pH 4.5). The proline tolerant tripeptidyl peptidase. TRI083 (10 ug) was added to the peptide solution. The hydrolysis was terminated after 1, 3, 5, 15, 30, and 60 min as well as 20 h by addition of 5% TFA (50 ul) to 100 ul of the solution. The proline tolerant tripeptidyl peptidase was heat inactivated at 80° C. for 10 minutes. The solution was stored at −20° C. until further LC-MS/MS analysis performed as described in Example 16.

Results

The proline tolerant tripeptidyl peptidase efficiently digests the bradykinin peptide having the amino acid sequence RPPGFSPFR (see FIG. 24). This shows that the proline tolerant tripeptidyl peptidase is capable of degrading substrates having proline in the P1 and P2 position, a characteristic which is highly surprising for tripeptidyl peptidases.

### Example 19

Hydrolysis of AAXF-NH₂ Substrate with Proline Tolerant Tripeptidyl Peptidase TRI079

Description of Hydrolysis Procedure and LC-MS Analysis

An AAXF-NH2 substrate library (Synthesized by Schaefer, Copenhagen) with X representing all amino acids except Cys and Met, was solubilized in 20 mM Mcllvaine buffer, pH 4.5 (1 mg in 1 ml) The peptide solution was hydrolysed at 25° C. for 1 or 2 h with TRI079 (SEQ ID NO: 57), 10 ul of 1 mg/ml produced as described in EXAMPLE 1. Subsequently, the hydrolysis was stopped by addition of 5% TFA (50 ul) to 50 ul of the peptide solution. The solution was stored at −20° C. until further LC-MS/MS analysis.

Nano LC-MS/MS analyses were performed using an Easy LC system (Thermo Scientific, Odense, DK) interfaced to a LTQ Orbitrap Classic hybrid mass spectrometer (Thermo Scientific, Bremen, Germany). Samples were loaded onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle. Separation was performed at a flow rate of 300 nL/min using a 10 min gradient of 0-34% Solvent B (H2O/CH3CN/TFE/HCOOH (100/800//100/1 v/v/v/v)) into the nanoelectrospray ion source (Thermo Scientific, Odense, DK). The LTQ Orbitrap Classic instrument was operated in a data-dependent MS/MS mode. The peptide masses were measured by the Orbitrap (MS scans were obtained with a resolution of 60 000 at m/z 400), and up to 2 of the most intense peptide m/z were selected and subjected to fragmentation using CID in the linear ion trap (LTQ). Dynamic exclusion was enabled with a list size of 500 masses, duration of 40 s, and an exclusion mass width of ±10 ppm relative to masses on the list.

The RAW files were accessed with the open source program Skyline 1.4.0.4421 which can use the MS1 intensities to build chromatograms. The precursor isotopic import filter was set to a count of three, (M, M+1, and M+2) at a resolution of 60,000 and the most intense charge state was used. Peptide sequences of the substrate as well as a cleavage product was typed into Skyline and intensities were calculated in each sample.

Results

The LC-MS analysis showed that TRI079 could cleave all the AAXF-NH₂ peptides into tripeptides irrespective of which amino acid was in P1. With proline present in P1 the cleavage product AAP was observed to increase over time showing that TRI079 is tolerant to proline in the P1 position.

### Example 20

Whey Protein Hydrolysis (WPI) Employing Proline Tolerant Tripeptidyl Peptidase TRI079 in Combination with Endopeptidases and General Aminopeptidase PepN

For WPI hydrolysis, Lacprodan-9224 (Arla Food Ingredients, Denmark) was employed, a 15% (w/w) WPI suspension was prepared in H₂O_{d} and adjusted to pH 6 with sodium hydroxide. To prevent microbial growth, 0.0285% (w/w) NaN₃ was added. Subsequently, 0.5% (w/w on protein substrate) Food Pro® Alkaline Protease and 0.5% (w/w on protein substrate) Food Pro® PNL was added and a volume of 200 μl of the WPI suspension was transferred into each of the 96 wells of a microtiter plate (MTP; VWR, Denmark). Following this, 5 μl of the proline tolerant tripeptidyl peptidase TRI079 containing either 0 or 2188 nkat/mL and 5 ul of PepN (described in EXAMPLE 10) containing either 0, 197 or 393 nkat/mL were added to the particular wells of the MTP. Then, the MTP was sealed and placed in an incubator at 50° C. (iEMS incubator/shaker HT, Thermo Scientific, Denmark).

After 24 h of incubation and shaking at 400 rpm, the hydrolysis was stopped by addition of 20 μl of 2 M trichloroacetic acid (TCA; Sigma-Aldrich, Denmark), except for the reference (0 h) to which the TCA was added prior to endo- and exopeptidase addition. Unhydrolyzed, precipitated WPI was removed by filtration (0.22 μm; Corning 3504 filter plate, Corning Incorporated, USA). The filtered WPI hydrolysate was employed for o-phthaldehyde (OPA) derivatization (Nielsen, Petersen et al. 2001). The OPA derivatization was conducted according to Nielsen et al. (2001) with minor modifications. A sample volume of 25 μl was transferred to a well and 175 μl of OPA-reagent, dissolved in trisodium phosphate-dodecahydrate, was added subsequently. The measured absorptions at 340 nm in a MTP reader (VersaMax, Molecular Devices, Denmark) were transformed into serine equivalents employing a serine calibration curve (0-2 mM).

As shown in Table 12 the Tripeptidyl amino-peptidase TRI079 (SEQ ID No. 57) as well as PepN strongly increased the measured degree of hydrolysis compared to the reference with only endopeptidases. An additional increase in the degree of hydrolysis was obtained, when TRI079 and PepN were combined.

TABLE 12
Analysis of DH (in %) of WPI hydrolysate prepared
24 h at 50° C., pH 6.0 with a combination of TRI079 and PepN

TRI079 [nkat/mL]

| PepN [nkat/mL] | 0 | 2188 |
|---|---|---|
| 0 | 4.2 | 11.0 |
| 197 | 18.2 | 21.7 |
| 393 | 18.8 | 25.1 |

### Example 21

Hydrolysis of AAFPA-NH₂ Substrate with Proline Tolerant Tripeptidyl Peptidase TRI079

Description of Hydrolysis Procedure and LC-MS Analysis

AAFPA-NH2 substrate (synthesized by Schaefer, Copenhagen) was solubilized in 20 mM Mcllvaine buffer, pH 4.5 (1 mg in 1 ml) and hydrolysed at 25° C. for up to 8 h with TRI079 (10 ul of 1 mg/ml TRI079 solution) produced as described in EXAMPLE 1. Subsequently, the hydrolysis was stopped by addition of 5% TFA (50 ul) to 50 ul of the peptide solution. The solution was stored at −20° C. until further LC-MS/MS analysis.

Nano LC-MS/MS analyses were performed using an Easy LC system (Thermo Scientific, Odense, DK) interfaced to a LTQ Orbitrap Classic hybrid mass spectrometer (Thermo Scientific, Bremen, Germany). Samples were loaded onto a custom-made 2 cm trap column (100 μm i.d., 375 μm o.d., packed with Reprosil C18, 5 μm reversed phase particles (Dr. Maisch GmbH, Ammerbuch-Entringen, Germany)) connected to a 10 cm analytical column (75 μm i.d., 375 μm o.d., packed with Reprosil C18, 3 μm reversed phase particles (Dr. Maisch GmbH, Ammerbuch-Entringen, Germany)) with a steel needle. Separation was performed at a flow rate of 300 nL/min using a 10 min gradient of 0-34% Solvent B (H2O/CH3CN/TFE/HCOOH (100/800//100/1 v/v/v/v)) into the nanoelectrospray ion source (Thermo Scientific, Odense, DK). The LTQ Orbitrap Classic instrument was operated in a data-dependent MS/MS mode. The peptide masses were measured by the Orbitrap (MS scans were obtained with a resolution of 60 000 at m/z 400), and up to 2 of the most intense peptide m/z were selected and subjected to fragmentation using CID in the linear ion trap (LTQ). Dynamic exclusion was enabled with a list size of 500 masses, duration of 40 s, and an exclusion mass width of ±10 ppm relative to masses on the list.

The RAW files were accessed with the open source program Skyline 1.4.0.4421 which can use the MS1 intensities to build chromatograms. The precursor isotopic import filter was set to a count of three, (M, M+1, and M+2) at a resolution of 60,000 and the most intense charge state was used. Peptide sequences of the substrate as well as a cleavage product was typed into Skyline and intensities were calculated in each sample.

Results

The LC-MS analysis showed a reduction of AAFPA-NH₂ and accumulation of AAF over time indicating that TRI079 could hydrolyze AAFPA-NH₂ and is tolerant to proline in the P1′ position.

### Example 22

Stability of TRI045 (SEQ ID No. 99) Tripeptidyl Peptidase in the Presence of Pepsin

Material and Methods

Pepsin solution: Swine pepsin from Sigma (P7000, 674 Sigma units/mg, www.sigma.com) was used in this example and prepared in MilliQ water at 10000 Sigma unit/ml at 10000 Sigma unit/mL

Pre-incubation mixture contained: 2.5 μl TRI045 sample, 95 μl GAT buffer (50 mM glycine-50 mM acetic acid-50 mM Tris, pH3.5), 5 μl pepsin in milliQ water (10000 Sigma unit/ml) (final pepsin unit concentration: 500 Sigma unit/ml reaction mixture) in a half bottom area 96 well Corning MTP. For control, the pre-incubation mixture contained 5 μl water instead of 5 μl pepsin. The mixture was incubated at 40° C. for 60 min and then kept on ice. For assaying the residual activity, the assay mixture contained the pre-incubation mixture 5 μl, 0.1M acetic acid-sodium acetate (pH4.0) 85 μl, 5 μl H-Ala-Ala-Phe-pNA (2.5 mg/ml) from BACHEM.com (L-1095.0250). The 410 nm reading was followed in a microplate reader every 0.5 min at 30° C. N=2.

Results

Under the assay conditions and pre-incubation at 40° C. for 60 min, the residual activity was found to be over 90% for TRI045 in the presence of 500 units pepsin/mL pre-incubation mixture.

### Example 23

TRI045 (SEQ ID No. 99) as Feed Enzyme to Promote Feed Protein Hydrolysis

Animals produce and secrete proteases into their digestive tracts for feed digestion. These proteases include endoproteases of pepsin, trypsin and chymotrypsin and the exopeptidases of Carboxypeptidase A and B etc. However, animals do not produce aminopeptidases as digestive enzymes or at least not in appreciable amount. As shown in this example, the addition of the tripeptidyl peptidase TRI045 (sedolisin) promotes protein digestion in an in vitro system for corn soy based feed. This example shows that TRI045 had high activity between pH 4 and 6.5

Material and Methods

The tripeptide substrate H-Ala-Ala-Phe-pNA (AAF-pNA from BACHEM.com, L-1095.0250) for sedolisin was prepared at 2.5 mg/ml in DMSO. The reaction was prepared by mixing 8.5 μl AAF-pNA, 7 μl DMSO, 32 μl water and 50 μl buffer with pH4.14-6.57, and 0.8 μl or 1.5 ul TRI045. The reaction was started at 30° C. by the addition of the enzyme TRI045. The initial reaction rate was recorded using a microplate reader at every 0.5 min interval at 410 nm in 96 well plate.

Results

Table 13 shows that TRI045 had optimal activity around pH5.0, but still had around 50% activity at around pH4 and pH6.5, which is ideal for gastric feed digestion. FIG. 22 furthers shows that the final reaction degree reached at pH5, pH5.5 and pH6 are very similar. The high pH optimum is ideal for animal feed as in such situation pancreatin would have time to make more oligopeptide substrates for TRI045.

TABLE 13
Effect of pH on the TRI045 activity using H-Ala-Ala-Phe-pNA
as substrate (values are the average one test with 0.8 μl TRI045
(n = 2) and one test with 1.5 μl TRI045 dose (n = 1).

| | 0.1M | 0.1M | 0.1M | 0.1M | 0.1M | 0.1M |
|---|---|---|---|---|---|---|
| | HAC- | HAC- | HAC- | HAC- | Mes- | Mes- |
| Buffer | NaAC | NaAC | NaAC | NaAC | NaOH | NaOH |
| pH | 4.1 | 4.6 | 5.0 | 5.6 | 6.0 | 6.6 |
| Relative activ- | 59.4 | 81.7 | 100.0 | 80.7 | 72.2 | 48.5 |
| ity with pH | | | | | | |
| 5.01 as 100% | | | | | | |

### Example 24

Effect of TRI045 (SEQ ID No. 99) Plus Pepsin and Pancreatin on Hydrolysis of Corn Soy Feed Substrate.

The in vitro reaction system contained 140 μl 10% (w/v) corn soy feed slurry (14 mg corn soy feed), 10 μl TRI045 in 50 m M MES-NaOH pH 6.0, 10 μl swine pepsin (1.14 U/μL in water). The reaction was incubated with shaking at 40° C. for 45 min and subsequently 34 μl swine pancreatin (0.4636 mg/mL in 1M Na-bicarbonate) were added and further incubated for additional 60 min. After incubation, the 96-well plate was centrifuged and supernatants were used for residual TRI045 activity assay and for OPA and BCA assays (see below).

The degree of Hydrolysis measurements of soluble protein is based on the reaction of primary amino groups with o-phthaldialdehyde (OPA—assay). Reference: P. M. Nielsen, D. Petersen and C. Dambmann. Improved Method for Determining Food Protein Degree of Hydrolysis. Journal of Food Science. 66 (2001) 642-646.

For OPA assay the following procedure was carried out. 10-25 μl feed sample treated by enzyme from master plate was transferred to the new plate, then 175 μl of OPA reagent containing sodium borate, dodecyl sulfate and dithiothreitol, were added to the plate. The end point measurements of optical density at 340 nm were performed right after 2 min and 5 second mixing.

To quantify the protein concentrations of each protease samples, the Pierce BCA Protein Assay Reagent Kit (Thermo Scientific, cat no. 23228) was used. The TRI045 sample was not purified before quantification. The Pierce BCA Protein Assay Kit is a detergent-compatible formulation based on bicinchoninic acid (BCA) for colorimetric detection and quantification of total proteins. This method combines the well-known reduction of Cu²⁺ to Cu¹⁺ by protein in an alkaline medium with the highly sensitive and selective colorimetric detection of the cuprous cat ion (Cu⁺¹) using a unique reagent containing bicinchoninic acid. The purple-coloured reaction product of this assay is formed by the chelation of two molecules of BCA with one cuprous ion. This water soluble complex exhibits a strong absorbance at 562 nm that is nearly linear with increasing protein concentration over a broad working range (20-2000 μg/mL). The macromolecular structure of protein, the number of peptide bonds and the presence of four particular amino acids Cysteine, Cystine, Tryptophan and Tyrosine are reported to be responsible for colour formation with BCA.

For OPA (total amino group released) and BCA (total protein in the soluble fraction) determinations the supernatants were diluted 20 times and 10 ul was used.

In the system containing TRI045 at 1000 ppm, in the presence of swine pepsin and pancreatin, the release of free amino groups from corn soy feed (as a measure of protein hydrolysis (OPA value)) increased by 9% and the protein solubility increased by 5%. In order to test the stability of TRI045 under the in vitro assay conditions described above (incubation in the presence of pepsin at pH3 for 45 min at 40° C. and subsequently in the presence of pancreatin for 60 min at 40° C.). The residual activity of TRI045 was subsequently assayed. The reaction mixture contained 50 μl buffer 0.1M HAC-NaAC (pH5.0), 10 μl the supernatant, and 5 μl AAF-pNA (5 mg/ml in DMSO). The reaction rate was followed at 410 nm and 30° C. every 30 seconds using a microplate reader. As a control, a commercial protease at the same concentration of 1000 ppm was used.

Conclusion for Examples 22-24

The reaction rate for the control (pepsin and pancreatin only) was 4.3 mOD/min, for the commercial protease it was 11.0 mOD/min, and for TRI045 it was 19.3 mOD/min. After the subtraction of the control (4.3 mOD/min), the residual activity of TRI045 on AAF-pNA substrate was 2.2 times higher than for the commercial protease. These results indicate that TRI045 is stable to pepsin and pancreatin at 40° C. for at least 100 minutes.

In conclusion, this example demonstrates that TRI045 is stable to pepsin and pancreatin when incubated for 105 min at 40° C. in the presence of corn soy feed at a pH range of 3 to 7. It has also the additional effect of increasing protein solubilization and protein hydrolysis in the presence of pepsin and pancreatin when corn soy feed was used as the substrate under conditions mimicking the monogastric digestion system (BEDFORD, M. R., & CLASSEN, H. L. (1993) “An in vitro assay for prediction of broiler intestinal viscosity and growth when fed rye-based diets in the presence of exogenous enzymes”. Poultry Science, 72: 137-143)., i.e., the reaction was carried out at 40° C. at pH3.0-3.3 in the presence of pepsin for 45 min and then pH raised to pH6.5-7.0 and addition of pancreatin for additional 60 min incubation,

### LITERATURE

Nakadai, T., et al. (1973). “Purification and properties of leucine amino-peptidase I from Aspergillus oryzae.” Agricultural and Biological Chemistry 37(4): 757-765.
Nielsen, P. M., et al. (2001). “Improved method for determining food protein degree of hydrolysis.” Journal of Food Science 66(5): 642-646.
Stressler, T., et al. (2013). “Characterization of the Recombinant Exopeptidases PepX and PepN from Lactobacillus helveticus ATCC 12046 Important for Food Protein Hydrolysis.” PLoS ONE 8(7) e70055.
Wang, F., et al. (2012). “Biochemical and conformational characterization of a leucine amino-peptidase from Geobacillus thermodenitrificans NG80-2.” World Journal of Microbiology and Biotechnology 28(11): 3227-3237.

Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A method for the production of a hydrolysate comprising:
(a) admixing at least one protein or a portion thereof with:
(A) at least one endoprotease; and
(B) (a′) at least one proline tolerant tripeptidyl peptidase or fermentate comprising a proline tolerant tripeptidyl peptidase predominantly having exopeptidase activity wherein said proline tolerant tripeptidyl peptidase is capable of cleaving tri-peptides from the N-terminus of peptides having:
Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(b′) at least one proline tolerant tripeptidyl peptidase having exopeptidase activity wherein said proline tolerant tripeptidyl peptidase is capable of cleaving tri-peptides from the N-terminus of peptides having:
Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′; and
(b) recovering the hydrolysate.

2. A method for the production of a hydrolysate comprising:
(a) admixing at least one protein or a portion thereof selected from the group consisting of: a plant protein, a milk-based protein, or an egg protein with:
(i) at least one endopeptidase;
(ii) at least one exo-tripeptidyl peptidase of the S53 family; and
(iii) one or more aminopeptidase, and
(b) recovering the hydrolysate.

3. Use of (i) at least one endopeptidase; (ii) at least one exo-tripeptidyl peptidase of the S53 family; and (iii) one or more aminopeptidase, in the manufacture of a hydrolysate from at least one protein or a portion thereof selected from the group consisting of: a plant protein, a milk based protein, or an egg protein, for reducing the immunogenicity in a subject predisposed to having an immune reaction to an untreated hydrolysate or for reducing bitterness of the hydrolysate.

4. The method or use according to any one of the preceding claims wherein the at least one protein is selected from the group consisting of: a plant protein, preferably wherein the protein is one or more of a gliadin, an immunogenic fragment of a gliadin, a grain protein, gluten, a soy protein.

5. The method or use according to any one of the preceding claims wherein the at least one protein is selected from the group consisting of: a milk-based protein, preferably wherein the protein is one or more of a casein, e.g. beta-casein; a lactoglobulin, e.g. beta-lactoglobulin; or a whey protein.

6. The method or use according to any one of the preceding claims wherein the at least one protein is an egg protein.

7. The method according to any one of claims 2-6, wherein the order of admixing in step (a) is carried out in a specific order namely (i) is carried out before (ii) and/or (iii).

8. The method or use according to any one of claims 2-7 wherein the tripeptidyl peptidase in (ii) is a proline tolerant tripeptidyl peptidase.

9. The method or use according to claim 8 wherein the proline tolerant tripeptidyl peptidase predominantly has exopeptidase activity and
a) is capable of cleaving tri-peptides from the N-terminus of peptides having:
Proline at P1; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(b′) is capable of cleaving tri-peptides from the N-terminus of peptides having: Proline at P1′; and an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

10. The method or use according to any one of claims 2-9 wherein said aminopeptidase is an aminopeptidase obtainable from Lactobacillus, more preferably Lactobacillus helveticus.

11. A method or use according to claim any one of the preceding claims, wherein said at least one tripeptidyl peptidase or at least one exo-tripeptidyl peptidase of the S53 family is capable of cleaving tri-peptides from the N-terminus of peptides having one or more amino acids at P1 selected from the group consisting of: lysine, arginine and glycine.

12. A method or use according to any one of the preceding claims, wherein the at least one proline tolerant tripeptidyl peptidase or at least one exo-tripeptidyl peptidase of the S53 family:
(a) comprises the amino acid sequence SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof;
(b) comprises an amino acid having at least 70% identity to SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof;
(c) is encoded by a nucleotide sequence comprising the sequence SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97;
(d) is encoded by a nucleotide sequence comprising at least about 70% sequence identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97;
(e) is encoded by a nucleotide sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 under medium stringency conditions; or
(f) is encoded by a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 due to degeneracy of the genetic code.

13. A method or use according to any one of the preceding claims, wherein said at least one proline tolerant tripeptidyl peptidase or at least one exo-tripeptidyl peptidase of the S53 family is encoded by a nucleotide sequence comprising SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96, SEQ ID No. 97 or a nucleotide sequence having at least 90% identity thereto or a sequence which hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 under high stringency conditions.

14. A method or use according to any one of the preceding claims, wherein the proline tolerant tripeptidyl peptidase or at least one exo-tripeptidyl peptidase of the S53 family is capable of cleaving tri-peptides from the N-terminus of peptides having:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; and
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′.

15. A method or use according to any one of the preceding claims, wherein the proline tolerant tripeptidyl peptidase or at least one exo-tripeptidyl peptidase of the S53 family is further capable of cleaving tri-peptides from the N-terminus of a peptide having Proline at P1 and Proline at P1′.

16. A method according to any one of the preceding claims, wherein the method further comprises admixing the recovered hydrolysate with at least one feed or food ingredient.

17. A method according to claim 1 or 11-16, wherein step (A) and step (B) are carried out simultaneously.

18. A method according to any one of the preceding claims wherein the at least one endoprotease and at least one proline tolerant tripeptidyl peptidase or at least one exo-tripeptidyl peptidase of the S53 family are active at a similar pH range.

19. A method or use according to any one of the preceding claims, wherein the endoprotease is an acid endoprotease.

20. A method or use according to any one of the preceding claims, wherein the at least one endoprotease is an alkaline endoprotease, preferably selected from one or more of: a trypsin or a chymotrypsin.

21. A method or use according to any one of the preceding claims wherein the hydrolysate has a reduced immunogenicity in a subject predisposed to having an immune response to the at least one protein or portion thereof.

22. A method or use according to any claim 1 or 11-21 wherein the at least one protein is an animal protein or a plant protein, preferably wherein the protein is one or more of a gliadin, a beta-casein, a beta-lactoglobulin or an immunogenic fragment of a gliadin, a beta-casein, a beta-lactoglobulin, whey protein, fish protein, meat protein, egg protein, soy protein, a hordein or grain protein.

23. An isolated nucleic acid comprising:
(a) a nucleotide sequence as shown herein as SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97;
(b) a nucleotide sequence which has at least about 70% identity to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97; or
(c) a sequence that hybridises to SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 under medium stringency conditions; or
(c) a nucleotide sequence which differs from SEQ ID No. 56, SEQ ID No. 57, SEQ ID No. 58, SEQ ID No. 59, SEQ ID No. 60, SEQ ID No. 61, SEQ ID No. 62, SEQ ID No. 63, SEQ ID No. 64, SEQ ID No. 65, SEQ ID No. 66, SEQ ID No. 67, SEQ ID No. 68, SEQ ID No. 69, SEQ ID No. 70, SEQ ID No. 71, SEQ ID No. 72, SEQ ID No. 73, SEQ ID No. 74, SEQ ID No. 75, SEQ ID No. 76, SEQ ID No. 77, SEQ ID No. 78, SEQ ID No. 79, SEQ ID No. 80, SEQ ID No. 81, SEQ ID No. 82, SEQ ID No. 83, SEQ ID No. 84, SEQ ID No. 85, SEQ ID No. 86, SEQ ID No. 87, SEQ ID No. 88, SEQ ID No. 89, SEQ ID No. 90, SEQ ID No. 91, SEQ ID No. 92, SEQ ID No. 93, SEQ ID No. 94, SEQ ID No. 95, SEQ ID No. 96 or SEQ ID No. 97 due to degeneracy of the genetic code.

24. A vector (e.g. a plasmid) comprising a nucleic acid according to claim 23.

25. A host cell comprising a nucleic acid sequence according to claim 23 or a vector according to claim 24.

26. A host cell according to claim 25, wherein the host cell is a Trichoderma cell, preferably a Trichoderma reesei cell.

27. A method for the expression of a proline tolerant tripeptidyl peptidase predominantly having exopeptidase activity wherein said proline tolerant tripeptidyl peptidase is capable of cleaving tri-peptides from the N-terminus of peptides having the consensus sequence cleavage sites:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′:
said method comprising:
(a) transforming a host cell with a nucleic acid or a vector comprising a nucleotide sequence encoding a proline tolerant tripeptidyl peptidase;
(b) expressing the nucleic acid sequence or vector of step (a); and
(c) obtaining the proline tolerant tripeptidyl peptidase or a fermentate comprising said proline tolerant tripeptidyl peptidase and optionally isolating and/or purifying and/or packaging.

28. The method of claim 27, wherein the host cell is a Trichoderma cell, preferably a Trichoderma reesei cell.

29. A method according to claim 27 or claim 28 wherein the host cell is transformed with an isolated nucleic acid according to claim 23 or a vector according to claim 24.

30. Use of at least one endoprotease and at least one proline tolerant tripeptidyl peptidase or fermentate comprising a proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;
or obtainable by any one of methods 19-21; in the manufacture of a hydrolysate for reducing the immunogenicity in a subject predisposed to having an immune reaction to an untreated hydrolysate or for reducing bitterness of the hydrolysate.

31. The method according to any one of claim 1 or 11-29 or a use according to claim 30 which further comprises the addition of one or more further protease(s) selected from the group consisting of: an aminopeptidase and a carboxypeptidase.

32. The method or use according to claim 31 wherein at the least one further protease is an aminopeptidase, preferably an aminopeptidase obtainable from Lactobacillus, more preferably Lactobacillus helveticus.

33. The method according to any one of claim 1-22 or 31-32 or a use according to claim 30-32 wherein the at least one protein or a portion thereof is admixed with the at least one endoprotease before adding the at least one proline tolerant tripeptidyl peptidase (and optionally the at least one further protease).

34. A hydrolysate comprising at least one endoprotease and a proline tolerant tripeptidyl peptidase predominantly having exopeptidase activity wherein said proline tolerant tripeptidyl peptidase is capable of cleaving tri-peptides from the N-terminus of peptides having:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;
or obtainable by any one of methods 27-29.

35. The hydrolysate according to claim 34 wherein the hydrolysate is substantially enriched in one or more tripeptides.

36. The hydrolysate according to claim 34 or claim 35 wherein the hydrolysate is a milk protein hydrolysate, a gliadin hydrolysate or a soy protein hydrolysate.

37. A composition comprising at least one proline tolerant tripeptidyl peptidase predominantly having exopeptidase wherein said proline tolerant tripeptidyl peptidase is capable of cleaving tri-peptides from the N-terminus of peptides having:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;
or obtainable by any one of methods 27-29, and one or more ingredients selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other food grade salts; a preservative, e.g. sodium benzoate and/or potassium sorbate; or combinations thereof.

38. A composition according to claim 37 wherein said composition further comprises at least one endoprotease.

39. A method for producing a feedstuff or foodstuff comprising contacting a feed component or food component with a hydrolysate according to any one of claims 34 to 36 or a composition according to claim 37 or 38.

40. A food additive composition or feed additive composition comprising at least one proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;
or obtainable by any one of methods 27-29 or a hydrolysate according to any one of claims 32 to 34 optionally further comprising one or more ingredients selected from the group consisting of: polyols, such as glycerol and/or sorbitol; sugars, such as glucose, fructose, sucrose, maltose, lactose and trehalose; salts, such as NaCl, KCl, CaCl₂, Na₂SO₄ or other food grade salts; a preservative, e.g. sodium benzoate and/or potassium sorbate; or combinations thereof.

41. A food additive composition or feed additive composition according to claim 40 wherein said composition further comprises at least one endoprotease.

42. A food additive or feed additive composition comprising a hydrolysate according any one of claims 34-36.

43. A foodstuff or feedstuff comprising: at least one proline tolerant tripeptidyl peptidase comprising an amino acid sequence selected from SEQ ID No. 29, SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27, SEQ ID No. 28, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41, SEQ ID No. 42, SEQ ID No. 43, SEQ ID No. 44, SEQ ID No. 45, SEQ ID No. 46, SEQ ID No. 47, SEQ ID No. 48, SEQ ID No. 49, SEQ ID No. 50, SEQ ID No. 51, SEQ ID No. 52, SEQ ID No. 53, SEQ ID No. 54, SEQ ID No. 55, SEQ ID No. 98, SEQ ID No. 99 or a functional fragment thereof or an amino acid sequence having at least 70% identity thereto
or obtainable by any one of methods 27-29; or a hydrolysate according to any one of claims 34 to 36 and optionally at least one food or feed ingredient.

44. A feedstuff or foodstuff according to claim 43, wherein the feed, feedstuff, foodstuff or food is a dairy product, (preferably a milk-based product), a whey-protein product, a bakery product (preferably a bread product), a fermentation product (preferably a soy-based fermentation product), a sports nutrition product, a performance food, a beverage, a baby food, a food for elderly, a food for people in medical care, a shake, or a casing (preferably, a casing for beer or dairy).

45. A kit comprising at least one proline tolerant tripeptidyl peptidase wherein said proline tolerant tripeptidyl peptidase predominantly has exopeptidase activity and is capable of cleaving tri-peptides from the N-terminus of peptides having:
(i) Proline at P1; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1; or
(ii) Proline at P1′; and
an amino acid selected from alanine, arginine, asparagine, aspartic acid, cysteine, glutamine, glutamic acid, glycine, histidine, isoleucine, leucine, lysine, methionine, phenylalanine, serine, threonine, tryptophan, tyrosine, valine or synthetic amino acids at P1′;
or obtainable by any one of methods 27-29; at least one endoprotease; and instructions for co-administering same.

46. A kit comprising:
(i) at least one endopeptidase;
(ii) at least one exo-tripeptidyl peptidase of the S53 family; and
(iii) one or more aminopeptidase, and instructions for co-administering same.

47. A nonfood product comprising the hydrolysate of according to any one of claims 34-36, wherein the nonfood product is a cosmetic, a lotion, or a cleanser for use on human skin.

48. A hydrolysate enriched in:
(a) a tripeptide having a Proline at its N-terminus; or
(b) a tripeptide having a Proline at its N-terminus and at its C-terminus.

49. A method, use, feed additive composition, feed, feedstuff, foodstuff or food, hydrolysate, composition, kit, nucleic acid, vector, host cell or non-food product as described herein with reference to the description, examples and figures.


SEQUENCE LISTING (123 sequences)

SEQ ID NO: 1 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 2 - protein, 590 aa, Trichoderma reesei
SVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 3 - protein, 578 aa, Aspergillus oryzae
EAFEKLSAVPKGWHYSSTPKGNTEVCLKIALAQKDAAGFEKTVLEMSDPDHPSYGQHFTTHDEMKRMLLPRDDTVDAVRQWLENGGVTDFTQDADWINFCTTVDTANKLLNAQFKWYVSDVKHIRRLRTLQYDVPESVTPHINTIQPTTRFGKISPKKAVTHSKPSQLDVTALAAAVVAKNISHCDSIITPTCLKELYNIGDYQADANSGSKIAFASYLEEYARYADLENFENYLAPWAKGQNFSVTTFNGGLNDQNSSSDSGEANLDLQYILGVSAPLPVTEFSTGGRGPLVPDLTQPDPNSNSNEPYLEFFQNVLKLDQKDLPQVISTSYGENEQEIPEKYARTVCNLIAQLGSRGVSVLFSSGDSGVGEGCMTNDGTNRTHFPPQFPAACPWVTSVGATFKTTPERGTYFSSGGFSDYWPRPEWQDEAVSSYLETIGDTFKGLYNSSGRAFPDVAAQGMNFAVYDKGTLGEFDGTSASAPAFSAVIALLNDARLRAGKPTLGFLNPWLYKTGRQGLQDITLGASIGCTGRARFGGAPDGGPVVPYASWNATQGWDPVTGLGTPDFAELKKLALGN

SEQ ID NO: 4 - protein, 574 aa, Phaeosphaeria nodorum
EPFEKLFSTPEGWKMQGLATNEQIVKLQIALQQGDVAGFEQHVIDISTPSHPSYGAHYGSHEEMKRMIQPSSETVASVSAWLKAAGINDAEIDSDWVTFKTTVGVANKMLDTKFAWYVSEEAKPRKVLRTLEYSVPDDVAEHINLIQPTTRFAAIRQNHEVAHEIVGLQFAALANNTVNCDATITPQCLKTLYKIDYKADPKSGSKVAFASYLEQYARYNDLALFEKAFLPEAVGQNFSVVQFSGGLNDQNTTQDSGEANLDLQYIVGVSAPLPVTEFSTGGRGPWVADLDQPDEADSANEPYLEFLQGVLKLPQSELPQVISTSYGENEQSVPKSYALSVCNLFAQLGSRGVSVIFSSGDSGPGSACQSNDGKNTTKFQPQYPAACPFVTSVGSTRYLNETATGFSSGGFSDYWKRPSYQDDAVKAYFHHLGEKFKPYFNRHGRGFPDVATQGYGFRVYDQGKLKGLQGTSASAPAFAGVIGLLNDARLKAKKPTLGFLNPLLYSNSDALNDIVLGGSKGCDGHARFNGPPNGSPVIPYAGWNATAGWDPVTGLGTPNFPKLLKAAVPSRYRA

SEQ ID NO: 5 - protein, 590 aa, Trichoderma atroviride
NAAVLLDSLDKVPVGWQAASAPAPSSKITLQVALTQQNIDQLESKLAAVSTPNSSNYGKYLDVDEINQIFAPSSASTAAVESWLKSYGVDYKVQGSSIWFQTDVSTANKMLSTNFHTYTDSVGAKKVRTLQYSVPETLADHIDLISPTTYFGTSKAMRALKIQNAASAVSPLAARQEPSSCKGTIEFENRTFNVFQPDCLRTEYSVNGYKPSAKSGSRIGFGSFLNQSASSSDLALFEKHFGFASQGFSVELINGGSNPQPPTDANDGEANLDAQNIVSFVQPLPITEFIAGGTAPYFPDPVEPAGTPDENEPYLEYYEYLLSKSNKELPQVITNSYGDEEQTVPQAYAVRVCNLIGLMGLRGISILESSGDEGVGASCLATNSTTTPQFNPIFPATCPYVTSVGGTVSFNPEVAWDGSSGGFSYYFSRPWYQEAAVGTYLNKYVSEETKEYYKSYVDFSGRGFPDVAAHSVSPDYPVFQGGELTPSGGTSAASPIVASVIALLNDARLRAGKPALGFLNPLIYGYAYKGFTDITSGQAVGCNGNNTQTGGPLPGAGVIPGAFWNATKGWDPTTGFGVPNFKKLLELVRY

SEQ ID NO: 6 - protein, 580 aa, Arthroderma benhamiae
KPTPGASHKVIEHLDFVPEGWQMVGAADPAAIIDFWLAIERENPEKLYDTIYDVSTPGRAQYGKHLKREELDDLLRPRAETSESIINWLTNGGVNPQHIRDEGDWVRFSTNVKTAETLMNTRFNVFKDNLNSVSKIRTLEYSVPVAISAHVQMIQPTTLFGRQKPQNSLILNPLTKDLESMSVEEFAASQCRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVLP

SEQ ID NO: 7 - protein, 580 aa, Fusarium graminearum
KSYSHHAEAPKGWKVDDTARVASTGKQQVFSIALTMQNVDQLESKLLDLSSPDSKNYGQWMSQKDVTTAFYPSKEAVSSVTKWLKSKGVKHYNVNGGFIDFALDVKGANALLDSDYQYYTKEGQTKLRTLSYSIPDDVAEHVQFVDPSTNFGGTLAFAPVTHPSRTLTERKNKPTKSTVDASCQTSITPSCLKQMYNIGDYTPKVESGSTIGFSSFLGESAIYSDVFLFEEKFGIPTQNFTTVLINNGTDDQNTAHKNFGEADLDAENIVGIAHPLPFTQYITGGSPPFLPNIDQPTAADNQNEPYVPFFRYLLSQKEVPAVVSTSYGDEEDSVPREYATMTCNLIGLLGLRGISVIFSSGDIGVGAGCLGPDHKTVEFNAIFPATCPYLTSVGGTVDVTPEIAWEGSSGGFSKYFPRPSYQDKAVKTYMKTVSKQTKKYYGPYTNWEGRGFPDVAGHSVSPNYEVIYAGKQSASGGTSAAAPVWAAIVGLLNDARFRAGKPSLGWLNPLVYKYGPKVLTDITGGYAIGCDGNNTQSGKPEPAGSGIVPGARWNATAGWDPVTGYGTPDFGKLKDLVLSF

SEQ ID NO: 8 - protein, 603 aa, Acremonium alcalophilum
AVVIRAAVLPDAVKLMGKAMPDDIISLQFSLKQQNIDQLETRLRAVSDPSSPEYGQYMSESEVNEFFKPRDDSFAEVIDWVAASGFQDIHLTPQAAAINLAATVETADQLLGANFSWFDVDGTRKLRTLEYTIPDRLADHVDLISPTTYFGRARLDGPRETPTRLDKRQRDPVADKAYFHLKWDRGTSNCDLVITPPCLEAAYNYKNYMPDPNSGSRVSFTSFLEQAAQQSDLTKFLSLTGLDRLRPPSSKPASFDTVLINGGETHQGTPPNKTSEANLDVQWLAAVIKARLPITQWITGGRPPFVPNLRLRHEKDNTNEPYLEFFEYLVRLPARDLPQVISNSYAEDEQTVPEAYARRVCNLIGIMGLRGVTVLTASGDSGVGAPCRANDGSDRLEFSPQFPTSCPYITAVGGTEGWDPEVAWEASSGGFSHYFLRPWYQANAVEKYLDEELDPATRAYYDGNGFVQFAGRAYPDLSAHSSSPRYAYIDKLAPGLTGGTSASCPVVAGIVGLLNDARLRRGLPTMGFINPWLYTRGFEALQDVTGGRASGCQGIDLQRGTRVPGAGIIPWASWNATPGWDPATGLGLPDFWAMRGLALGRGT

SEQ ID NO: 9 - protein, 614 aa, Sodiomyces alkalinus
AVVIRAAPLPESVKLVRKAAAEDGINLQLSLKRQNMDQLEKFLRAVSDPFSPKYGQYMSDAEVHEIFRPTEDSFDQVIDWLTKSGFGNLHITPQAAAINVATTVETADQLFGANFSWFDVDGTPKLRTGEYTIPDRLVEHVDLVSPTTYFGRMRPPPRGDGVNDWITENSPEQPAPLNKRDTKTESDQARDHPSWDSRTPDCATIITPPCLETAYNYKGYIPDPKSGSRVSFTSFLEQAAQQADLTKFLSLTRLEGFRTPASKKKTFKTVLINGGESHEGVHKKSKTSEANLDVQWLAAVTQTKLPITQWITGGRPPFVPNLRIPTPEANTNEPYLEFLEYLFRLPDKDLPQVISNSYAEDEQSVPEAYARRVCGLLGIMGLRGVTVLTASGDSGVGAPCRANDGSGREEFSPQFPSSCPYITTVGGTQAWDPEVAWKGSSGGFSNYFPRPWYQVAAVEKYLEEQLDPAAREYYEENGFVRFAGRAFPDLSAHSSSPKYAYVDKRVPGLTGGTSASCPVVAGIVGLLNDARLRRGLPTMGFINPWLYAKGYQALEDVTGGAAVGCQGIDIQTGKRVPGAGIIPGASWNATPDWDPATGLGLPNFWAMRELALED

SEQ ID NO: 10 - protein, 575 aa, Aspergillus kawachii
VVHEKLAAVPSGWHHLEDAGSDHQISLSIALARKNLDQLESKLKDLSTPGESQYGQWLDQEEVDTLFPVASDKAVISWLRSANITHIARQGSLVNFATTVDKVNKLLNTTFAYYQRGSSQRLRTTEYSIPDDLVDSIDLISPTTFFGKEKTSAGLTQRSQKVDNHVAKRSNSSSCADTITLSCLKEMYNFGNYTPSASSGSKLGFASFLNESASYSDLAKFERLFNLPSQNFSVELINGGVNDQNQSTASLTEADLDVELLVGVGHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNSTQFNPIFPATCPYVTAVGGTMSYAPEIAWEASSGGFSNYFERAWFQKEAVQNYLANHITNETKQYYSQFANFSGRGFPDVSAHSFEPSYEVIFYGARYGSGGTSAACPLFSALVGMLNDARLRAGKSTLGFLNPLLYSKGYKALTDVTAGQSIGCNGIDPQSDEAVAGAGIIPWAHWNATVGWDPVTGLGLPDFEKLRQLVLSL

SEQ ID NO: 11 - protein, 582 aa, Talaromyces stipitatus
AAALVGHESLAALPVGWDKVSTPAAGTNIQLSVALALQNIEQLEDHLKSVSTPGSASYGQYLDSDGIAAQYGPSDASVEAVTNWLKEAGVTDIYNNGQSIHFATSVSKANSLLGADFNYYSDGSATKLRTLAYSVPSDLKEAIDLVSPTTYFGKTTASRSIQAYKNKRASTTSKSGSSSVQVSASCQTSITPACLKQMYNVGNYTPSVAHGSRVGFGSFLNQSAIFDDLFTYEKVNDIPSQNFTKVIIANASNSQDASDGNYGEANLDVQNIVGISHPLPVTEFLTGGSPPFVASLDTPTNQNEPYIPYYEYLLSQKNEDLPQVISNSYGDDEQSVPYKYAIRACNLIGLTGLRGISVLESSGDLGVGAGCRSNDGKNKTQFDPIFPATCPYVTSVGGTQSVTPEIAWVASSGGFSNYFPRTWYQEPAIQTYLGLLDDETKTYYSQYTNFEGRGFPDVSAHSLTPDYQVVGGGYLQPSGGTSAASPVFAGIIALLNDARLAAGKPTLGFLNPFFYLYGYKGLNDITGGQSVGCNGINGQTGAPVPGGGIVPGAAWNSTTGWDPATGLGTPDFQKLKELVLSF

SEQ ID NO: 12 - protein, 579 aa, Fusarium oxysporum
KSFSHHAEAPQGWQVQKTAKVASNTQHVFSLALTMQNVDQLESKLLDLSSPDSANYGNWLSHDELTSTFSPSKEAVASVTKWLKSKGIKHYKVNGAFIDFAADVEKANTLLGGDYQYYTKDGQTKLRTLSYSIPDDVAGHVQFVDPSTNFGGTVAFNPVPHPSRTLQERKVSPSKSTVDASCQTSITPSCLKQMYNIGDYTPDAKSGSEIGFSSFLGQAAIYSDVFKFEELFGIPKQNYTTILINNGTDDQNTAHGNFGEANLDAENIVGIAHPLPFKQYITGGSPPFVPNIDQPTEKDNQNEPYVPFFRYLLGQKDLPAVISTSYGDEEDSVPREYATLTCNMIGLLGLRGISVIFSSGDIGVGSGCLAPDYKTVEFNAIFPATCPYLTSVGGTVDVTPEIAWEGSSGGFSKYFPRPSYQDKAIKKYMKTVSKETKKYYGPYTNWEGRGFPDVAGHSVAPDYEVIYNGKQARSGGTSAAAPVWAAIVGLLNDARFKAGKKSLGWLNPLIYKHGPKVLTDITGGYAIGCDGNNTQSGKPEPAGSGLVPGARWNATAGWDPTTGYGTPNFQKLKDLVLSL

SEQ ID NO: 13 - protein, 590 aa, Trichoderma virens
SVLVESLEKLPHGWKAASAPSPSSQITLQVALTQQNIDQLESRLAAVSTPNSKTYGNYLDLDEINEIFAPSDASSAAVESWLHSHGVTKYTKQGSSIWFQTEVSTANAMLSTNFHTYSDAAGVKKLRTLQYSIPESLVGHVDLISPTTYFGTSNAMRALRSKSVASVAQSVAARQEPSSCKGTLVFEGRTFNVFQPDCLRTEYNVNGYTPSAKSGSRIGFGSFLNQSASFSDLALFEKHFGFSSQNFSVVLINGGTDLPQPPSDDNDGEANLDVQNILTIAHPLPITEFITAGSPPYFPDPVEPAGTPDENEPYLQYFEYLLSKPNRDLPQVITNSYGDEEQTVPQAYAVRVCNLIGLMGLRGISILESSGDEGVGASCVATNSTTPQFNPIFPATCPYVTSVGGTVNFNPEVAWDGSSGGFSYYFSRPWYQEEAVGNYLEKHVSAETKKYYGPYVDFSGRGFPDVAAHSVSPDYPVFQGGQLTPSGGTSAASPVVASIIALLNDARLREGKPTLGFLNPLIYQYAYKGFTDITSGQSDGCNGNNTQTDAPLPGAGVVLGAHWNATKGWDPTTGFGVPNFKKLLELIRYI

SEQ ID NO: 14 - protein, 569 aa, Trichoderma atroviride
AVLVESLKQVPNGWNAVSTPDPSTSIVLQIALAQQNIDELEWRLAAVSTPNSGNYGKYLDIGEIEGIFAPSNASYKAVASWLQSHGVKNFVKQAGSIWFYTTVSTANKMLSTDFKHYSDPVGIEKLRTLQYSIPEELVGHVDLISPTTYFGNNHPATARTPNMKAINVTYQIFHPDCLKTKYGVDGYAPSPRCGSRIGFGSFLNETASYSDLAQFEKYFDLPNQNLSTLLINGAIDVQPPSNKNDSEANMDVQTILTFVQPLPITEFVVAGIPPYIPDAALPIGDPVQNEPWLEYFEFLMSRTNAELPQVIANSYGDEEQTVPQAYAVRVCNQIGLLGLRGISVIASSGDTGVGMSCMASNSTTPQFNPMFPASCPYITTVGGTQHLDNEIAWELSSGGFSNYFTRPWYQEDAAKTYLERHVSTETKAYYERYANFLGRGFPDVAALSLNPDYPVIIGGELGPNGGTSAAAPVVASIIALLNDARLCLGKPALGFLNPLIYQYADKGGFTDITSGQSWGCAGNTTQTGPPPPGAGVIPGAHWNATKGWDPVTGFGTPNFKKLLSLALSV

SEQ ID NO: 15 - protein, 565 aa, Agaricus bisporus
SPLARRWDDFAEKHAWVEVPRGWEMVSEAPSDHTFDLRIGVKSSGMEQLIENLMQTSDPTHSRYGQHLSKEELHDFVQPHPDSTGAVEAWLEDFGISDDFIDRTGSGNWVTVRVSVAQAERMLGTKYNVYRHSESGESVVRTMSYSLPSELHSHIDVVAPTTYFGTMKSMRVTSFLQPEIEPVDPSAKPSAAPASCLSTTVITPDCLRDLYNTADYVPSATSRNAIGIAGYLDRSNRADLQTFFRRFRPDAVGFNYTTVQLNGGGDDQNDPGVEANLDIQYAAGIAFPTPATYWSTGGSPPFIPDTQTPTNTNEPYLDWINFVLGQDEIPQVISTSYGDDEQTVPEDYATSVCNLFAQLGSRGVTVFFSSGDFGVGGGDCLTNDGSNQVLFQPAFPASCPFVTAVGGTVRLDPEIAVSFSGGGFSRYFSRPSYQNQTVAQFVSNLGNTFNGLYNKNGRAYPDLAAQGNGFQVVIDGIVRSVGGTSASSPTVAGIFALLNDFKLSRGQSTLGFINPLIYSSATSGFNDIRAGTNPGCGTRGFTAGTGWDPVTGLGTPDFLRLQGLI

SEQ ID NO: 16 - protein, 583 aa, Magnaporthe oryzae
RVFDSLPHPPRGWSYSHAAESTEPLTLRIALRQQNAAALEQVVLQVSNPRHANYGQHLTRDELRSYTAPTPRAVRSVTSWLVDNGVDDYTVEHDWVTLRTTVGAADRLLGADFAWYAGPGETLQLRTLSYGVDDSVAPHVDLVQPTTRFGGPVGQASHIFKQDDFDEQQLKTLSVGFQVMADLPANGPGSIKAACNESGVTPLCLRTLYRVNYKPATTGNLVAFASFLEQYARYSDQQAFTQRVLGPGVPLQNFSVETVNGGANDQQSKLDSGEANLDLQYVMAMSHPIPILEYSTGGRGPLVPTLDQPNANNSSNEPYLEFLTYLLAQPDSAIPQTLSVSYGEEEQSVPRDYAIKVCNMFMQLGARGVSVMFSSGDSGPGNDCVRASDNATFFGSTFPAGCPYVTSVGSTVGFEPERAVSFSSGGFSIYHARPDYQNEVVPKYIESIKASGYEKFFDGNGRGIPDVAAQGARFVVIDKGRVSLISGTSASSPAFAGMVALVNAARKSKDMPALGFLNPMLYQNAAAMTDIVNGAGIGCRKQRTEFPNGARFNATAGWDPVTGLGTPLFDKLLAVGAPGVPNA

SEQ ID NO: 17 - protein, 594 aa, Togninia minima
SDVVLESLREVPQGWKRLRDADPEQSIKLRIALEQPNLDLFEQTLYDISSPDHPKYGQHLKSHELRDIMAPREESTAAVIAWLQDAGLSGSQIEDDSDWINIQTTVAQANDMLNTTFGLFAQEGTEVNRIRALAYSVPEEIVPHVKMIAPIIRFGQLRPQMSHIFSHEKVEETPSIGTIKAAAIPSVDLNVTACNASITPECLRALYNVGDYEADPSKKSLFGVCGYLEQYAKHDQLAKFEQTYAPYAIGADFSVVTINGGGDNQTSTIDDGEANLDMQYAVSMAYKTPITYYSTGGRGPLVPDLDQPDPNDVSNEPYLDFVSYLLKLPDSKLPQTITTSYGEDEQSVPRSYVEKVCTMFGALGARGVSVIFSSGDTGVGSACQTNDGKNTTRFLPIFPAACPYVTSVGGTRYVDPEVAVSFSSGGFSDIFPTPLYQKGAVSGYLKILGDRWKGLYNPHGRGFPDVSGQSVRYHVFDYGKDVMYSGTSASAPMFAALVSLLNNARLAKKLPPMGFLNPWLYTVGFNGLTDIVHGGSTGCTGTDVYSGLPTPFVPYASWNATVGWDPVTGLGTPLFDKLLNLSTPNFHLPHIGGH

SEQ ID NO: 18 - protein, 595 aa, Bipolaris maydis
STTSHVEGEVVERLHGVPEGWSQVGAPNPDQKLRFRIAVRSADSELFERTLMEVSSPSHPRYGQHLKRHELKDLIKPRAKSTSNILNWLQESGIEARDIQNDGEWISFYAPVKRAEQMMSTTFKTYQNEARANIKKIRSLDYSVPKHIRDDIDIIQPTTRFGQIQPERSQVFSQEEVPFSALVVNATCNKKITPDCLANLYNFKDYDASDANVTIGVSGFLEQYARFDDLKQFISTFQPKAAGSTFQVTSVNAGPFDQNSTASSVEANLDIQYTTGLVAPDIETRYFTVPGRGILIPDLDQPTESDNANEPYLDYFTYLNNLEDEELPDVLTTSYGESEQSVPAEYAKKVCNLIGQLGARGVSVIFSSGDTGPGSACQTNDGKNTTRFLPIFPASCPYVTSVGGTVGVEPEKAVSFSSGGFSDLWPRPAYQEKAVSEYLEKLGDRWNGLYNPQGRGFPDVAAQGQGFQVFDKGRLISVGGTSASAPVFASVVALLNNARKAAGMSSLGFLNPWIYEQGYKGLTDIVAGGSTGCTGRSIYSGLPAPLVPYASWNATEGWDPVTGYGTPDFKQLLTLATAPKSGERRVRRGGLGGQA

SEQ ID NO: 19 - protein, 613 aa, Aspergillus kawachii
MLSSFLSQGAAVSLALLSLLPSPVAAEIFEKLSGVPNGWRYANNPHGNEVIRLQIALQQHDVAGFEQAVMDMSTPGHADYGKHFRTHDEMKRMLLPSDTAVDSVRDWLESAGVHNIQVDADWVKFHTTVNKANALLDADFKWYVSEAKHIRRLRTLQYSIPDALVSHINMIQPTTRFGQIQPNRATMRSKPKHADETFLTAATLAQNTSHCDSIITPHCLKQLYNIGDYQADPKSGSKVGFASYLEEYARYADLERFEQHLAPNAIGQNFSVVQFNGGLNDQLSLSDSGEANLDLQYILGVSAPVPVTEYSTGGRGELVPDLSSPDPNDNSNEPYLDFLQGILKLDNSDLPQVISTSYGEDEQTIPVPYARTVCNLYAQLGSRGVSVIFSSGDSGVGAACLTNDGTNRTHFPPQFPASCPWVTSVGATSKTSPEQAVSFSSGGFSDLWPRPSYQQAAVQTYLTQHLGNKFSGLFNASGRAFPDVAAQGVNYAVYDKGMLGQFDGTSCSAPTFSGVIALLNDARLRAGLPVMGFLNPFLYGVGSESGALNDIVNGGSLGCDGRNRFGGTPNGSPVVPFASWNATTGWDPVSGLGTPDFAKLRGVALGEAKAYGN

SEQ ID NO: 20 - protein, 658 aa, Aspergillus nidulans
MAATGRFTAFWNVASVPALIGILPLAGSHLRAVLCPVCIWRHSKAVCAPDTLQAMRAFTRVTAISLAGFSCFAAAAAAAFESLRAVPDGWIYESTPDPNQPLRLRIALKQHNVAGFEQALLDMSTPGHSSYGQHFGSYHEMKQLLLPTEEASSSVRDWLSAAGVEFEQDADWINFRTTVDQANALLDADFLWYTTTGSTGNPTRILRTLSYSVPSELAGYVNMIQPTTRFGGTHANRATVRAKPIFLETNRQLINAISSGSLEHCEKAITPSCLADLYNTEGYKASNRSGSKVAFASFLEEYARYDDLAEFEETYAPYAIGQNFSVISINGGLNDQDSTADSGEANLDLQYIIGVSSPLPVTEFTTGGRGKLIPDLSSPDPNDNTNEPFLDFLEAVLKLDQKDLPQVISTSYGEDEQTIPEPYARSVCNLYAQLGSRGVSVLFSSGDSGVGAACQTNDGKNTTHFPPQFPASCPWVTAVGGTNGTAPESGVYFSSGGFSDYWARPAYQNAAVESYLRKLGSTQAQYFNRSGRAFPDVAAQAQNFAVVDKGRVGLFDGTSCSSPVFAGIVALLNDVRLKAGLPVLGFLNPWLYQDGLNGLNDIVDGGSTGCDGNNRFNGSPNGSPVIPYAGWNATEGWDPVTGLGTPDFAKLKALVLDA

SEQ ID NO: 21 - protein, 604 aa, Aspergillus ruber
MLSFVRRGALSLALVSLLTSSVAAEVFEKLHVVPEGWRYASTPNPKQPIRLQIALQQHDVTGFEQSLLEMSTPDHPNYGKHFRTHDEMKRMLLPNENAVHAVREWLQDAGISDIEEDADWVRFHTTVDQANDLLDANFLWYAHKSHRNTARLRTLEYSIPDSIAPQVNVIQPTTRFGQIRANRATHSSKPKGGLDELAISQAATADDDSICDQITTPHCLRKLYNVNGYKADPASGSKIGFASFLEEYARYSDLVLFEENLAPFAEGENFTVVMYNGGKNDQNSKSDSGEANLDLQYIVGMSAGAPVTEFSTAGRAPVIPDLDQPDPSAGTNEPYLEFLQNVLHMDQEHLPQVISTSYGENEQTIPEKYARTVCNMYAQLGSRGVSVIFSSGDSGVGSACMTNDGTNRTHFPPQFPASCPWVTSVGATEKMAPEQATYFSSGGFSDLFPRPKYQDAAVSSYLQTLGSRYQGLYNGSNRAFPDVSAQGTNFAVYDKGRLGQFDGTSCSAPAFSGIIALLNDVRLQNNKPVLGFLNPWLYGAGSKGLNDVVHGGSTGCDGQERFAGKANGSPVVPYASWNATQGWDPVTGLGTPDFGKLKDLALSA

SEQ ID NO: 22 - protein, 600 aa, Aspergillus terreus
MLPSLVNNGALSLAVLSLLTSSVAGEVFEKLSAVPKGWHFSHAAQADAPINLKIALKQHDVEGFEQALLDMSTPGHENYGKHFHEHDEMKRMLLPSDSAVDAVQTWLTSAGITDYDLDADWINLRTTVEHANALLDTQFGWYENEVRHITRLRTLQYSIPETVAAHINMVQPTTRFGQIRPDRATFHAHHTSDARILSALAAASNSTSCDSVITPKCLKDLYKVGDYEADPDSGSQVAFASYLEEYARYADMVKFQNSLAPYAKGQNFSVVLYNGGVNDQSSSADSGEANLDLQTIMGLSAPLPITEYITGGRGKLIPDLSQPNPNDNSNEPYLEFLQNILKLDQDELPQVISTSYGEDEQTIPRGYAESVCNMLAQLGSRGVSVVFSSGDSGVGAACQTNDGRNQTHFNPQFPASCPWVTSVGATTKTNPEQAVYFSSGGFSDFWKRPKYQDEAVAAYLDTLGDKFAGLFNKGGRAFPDVAAQGMNYAIYDKGTLGRLDGTSCSAPAFSAIISLLNDARLREGKPTMGFLNPWLYGEGREALNDVVVGGSKGCDGRDRFGGKPNGSPVVPFASWNATQGWDPVTGLGTPNFAKMLELAP

SEQ ID NO: 23 - protein, 601 aa, Penicillium digitatum
MIASLFNRRALTLALLSLFASSATADVFESLSAVPQGWRYSRTPSANQPLKLQIALAQGDVAGFEAAVIDMSTPDHPSYGNHFNTHEEMKRMLQPSAESVDSIRNWLESAGISKIEQDADWMTFYTTVKTANELLAANFQFYINGVKKIERLRTLKYSVPDALVSHINMIQPTTRFGQLRAQRAILHTEVKDNDEAFRSNAMSANPDCNSIITPQCLKDLYSIGDYEADPTNGNKVAFASYLEEYARYSDLALFEKNIAPFAKGQNFSVVQYNGGGNDQQSSSGSSEANLDLQYIVGVSSPVPVTEFSTGGRGELVPDLDQPNPNDNNNEPYLEFLQNVLKLHKKDLPQVISTSYGEDEQSVPEKYARAVCNLYSQLGSRGVSVIFSSGDSGVGAACQTNDGRNATHFPPQFPAACPWVTSVGATTHTAPERAVYFSSGGFSDLWDRPTWQEDAVSEYLENLGDRWSGLFNPKGRAFPDVAAQGENYAIYDKGSLISVDGTSCSAPAFAGVIALLNDARIKANRPPMGFLNPWLYSEGRSGLNDIVNGGSTGCDGHGRFSGPTNGGTSIPGASWNATKGWDPVSGLGSPNFAAMRKLANAE

SEQ ID NO: 24 - protein, 601 aa, Penicillium oxalicum
MHVPLLNQGALSLAVVSLLASTVSAEVFDKLVAVPEGWRFSRTPSGDQPIRLQVALTQGDVEGFEKAVLDMSTPDHPNYGKHFKSHEEVKRMLQPAGESVEAIHQWLEKAGITHIQQDADWMTFYTTVEKANNLLDANFQYYLNENKQVERLRTLEYSVPDELVSHINLVTPTTRFGQLHAEGVTLHGKSKDVDEQFRQAATSPSSDCNSAITPQCLKDLYKVGDYKASASNGNKVAFTSYLEQYARYSDLALFEQNIAPYAQGQNFTVIQYNGGLNDQSSPADSSEANLDLQYIIGTSSPVPVTEFSTGGRGPLVPDLDQPDINDNNNEPYLDFLQNVIKMSDKDLPQVISTSYGEDEQSVPASYARSVCNLIAQLGGRGVSVIFSSGDSGVGSACQTNDGKNTTRFPAQFPAACPWVTSVGATTGISPERGVFFSSGGFSDLWSRPSWQSHAVKAYLHKLGKRQDGLFNREGRAFPDVSAQGENYAIYAKGRLGKVDGTSCSAPAFAGLVSLLNDARIKAGKSSLGFLNPWLYSHPDALNDITVGGSTGCDGNARFGGRPNGSPVVPYASWNATEGWDPVTGLGTPNFQKLLKSAVKQK

SEQ ID NO: 25 - protein, 601 aa, Penicillium roqueforti
MIASLFSRGALSLAVLSLLASSAAADVFESLSAVPQGWRYSRRPRADQPLKLQIALTQGDTAGFEEAVMEMSTPDHPSYGHHFTTHEEMKRMLQPSAESAESIRDWLEGAGITRIEQDADWMTFYTTVETANELLAANFQFYVSNVRHIERLRTLKYSVPKALVPHINMIQPTTRFGQLRAHRGILHGQVKESDEAFRSNAVSAQPDCNSIITPQCLKDIYNIGDYQANDTNGNKVGFASYLEEYARYSDLALFEKNIAPSAKGQNFSVTRYNGGLNDQSSSGSSSEANLDLQYIVGVSSPVPVTEFSVGGRGELVPDLDQPDPNDNNNEPYLEFLQNVLKLDKKDLPQVISTSYGEDEQSIPEKYARSVCNLYSQLGSRGVSVIFSSGDSGVGSACLTNDGRNATRFPPQFPAACPWVTSVGATTHTAPEQAVYFSSGGFSDLWARPKWQEEAVSEYLEILGNRWSGLFNPKGRAFPDVTAQGRNYAIYDKGSLTSVDGTSCSAPAFAGVVALLNDARLKVNKPPMGFLNPWLYSTGRAGLKDIVDGGSTGCDGKSRFGGANNGGPSIPGASWNATKGWDPVSGLGSPNFATMRKLANAE

SEQ ID NO: 26 - protein, 601 aa, Penicillium rubens Wisconsin
MIASLFNRGALSLAVLSLLASSASADVFESLSAVPQGWRYSRRPRADQPLKLQIALAQGDTAGFEEAVMDMSTPDHPSYGNHFHTHEEMKRMLQPSAESADSIRDWLESAGINRIEQDADWMTFYTTVETANELLAANFQFYANSAKHIERLRTLQYSVPEALMPHINMIQPTTRFGQLRVQGAILHTQVKETDEAFRSNAVSTSPDCNSIITPQCLKNMYNVGDYQADDDNGNKVGFASYLEEYARYSDLELFEKNVAPFAKGQNFSVIQYNGGLNDQHSSASSSEANLDLQYIVGVSSPVPVTEFSVGGRGELVPDLDQPDPNDNNNEPYLEFLQNVLKMEQQDLPQVISTSYGENEQSVPEKYARTVCNLFSQLGSRGVSVIFASGDSGVGAACQTNDGRNATRFPAQFPAACPWVTSVGATTHTAPEKAVYFSSGGFSDLWDRPKWQEDAVSDYLDTLGDRWSGLFNPKGRAFPDVSAQGQNYAIYDKGSLTSVDGTSCSAPAFAGVIALLNDARLKANKPPMGFLNPWLYSTGRDGLNDIVHGGSTGCDGNARFGGPGNGSPRVPGASWNATKGWDPVSGLGSPNFATMRKLANGE

SEQ ID NO: 27 - protein, 594 aa, Neosartorya fischeri
MLSSTLYAGLLCSLAAPALGVVHEKLSAVPSGWTLVEDASESDTTTLSIALARQNLDQLESKLTTLATPGNAEYGKWLDQSDIESLFPTASDDAVIQWLKDAGVTQVSRQGSLVNFATTVGTANKLFDTKFSYYRNGASQKLRTTQYSIPDSLTESIDLIAPTVFFGKEQDSALPPHAVKLPALPRRAATNSSCANLITPDCLVEMYNLGDYKPDASSGSRVGFGSFLNQSANYADLAAYEQLFNIPPQNFSVELINGGANDQNWATASLGEANLDVELIVAVSHALPVVEFITGGSPPFVPNVDEPTAADNQNEPYLQYYEYLLSKPNSHLPQVISNSYGDDEQTVPEYYARRVCNLIGLMGLRGITVLESSGDTGIGSACMSNDGTNTPQFTPTFPGTCPFITAVGGTQSYAPEVAWDASSGGFSNYFSRPWYQYFAVENYLNNHITKDTKKYYSQYTNFKGRGFPDVSAHSLTPDYEVVLTGKHYKSGGTSAACPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 28 - protein, 568 aa, Aspergillus fumigatus
MLSSTLYAGWLLSLAAPALCVVQEKLSAVPSGWTLIEDASESDTITLSIALARQNLDQLESKLTTLATPGNPEYGKWLDQSDIESLFPTASDDAVLQWLKAAGITQVSRQGSLVNFATTVGTANKLFDTKFSYYRNGASQKLRTTQYSIPDHLTESIDLIAPTVFFGKEQNSALSSHAVKLPALPRRAATNSSCANLITPDCLVEMYNLGDYKPDASSGSRVGFGSFLNESANYADLAAYEQLFNIPPQNFSVELINRGVNDQNWATASLGEANLDVELIVAVSHPLPVVEFITGALPPVLRVLALQTQLPSSSGDFQLTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLSL

SEQ ID NO: 29 - protein, 415 aa, Trichoderma reesei
QEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 30 - protein, 391 aa, Aspergillus oryzae
CDSIITPTCLKELYNIGDYQADANSGSKIAFASYLEEYARYADLENFENYLAPWAKGQNFSVTTFNGGLNDQNSSSDSGEANLDLQYILGVSAPLPVTEFSTGGRGPLVPDLTQPDPNSNSNEPYLEFFQNVLKLDQKDLPQVISTSYGENEQEIPEKYARTVCNLIAQLGSRGVSVLFSSGDSGVGEGCMTNDGTNRTHFPPQFPAACPWVTSVGATFKTTPERGTYFSSGGFSDYWPRPEWQDEAVSSYLETIGDTFKGLYNSSGRAFPDVAAQGMNFAVYDKGTLGEFDGTSASAPAFSAVIALLNDARLRAGKPTLGFLNPWLYKTGRQGLQDITLGASIGCTGRARFGGAPDGGPVVPYASWNATQGWDPVTGLGTPDFAELKKLA

SEQ ID NO: 31 - protein, 388 aa, Phaeosphaeria nodorum
CDATITPQCLKTLYKIDYKADPKSGSKVAFASYLEQYARYNDLALFEKAFLPEAVGQNFSVVQFSGGLNDQNTTQDSGEANLDLQYIVGVSAPLPVTEFSTGGRGPWVADLDQPDEADSANEPYLEFLQGVLKLPQSELPQVISTSYGENEQSVPKSYALSVCNLFAQLGSRGVSVIFSSGDSGPGSACQSNDGKNTTKFQPQYPAACPFVTSVGSTRYLNETATGFSSGGFSDYWKRPSYQDDAVKAYFHHLGEKFKPYFNRHGRGFPDVATQGYGFRVYDQGKLKGLQGTSASAPAFAGVIGLLNDARLKAKKPTLGFLNPLLYSNSDALNDIVLGGSKGCDGHARFNGPPNGSPVIPYAGWNATAGWDPVTGLGTPNFPKLLKAA

SEQ ID NO: 32 - protein, 395 aa, Trichoderma atroviride
VFQPDCLRTEYSVNGYKPSAKSGSRIGFGSFLNQSASSSDLALFEKHFGFASQGFSVELINGGSNPQPPTDANDGEANLDAQNIVSFVQPLPITEFIAGGTAPYFPDPVEPAGTPDENEPYLEYYEYLLSKSNKELPQVITNSYGDEEQTVPQAYAVRVCNLIGLMGLRGISILESSGDEGVGASCLATNSTTTPQFNPIFPATCPYVTSVGGTVSFNPEVAWDGSSGGFSYYFSRPWYQEAAVGTYLNKYVSEETKEYYKSYVDFSGRGFPDVAAHSVSPDYPVFQGGELTPSGGTSAASPIVASVIALLNDARLRAGKPALGFLNPLIYGYAYKGFTDITSGQAVGCNGNNTQTGGPLPGAGVIPGAFWNATKGWDPTTGFGVPNFKKLLELV

SEQ ID NO: 33 - protein, 389 aa, Arthroderma benhamiae
CRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVL

SEQ ID NO: 34 - protein, 397 aa, Fusarium graminearum
CQTSITPSCLKQMYNIGDYTPKVESGSTIGFSSFLGESAIYSDVFLFEEKFGIPTQNFTTVLINNGTDDQNTAHKNFGEADLDAENIVGIAHPLPFTQYITGGSPPFLPNIDQPTAADNQNEPYVPFFRYLLSQKEVPAVVSTSYGDEEDSVPREYATMTCNLIGLLGLRGISVIFSSGDIGVGAGCLGPDHKTVEFNAIFPATCPYLTSVGGTVDVTPEIAWEGSSGGFSKYFPRPSYQDKAVKTYMKTVSKQTKKYYGPYTNWEGRGFPDVAGHSVSPNYEVIYAGKQSASGGTSAAAPVWAAIVGLLNDARFRAGKPSLGWLNPLVYKYGPKVLTDITGGYAIGCDGNNTQSGKPEPAGSGIVPGARWNATAGWDPVTGYGTPDFGKLKDLVLS

SEQ ID NO: 35 - protein, 408 aa, Acremonium alcalophilum
CDLVITPPCLEAAYNYKNYMPDPNSGSRVSFTSFLEQAAQQSDLTKFLSLTGLDRLRPPSSKPASFDTVLINGGETHQGTPPNKTSEANLDVQWLAAVIKARLPITQWITGGRPPFVPNLRLRHEKDNTNEPYLEFFEYLVRLPARDLPQVISNSYAEDEQTVPEAYARRVCNLIGIMGLRGVTVLTASGDSGVGAPCRANDGSDRLEFSPQFPTSCPYITAVGGTEGWDPEVAWEASSGGFSHYFLRPWYQANAVEKYLDEELDPATRAYYDGNGFVQFAGRAYPDLSAHSSSPRYAYIDKLAPGLTGGTSASCPVVAGIVGLLNDARLRRGLPTMGFINPWLYTRGFEALQDVTGGRASGCQGIDLQRGTRVPGAGIIPWASWNATPGWDPATGLGLPDFWAMRGL

SEQ ID NO: 36 - protein, 410 aa, Sodiomyces alkalinus
CATIITPPCLETAYNYKGYIPDPKSGSRVSFTSFLEQAAQQADLTKFLSLTRLEGFRTPASKKKTFKTVLINGGESHEGVHKKSKTSEANLDVQWLAAVTQTKLPITQWITGGRPPFVPNLRIPTPEANTNEPYLEFLEYLFRLPDKDLPQVISNSYAEDEQSVPEAYARRVCGLLGIMGLRGVTVLTASGDSGVGAPCRANDGSGREEFSPQFPSSCPYITTVGGTQAWDPEVAWKGSSGGFSNYFPRPWYQVAAVEKYLEEQLDPAAREYYEENGFVRFAGRAFPDLSAHSSSPKYAYVDKRVPGLTGGTSASCPVVAGIVGLLNDARLRRGLPTMGFINPWLYAKGYQALEDVTGGAAVGCQGIDIQTGKRVPGAGIIPGASWNATPDWDPATGLGLPNFWAMRELA

SEQ ID NO: 37 - protein, 400 aa, Aspergillus kawachii
CADTITLSCLKEMYNFGNYTPSASSGSKLGFASFLNESASYSDLAKFERLFNLPSQNFSVELINGGVNDQNQSTASLTEADLDVELLVGVGHPLPVTEFITSGEPPFIPDPDEPSAADNENEPYLQYYEYLLSKPNSALPQVISNSYGDDEQTVPEYYAKRVCNLIGLVGLRGISVLESSGDEGIGSGCRTTDGTNSTQFNPIFPATCPYVTAVGGTMSYAPEIAWEASSGGFSNYFERAWFQKEAVQNYLANHITNETKQYYSQFANFSGRGFPDVSAHSFEPSYEVIFYGARYGSGGTSAACPLFSALVGMLNDARLRAGKSTLGFLNPLLYSKGYKALTDVTAGQSIGCNGIDPQSDEAVAGAGIIPWAHWNATVGWDPVTGLGLPDFEKLRQLVLS

SEQ ID NO: 38 - protein, 396 aa, Talaromyces stipitatus
CQTSITPACLKQMYNVGNYTPSVAHGSRVGFGSFLNQSAIFDDLFTYEKVNDIPSQNFTKVIIANASNSQDASDGNYGEANLDVQNIVGISHPLPVTEFLTGGSPPFVASLDTPTNQNEPYIPYYEYLLSQKNEDLPQVISNSYGDDEQSVPYKYAIRACNLIGLTGLRGISVLESSGDLGVGAGCRSNDGKNKTQFDPIFPATCPYVTSVGGTQSVTPEIAWVASSGGFSNYFPRTWYQEPAIQTYLGLLDDETKTYYSQYTNFEGRGFPDVSAHSLTPDYQVVGGGYLQPSGGTSAASPVFAGIIALLNDARLAAGKPTLGFLNPFFYLYGYKGLNDITGGQSVGCNGINGQTGAPVPGGGIVPGAAWNSTTGWDPATGLGTPDFQKLKELVLS

SEQ ID NO: 39 - protein, 397 aa, Fusarium oxysporum
CQTSITPSCLKQMYNIGDYTPDAKSGSEIGFSSFLGQAAIYSDVFKFEELFGIPKQNYTTILINNGTDDQNTAHGNFGEANLDAENIVGIAHPLPFKQYITGGSPPFVPNIDQPTEKDNQNEPYVPFFRYLLGQKDLPAVISTSYGDEEDSVPREYATLTCNMIGLLGLRGISVIFSSGDIGVGSGCLAPDYKTVEFNAIFPATCPYLTSVGGTVDVTPEIAWEGSSGGFSKYFPRPSYQDKAIKKYMKTVSKETKKYYGPYTNWEGRGFPDVAGHSVAPDYEVIYNGKQARSGGTSAAAPVWAAIVGLLNDARFKAGKKSLGWLNPLIYKHGPKVLTDITGGYAIGCDGNNTQSGKPEPAGSGLVPGARWNATAGWDPTTGYGTPNFQKLKDLVLS

SEQ ID NO: 40 - protein, 395 aa, Trichoderma virens
VFQPDCLRTEYNVNGYTPSAKSGSRIGFGSFLNQSASFSDLALFEKHFGFSSQNFSVVLINGGTDLPQPPSDDNDGEANLDVQNILTIAHPLPITEFITAGSPPYFPDPVEPAGTPDENEPYLQYFEYLLSKPNRDLPQVITNSYGDEEQTVPQAYAVRVCNLIGLMGLRGISILESSGDEGVGASCVATNSTTPQFNPIFPATCPYVTSVGGTVNFNPEVAWDGSSGGFSYYFSRPWYQEEAVGNYLEKHVSAETKKYYGPYVDFSGRGFPDVAAHSVSPDYPVFQGGQLTPSGGTSAASPVVASIIALLNDARLREGKPTLGFLNPLIYQYAYKGFTDITSGQSDGCNGNNTQTDAPLPGAGVVLGAHWNATKGWDPTTGFGVPNFKKLLELI

SEQ ID NO: 41 - protein, 398 aa, Trichoderma atroviride
QIFHPDCLKTKYGVDGYAPSPRCGSRIGFGSFLNETASYSDLAQFEKYFDLPNQNLSTLLINGAIDVQPPSNKNDSEANMDVQTILTFVQPLPITEFVVAGIPPYIPDAALPIGDPVQNEPWLEYFEFLMSRTNAELPQVIANSYGDEEQTVPQAYAVRVCNQIGLLGLRGISVIASSGDTGVGMSCMASNSTTPQFNPMFPASCPYITTVGGTQHLDNEIAWELSSGGFSNYFTRPWYQEDAAKTYLERHVSTETKAYYERYANFLGRGFPDVAALSLNPDYPVIIGGELGPNGGTSAAAPVVASIIALLNDARLCLGKPALGFLNPLIYQYADKGGFTDITSGQSWGCAGNTTQTGPPPPGAGVIPGAHWNATKGWDPVTGFGTPNFKKLLSLALS

SEQ ID NO: 42 - protein, 363 aa, Agaricus bisporus
TVITPDCLRDLYNTADYVPSATSRNAIGIAGYLDRSNRADLQTFFRRFRPDAVGFNYTTVQLNGGGDDQNDPGVEANLDIQYAAGIAFPTPATYWSTGGSPPFIPDTQTPTNTNEPYLDWINFVLGQDEIPQVISTSYGDDEQTVPEDYATSVCNLFAQLGSRGVTVFFSSGDFGVGGGDCLTNDGSNQVLFQPAFPASCPFVTAVGGTVRLDPEIAVSFSGGGFSRYFSRPSYQNQTVAQFVSNLGNTFNGLYNKNGRAYPDLAAQGNGFQVVIDGIVRSVGGTSASSPTVAGIFALLNDFKLSRGQSTLGFINPLIYSSATSGFNDIRAGTNPGCGTRGFTAGTGWDPVTGLGTPDFLRLQ

SEQ ID NO: 43 - protein, 376 aa, Magnaporthe oryzae
GVTPLCLRTLYRVNYKPATTGNLVAFASFLEQYARYSDQQAFTQRVLGPGVPLQNFSVETVNGGANDQQSKLDSGEANLDLQYVMAMSHPIPILEYSTGGRGPLVPTLDQPNANNSSNEPYLEFLTYLLAQPDSAIPQTLSVSYGEEEQSVPRDYAIKVCNMFMQLGARGVSVMFSSGDSGPGNDCVRASDNATFFGSTFPAGCPYVTSVGSTVGFEPERAVSFSSGGFSIYHARPDYQNEVVPKYIESIKASGYEKFFDGNGRGIPDVAAQGARFVVIDKGRVSLISGTSASSPAFAGMVALVNAARKSKDMPALGFLNPMLYQNAAAMTDIVNGAGIGCRKQRTEFPNGARFNATAGWDPVTGLGTPLFDKLLA

SEQ ID NO: 44 - protein, 388 aa, Togninia minima
CNASITPECLRALYNVGDYEADPSKKSLFGVCGYLEQYAKHDQLAKFEQTYAPYAIGADFSVVTINGGGDNQTSTIDDGEANLDMQYAVSMAYKTPITYYSTGGRGPLVPDLDQPDPNDVSNEPYLDFVSYLLKLPDSKLPQTITTSYGEDEQSVPRSYVEKVCTMFGALGARGVSVIFSSGDTGVGSACQTNDGKNTTRFLPIFPAACPYVTSVGGTRYVDPEVAVSFSSGGFSDIFPTPLYQKGAVSGYLKILGDRWKGLYNPHGRGFPDVSGQSVRYHVFDYGKDVMYSGTSASAPMFAALVSLLNNARLAKKLPPMGFLNPWLYTVGFNGLTDIVHGGSTGCTGTDVYSGLPTPFVPYASWNATVGWDPVTGLGTPLFDKLLNL

SEQ ID NO: 45 - protein, 390 aa, Bipolaris maydis
CNKKITPDCLANLYNFKDYDASDANVTIGVSGFLEQYARFDDLKQFISTFQPKAAGSTFQVTSVNAGPFDQNSTASSVEANLDIQYTTGLVAPDIETRYFTVPGRGILIPDLDQPTESDNANEPYLDYFTYLNNLEDEELPDVLTTSYGESEQSVPAEYAKKVCNLIGQLGARGVSVIFSSGDTGPGSACQTNDGKNTTRFLPIFPASCPYVTSVGGTVGVEPEKAVSFSSGGFSDLWPRPAYQEKAVSEYLEKLGDRWNGLYNPQGRGFPDVAAQGQGFQVFDKGRLISVGGTSASAPVFASVVALLNNARKAAGMSSLGFLNPWIYEQGYKGLTDIVAGGSTGCTGRSIYSGLPAPLVPYASWNATEGWDPVTGYGTPDFKQLLTLAT

SEQ ID NO: 46 - protein, 393 aa, Aspergillus kawachii
CDSIITPHCLKQLYNIGDYQADPKSGSKVGFASYLEEYARYADLERFEQHLAPNAIGQNFSVVQFNGGLNDQLSLSDSGEANLDLQYILGVSAPVPVTEYSTGGRGELVPDLSSPDPNDNSNEPYLDFLQGILKLDNSDLPQVISTSYGEDEQTIPVPYARTVCNLYAQLGSRGVSVIFSSGDSGVGAACLTNDGTNRTHFPPQFPASCPWVTSVGATSKTSPEQAVSFSSGGFSDLWPRPSYQQAAVQTYLTQHLGNKFSGLFNASGRAFPDVAAQGVNYAVYDKGMLGQFDGTSCSAPTFSGVIALLNDARLRAGLPVMGFLNPFLYGVGSESGALNDIVNGGSLGCDGRNRFGGTPNGSPVVPFASWNATTGWDPVSGLGTPDFAKLRGV

SEQ ID NO: 47 - protein, 392 aa, Aspergillus nidulans
CEKAITPSCLADLYNTEGYKASNRSGSKVAFASFLEEYARYDDLAEFEETYAPYAIGQNFSVISINGGLNDQDSTADSGEANLDLQYIIGVSSPLPVTEFTTGGRGKLIPDLSSPDPNDNTNEPFLDFLEAVLKLDQKDLPQVISTSYGEDEQTIPEPYARSVCNLYAQLGSRGVSVLFSSGDSGVGAACQTNDGKNTTHFPPQFPASCPWVTAVGGTNGTAPESGVYFSSGGFSDYWARPAYQNAAVESYLRKLGSTQAQYFNRSGRAFPDVAAQAQNFAVVDKGRVGLFDGTSCSSPVFAGIVALLNDVRLKAGLPVLGFLNPWLYQDGLNGLNDIVDGGSTGCDGNNRFNGSPNGSPVIPYAGWNATEGWDPVTGLGTPDFAKLKALVL

SEQ ID NO: 48 - protein, 392 aa, Aspergillus ruber
CDQITTPHCLRKLYNVNGYKADPASGSKIGFASFLEEYARYSDLVLFEENLAPFAEGENFTVVMYNGGKNDQNSKSDSGEANLDLQYIVGMSAGAPVTEFSTAGRAPVIPDLDQPDPSAGTNEPYLEFLQNVLHMDQEHLPQVISTSYGENEQTIPEKYARTVCNMYAQLGSRGVSVIFSSGDSGVGSACMTNDGTNRTHFPPQFPASCPWVTSVGATEKMAPEQATYFSSGGFSDLFPRPKYQDAAVSSYLQTLGSRYQGLYNGSNRAFPDVSAQGTNFAVYDKGRLGQFDGTSCSAPAFSGIIALLNDVRLQNNKPVLGFLNPWLYGAGSKGLNDVVHGGSTGCDGQERFAGKANGSPVVPYASWNATQGWDPVTGLGTPDFGKLKDLAL

SEQ ID NO: 49 - protein, 391 aa, Aspergillus terreus
CDSVITPKCLKDLYKVGDYEADPDSGSQVAFASYLEEYARYADMVKFQNSLAPYAKGQNFSVVLYNGGVNDQSSSADSGEANLDLQTIMGLSAPLPITEYITGGRGKLIPDLSQPNPNDNSNEPYLEFLQNILKLDQDELPQVISTSYGEDEQTIPRGYAESVCNMLAQLGSRGVSVVFSSGDSGVGAACQTNDGRNQTHFNPQFPASCPWVTSVGATTKTNPEQAVYFSSGGFSDFWKRPKYQDEAVAAYLDTLGDKFAGLFNKGGRAFPDVAAQGMNYAIYDKGTLGRLDGTSCSAPAFSAIISLLNDARLREGKPTMGFLNPWLYGEGREALNDVVVGGSKGCDGRDRFGGKPNGSPVVPFASWNATQGWDPVTGLGTPNFAKMLELA

SEQ ID NO: 50 - protein, 391 aa, Penicillium digitatum
CNSIITPQCLKDLYSIGDYEADPTNGNKVAFASYLEEYARYSDLALFEKNIAPFAKGQNFSVVQYNGGGNDQQSSSGSSEANLDLQYIVGVSSPVPVTEFSTGGRGELVPDLDQPNPNDNNNEPYLEFLQNVLKLHKKDLPQVISTSYGEDEQSVPEKYARAVCNLYSQLGSRGVSVIFSSGDSGVGAACQTNDGRNATHFPPQFPAACPWVTSVGATTHTAPERAVYFSSGGFSDLWDRPTWQEDAVSEYLENLGDRWSGLFNPKGRAFPDVAAQGENYAIYDKGSLISVDGTSCSAPAFAGVIALLNDARIKANRPPMGFLNPWLYSEGRSGLNDIVNGGSTGCDGHGRFSGPTNGGTSIPGASWNATKGWDPVSGLGSPNFAAMRKLA

SEQ ID NO: 51 - protein, 391 aa, Penicillium oxalicum
CNSAITPQCLKDLYKVGDYKASASNGNKVAFTSYLEQYARYSDLALFEQNIAPYAQGQNFTVIQYNGGLNDQSSPADSSEANLDLQYIIGTSSPVPVTEFSTGGRGPLVPDLDQPDINDNNNEPYLDFLQNVIKMSDKDLPQVISTSYGEDEQSVPASYARSVCNLIAQLGGRGVSVIFSSGDSGVGSACQTNDGKNTTRFPAQFPAACPWVTSVGATTGISPERGVFFSSGGFSDLWSRPSWQSHAVKAYLHKLGKRQDGLFNREGRAFPDVSAQGENYAIYAKGRLGKVDGTSCSAPAFAGLVSLLNDARIKAGKSSLGFLNPWLYSHPDALNDITVGGSTGCDGNARFGGRPNGSPVVPYASWNATEGWDPVTGLGTPNFQKLLKSAV

SEQ ID NO: 52 - protein, 391 aa, Penicillium roqueforti
CNSIITPQCLKDIYNIGDYQANDTNGNKVGFASYLEEYARYSDLALFEKNIAPSAKGQNFSVTRYNGGLNDQSSSGSSSEANLDLQYIVGVSSPVPVTEFSVGGRGELVPDLDQPDPNDNNNEPYLEFLQNVLKLDKKDLPQVISTSYGEDEQSIPEKYARSVCNLYSQLGSRGVSVIFSSGDSGVGSACLTNDGRNATRFPPQFPAACPWVTSVGATTHTAPEQAVYFSSGGFSDLWARPKWQEEAVSEYLEILGNRWSGLFNPKGRAFPDVTAQGRNYAIYDKGSLTSVDGTSCSAPAFAGVVALLNDARLKVNKPPMGFLNPWLYSTGRAGLKDIVDGGSTGCDGKSRFGGANNGGPSIPGASWNATKGWDPVSGLGSPNFATMRKLA

SEQ ID NO: 53 - protein, 391 aa, Penicillium rubens Wisconsin
CNSIITPQCLKNMYNVGDYQADDDNGNKVGFASYLEEYARYSDLELFEKNVAPFAKGQNFSVIQYNGGLNDQHSSASSSEANLDLQYIVGVSSPVPVTEFSVGGRGELVPDLDQPDPNDNNNEPYLEFLQNVLKMEQQDLPQVISTSYGENEQSVPEKYARTVCNLFSQLGSRGVSVIFASGDSGVGAACQTNDGRNATRFPAQFPAACPWVTSVGATTHTAPEKAVYFSSGGFSDLWDRPKWQEDAVSDYLDTLGDRWSGLFNPKGRAFPDVSAQGQNYAIYDKGSLTSVDGTSCSAPAFAGVIALLNDARLKANKPPMGFLNPWLYSTGRDGLNDIVHGGSTGCDGNARFGGPGNGSPRVPGASWNATKGWDPVSGLGSPNFATMRKLA

SEQ ID NO: 54 - protein, 400 aa, Neosartorya fischeri
CANLITPDCLVEMYNLGDYKPDASSGSRVGFGSFLNQSANYADLAAYEQLFNIPPQNFSVELINGGANDQNWATASLGEANLDVELIVAVSHALPVVEFITGGSPPFVPNVDEPTAADNQNEPYLQYYEYLLSKPNSHLPQVISNSYGDDEQTVPEYYARRVCNLIGLMGLRGITVLESSGDTGIGSACMSNDGTNTPQFTPTFPGTCPFITAVGGTQSYAPEVAWDASSGGFSNYFSRPWYQYFAVENYLNNHITKDTKKYYSQYTNFKGRGFPDVSAHSLTPDYEVVLTGKHYKSGGTSAACPVFAGIVGLLNDARLRAGKSTLGFLNPLLYSILAEGFTDITAGSSIGCNGINPQTGKPVPGGGIIPYAHWNATAGWDPVTGLGVPDFMKLKELVLS

SEQ ID NO: 55 - protein, 374 aa, Aspergillus fumigatus
CANLITPDCLVEMYNLGDYKPDASSGSRVGFGSFLNESANYADLAAYEQLFNIPPQNFSVELINRGVNDQNWATASLGEANLDVELIVAVSHPLPVVEFITGALPPVLRVLALQTQLPSSSGDFQLTVPEYYARRVCNLIGLMGLRGITVLESSGDTGIGSACMSNDGTNKPQFTPTFPGTCPFITAVGGTQSYAPEVAWDGSSGGFSNYFSRPWYQSFAVDNYLNNHITKDTKKYYSQYTNFKGRGFPDVSAHSLTPYYEVVLTGKHYKSGGTSAASPVFAGIVGLLNDARLRAGKSTLGFLNPLLYSILAEGFTDITAGSSIGCNGINPQTGKPVPGGGIIPYAHWNATAGWDPVTGLGVPDFMKLKELVLS

SEQ ID NO: 56 - DNA, 2027 bp, Trichoderma reesei
atggcaaagttgagcactctccggcttgcgagccttctttcccttgtcagtgtgcaggtatctgcctctgtccatctattggagagtctggagaagctgcctcatggatggaaagcagctgaaaccccgagcccttcgtctcaaatcgtcttgcaggttgctctgacgcagcagaacattgaccagcttgaatcgaggctcgcagctgtatccacacccacttctagcacctacggcaaatacttggatgtagacgagatcaacagcatcttcgctccaagtgatgctagcagttctgccgtcgagtcttggcttcagtcccacggagtgacgagttacaccaagcaaggcagcagcatttggtttcaaacaaacatctccactgcaaatgcgatgctcagcaccaatttccacacgtacagcgatctcaccggcgcgaagaaggtgcgcactctcaagtactcgatcccggagagcctcatcggccatgtcgatctcatctctcccacgacctattttggcacgacaaaggccatgaggaagttgaaatccagtggcgtgagcccagccgctgatgctctagccgctcgccaagaaccttccagctgcaaaggaactctagtctttgagggagaaacgttcaatgtctttcagccagactgtctcaggaccgagtatagtgttgatggatacaccccgtctgtcaagtctggcagcagaattgggtttggttcctttctcaatgagagcgcaagcttcgcagatcaagcactctttgagaagcacttcaacatccccagtcaaaacttctccgttgtcctgatcaacggtggaacggatctccctcagccgccttctgacgccaacgatggcgaagccaacctggacgctcaaaccattttgaccatcgcacatcctctccccatcaccgaattcatcaccgccggcagtccgccatacttccccgatccagttgaacctgcgggaacacccaacgagaacgagccttatttacagtattacgaatttctgttgtccaagtccaacgctgaaattccgcaagtcattaccaactcctacggcgacgaggagcaaactgtgccgcggtcatatgccgttcgagtttgcaatctgattggtctgctaggactacgcggtatctctgtccttcattcctcgggcgacgagggtgtgggcgcctcttgcgttgctaccaacagcaccacgcctcagtttaaccccatctttcctgtaggtcttctacgtcaacacttccagacaaccattttctcctactaaccactctaccctactctctgttcacataggctacatgtccttatgttacaagtgttggcggaaccgtgagcttcaatcccgaggttgcctgggctggttcatctggaggtttcagctactacttctctagaccctggtaccagcaggaagctgtgggtacttaccttgagaaatatgtcagtgctgagacaaagaaatactatggaccttatgtcgatttctccggacgaggtttccccgatgttgcagcccacagcgtcagccccgagtgagttctattcctacctatgcaaatcatagaatgtatgctaactcgccatgaagctatcctgtgtttcagggcggtgaactcaccccaagcggaggcacttcagcagcctctcctgtcgtagcagccatcgtggcgctgttgaacgatgcccgtctccgcgaaggaaaacccacgcttggatttctcaatccgctgatttacctacacgcctccaaagggttcaccgacatcacctcgggccaatctgaagggtgcaacggcaataacacccagacgggcagtcctctcccaggagtatgcagaacatcaagaagccttctatcagacgccaatgctaacttgtggataggccggcttcattgcaggcgcacactggaacgcgaccaagggatgggacccgacgactggatttggtgttccaaacctcaaaaagctcctcgcacttgtccggttctaa

SEQ ID NO: 57 - DNA, 1803 bp, Aspergillus oryzae
atgttcttcagtcgtggagcgctttcgctcgcagtgctttcactgctcagctcctccgccgcaggggaggcttttgagaagctgtctgccgttccaaagggatggcactattctagtacccctaaaggcaacactgaggtttgtctgaagatcgccctcgcgcagaaggatgctgctgggttcgaaaagaccgtcttggagatgtcggatcccgaccaccccagctacggccagcacttcaccacccacgacgagatgaagcgcatgcttcttcccagagatgacaccgttgatgccgttcgacaatggctcgaaaacggcggcgtgaccgactttacccaggatgccgactggatcaacttctgtactaccgtcgataccgcgaacaaactcttgaatgcccagttcaaatggtacgtcagcgatgtgaagcacatccgccgtctcagaacactgcagtacgacgtccccgagtcggtcacccctcacatcaacaccatccaaccgaccacccgttttggcaagattagccccaagaaggccgttacccacagcaagccctcccagttggacgtgaccgcccttgctgccgctgtcgttgcaaagaacatctcgcactgtgattctatcattacccccacctgtctgaaggagctttacaacattggtgattaccaggccgatgcaaactcgggcagcaagatcgccttcgccagctatctggaggagtacgcgcgctacgctgacctggagaactttgagaactaccttgctccctgggctaagggccagaacttctccgttaccaccttcaacggcggtctcaatgatcagaactcctcgtccgatagcggtgaggccaacctggacctgcagtacattcttggtgtcagcgctccactgcccgttactgaattcagcaccggaggccgtggtcccctcgttcctgatctgacccagccggatcccaactctaacagcaatgagccgtaccttgagttcttccagaatgtgttgaagctcgaccagaaggacctcccccaggtcatctcgacctcctatggagagaacgaacaggaaatccccgaaaagtacgctcgcaccgtctgcaacctgatcgctcagcttggcagccgcggtgtctccgttctcttctcctccggtgactctggtgttggcgagggctgcatgaccaacgacggcaccaaccggactcacttcccaccccagttccccgccgcttgcccgtgggtcacctccgtcggcgccaccttcaagaccactcccgagcgcggcacctacttctcctcgggcggtttctccgactactggccccgtcccgaatggcaggatgaggccgtgagcagctacctcgagacgatcggcgacactttcaagggcctctacaactcctccggccgtgctttccccgacgtcgcagcccagggcatgaacttcgccgtctacgacaagggcaccttgggcgagttcgacggcacctccgcctccgccccggccttcagcgccgtcatcgctctcctgaacgatgcccgtctccgcgccggcaagcccactctcggcttcctgaacccctggttgtacaagaccggccgccagggtctgcaagatatcaccctcggtgctagcattggctgcaccggtcgcgctcgcttcggcggcgcccctgacggtggtcccgtcgtgccttacgctagctggaacgctacccagggctgggatcccgtcactggtctcggaactcccgatttcgccgagctcaagaagcttgcccttggcaactaa

SEQ ID NO: 58 - DNA, 1782 bp, Phaeosphaeria nodorum
atggcgcccatcctctcgttccttgttggctctctcctggcggttcgcgctcttgctgagccatttgagaagctgttcagcaccccggaaggatggaagatgcaaggtcttgctaccaatgagcagatcgtcaagctccagattgctcttcagcaaggcgatgttgcaggtttcgagcaacatgtgattgacatctcaacgcctagccacccgagctatggtgctcactatggctcgcatgaggagatgaagaggatgatccagccaagcagcgagacagtcgcttctgtgtctgcatggctgaaggccgccggtatcaacgacgctgagattgacagcgactgggtcaccttcaagacgaccgttggcgttgccaacaagatgctcgacaccaagttcgcttggtacgtgagcgaggaggccaagccccgcaaggtccttcgcacactcgagtactctgtaccagatgatgttgcagaacacatcaacttgatccagcccactactcggtttgctgcgatccgccaaaaccacgaggttgcgcacgagattgttggtcttcagttcgctgctcttgccaacaacaccgttaactgcgatgccaccatcactccccagtgcttgaagactctttacaagattgactacaaggccgatcccaagagtggttccaaggtcgcttttgcttcgtatttggagcagtacgcgcgttacaatgacctcgccctcttcgagaaggccttcctccccgaagcagttggccagaacttctctgtcgtccagttcagcggcggtctcaacgaccagaacaccacgcaagacagtggcgaggccaacttggacttgcagtacattgtcggtgtcagcgctcctcttcccgtcaccgagttcagcaccggtggtcgcggcccatgggtcgctgacctagaccaacctgacgaggcggacagcgccaacgagccctaccttgaattccttcagggtgtgctcaaacttccccagtctgagctacctcaggtcatctccacatcctatggcgagaatgagcagagtgtacctaagtcatacgctctctccgtctgcaacttgttcgcccaactcggttcccgtggcgtctccgtcatcttctcttctggtgacagcggccctggatccgcatgccagagcaacgacggcaagaacacgaccaagttccagcctcagtaccccgctgcctgcccctttgtcacctcggttggatcgactcgctacctcaacgagaccgcaaccggcttctcatctggtggtttctccgactactggaagcgcccatcgtaccaggacgatgctgttaaggcgtatttccaccacctcggtgagaaattcaagccatacttcaaccgccacggccgtggattccccgacgttgcaacccagggatatggcttccgcgtctacgaccagggcaagctcaagggtctccaaggtacttctgcctccgcgcctgcattcgccggtgtgattggtctcctcaacgacgcgcgattgaaggcgaagaagcctaccttgggattcctaaacccactgctttactctaactcagacgcgctaaatgacattgttctcggtggaagcaagggatgcgatggtcatgctcgctttaacgggccgccaaatggcagcccagtaatcccatatgcgggatggaacgcgactgctgggtgggatccagtgactggtcttggaacgccgaacttccccaagcttcttaaggctgcggtgcctagccggtacagggcgtga

SEQ ID NO: 59 - DNA, 1830 bp, Trichoderma atroviride
atggcgaaactgacagctcttgccggtctcctgacccttgccagcgtgcaggcaaatgccgccgtgctcttggacagcctcgacaaggtgcctgttggatggcaggctgcttcggccccggccccgtcatccaagatcaccctccaagttgccctcacgcagcagaacattgatcagttggaatcaaagctcgctgccgtctccacgcccaactccagcaactatggaaagtacctggatgtcgatgagattaaccaaatcttcgctcccagcagcgccagcaccgctgctgttgagtcctggctcaagtcgtacggcgtggactacaaggtgcagggcagcagcatctggttccagacggatgtctccacggccaacaagatgctcagcacaaacttccacacttacaccgactcggttggtgccaagaaagtgcgaactctccagtactcggtccccgagaccctggccgaccacatcgatctgatttcgcccacaacctactttggcacgtccaaggccatgcgggcgttgaagatccagaacgcggcctctgccgtctcgcccctggctgctcgtcaggagccctccagctgcaagggcacaattgagtttgagaaccgcacattcaacgtcttccagcccgactgtctcaggaccgagtacagcgtcaacggatacaagccctcagccaagtccggtagcaggattggcttcggctctttcctgaaccagagcgccagctcctcagatctcgctctgttcgagaagcactttggctttgccagccagggcttctccgtcgagctcatcaatggcggatcaaacccccagccgcccacagacgccaatgacggcgaggccaacctggacgcccagaacattgtgtcgtttgtgcagcctctgcccatcaccgagtttattgctggaggaactgcgccgtacttcccagaccccgttgagccggctggaactcccgatgagaacgagccttacctcgagtactacgagtacctgctctccaagtcaaacaaggagcttccccaagtcatcaccaactcctacggtgatgaggagcagactgttccccaggcatatgccgtccgcgtgtgcaacctcattggattgatgggccttcgtggtatctctatcctcgagtcatccggtgatgagggtgttggtgcctcttgtctcgctaccaacagcaccaccactccccagttcaaccccatcttcccggctacatgcccctatgtcaccagtgttggtggaaccgtcagcttcaaccccgaggttgcctgggacggctcatccggaggcttcagctactacttctcaagaccttggtaccaggaggccgcagtcggcacataccttaacaagtatgtcagcgaggagaccaaggaatactacaagtcgtatgtcgacttttccggacgtggcttccccgatgttgcagctcacagcgtgagccccgattaccccgtgttccaaggcggcgagcttacccccagcggcggtacttctgcggcctctcccatcgtggccagtgttattgccctcctgaacgatgctcgtctccgtgcaggcaagcctgctctcggattcttgaaccctctgatctacggatatgcctacaagggctttaccgatatcacgagtggccaagctgtcggctgcaacggcaacaacactcaaactggaggccctcttcctggtgcgggtgttattccaggtgctttctggaacgcgaccaagggctgggatcctacaactggattcggtgtccccaacttcaagaagctgcttgagcttgtccgatacatttag

SEQ ID NO: 60 - DNA, 1791 bp, Arthroderma benhamiae
atgcgtcttctcaaatttgtgtgcctgttggcatcagttgccgccgcaaagcctactccaggggcgtcacacaaggtcattgaacatcttgactttgttccagaaggatggcagatggttggtgccgcggaccctgctgctatcattgatttctggcttgccatcgagcgcgaaaacccagaaaagctctacgacaccatctatgacgtctccacccctggacgcgcacaatatggcaaacatttgaagcgtgaggaattggatgacttactacgcccaagggcagagacgagtgagagcatcatcaactggctcaccaatggtggagtcaacccacaacatattcgggatgaaggggactgggtcagattctctaccaatgtcaagactgccgaaacgttgatgaatacccgcttcaacgtcttcaaggacaacctaaattccgtttcaaaaattcgaactttggagtattccgtccctgtagctatatcagctcatgtccaaatgatccagccaactaccttatttggacgacagaagccacagaacagtttgatcctaaaccccttgaccaaggatctagaatccatgtccgttgaagaatttgctgcttctcagtgcaggtccttagtgactactgcctgccttcgagaattgtacggacttggtgaccgtgtcactcaggctagggatgacaaccgtattggagtatccggctttttggaggagtacgcccaataccgcgatcttgagctcttcctctctcgctttgagccatccgccaaaggatttaatttcagtgaaggccttattgccggaggaaagaacactcagggtggtcctggaagctctactgaggccaaccttgatatgcaatatgtcgtcggtctgtcccacaaggcaaaggtcacctattactccaccgctggccgtggcccattaattcccgatctatctcagccaagccaagcttcaaacaacaacgaaccataccttgaacagctgcggtacctcgtaaagctccccaagaaccagcttccatctgtattgacaacttcctatggagacacagaacagagcttgcccgccagctataccaaagccacttgcgacctctttgctcagctaggaactatgggtgtgtctgttatcttcagcagtggtgataccgggcccggaagctcatgccagaccaacgatggcaagaatgcgactcgcttcaaccctatctacccagcttcttgcccgtttgtgacctccatcggtggaaccgttggtaccggtcctgagcgtgcagtttcattctcctctggtggcttctcagacaggttcccccgcccacaatatcaggataacgctgttaaagactacctgaaaattttgggcaaccagtggagcggattgtttgaccccaacggccgtgctttcccagatatcgcagctcagggatcaaattatgctgtctatgacaagggaaggatgactggagtctccggcaccagtgcatccgcccctgccatggctgccatcattgcccagcttaacgatttccgactggcaaagggctctcctgtgctgggattcttgaacccatggatatattccaagggtttctctggctttacagatattgttgatggcggttccaggggttgcactggttacgatatatacagcggcttgaaagcgaagaaggttccctacgcaagctggaatgcaactaagggatgggacccagtaacgggatttggtactcccaacttccaagctctcactaaagtgctgccctaa

SEQ ID NO: 61 - DNA, 1803 bp, Fusarium graminearum
atgtatatcacctcatcccgcctcgtgctggccttagcggcacttccgacagcatttggtaaatcatactcccaccatgccgaagcaccaaagggatggaaggtcgacgacaccgctcgtgttgcctccaccggtaaacaacaggtcttcagcatcgcactgaccatgcaaaatgttgatcagctcgagtccaagctccttgacctctccagccccgacagcaagaactatggccagtggatgtctcaaaaggacgtaacaactgctttctatccttcgaaagaagctgtttccagtgtgacaaagtggctcaagtccaagggtgtcaagcactacaacgtcaacggtggtttcattgactttgctctcgatgtcaagggtgccaatgcgctacttgatagtgactatcaatactacaccaaagagggccagaccaagttgcgaactctgtcttactctatccctgatgatgtagccgaacacgttcagttcgtcgacccaagcaccaactttggcggcacactggctttcgcccctgtcactcacccatcgcgtactctaaccgagcgcaagaacaagcccaccaagagcacagtcgatgcttcatgccaaaccagcatcacaccctcatgcttgaagcagatgtacaacattggtgactacactcccaaggtcgagtctggaagcactattggtttcagcagcttccttggcgagtccgccatctactccgatgttttcctgtttgaggagaagtttggaattcccacgcagaactttaccactgttctcatcaacaacggcactgatgaccagaacactgctcacaagaactttggcgaggctgacttggatgccgagaacattgttggaattgcccaccctcttcccttcacccagtacatcactggcggttcaccaccttttcttcccaacatcgatcagccaactgctgccgataaccagaacgagccttatgtgcctttcttccgctaccttctatcgcagaaggaagtccctgcagttgtctctacctcgtatggtgacgaagaagatagcgtccctcgcgaatatgctaccatgacctgcaacctgattggtcttctcggacttcgaggaatcagtgtcatcttctcctctggcgatatcggcgttggtgctggatgtctcggccctgaccacaagactgtcgagttcaacgccatcttccctgccacctgcccttacctcacctccgtcggcggtaccgttgatgtcacccccgaaatcgcctgggaaggttcttctggtggtttcagcaagtacttcccccgacccagctaccaggacaaggctgtcaagacgtacatgaagactgtctccaagcagacaaagaagtactacggcccttacaccaactgggaaggccgaggcttccctgatgttgctggccacagtgtctctcccaactatgaggttatctatgctggtaagcagagtgcaagcggaggtaccagtgctgctgctcctgtttgggctgccattgtcggtctgctcaacgatgcccgtttcagagctgggaagccaagcttgggatggttgaaccctcttgtttacaagtatggaccaaaggtgttgactgacatcactggtggttacgccattggatgtgatggcaacaacacccagtccggaaagcctgagcctgcaggatccggtattgtgcccggtgccagatggaatgccactgccggatgggatcctgtcactggttatggtacacccgactttggaaagttgaaggatttggttcttagcttctaa

SEQ ID NO: 62 - DNA, 1788 bp, Aspergillus kawachii
atgcgttcctccggtctttacgcagcactgctgtgctctctggccgcatcgaccaacgcagttgttcatgagaagctcgccgcggtcccctcgggctggcaccatctcgaagatgctggctccgatcaccagattagcctgtcgatcgcattggcacgcaagaacctcgatcagcttgaatccaagctgaaagacttgtccacaccaggtgaatcgcagtatggccagtggctggatcaagaggaagtcgacacactgttcccagtggccagcgacaaggccgtgatcagctggttgcgcagcgccaacatcacccatattgcccggcagggcagcttggtgaactttgcgaccaccgtcgacaaggtgaacaagcttctcaacaccacttttgcttactaccaaagaggttcttcccagagactgcgcacgacagagtactccattcccgatgatctggtcgactcgatcgacctcatctccccgacaacctttttcggcaaggaaaagaccagtgctggcctgacccagcggtcgcagaaagtcgacaaccatgtggccaaacgctccaacagctcgtcctgcgccgataccatcacgttatcctgcctgaaggagatgtacaactttggcaactacactcccagcgcctcgtcaggaagcaagctgggattcgccagcttcctgaacgagtccgcctcgtattccgatcttgccaagttcgagagactgttcaacttgccgtctcagaacttctccgtggagctgatcaacggcggcgtcaatgaccagaaccaatcgacggcttctctgaccgaggctgacctcgatgtggaattgctcgttggcgtaggtcatcctcttccggtgaccgagtttatcacttctggcgaacctcctttcattcccgaccccgatgagccgagtgccgccgataatgagaatgagccttaccttcagtactacgagtacctcctctccaagcccaactcggccctgccccaagtgatttccaactcctacggtgacgacgaacagaccgttccagaatactacgccaagcgagtctgcaacctgatcggactggtcggcctgcgcggcatcagcgtcctggaatcatccggtgacgaaggaattggatctggctgccgcaccaccgacggcactaacagcacccaattcaatcccatcttccccgccacctgtccctacgtgaccgccgtaggaggcaccatgtcctacgcgcccgaaattgcctgggaagccagttccggtggtttcagcaactacttcgagcgagcctggttccagaaggaagccgtgcagaactacctggcgaaccacatcaccaacgagacgaagcagtattactcacaattcgctaactttagcggtcgcggatttcccgatgtttcggcccatagctttgagccttcgtacgaagttatcttctacggcgcccgttacggctccggcggtacttccgccgcatgtcctctgttctctgcgctagtgggcatgttgaacgatgctcgtctgcgggcgggcaagtccacgcttggtttcttgaaccccctgctgtacagtaaggggtacaaggcgctgacagatgtcacggcgggacaatcgatcgggtgcaatggcattgatccgcagagtgatgaggctgttgcgggcgcgggcattatcccgtgggcgcattggaatgccacagtcggatgggatccggtgacgggattgggacttcctgattttgagaagttgaggcagttggtgctgtcgttgtag

SEQ ID NO: 63 - DNA, 1818 bp, Talaromyces stipitatus
atgagtcgaaatctcctcgttggtgctggcctgttggccctcgcccaattgagcggtcaagctctcgctgccgctgccctcgtcggccatgaatccctagctgcgctgccagttggctgggataaggtcagcacgccagctgcagggacgaacattcaattgtccgtcgccctcgctctgcaaaacatcgagcagctggaagaccacttgaagtctgtgtcaacccccggttctgccagctacggtcagtacctggattccgacggtattgccgctcaatacggtcccagcgacgcatccgttgaggctgtcaccaactggctgaaggaggccggtgtcactgacatctacaacaacggccagtcgattcacttcgcaaccagtgtcagcaaggccaacagcttgctcggggccgatttcaactactattctgatggtagtgcgaccaagttgcgtaccttagcttattccgttcccagtgacctcaaagaggccatcgaccttgtctcgcccaccacctatttcggcaagaccactgcttctcgtagcatccaggcttacaagaacaagcgcgcctctactacttccaagtctggatcgagctctgtgcaagtatctgcttcctgccagaccagcatcactcctgcctgcttgaaacagatgtacaatgttggcaactacacacccagcgtcgctcacggcagtcgtgtcggattcggtagcttcttgaatcaatctgccatctttgacgacttgttcacctacgaaaaggtcaatgatattccatcacagaatttcactaaggtgattattgcaaatgcatccaacagccaagatgccagcgatggcaactacggcgaagccaaccttgacgtgcaaaacattgtcggcatctctcatcctctccccgtgactgaattcctcactggtggctcacctcccttcgttgctagcctcgacacccctaccaaccagaacgagccatatattccttactacgaatatcttttgtctcagaagaacgaggatctcccccaggtcatttccaactcttacggagacgacgagcagtctgtgccgtacaagtatgccatccgtgcatgcaacctgatcggcctgacaggtttacgaggtatctcggtcttggaatccagcggtgatctcggcgttggagccggctgtcgcagcaacgatggcaagaacaagactcaatttgaccccatcttccctgccacttgcccctacgttacctctgttggtggtacccaatccgttacccctgaaattgcctgggtcgccagctccggtggtttcagcaactacttccctcgtacctggtaccaggaacccgcaattcagacctatctcggactccttgacgatgagaccaagacatactattctcaatacaccaactttgaaggccgtggtttccccgatgtttccgcccacagcttgacccctgattaccaggtcgtcggtggtggctatctccagccaagcggtggtacttccgctgcttctcctgtctttgccggcatcattgcgcttttgaacgacgctcgtctcgctgctggcaagcccactcttggcttcttgaacccgttcttctacctttatggatacaagggtttaaacgatatcactggaggacagtcagtgggttgcaacggtatcaacggccaaactggggctcctgttcccggtggtggcattgttcctggagcggcctggaactctactactggctgggacccagccactggtctcggaacacccgacttccagaagttgaaagaactcgtacttagcttttaa

SEQ ID NO: 64 - DNA, 1800 bp, Fusarium oxysporum
atgtatatctcctcccaaaatctggtactcgccttatcggcgctgccttcagcatttggcaaatccttctctcaccatgctgaagctcctcaaggctggcaagtccaaaagactgccaaagtcgcttccaacacgcagcatgtcttcagtcttgcactaaccatgcaaaacgtggatcagctcgaatccaagcttcttgacctctccagccccgacagcgccaactacggtaactggctctcccacgatgagctcacaagcactttctctccttccaaggaggcggtggctagtgtgacaaagtggctcaagtcaaagggcatcaagcactacaaggtcaacggtgctttcattgactttgctgctgatgttgagaaggccaatacgcttctcggaggtgattaccagtactacactaaggatggtcagacgaagctgagaacgctgtcttactccattcctgatgatgtcgccggtcacgttcaatttgttgatcctagcacaaacttcggtggcaccgttgcgttcaaccctgtgcctcacccctcgcgcaccctccaagagcgcaaggtctctccctccaagagcaccgttgatgcttcatgccagacaagcatcaccccttcttgcctcaagcagatgtacaacattggagactacactcccgatgccaagtctggaagtgagattggtttcagcagctttctcggccaggctgctatttactctgatgtcttcaagtttgaggagctgtttggtattcctaagcagaactacaccactattctgatcaacaatggcaccgatgatcagaatactgcgcatggaaactttggagaggctaaccttgatgctgagaacattgttggaatcgctcatcctcttcctttcaagcagtacattactggaggttcaccacctttcgttcccaacatcgatcagcccaccgagaaggataaccagaacgagccctacgtgcctttcttccgttacctcttgggccagaaggatctcccagccgtcatctccacttcctacggcgatgaagaagacagcgttcctcgtgagtatgctacactcacctgcaacatgatcggtcttctcggtctccgtggcatcagtgtcatcttctcttccggtgacatcggtgtcggttccggctgccttgctcccgactacaagaccgtcgagttcaatgccatcttccccgccacatgcccctacctcacctccgtcggcggtaccgtcgacgtcacccccgagatcgcctgggagggatcctccggcggattcagcaagtacttcccccgacccagctaccaggacaaggccatcaagaagtacatgaagacagtctccaaggagaccaagaagtactacggcccttacaccaactgggagggccgaggtttccctgatgtcgctggacacagtgttgcgcctgactacgaggttatctacaatggtaagcaggctcgaagtggaggtaccagcgctgctgcccctgtttgggctgctatcgttggtctgttgaacgatgcccgcttcaaggctggtaagaagagcttgggatggttgaaccctcttatctacaagcatggacccaaggtcttgactgacatcaccggtggctatgctattggatgtgacggtaacaacactcagtctggaaagcccgagcccgctggatctggtcttgttcccggtgctcgatggaacgccacagctggatgggatcctaccactggctatggaactcccaacttccagaagttgaaggacttggttctcagcttgtaa

SEQ ID NO: 65 - DNA, 1839 bp, Trichoderma virens
atgcctaagtccacagcgcttcggcttgttagcctcctttccctggccagtgtgccgatatctgcctccgtccttgtggaaagtctcgaaaagctgcctcacggatggaaagctgcttcggctcctagcccttcctcccagataaccctacaagtcgctcttacgcagcagaacatcgatcagctggaatcgaggctcgcggctgtatccacaccaaattccaagacatacggaaattatctggatcttgatgagatcaatgagatcttcgcgccaagcgatgccagcagcgcagccgtggagtcttggctccattctcacggtgtgacaaaatacacgaagcaaggcagcagtatctggttccaaaccgaagtttctacagcaaatgcaatgttgagcacaaacttccacacttacagtgatgctgctggcgttaagaagttgcgaactcttcagtattcaattccggagagtcttgtgggccatgtcgatctcatctcacccacgacctactttggcacctctaacgctatgagagctttgagatctaaaagcgtggcttcagttgctcaaagtgtggcagcccgccaagaaccttctagctgcaagggaactctggttttcgaaggaagaacgttcaatgtcttccaaccagattgtcttaggacagagtacaatgtcaatggatacactccatcagccaagtctggtagtagaataggatttggttccttcttaaaccaaagtgcaagcttttcagacctcgcactctttgaaaaacactttgggttttccagccaaaatttctccgtcgttctgatcaatggtggaacggacctgccccaaccaccctctgacgacaacgatggcgaggccaatttggatgtccaaaacattttgacaatcgcacaccctctgcccatcactgaattcatcactgccggaagcccgccgtacttcccagatcccgttgaacctgcaggaactcccgatgagaacgagccttacttgcagtactttgagtatctgttgtcgaagcccaacagagatcttcctcaggtcattaccaactcttacggtgatgaggagcaaacagtacctcaggcttatgctgtccgagtgtgcaacctaattggattgatgggactgcgtggtatcagtatcctcgagtcctccggcgatgagggagtgggtgcttcctgcgttgctaccaacagcaccactcctcaatttaaccccattttcccggcaacatgcccctatgtcactagcgtaggtggaactgtgaacttcaacccagaagttgcctgggacggttcatctggaggtttcagctactatttctccaggccatggtaccaagaggaagcagttggaaactacctagagaagcatgtcagcgccgaaacaaagaagtactacgggccttatgtcgatttctctggacgtggcttccctgatgttgcagctcacagcgtgagccccgattatcctgtgttccaaggcggccagctcactcctagcggaggcacttctgcggcttctcccgtcgtagccagtatcattgcccttctgaacgatgcacgcctccgtgaaggcaagcccacacttgggttcctgaacccgctgatttaccaatatgcttacaagggtttcacggatatcacatccggccagtctgatggctgcaatggcaacaacacccaaacggatgcccctcttcctggagctggcgttgtcctaggagcacactggaatgcgaccaaaggatgggatcctacgacaggatttggtgtccctaactttaagaagctactcgagctgatccgatatatatag

SEQ ID NO: 66 - DNA, 1776 bp, Trichoderma atroviride
atggctaaactgacggcacttcggctcgtcagccttctttgccttgcggctgcgcaggcctctgctgctgtgctcgtggaaagcctcaaacaagtgcccaacgggtggaatgcagtctcgaccccagacccttcgacatcgattgtcttgcaaatcgccctcgcgcaacagaatatcgatgaattggaatggcgtctcgcggctgtatccacgcccaactctggcaattatggcaaatacctggatattggagagattgaaggaattttcgccccaagcaatgcctcttacaaagccgtggcatcgtggctccagtctcatggggtgaagaacttcgtcaaacaagccggcagtatttggttctacactactgtctctaccgcaaacaagatgcttagcacagatttcaaacactatagcgatcctgttggcattgagaagctgcgtactcttcagtactcgatcccagaagaactagtcggccatgttgatctcatctcgcctacaacatattttggaaacaaccaccccgcgacagcgagaacacccaacatgaaggccattaacgtaacctaccaaatctttcacccagactgccttaaaacgaaatacggcgttgatggctatgccccatctccaagatgtggcagcaggattggttttggctcattcctcaacgaaactgccagttattcggatcttgcgcagtttgagaagtactttgaccttcccaaccaaaacctttccaccttattgatcaatggcgcaatcgacgttcagccaccttccaacaaaaacgacagcgaggccaacatggacgttcagaccatcttgacctttgtccaacctcttcctattactgagtttgttgttgccggaatcccgccgtatattcctgatgcggctttgccgatcggcgaccctgtccaaaacgagccgtggctggaatactttgagtttttgatgtccaggaccaacgcagagcttccccaggtcattgccaactcatacggtgacgaggaacaaacggtaccacaggcgtatgccgtccgagtatgcaaccagattgggctgttgggccttcgcggtatatccgttatcgcatcatctggcgatacgggtgttggaatgtcttgtatggcttcgaacagcactactcctcagtttaaccccatgttcccggcttcgtgtccttatatcaccactgtcggtggaactcagcaccttgataatgagattgcttgggagctttcatcgggaggcttcagtaactatttcacaaggccatggtatcaagaagacgcagccaaaacatatcttgaacgtcatgtcagcaccgagacaaaggcatattacgaacgttacgccaatttcttgggacgcggctttcccgacgttgcagcacttagtctcaaccccgattatccagtgattattggcggagaacttggtcccaatggaggcacttctgcggccgcacccgtcgtcgctagtattattgcactcttgaacgatgcacgcctttgcctaggcaaacctgcccttgggttcttgaaccccctgatctatcaatatgctgataagggtggcttcacggatatcacgtccggccagtcttggggctgtgccggaaataccactcagacggggcctcctccccctggagctggtgtcattccgggggcacactggaatgcgaccaagggatgggatcctgtaacaggatttggaaccccgaacttcaagaaattactctcactggccctgtccgtctaa

SEQ ID NO: 67 - DNA, 1749 bp, Agaricus bisporus
atgttttggcgtccagcttttgtccttcttctcgctcagcttgtcactgctagtcctttagctcgacgctgggatgatttcgcagaaaaacatgcctgggttgaagttcctcgcgggtgggaaatggtctccgaggctcccagtgaccatacctttgatcttcgcattggagtaaagtcaagtggcatggagcagctcattgaaaacttgatgcaaaccagcgatcctactcattccagatatggtcaacatcttagtaaagaagagctccatgatttcgttcagcctcatcctgattctaccggagcggtcgaagcatggcttgaagatttcggtatctccgatgatttcattgatcgtactggaagtggcaactgggttactgttcgagtttcagtagcccaggctgaacgtatgcttggtaccaagtataacgtctaccgccattctgaatcaggggaatcggttgtacgaacaatgtcttattcgcttcccagcgaacttcactcccacatagatgttgtcgcacccaccacttatttcggcacgatgaaaagcatgcgggtgaccagcttcttacagccggaaatagagcctgttgacccaagcgctaaaccatcggctgctccagcttcctgtttgagtaccactgtcataacccccgattgcctccgtgacctttataatacggctgactacgttccttccgccacttcacggaatgccattggtattgctgggtacttggatcgttcaaatcgtgcagatcttcagactttcttccgacgcttccggcccgatgccgttggcttcaattacacgactgtccaactaaatggcggaggagacgaccagaatgatcccggtgtagaggccaacctcgatattcaatacgccgctggtattgctttccccacaccagctacatactggagtactggcggctctccacctttcattccagatactcaaaccccgacaaacaccaatgagccctacctggattggatcaattttgtcctaggccaggacgagattccacaggtgatttcaacgtcctatggtgacgacgagcaaacagttcctgaagattacgctactagcgtgtgtaatctcttcgcgcaactcggcagccgtggcgttacagtattcttctccagcggtgactttggtgttggtggtggagattgcctcacgaatgatggctcaaaccaagtccttttccagccggctttccccgcttcctgcccattcgtaacagctgttggcggaactgtcaggcttgatcctgagattgctgtcagtttctctggaggaggcttttcccgttacttctccaggccatcgtaccagaatcaaactgtggctcaatttgtttctaatcttgggaatacattcaacggactctacaataaaaatggaagggcctacccagatcttgcagcacagggcaatggcttccaagttgttatagacggcatcgtccgttcggttggagggaccagcgccagctctccgacggttgccggtatctttgcgcttttgaatgacttcaagctctcaagaggccagtcgacactcggatttatcaacccacttatatactcctccgctacatccggcttcaatgacatcagggcgggtacaaaccctggttgtggtactcgcggatttaccgctggtactggttgggatccggtcactggtctgggcactcccgattttttgaggcttcagggacttatttaa

SEQ ID NO: 68 - DNA, 1806 bp, Magnaporthe oryzae
atgctcgaccgtatccttctccccctcggcctcctggcctcccttgccaccgctcgtgtctttgacagcctaccccaccctccccgaggctggtcatactcgcacgcggcggaatcgacggagccgctgaccctgcgcatcgccctccgccagcaaaatgccgccgccctggagcaggtggtgctgcaggtctcgaaccccaggcacgccaattacggccagcacctgacgcgcgacgagctgcgcagctacacggcgcccacgccgcgggccgtccgcagcgtgacgtcgtggctggtcgacaacggcgtcgacgactacacggtcgagcacgactgggtgacgctgcgcacgacggtcggggccgcggacaggctgctcggcgcagactttgcctggtatgccggcccgggcgagacgctgcagctgcggacgctctcgtacggcgtcgacgactcggtggcgccgcacgtcgacctcgtgcagcccacgacgcggtttggcggtcccgtcgggcaggcgtcgcacatcttcaagcaggacgactttgacgagcagcagctcaagaccttgtcggtggggttccaggtcatggctgacctgccggccaacgggcctgggtcgatcaaggcggcatgtaacgagtctggcgtgacgcccctgtgcctgcgaactctgtacagggtcaactacaagccggcaaccacggggaacctggtcgctttcgcgtcgttcctggagcagtacgccaggtacagtgatcagcaggcattcactcagcgggtccttggccctggtgttccgttgcagaacttttcggtcgaaacggtcaacggtggagccaatgaccagcagagcaaacttgacagcggcgaggcgaacctcgatctgcagtacgtcatggcaatgagccaccctattccaattttggagtacagcactggaggcagaggacccctcgtcccaactctggaccagcccaacgccaacaacagcagcaatgagccttacctggagttcctgacgtacctcctggcccaacccgactcagccatccctcagaccctgtcggtgtcgtatggcgaggaggaacagtcggtgccgcgcgactacgccatcaaggtttgcaacatgttcatgcagctcggcgcccgcggcgtgtcggttatgttttcgtcgggcgactcgggcccgggtaatgactgtgttcgagcctcggacaacgcaaccttttttggctcaacattccccgcaggctgcccctacgtcacgtcggtgggctccaccgtcggcttcgagccggagcgcgccgtctccttttcctcgggcggcttcagcatttaccacgctcgccccgactaccaaaacgaagtggtccccaagtacattgaatcgatcaaggcttcgggctacgaaaagttctttgacggcaacggccgcggaattcccgacgtggctgcccagggcgcccgcttcgtcgtcatcgacaagggccgcgtttctctaatctcggggaccagcgccagctcacctgcgtttgctggcatggtggcgctcgtcaacgccgcccgcaagtcaaaggacatgccggccttgggcttcctcaaccccatgctgtaccagaacgccgcggccatgacggacattgtcaacggcgctggcatcggctgcaggaagcaacgtacagaattcccgaatggcgccaggttcaacgccacggccggctgggatcccgtcacagggctggggacgccgttgtttgacaagctgctggctgttggcgcacctggagttcccaacgcgtga

SEQ ID NO: 69 - DNA, 1845 bp, Togninia minima
atgcgtagccagttgctcttctgcacagcatttgctgctctccagtcgcttgtggagggcagcgatgtggtgttggagtcattgcgagaggtccctcagggctggaagaggcttcgagatgcggaccccgagcagtccatcaagctgcgcattgcgcttgagcagcctaacctggacctgttcgagcagaccctctacgacatctcgtcaccggatcacccaaaatatggccagcatctcaagagccacgagttacgggatattatggcacctcgcgaggagtcaactgctgctgtcatcgcttggctgcaagacgctgggctttctggctcgcagattgaggacgacagcgactggatcaacatccagacgacagtcgcccaagccaacgacatgctgaacacgactttcggtctcttcgcccaggaaggcaccgaggtcaatcgaattcgagctctggcatattccgtgcctgaggagatcgtccctcacgtcaagatgattgctcccatcatccgcttcggtcagttgagacctcagatgagccacatcttctcgcatgagaaagtcgaggagaccccgtctattggcaccatcaaggccgccgctatcccatctgtggatcttaacgtcaccgcttgcaatgccagcatcacccccgagtgcctccgagcgctttacaacgttggtgattacgaggcggacccatcgaagaagtctcttttcggagtctgtggctacttggagcaatatgccaagcacgatcagctggccaagtttgagcagacctacgctccgtatgctatcggtgccgacttcagcgtcgtgaccatcaatggcggaggcgacaaccagaccagtacgatcgatgatggagaagccaacctggatatgcagtatgctgtcagcatggcatacaagacgccaatcacatactattcaactgggggtcgaggacctcttgttccagatctcgaccaacctgatcccaacgacgtctcaaacgagccgtaccttgattttgtgagctaccttctcaagctgcccgactccaaattgccgcagaccatcacaacttcgtacggagaggatgagcaatccgttccacgctcctacgtggagaaggtctgcaccatgttcggcgcgctcggtgcccgaggcgtgtctgtgatcttctcctctggtgataccggtgtcggctcagcgtgccagaccaacgacggcaagaacaccacccgcttcctgcctatattccctgctgcgtgcccttatgtgacctcggttggaggcactcgctatgtcgacccggaagtcgctgtgtccttctcgtctggaggcttctcggacatcttccctacgccactctaccagaagggcgctgtctctggctacctgaagatcctcggcgatcgctggaagggcctctataaccctcacggccgcggtttccctgacgtctccggacagagtgtcagataccacgtcttcgactacggcaaggacgtcatgtactctggcacaagtgcctctgcaccgatgttcgccgcgcttgtctcgctgctgaacaacgcccgtctcgcaaagaagttgccgcccatgggattcctgaatccctggctgtataccgttggttttaacgggctgacggatattgtgcacggtggatctactgggtgcactggcacagacgtgtacagcggcctgcccacacctttcgttccgtatgcgtcttggaacgcaaccgtgggatgggaccccgttactggacttggcacgcctctctttgataagctgctcaatttgagcacgccaaacttccacttgccgcacattggcggtcactag

SEQ ID NO: 70 - DNA, 1914 bp, Bipolaris maydis
atgaagtacaacacactcctcaccggcctgctggctgttgcccatggcagtgccgtttccgcttcaactacttcacatgtcgagggtgaagttgtcgagcgacttcatggcgttcctgagggttggagtcaagtgggcgcccccaatccagaccagaagctgcgctttcgcatcgcagtacgctcggtgagtaattgcttttgtgaacccatgtttgaatcttgcggtgctttttactgaacataacaggcggatagcgagctgtttgagaggacgcttatggaggtttcttctcccagccatcctcgctacggacagcacctaaagcgacacgaactcaaggacctcatcaaaccgcgcgccaagtcaacttcaaacatcctgaactggctgcaagagtctggaattgaggccagagatatccagaacgatggcgagtggatcagcttctatgctccggttaaacgtgccgagcaaatgatgagcactacattcaagacctatcagaacgaggcccgagcgaatatcaagaagatccgctctctagactactcggtgccgaagcacattcgagatgacatcgacatcatccagcctacgactcgcttcggccagatccaaccggagcgtagccaagtctttagtcaagaagaggtcccattctcagcgcttgttgtcaatgcgacgtgtaacaagaaaatcactcccgactgcctcgccaacctctacaacttcaaagactatgatgccagcgatgccaatgtcactatcggagtcagcggcttcctggagcaatatgctcgctttgacgacttgaagcaattcatcagcactttccaaccaaaagcagctggttccacattccaagttacatctgtcaatgcagggccttttgaccagaactcgacagccagcagtgttgaagccaatcttgacattcagtacacaacaggtcttgttgcgcccgacattgaaacccgctacttcactgttcccggtcgcggtatcctgatccctgatctggaccagcctacggagagcgacaacgctaatgagccgtatctggattactttacatatcttaataacctcgaagacgaagaactccccgacgtgctgaccacatcttacggcgagagcgagcagagtgtacccgccgaatatgcaaaaaaggtgtgcaatttgatcggccagttgggtgctcgtggtgtgtccgtcatcttctccagcggtgatactggccctggctctgcatgccaaaccaatgatggaaaaaacacgacacgtttcttgcccatcttccctgcttcttgcccctacgtcacttcggttggcggcactgttggtgttgagcccgaaaaggctgtcagcttctcttcgggcggcttttctgacctatggcctcgacccgcttatcaagagaaggccgtatcagaatatcttgaaaagctcggagaccgctggaacgggctttacaaccctcaaggacgcggatttcctgatgtagctgctcagggccaaggcttccaggtgtttgacaagggcaggctgatttcggtcggaggaacgagcgcttcagctcctgttttcgcatccgtagtcgcactcctgaacaatgctcgcaaggctgccggcatgtcttcactcggcttcttgaacccatggatctacgagcaaggctacaagggcttgaccgatatcgttgctggaggctcgacaggatgcacaggaagatccatctattcaggcctcccagcaccactcgtgccgtatgcttcttggaatgccaccgaaggatgggatccggtgacgggctatggtacacctgatttcaagcaattgctcaccctcgcgacggcacccaagtctggcgagcgtcgcgttcgtcgtggcggtctcggtggccaggcttag

SEQ ID NO: 71 - DNA, 1842 bp, Aspergillus kawachii
atgttatcttccttccttagccagggagcagccgtatccctcgcgttattgtcgctgctcccttcgcctgtagccgcggagatcttcgagaagctgtccggcgtccccaatggctggagatacgccaacaatcctcacggcaacgaggtcattcgccttcaaatcgcccttcagcagcacgatgttgccggtttcgaacaagccgtgatggacatgtccacccccggtcacgccgactatggaaagcatttccgcacacatgatgagatgaagcgcatgctgctccccagcgacactgccgtcgactcagttcgcgactggctggaatccgccggagtccacaatatccaggtcgacgccgactgggtcaagttccataccaccgtcaacaaggccaatgccctgctggatgccgacttcaagtggtatgtcagcgaggccaagcacattcgtcgtctacgcaccctgcaatactccatccccgacgccctggtctcgcacatcaacatgatccagcccaccactcgctttggccagatccagccgaaccgtgccaccatgcgcagcaagcccaagcacgccgacgagacattcctgaccgcagccaccttggcccagaacacctcccactgcgactccatcatcacgccgcactgtctgaagcagctctacaacatcggtgactaccaggccgaccccaagtccggtagcaaggtcggcttcgccagctacctcgaagaatacgcccggtatgccgatctcgaaaggttcgagcagcacctggctcccaacgccatcggccagaacttcagcgtcgttcaattcaacggcggcctcaacgaccagctttcattgagcgacagcggcgaagccaacctcgacctgcagtacatcctgggcgtcagcgctcccgtcccggtcactgaatacagcactggcggacgcggcgaactggtccccgacctgagctccccggaccccaacgacaacagcaacgagccctacctcgacttcctccagggtattctcaaactcgacaattccgacctcccccaagtcatctctacctcctacggcgaagacgaacagaccatccccgtcccctacgcccgcacagtctgcaatctctacgcccaactcggcagccgcggtgtctccgtgatcttctcgagcggcgactccggcgtcggcgccgcctgcctcaccaacgacggcaccaaccgcacccacttccctcctcaattcccggcctcctgcccctgggtaacctccgtcggtgccaccagcaaaacctccccggagcaagccgtctccttctcctcaggaggcttctccgacctctggccccgcccctcctaccaacaggctgccgtccaaacctacctcacccagcacctgggcaacaagttctcaggcctcttcaacgcctccggccgcgccttccccgacgtcgccgcgcagggcgtcaactacgccgtctacgacaagggcatgcttggccagttcgatggaaccagttgctccgcgccgacgttcagtggtgtcattgccttgttgaatgacgccagactgagggcgggtttgcccgttatgggattcctgaacccgttcctctatggagttggtagtgagagtggcgcgttgaatgatattgtcaacggcgggagcctgggttgtgatggtaggaatcgatttggaggcacgcccaatggaagtcccgttgtgccgtttgctagttggaatgcgaccaccgggtgggatccggtttctgggctgggaacgccggattttgcgaagttgaggggtgtggcgttgggtgaagctaaggcgtatggtaattaa

SEQ ID NO: 72 - DNA, 1977 bp, Aspergillus nidulans
auggcagcgacuggacgauucacugccuucuggaaugucgcgagcgugcccgccuugauuggcauucucccccuugcuggaucucauuuaagagcuguccuuugcccugucuguaucuggcgucacucgaaggccguuugugcaccagacacuuugcaagccaugcgcgccuucacccguguaacggccaucucccuggccgguuucuccugcuucgcugcugcggcggcugcggcuuuugagagccugcgagcugucccugacggcuggaucuacgagagcacccccgacccuaaccaaccgcugcgucuacgcaucgcgcugaaacagcacaaugucgccggcuucgagcaggcacugcuggauauguccacacccggucacuccagcuacgggcagcauuucggcuccuaccacgagaugaagcagcugcuucucccuaccgaggaggcguccuccucggugcgagacuggcucucggcggcgggcguugaguucgaacaggacgccgacuggaucaacuuccgcacgaccgucgaccaggcuaacgcccuccucgacgccgauuuccucugguacacaacgaccggcucgacgggcaacccgacgcggauccuccgaacccucuccuacagcguucccagcgagcucgcuggauacgucaacaugauccagccgacuacgcguuucggcggcacgcaugccaaccgggccaccguucgcgcgaagccgaucuuccucgagaccaaccggcagcucaucaacgccaucuccucuggcucgcucgagcacugcgagaaggccaucaccccaucgugccuggcggaucuguacaacacugaaggguacaaggcguccaaccgcagcgggagcaagguggccuuugccuccuuccucgaagaguacgcgcgcuacgacgaucucgccgaguucgaggagaccuacgcucccuaugcgaucgggcagaacuucucgguuaucuccaucaacggcggccucaacgaccaggacuccacggccgacagcggcgaggcgaaccucgaccugcaguacaucaucggcgucucgucgccgcuaccugugaccgaguucacaaccgguggccgcggcaagcucauuccugaccucuccucccccgacccgaaugacaacaccaacgagccuuuccuugacuuccuugaggccguccucaagcucgaucagaaagaccugccccaggucaucucgaccuccuacggcgaggacgagcagacaaucccugagccguacgcccgcuccgucugcaaccuguacgcucagcucgguucccgcggcgugucugugcucuucuccucgggugacucuggcgucggcgccgccugccagaccaacgauggcaaaaacacgacgcacuucccgccgcaguucccggccucuugccccugggugaccgccgucggcggcacgaacggcacagcgcccgaauccgguguauacuucuccagcggcggguucuccgacuacugggcgcgcccggcguaccagaacgccgcgguugagucauaccugcgcaaacucgguagcacacaggcgcaguacuucaaccgcagcggacgcgccuucccggacgucgcagcgcaggcgcagaacuucgcugucgucgacaagggccgugucggucucuucgacggaacgagcugcaguucgccuguauuugcgggcaucguggcguugcucaacgacgugcgucugaaggcaggccugcccgugcugggauuccucaacccuuggcucuaccaggauggccugaacgggcucaacgauaucguggauggagggagcaccggcugcgacgggaacaaccgguuuaacggaucgccaaaugggagccccguaaucccguaugcggguuggaacgcgacggaggggugggauccugugacggggcugggaacgccggauuucgcgaagcugaaagcgcucgugcuugaugcuuag

SEQ ID NO: 73 - DNA, 1815 bp, Aspergillus ruber
atgttgtcatttgttcgtcggggagctctctccctcgctctcgtttcgctgttgacctcgtctgtcgccgccgaggtcttcgagaagctgcatgttgtgcccgaaggttggagatatgcctccactcctaaccccaaacaacccattcgtcttcagatcgctctgcagcagcacgatgtcaccggtttcgaacagtccctcttggagatgtcgactcccgaccatcccaactacggaaaacacttccgcacccacgatgagatgaagcgcatgcttctccccaatgaaaatgccgttcacgccgtccgcgaatggctgcaagacgccggaatcagcgacatcgaagaagacgccgattgggtccgtttccacaccaccgtggaccaggccaacgacctcctcgacgccaacttcctctggtacgcgcacaagagccatcgtaacacggcgcgtctccgcactctcgagtactcgatcccagactctattgcgccgcaggtcaacgtgatccagccaaccacgcgattcggacagatccgtgccaaccgggctacgcatagcagcaagcccaagggtgggcttgacgagttggctatctcgcaggcagctacggcggatgatgatagcatttgtgaccagatcaccaccccacactgtctgcggaagctgtacaatgtcaatggctacaaggccgatcccgctagtggtagcaagatcggttttgctagtttcctggaggaatacgcgcggtactctgatctggtactgttcgaggagaacctggcaccgtttgcggagggtgagaactttactgtcgtcatgtacaacggcggcaagaatgaccagaactccaagagcgacagcggcgaggccaacctcgatctgcagtacatcgtgggaatgagcgcgggcgcgcccgtgaccgagttcagcaccgccggtcgcgcacccgtcatcccggacctggaccagcccgaccccagcgccggtaccaacgagccgtacctcgagttcctgcagaacgtgctacacatggaccaggagcacctgccgcaggtgatctctacttcctacggtgagaacgaacagaccatccccgaaaagtacgcccgcaccgtttgcaacatgtacgcgcagctgggcagccgcggtgtgtcggtgattttctcgtcgggcgactccggcgtcggctctgcctgtatgaccaacgacggtacaaaccgcacccacttccccccgcagttcccggcgtcctgcccctgggtgacatcggtcggggccactgagaagatggcccccgagcaagcgacatatttctcctcgggcggcttctctgacctcttcccgcgcccaaagtaccaggacgctgctgtcagcagctaccttcagaccctcggatcccggtaccagggcttgtacaacggttccaaccgtgcattccctgacgtctcggcgcagggtaccaactttgctgtgtacgacaagggccgtctaggccagttcgatggtacttcttgctctgctcccgcgtttagcggtatcatcgccttgctcaacgacgtccgtctccagaacaacaagcccgtcctgggcttcttgaacccctggttgtatggcgctgggagcaagggcctgaacgacgtcgtgcacggtggcagtacaggatgcgatggacaggagcggtttgcaggaaaggccaatggaagccccgtcgtgccgtacgctagctggaatgctacgcaaggctgggatccagtcactggccttggaacgccggatttcggcaagttgaaggatttggctctgtcggcttaa

SEQ ID NO: 74 - DNA, 1803 bp, Aspergillus terreus
auguugcccucucuuguaaacaacggggcgcugucccuggcugugcuuucgcugcucaccucguccgucgccggcgagguguuugagaagcugucggccgugccgaaaggauggcacuucucccacgcugcccaggccgacgcccccaucaaccugaagaucgcccugaagcagcaugaugucgagggcuucgagcaggcccugcuggacauguccaccccgggccacgagaacuacggcaagcacuuccacgagcacgacgagaugaaacgcaugcugcuccccagcgacuccgccgucgacgccguccagaccuggcugaccuccgccggcaucaccgacuacgaccucgacgccgacuggaucaaccugcgcaccaccgucgagcacgccaacgcccugcuggacacgcaguucggcugguacgagaacgaagugcgccacaucacgcgccugcgcacccugcaauacuccauccccgagaccgucgccgcgcacaucaacauggugcagccgaccacgcgcuuuggccagauccggcccgaccgcgcgaccuuccacgcgcaccacaccuccgacgcgcgcauccuguccgcccuggccgccgccagcaacagcaccagcugcgacucagucaucacccccaagugccucaaggaccucuacaaggucggcgacuacgaggccgacccggacucgggcagccaggucgccuucgccagcuaccucgaggaauacgcccgcuacgccgacauggucaaguuccagaacucgcucgcccccuacgccaagggccagaacuucucggucguccuguacaacggcggcgucaacgaccagucguccagcgccgacuccggcgaggccaaccucgaccugcagaccaucaugggccucagcgcgccgcuccccaucaccgaguacaucaccggcggccgcggcaagcucauccccgaucucagccagcccaaccccaacgacaacagcaacgagcccuaccucgaguuccuccagaacauccucaagcuggaccaggacgagcugccgcaggugaucucgaccuccuacggcgaggacgagcagacaaucccccguggcuacgccgaauccgucugcaacaugcuggcccagcucggcagccgcggcgugucgguggucuucucgucaggcgauucgggcgucggcgccgccugccagaccaacgacggccgcaaccaaacccacuucaacccgcaguucccggccagcugcccgugggugacgucggucggggccacgaccaagaccaacccggagcaggcgguguacuucucgucgggcggguucucggacuucuggaagcgcccgaaguaccaggacgaggcgguggccgcguaccuggacacgcugggcgacaaguucgcggggcuguucaacaagggcgggcgcgcguucccggacgucgcggcgcagggcaugaacuacgccaucuacgacaagggcacgcugggccggcuggacggcaccucgugcucggcgccggccuucucggccaucaucucgcugcugaacgaugcgcgccugcgcgaggguaagccgaccaugggcuucuugaacccguggcuguauggugagggccgcgaggcgcugaaugauguugucgugggugggagcaagggcugugaugggcgcgaccgguuuggcggcaagcccaaugggagcccugucgugccuuuugcuagcuggaaugcuacgcagggcugggacccgguuacugggcuggggacgccgaacuuugcgaagauguuggagcuggcgccauag

SEQ ID NO: 75 - DNA, 1806 bp, Penicillium digitatum
atgattgcatcattattcaaccgtagggcattgacgctcgctttattgtcactttttgcatcctctgccacagccgatgtttttgagagtttgtctgctgttcctcagggatggagatattctcgcacaccgagtgctaatcagcccttgaagctacagattgctctggctcagggagatgttgctgggttcgaggcagctgtgatcgatatgtcaacccccgaccaccccagttacgggaaccacttcaacacccacgaggaaatgaagcggatgctgcagcctagcgcggagtccgtagactcgatccgtaactggctcgaaagtgccggtatttccaagatcgaacaggacgctgactggatgaccttctataccaccgtgaagacagcgaatgagctgctggcagccaacttccagttctacatcaatggagtcaagaaaatagagcgtctccgcacactcaagtactctgtcccggacgctttggtgtcccacattaacatgatccagccaaccacccgtttcggccagctgcgcgcccagcgcgccattttacacaccgaggtcaaggataacgacgaggctttccgctcaaatgccatgtccgctaatccggactgcaacagcatcatcactccccagtgtctcaaggatttgtacagtatcggtgactatgaggccgaccccaccaatgggaacaaggtcgcgtttgccagctacctagaggagtatgcccgatactccgatctcgcattatttgagaaaaacatcgccccctttgccaagggacagaatttctccgttgtccagtataacggcggtggtaatgatcaacaatcgagcagtggcagtagtgaggcgaatcttgacttgcagtacatcgttggagtcagctctcctgttcccgttacagagtttagcactggaggtcgcggtgaacttgttccggatctcgaccagccgaatcccaatgacaacaacaacgagccataccttgaattcctccagaacgtgctcaagttgcacaagaaggacctcccccaggtgatttccacctcttatggcgaggacgagcagagcgttccagagaagtacgcccgcgccgtttgcaacctgtactcccaactcggtagccgtggtgtgtccgtaatcttttcatccggcgactctggcgttggcgccgcgtgtcagacgaacgacggccggaacgcgacccacttcccaccccagttcccggccgcctgcccctgggtgacatcagtcggtgcgacaacccacactgcgcccgaacgagccgtttacttctcatctggcggtttctccgatctctgggatcgccctacgtggcaagaagatgctgtgagtgagtacctcgagaacctgggcgaccgctggtctggcctcttcaaccctaagggccgtgccttccccgacgtcgcagcccagggtgaaaactacgccatctacgataagggttctttgatcagcgtcgatggcacctcttgctcggcacctgcgtttgccggagtcatcgccctcctcaacgacgcccgcatcaaggccaatagaccacccatgggcttcctcaacccttggctgtactctgaaggccgcagcggcctaaacgacattgtcaacggcggtagcactggctgcgacggtcatggccgcttctccggccccactaacggtggtacgtcgattccaggtgccagctggaacgctactaagggctgggaccctgtctccggtcttggatcgcccaactttgctgccatgcgcaaactcgccaacgctgagtag

SEQ ID NO: 76 - DNA, 1806 bp, Penicillium oxalicum
atgcatgttcctctgttgaaccaaggcgcgctgtcgctggccgtcgtctcgctgttggcctccacggtctcggccgaagtattcgacaagcttgtcgctgtccctgaaggatggcgattctcccgcactcccagtggagaccagcccatccgactgcaggttgccctcacacagggtgacgttgagggcttcgagaaggccgttctggacatgtcaactcccgaccaccccaactatggcaagcacttcaagtcacacgaggaagttaagcgcatgctgcagcctgcaggcgagtccgtcgaagccatccaccagtggctcgagaaggccggcatcacccacattcaacaggatgccgactggatgaccttctacaccaccgttgagaaggccaacaacctgctggatgccaacttccagtactacctcaacgagaacaagcaggtcgagcgtctgcgcaccttggagtactcggttcctgacgagctcgtctcgcacattaaccttgtcaccccgaccactcgcttcggccagctgcacgccgagggtgtgacgctgcacggcaagtctaaggacgtcgacgagcaattccgccaggctgctacttcccctagcagcgactgcaacagtgctatcaccccgcagtgcctcaaggacctgtacaaggtcggcgactacaaggccagtgcctccaatggcaacaaggtcgccttcaccagctacctggagcagtacgcccggtactcggacctggctctgtttgagcagaacattgccccctatgctcagggccagaacttcaccgttatccagtacaacggtggtctgaacgaccagagctcgcctgcggacagcagcgaggccaacctggatctccagtacattatcggaacgagctctcccgtccccgtgactgagttcagcaccggtggtcgtggtcccttggtccccgacttggaccagcctgacatcaacgacaacaacaacgagccttacctcgacttcttgcagaatgtcatcaagatgagcgacaaggatcttccccaggttatctccacctcgtacggtgaggacgagcagagcgtccccgcaagctacgctcgtagcgtctgcaacctcatcgctcagctcggcggccgtggtgtctccgtgatcttctcatctggtgattccggtgtgggctctgcctgtcagaccaacgacggcaagaacaccactcgcttccccgctcagttccccgccgcctgcccctgggtgacctctgttggtgctactaccggtatctcccccgagcgcggtgtcttcttctcctccggtggcttctccgacctctggagccgcccctcgtggcaaagccacgccgtcaaggcctaccttcacaagcttggcaagcgtcaagacggtctcttcaaccgcgaaggccgtgcgttccccgacgtgtcagcccagggtgagaactacgctatctacgcgaagggtcgtctcggcaaggttgacggcacttcctgctcggctcccgctttcgccggtctggtttctctgctgaacgacgctcgcatcaaggcgggcaagtccagcctcggcttcctgaacccctggttgtactcgcaccccgatgccttgaacgacatcaccgtcggtggaagcaccggctgcgacggcaacgctcgcttcggtggtcgtcccaacggcagtcccgtcgtcccttacgctagctggaacgctactgagggctgggaccccgtcaccggtctgggtactcccaacttccagaagctgctcaagtctgccgttaagcagaagtaa

SEQ ID NO: 77 - DNA, 1806 bp, Penicillium roqueforti
atgattgcatccctatttagtcgtggagcattgtcgctcgcggtcttgtcgcttctcgcgtcctctgctgcagccgatgtatttgagagtttgtctgctgttcctcaaggatggagatattctcgcaggccgcgtgctgatcagcccttgaagttacagatcgctctgacacagggggatactgccggcttcgaagaggctgtgatggagatgtcaacccccgatcaccctagctacgggcaccacttcaccacccacgaagaaatgaagcggatgctacagcccagtgcggagtccgcggagtcaatccgtgactggctcgaaggcgcgggtattaccaggatcgaacaggatgcagattggatgaccttctacaccaccgtggagacggcaaatgagctgctggcagccaatttccagttctacgtcagtaatgtcaggcacattgagcgtcttcgcacactcaagtactcagtcccgaaggctctggtgccacacatcaacatgatccagccaaccacccgtttcggccagctgcgcgcccatcggggcatattacacggccaggtcaaggaatccgacgaggctttccgctcaaacgccgtgtccgctcagccggattgcaacagtatcatcactcctcagtgtctcaaggatatatataatatcggtgattaccaggccaatgataccaatgggaacaaggtcgggtttgccagctacctagaggagtatgcacgatactccgatctggcactatttgagaaaaatatcgcgccctctgccaagggccagaacttctccgtcaccaggtacaacggcggtcttaatgatcaaagttccagcggtagcagcagcgaggcgaacctggacttgcagtacattgttggagtcagctctcctgttcccgtcaccgaatttagcgttggcggccgtggtgaacttgttcccgatctcgaccagcctgatcccaatgataacaacaacgagccataccttgaattcctccagaacgtgctcaagctggacaaaaaggaccttccccaggtgatttctacctcctatggtgaggacgagcagagcattcccgagaagtacgcccgcagtgtttgcaacttgtactcgcagctcggtagccgtggtgtatccgtcattttctcatctggcgactccggcgttgggtccgcgtgcctgacgaacgacggcaggaacgcgacccgcttcccaccccagttccccgccgcctgcccgtgggtgacatcagtcggcgcgacaacccataccgcgcccgaacaggccgtgtacttctcgtccggcggcttttccgatctctgggctcgcccgaaatggcaagaggaggccgtgagtgagtacctcgagatcctgggtaaccgctggtctggcctcttcaaccctaagggtcgtgccttccccgatgtcacagcccaaggtcgcaattacgctatatacgataagggctcgttgaccagcgtcgacggcacctcctgctcggcacctgccttcgccggagtcgtcgccctcctcaacgacgctcgcctcaaagtcaacaaaccaccaatgggcttccttaatccttggctgtactcgacagggcgcgccggcctaaaggacattgtcgatggcggcagcacgggttgcgatggcaagagccgcttcggtggtgccaataacggtggtccgtcgatcccaggtgctagctggaacgctactaagggttgggaccctgtttctggtctcgggtcgcccaactttgctaccatgcgcaagcttgcgaacgctgagtag

SEQ ID NO: 78 - DNA, 1806 bp, Penicillium rubens Wisconsin
augauugcaucucuauuuaaccguggagcauugucgcucgcgguauugucgcuucucgcgucuucggcuuccgcugauguauuugagaguuugucugcuguuccucaaggauggagauauucucgcagaccgcgugcugaucagccccugaagcuacagauugcucuggcacaaggggauacugccggauucgaagaggcugugauggacaugucaaccccugaucaccccagcuacgggaaccacuuccacacccacgaggaaaugaagcggaugcugcagcccagcgcggaguccgcagacucgauccgugacuggcuugaaagugcggguaucaauagaauugaacaggaugccgacuggaugacauucuacaccaccgucgagacggcaaaugagcugcuggcagccaauuuccaguucuaugccaacagugccaagcacauugagcgucuucgcacacuccaguacuccgucccggaggcucugaugccacacaucaacaugauccagccaaccacucguuucggccagcugcgcguccagggggccauauugcacacccaggucaaggaaaccgacgaggcuuuccgcucaaacgccguguccacuucaccggacugcaacaguaucaucacuccucagugucucaagaauauguacaaugugggugacuaccaggccgacgacgacaaugggaacaaggucggauuugccagcuaccuagaggaguaugcacgguacuccgauuuggaacuauuugagaaaaaugucgcacccuucgccaagggccagaacuucuccgucauccaguauaacggcggucuuaacgaucaacacucgagugcuagcagcagcgaggcgaaccuugacuuacaguacauuguuggaguuagcucuccuguuccaguuacagaguuuagcguuggcggucguggugaacuuguucccgaucuugaccagccugaucccaaugauaacaacaacgagccauaccuugaauuccuccagaacgugcucaagauggaacaacaggaccucccccaggugauuuccaccucuuauggcgagaacgagcagaguguucccgagaaauacgcccgcaccguaugcaacuuguucucgcagcuuggcagccgugguguguccgucaucuucgcaucuggcgacuccggcguuggcgccgcgugccagacgaaugacggcaggaacgcgacccgcuucccggcccaguucccugcugccugcccaugggugacaucggucggcgcgacaacccacaccgcgcccgagaaggccguguacuucucguccgguggcuucuccgaucuuugggaucgcccgaaauggcaagaagacgccgugagugacuaccucgacacccugggcgaccgcugguccggccucuucaauccuaagggccgugccuuccccgacgucucagcccaaggucaaaacuacgccauauacgauaagggcucguugaccagcgucgacggcaccucgugcucggcacccgccuucgccggugucaucgcccuccucaacgacgcccgccucaaggccaacaaaccacccaugggcuuccucaaucccuggcuguacucgacaggccgugacggccugaacgacauuguucauggcggcagcacuggcugugauggcaacgcccgcuucggcggccccgguaacggcaguccgaggguuccaggugccagcuggaacgcuacuaagggcugggacccuguuucuggucuuggaucacccaacuuugcuaccaugcgcaagcucgcgaacggugaguag

SEQ ID NO: 79 - DNA, 1785 bp, Neosartorya fischeri
augcuguccucgacucucuacgcaggguugcucugcucccucgcagccccagcccuugguguggugcacgagaagcucucagcuguuccuaguggcuggacacucgucgaggaugcaucggagagcgacacgaccacucucucaauugcccuugcucggcagaaccucgaccagcucgaguccaaguugaccacacuggcgaccccagggaacgcggaguacggcaaguggcuggaccaguccgacauugagucccuauuuccuacugcaagcgaugacgcuguuauccaauggcucaaggaugccggggucacccaagugucucgucagggcagcuuggugaacuuugccaccacugugggaacggcgaacaagcucuuugacaccaaguucuccuacuaccgcaauggugcuucccagaaacugcguaccacgcaguacuccauucccgauagccugacagagucgaucgaucugauugcccccacugucuucuuuggcaaggagcaagacagcgcacugccaccucacgcagugaagcuuccagcccuucccaggagggcagccaccaacaguucuugcgccaaccugaucacucccgacugccuaguggagauguacaaccucggcgacuacaagccugaugcaucuucgggcagucgagucggcuuugguagcuucuugaaucagucagccaacuaugcagaucuggcugcuuaugagcaacuguucaacaucccaccccagaauuucucagucgaauugauuaacggaggcgccaaugaucagaauugggccacugcuucccucggcgaggccaaucuggacguggaguugauuguagccgucagccacgcccugccaguaguggaguuuaucacuggcgguucaccuccguuuguucccaaugucgacgagccaaccgcugcggacaaccagaaugagcccuaccuccaguacuacgaguacuugcucuccaaacccaacucccaucuuccucaggugauuuccaacucguauggugacgaugaacagacuguucccgaguacuacgccaggagaguuugcaacuugaucggcuugaugggucuucgugguaucacugugcucgaguccucuggugauaccggaaucggcucggcgugcauguccaaugacggcaccaacacgccucaguucacuccuacauucccuggcaccugccccuucaucaccgcaguuggugguacacaguccuaugcuccugaaguugccugggacgccagcucggguggauucagcaacuacuucagccgucccugguaccaguauuucgcgguggagaacuaccucaauaaucacauuaccaaggacaccaagaaguacuauucgcaguacaccaacuucaagggccguggauucccugauguuucugcccauagcuugaccccugacuacgaggucguccuaacuggcaaacauuacaaguccgguggcacaucggccgccugccccgucuuugcugguaucgucggccuguugaaugacgcccgucugcgcgccggcaaguccacccuuggcuuccugaacccauugcuguauagcauacucgcggaaggauucaccgauaucacugccggaaguucuaucgguuguaaugguaucaacccacagaccggaaagccaguccccgguggugguaucauccccuacgcucacuggaacgcuacugccggcugggauccuguuacaggucuugggguuccugauuucaugaaguugaaggaguugguuuugucguuguaa

SEQ ID NO: 80 - DNA, 1707 bp, Aspergillus fumigatus
augcuguccucgacucucuacgcaggguggcuccucucccucgcagccccagcccuuuguguggugcaggagaagcucucagcuguuccuaguggcuggacacucaucgaggaugcaucggagagcgacacgaucacucucucaauugcccuugcucggcagaaccucgaccagcuugaguccaagcugaccacgcuggcgaccccagggaacccggaguacggcaaguggcuggaccaguccgacauugagucccuauuuccuacugcaagcgaugaugcuguucuccaauggcucaaggcggccgggauuacccaagugucucgucagggcagcuuggugaacuucgccaccacugugggaacagcgaacaagcucuuugacaccaaguucucuuacuaccgcaauggugcuucccagaaacugcguaccacgcaguacuccauccccgaucaccugacagagucgaucgaucugauugcccccacugucuucuuuggcaaggagcagaacagcgcacugucaucucacgcagugaagcuuccagcucuuccuaggagggcagccaccaacaguucuugcgccaaccugaucacccccgacugccuaguggagauguacaaccucggcgacuacaaaccugaugcaucuucgggaagucgagucggcuucgguagcuucuugaaugagucggccaacuaugcagauuuggcugcguaugagcaacucuucaacaucccaccccagaauuucucagucgaauugaucaacagaggcgucaaugaucagaauugggccacugcuucccucggcgaggccaaucuggacguggaguugauuguagccgucagccacccccugccaguaguggaguuuaucacuggcgcccuaccuccaguacuacgaguacuugcucuccaaacccaacucccaucuuccucaggugauuuccaacucacuguucccgaguacuacgccaggagaguuugcaacuugaucggcuugaugggucuucguggcaucacggugcucgaguccucuggugauaccggaaucggcucggcaugcauguccaaugacggcaccaacaagccccaauucacuccuacauucccuggcaccugccccuucaucaccgcaguuggugguacucaguccuaugcuccugaaguugcuugggacggcaguuccggcggauucagcaacuacuucagccgucccugguaccagucuuucgcgguggacaacuaccucaacaaccacauuaccaaggauaccaagaaguacuauucgcaguacaccaacuucaagggccguggauucccugauguuuccgcccauaguuugaccccuuacuacgaggucgucuugacuggcaaacacuacaagucuggcggcacauccgccgccagccccgucuuugccgguauugucggucugcugaacgacgcccgucugcgcgccggcaaguccacucuuggcuuccugaacccauugcuguauagcauccuggccgaaggauucaccgauaucacugccggaaguucaaucgguuguaaugguaucaacccacagaccggaaagccaguuccugguggugguauuauccccuacgcucacuggaacgcuacugccggcugggauccuguuacuggccuugggguuccugauuucaugaaauugaaggaguugguucugucguuguaa

SEQ ID NO: 81 - DNA, 1785 bp, Phaeosphaeria nodorum
atgcagaccttcggtgcttttctcgtttccttcctcgccgccagcggcctggccgcggccgagcccttcgagaagctctttagcacccccgagggctggaagatgcagggcctcgccaccaacgagcagatcgtcaagctccagatcgccctccagcagggcgacgtggccggctttgagcagcacgtcatcgacatcagcacccccagccaccccagctacggcgctcactacggcagccacgaagagatgaagcgcatgatccagcccagcagcgagactgtcgccagcgtcagcgcctggctcaaggccgctggcatcaacgacgccgagatcgacagcgactgggtcaccttcaagaccaccgtcggcgtcgccaacaagatgctcgacaccaagttcgcctggtacgtcagcgaggaagccaagccccgcaaggtcctccgcacccttgagtacagcgtccccgacgacgtcgccgagcacatcaacctcatccagcccaccacccgcttcgccgccatccgccagaaccacgaggtcgcccacgagatcgtcggcctccagtttgccgccctcgccaacaacaccgtcaactgcgacgccaccatcaccccccagtgcctcaagaccctctacaagatcgactacaaggccgaccccaagagcggcagcaaggtcgccttcgccagctaccttgagcagtacgcccgctacaacgacctcgccctcttcgagaaggccttcctgcctgaggccgtcggccagaacttcagcgtcgtccagttctctggcggcctcaacgaccagaacaccacccaggatagcggcgaggccaacctcgacctccagtacatcgtcggcgtcagcgcccctctgcccgtcaccgagtttagcactggcggccgaggcccttgggtcgccgatctcgatcagcctgacgaggccgacagcgccaacgagccctaccttgagttcctccagggcgtcctcaagctcccccagagcgagctgccccaggtcatcagcacctcgtacggcgagaacgagcagagcgtccccaagagctacgccctcagcgtctgcaacctcttcgcccagcttggctctcgcggcgtcagcgtcatcttcagcagcggcgatagcggccctggcagcgcctgccagtctaacgacggcaagaacaccaccaagttccagccccagtaccctgccgcctgccccttcgtcactagcgtcggctctacccgctacctcaacgagactgccaccggcttcagctccggcggcttcagcgactactggaagcgccccagctaccaggacgacgccgtcaaggcctacttccaccacctcggcgagaagttcaagccctacttcaaccgccacggccgaggcttccctgacgtcgccactcagggctacggcttccgcgtctacgaccagggcaagctcaagggcctccagggcacttctgccagcgcccctgccttcgccggcgtcattggcctgctcaacgacgcccgcctcaaggccaagaagcccaccctcggctttctcaaccccctgctctacagcaacagcgacgccctcaacgacatcgtcctcggcggctccaagggctgcgacggccacgctaggtttaacggccctcccaacggcagccccgtcatcccttacgccggctggaacgccactgccggctgggaccctgttaccggcctcggcacccccaacttccccaagctcctcaaggccgccgtcccctctcgataccgcgcttaa

SEQ ID NO: 82 - DNA, 1833 bp, Trichoderma atroviride
atgcagaccttcggtgcttttctcgtttccttcctcgccgccagcggcctggccgcggccaacgctgctgtcctcctcgacagcctcgacaaggtccccgtcggctggcaggctgcttctgcccctgctcccagcagcaagatcaccctccaggtcgccctcacccagcagaacatcgaccagcttgagagcaagctcgccgccgtcagcacccccaacagcagcaactacggcaagtacctcgacgtcgacgagatcaaccagatcttcgcccccagcagcgccagcactgccgctgtcgagagctggctcaagagctacggcgtcgactacaaggtccagggcagcagcatctggttccagaccgacgtcagcacggccaacaagatgctcagcaccaacttccacacctacaccgacagcgtcggcgccaagaaggtccgcaccctccagtacagcgtccccgagactctcgccgaccacatcgacctcatcagccccaccacctacttcggcaccagcaaggccatgcgagccctcaagatccagaacgccgccagcgccgtcagccctctcgctgctcgacaagagcccagcagctgcaagggcaccatcgagttcgagaaccgcaccttcaacgtctttcagcccgactgcctccgcaccgagtacagcgtcaacggctacaagcccagcgccaagagcggcagccgaatcggcttcggcagcttcctcaaccagagcgccagcagcagcgacctcgccctcttcgagaagcacttcggcttcgccagccagggcttcagcgtcgagctgatcaacggcggcagcaacccccagcctcccaccgatgctaacgacggcgaggccaacctcgacgcccagaacatcgtcagcttcgtccagcccctgcccatcaccgagtttatcgctggcggcaccgccccctacttccccgatcctgttgagcctgccggcacccccgacgagaacgagccctaccttgagtactacgagtacctcctcagcaagagcaacaaggaactcccccaggtcatcaccaacagctacggcgacgaggaacagaccgtcccccaggcctacgccgtccgcgtctgcaacctcatcggcctcatgggcctccgcggcatcagcatccttgagagcagcggcgacgagggcgtcggcgcttcttgcctcgccaccaacagcaccaccaccccccagttcaaccccatcttccccgccacgtgcccctacgtcactagcgtcggcggcaccgtcagcttcaaccccgaggtcgcttgggacggcagcagcggcggcttcagctactacttcagccgcccctggtatcaagaggccgccgtcggcacctacctcaacaagtacgtcagcgaggaaacgaaggaatattacaagagctacgtcgacttcagcggccgaggcttccctgacgtcgccgctcactctgtcagccccgactaccccgtctttcagggcggcgagctgactccttctggcggcacttctgccgccagccccatcgtcgccagcgtcattgccctgctcaacgacgcccgactccgagccggcaagcctgccctcggctttctcaaccccctcatctacggctacgcctacaagggcttcaccgacatcacctccggccaggccgttggctgcaacggcaacaacacccagaccggcggaccccttcctggcgctggcgttatccctggcgccttctggaacgccaccaagggctgggaccccaccaccggctttggcgtccccaacttcaagaagctccttgagctggtccgctacatc

SEQ ID NO: 83 - DNA, 1803 bp, Arthroderma benhamiae
atgcagaccttcggtgcttttctcgtttccttcctcgccgccagcggcctggccgcggccaagcctactcctggcgcttcccacaaggtcatcgagcacctcgacttcgtccccgagggctggcagatggtcggcgctgctgaccctgccgccatcatcgacttttggctcgccatcgagcgcgagaaccccgagaagctctacgacaccatctacgacgtcagcacccccggacgcgcccagtacggcaagcacctcaagcgcgaggaactcgacgacctcctccgccctcgcgccgagactagcgagagcatcatcaactggctcaccaacggcggcgtcaacccccagcacattcgcgacgagggcgactgggtccgcttcagcaccaacgtcaagaccgccgagactctcatgaacacccgcttcaacgtctttaaggacaacctcaacagcgtcagcaagatccgcacccttgagtacagcgtccccgtcgccatcagcgcccacgtccagatgatccagcccaccaccctcttcggccgccagaagccccagaacagcctcatcctcaaccccctcaccaaggaccttgagagcatgagcgtcgaagagttcgccgccagccagtgccgcagcctcgtcactactgcctgcctccgcgagctgtacggcctcggcgatcgagtcacccaggcccgcgacgacaaccgaattggcgtcagcggcttcctcgaagagtacgcccagtaccgcgaccttgagctgttcctcagccgcttcgagcccagcgccaagggcttcaacttcagcgagggcctgatcgctggcggcaagaacacccagggtggccctggctctagcaccgaggccaacctcgacatgcagtacgtcgtcggcctcagccacaaggccaaggtcacctactacagcactgccggccgaggccccctcatccctgatctctcacagcccagccaggccagcaacaacaacgagccctaccttgagcagctccgctacctcgtcaagctccccaagaaccagctccccagcgtcctcaccaccagctacggcgacaccgagcagagcctccccgccagctacaccaaggccacgtgcgacctcttcgcccagctcggcactatgggcgtcagcgtcatcttcagcagcggcgacactggccctggcagctcgtgccagaccaacgacggcaagaacgccacgcgcttcaaccccatctaccccgccagctgccccttcgtcaccagcattggcggcaccgtcggcaccggccctgagcgagctgtcagctttagcagcggcggcttcagcgaccgcttccctcgccctcagtaccaggacaacgccgtcaaggactacctcaagatcctcggcaaccagtggtccggcctcttcgaccctaacggccgagccttccccgacattgccgcccagggcagcaactacgccgtctacgacaagggccgcatgaccggcgttagcggcacttctgcttccgcccctgctatggccgccatcattgcccagctcaacgacttccgcctcgccaagggcagccccgtcctcggctttctcaacccctggatctacagcaagggcttcagcggcttcaccgacatcgtcgacggcggctctaggggctgcaccggctacgacatctacagcggcctcaaggccaagaaggtcccctacgccagctggaacgccaccaagggctgggaccccgtcaccggctttggcacccccaacttccaggccctgaccaaggtcctgccctaa

SEQ ID NO: 84 - DNA, 1803 bp, Fusarium graminearum
atgcagaccttcggtgcttttctcgtttccttcctcgccgccagcggcctggccgcggccaagagctactctcaccacgccgaggcccccaagggctggaaggtcgacgatactgcccgcgtcgccagcaccggcaagcagcaggtcttttcgatcgccctgaccatgcagaacgtcgaccagcttgagagcaagctcctcgacctcagcagccccgacagcaagaactacggccagtggatgagccagaaggacgtcaccaccgccttctaccccagcaaggaagccgtcagcagcgtcaccaagtggctcaagagcaagggcgtcaagcactacaacgtcaacggcggcttcatcgacttcgccctcgacgtgaagggcgccaacgccctcctcgacagcgactaccagtactacaccaaggaaggccagaccaagctccgcaccctcagctacagcatccccgacgacgtcgccgagcacgtccagttcgtcgaccccagcaccaacttcggcggcaccctcgcctttgcccccgtcactcaccctagccgcaccctcaccgagcgcaagaacaagcccaccaagagcaccgtcgacgccagctgccagaccagcatcacccccagctgcctcaagcagatgtacaacatcggcgactacacccccaaggtcgagagcggcagcacgatcggcttcagcagcttcctcggcgagagcgctatctacagcgacgtctttctgttcgaggaaaagttcggcatccccacccagaacttcaccaccgtcctcatcaacaacggcaccgacgaccagaacaccgcccacaagaacttcggcgaggccgacctcgacgccgagaacatcgtcggcattgcccaccccctgcccttcacccagtacatcactggcggcagcccccccttcctgcccaacatcgatcagcccactgccgccgacaaccagaacgagccctacgtccccttcttccgctacctcctcagccagaaggaagtccccgccgtcgtcagcaccagctacggcgacgaagaggacagcgtcccccgcgagtacgccaccatgacctgcaacctcatcggcctgctcggcctccgcggcatcagcgtcatcttcagcagcggcgacatcggcgtcggcgctggctgtcttggccccgaccacaagaccgtcgagttcaacgccatcttccccgccacgtgcccctacctcactagcgtcggcggcacggtcgacgtcacccccgagattgcttgggagggcagcagcggcggcttcagcaagtacttccctcgccccagctaccaggacaaggccgtcaagacctacatgaagaccgtcagcaagcagaccaagaagtactacggcccctacaccaactgggagggccgaggctttcctgacgtcgccggccacagcgtcagccccaactacgaggtcatctacgccggcaagcagagcgcctctggcggcacttctgctgccgcccctgtctgggctgccatcgtcggcctgctcaacgacgcccgattccgagccggcaagcctagcctcggctggctcaaccccctcgtctacaagtacggccccaaggtcctcaccgacatcaccggcggctacgccattggctgcgacggcaacaacacccagagcggcaagcccgagcctgccggctctggcattgtccctggcgcccgatggaacgccactgccggatgggaccctgtcaccggctacggcacccccgacttcggcaagctcaaggacctcgtcctcagcttctaa

SEQ ID NO: 85 - DNA, 1872 bp, Acremonium alcalophilum
atgcagaccttcggtgcttttctcgtttccttcctcgccgccagcggcctggccgcggccgccgtcgtcattcgcgccgccgtcctccccgacgccgtcaagctgatgggcaaggccatgcccgacgacattatttccctccagttttccctgaagcagcagaacatcgaccagctggagacccgcctccgcgccgtctcggaccccagctcccccgagtacggccagtacatgagcgagtccgaggtcaacgagttctttaagccccgcgacgactcgttcgccgaggtcattgactgggtcgccgccagcggctttcaggacatccacctgacgccccaggctgccgccattaacctcgccgccaccgtcgagacggccgaccagctcctgggcgccaacttcagctggtttgacgtcgacggcacccgcaagctccgcaccctggagtacacgatccccgaccgcctcgccgaccacgtcgacctgatttcccccaccacgtacttcggccgcgcccgactggacggcccccgcgagacccccacgcgcctcgacaagcgccagcgcgaccccgtcgccgacaaggcctacttccacctcaagtgggaccgcggcaccagcaactgcgacctggtcatcacgcccccctgcctggaggccgcctacaactacaagaactacatgcccgaccccaactcgggcagccgcgtctcgttcaccagctttctggagcaggccgcccagcagagcgacctcaccaagttcctctccctgacgggcctcgaccgcctgcgcccccccagcagcaagcccgccagcttcgacacggtcctgatcaacggcggcgagacccaccagggcacgccccccaacaagacctccgaggccaacctcgacgtccagtggctggccgccgtcattaaggcccgactccccatcacccagtggattacgggcggccgcccccccttcgtccccaacctccgcctgcgccacgagaaggacaacacgaacgagccctacctggagttctttgagtacctcgtccgcctgcccgcccgcgacctcccccaggtcatctccaactcgtacgccgaggacgagcagaccgtccccgaggcctacgcccgacgcgtctgcaacctcatcggcattatgggcctgcgcggcgtcaccgtcctcacggcctccggcgactcgggcgtcggcgccccctgccgcgccaacgacggcagcgaccgcctggagttctccccccagtttcccacctcgtgcccctacatcaccgccgtcggcggcacggagggctgggaccccgaggtcgcctgggaggcctcctcgggcggcttcagccactactttctccgcccctggtaccaggccaacgccgtcgagaagtacctcgacgaggagctggaccccgccacccgcgcctactacgacggcaacggcttcgtccagtttgccggccgagcctaccccgacctgtccgcccacagctcctcgccccgctacgcctacatcgacaagctcgcccccggcctgaccggcggcacgagcgcctcctgccccgtcgtcgccggcatcgtcggcctcctgaacgacgcccgactccgccgcggcctgcccacgatgggcttcattaacccctggctgtacacgcgcggctttgaggccctccaggacgtcaccggcggccgcgcctcgggctgccagggcatcgacctccagcgcggcacccgcgtccccggcgccggcatcattccctgggcctcctggaacgccacccccggctgggaccccgccacgggcctcggcctgcccgacttctgggccatgcgcggcctcgccctgggccgcggcacctaa

SEQ ID NO: 86 - DNA, 1905 bp, Sodiomyces alkalinus
atgcagaccttcggtgcttttctcgtttccttcctcgccgccagcggcctggccgcggccgccgtcgtcattcgcgccgcccccctccccgagagcgtcaagctcgtccgcaaggccgccgccgaggacggcattaacctccagctctccctgaagcgccagaacatggaccagctggagaagttcctccgcgccgtcagcgaccccttttcccccaagtacggccagtacatgtcggacgccgaggtccacgagatcttccgccccaccgaggactcctttgaccaggtcattgactggctcaccaagtcgggcttcggcaacctgcacatcacgccccaggctgccgccattaacgtcgccaccacggtcgagaccgccgaccagctgtttggcgccaacttctcctggtttgacgtcgacggcacgcccaagctccgcaccggcgagtacacgatccccgaccgcctcgtcgagcacgtcgacctggtcagccccaccacgtacttcggccgcatgcgccccccccctcgcggcgacggcgtcaacgactggatcaccgagaactcgcccgagcagcccgcccccctgaacaagcgcgacaccaagacggagagcgaccaggcccgcgaccacccctcctgggactcgcgcacccccgactgcgccaccatcattacgcccccctgcctggagacggcctacaactacaagggctacatccccgaccccaagtccggctcgcgcgtcagcttcaccagcttcctggagcaggccgcccagcaggccgacctgaccaagttcctcagcctgacgcgcctggagggctttcgcacccccgccagcaagaagaagaccttcaagacggtcctgatcaacggcggcgagtcccacgagggcgtccacaagaagtcgaagaccagcgaggccaacctcgacgtccagtggctggccgccgtcacccagacgaagctgcccatcacccagtggattacgggcggccgcccccccttcgtccccaacctccgcatccccacccccgaggccaacacgaacgagccctacctggagttcctggagtacctctttcgcctgcccgacaaggacctcccccaggtcatcagcaactcctacgccgaggacgagcagagcgtccccgaggcctacgcccgacgcgtctgcggcctcctgggcattatgggcctccgcggcgtcaccgtcctgacggcctccggcgactcgggcgtcggcgccccctgccgcgccaacgacggctcgggccgcgaggagttcagcccccagtttcccagctcctgcccctacatcaccacggtcggcggcacccaggcctgggaccccgaggtcgcctggaagggcagcagcggcggcttctccaactactttccccgcccctggtaccaggtcgccgccgtcgagaagtacctggaggagcagctggaccccgccgcccgcgagtactacgaggagaacggcttcgtccgctttgccggccgagccttccccgacctgagcgcccacagcagcagccccaagtacgcctacgtcgacaagcgcgtccccggcctcaccggcggcacgtcggccagctgccccgtcgtcgccggcatcgtcggcctcctgaacgacgcccgactccgccgcggcctgcccacgatgggcttcattaacccctggctctacgccaagggctaccaggccctggaggacgtcaccggcggcgccgccgtcggctgccagggcatcgacattcagacgggcaagcgcgtccccggcgccggcatcattcccggcgccagctggaacgccacccccgactgggaccccgccacgggcctcggcctgcccaacttctgggccatgcgcgagctcgccctggaggactaa

SEQ ID NO: 87 - DNA, 1788 bp, Aspergillus kawachii
atgcagaccttcggtgcttttctcgtttccttcctcgccgccagcggcctggccgcggccgtcgtccatgagaagctcgctgctgtccccagcggctggcaccaccttgaggatgccggcagcgaccaccagatcagcctctcgattgccctcgcccgcaagaacctcgaccagcttgagagcaagctcaaggacctcagcacccctggcgagagccagtacggccagtggctcgaccaagaggaagtcgacaccctgttccccgtcgccagcgacaaggccgtcatcagctggctccgcagcgccaacatcacccacattgcccgccagggcagcctcgtcaacttcgccaccaccgtcgacaaggtcaacaagctcctcaacaccaccttcgcctactaccagcgcggcagctctcagcgcctccgcaccaccgagtacagcatccccgacgacctcgtcgacagcatcgacctgatcagccccaccacgttcttcggcaaggaaaagacctctgccggcctcacccagcgcagccagaaggtcgataaccacgtcgccaagcgcagcaacagcagcagctgcgccgacaccatcaccctcagctgcctcaaggaaatgtacaacttcggcaactacacccccagcgccagcagcggcagcaagctcggcttcgccagcttcctcaacgagagcgccagctacagcgacctcgccaagttcgagcgcctcttcaacctccccagccagaacttcagcgtcgagctgatcaacggcggcgtcaacgaccagaaccagagcaccgccagcctcaccgaggccgacctcgatgtcgagctgcttgtcggcgtcggccaccccctgcccgtcaccgagtttatcaccagcggcgagccccccttcatccccgaccctgatgagccttctgccgccgacaacgagaacgagccctacctccagtactacgagtacctcctcagcaagcccaacagcgccctgccccaggtcatcagcaacagctacggcgacgacgagcagaccgtccccgagtactacgccaagcgcgtctgcaacctcatcggcctcgtcggcctccgcggcatcagcgtccttgagtctagcggcgacgagggcatcggctctggctgccgaaccaccgacggcaccaacagcacccagttcaaccccatcttccccgccacgtgcccctacgtcactgccgtcggcggcaccatgagctacgcccccgagattgcttgggaggccagctccggcggcttcagcaactacttcgagcgagcctggttccagaaggaagccgtccagaactacctcgccaaccacatcaccaacgagactaagcagtactacagccagttcgccaacttcagcggccgaggcttccccgacgtcagcgcccacagcttcgagcccagctacgaggtcatcttctacggcgctcgctacggcagcggcggcacttctgctgcctgccccctgttttctgccctcgtcggcatgctcaacgacgcccgactccgagccggcaagtcgaccctcggcttcctcaaccccctgctctacagcaagggctacaaggccctcaccgacgtcaccgctggccagagcattggctgcaacggcatcgacccccagagcgacgaggctgtcgctggcgctggcatcattccctgggcccactggaacgccaccgtcggctgggaccctgtcactggccttggcctccccgacttcgagaagctccgccagctcgtcctcagcctctaa

SEQ ID NO: 88 - DNA, 1809 bp, Talaromyces stipitatus
atgcagaccttcggtgcttttctcgtttccttcctcgccgccagcggcctggccgcggccgctgctgctcttgttggccacgagtctctcgccgccctccctgtcggctgggacaaggtcagcactcctgccgctggcaccaacatccagctcagcgtcgccctcgccctccagaacatcgagcagcttgaggaccacctcaagagcgtcagcacccccggctctgccagctacggccagtacctcgacagcgacggcattgccgcccagtacggcccttctgacgccagcgtcgaggccgtcaccaactggctcaaggaagccggcgtcaccgacatctacaacaacggccagagcatccacttcgccaccagcgtcagcaaggccaacagcctcctcggcgccgacttcaactactacagcgacggctccgccaccaagctccgcaccctcgcttacagcgtccccagcgacctgaaggaagccatcgacctcgtcagccccaccacctacttcggcaagaccaccgccagccgcagcatccaggcctacaagaacaagcgagccagcaccaccagcaagagcggcagcagcagcgtccaggtcagcgcctcttgccagaccagcatcacccccgcctgcctcaagcagatgtacaacgtcggcaactacacccccagcgtcgcccacggctctcgcgttggcttcggcagcttcctcaaccagagcgccatcttcgacgacctcttcacctacgagaaggtcaacgacatccccagccagaacttcaccaaggtcatcattgccaacgccagcaacagccaggacgccagcgacggcaactacggcgaggccaacctcgacgtccagaacattgtcggcatcagccaccccctgcccgtcaccgagtttctcactggcggcagcccacccttcgtcgccagcctcgacacccccaccaaccagaacgagccctacatcccctactacgagtacctcctcagccagaagaacgaggacctcccccaggtcatcagcaacagctacggcgacgacgagcagagcgtcccctacaagtacgccatccgcgcctgcaacctcatcggcctcactggcctccgcggcatcagcgtccttgagagcagcggcgatctcggcgttggcgctggctgccgatccaacgacggcaagaacaagacccagttcgaccccatcttccccgccacgtgcccctacgtcactagcgtcggcggcacccagagcgtcacccccgagattgcttgggtcgcttccagcggcggcttcagcaactacttcccccgcacctggtatcaagagcccgccatccagacctacctcggcctcctcgacgacgagactaagacctactacagccagtacaccaacttcgagggccgaggcttccccgacgtcagcgcccattctctcacccccgactaccaggtcgtcggcggaggctaccttcagccttctggcggcacttctgccgccagccctgtctttgccggcatcattgccctgctcaacgacgcccgactcgccgctggcaagcccaccctcggctttctcaaccccttcttctacctctacggctacaagggcctcaacgacatcactggcggccagagcgtcggctgcaacggcatcaacggccagactggcgcccctgttcccggcggaggaattgtccctggcgccgcttggaacagcaccaccggatgggaccctgccaccggccttggcacccccgactttcagaagctcaaggaactcgtcctcagcttctaa

SEQ ID NO: 89 - DNA, 1800 bp, Fusarium oxysporum
atgcagaccttcggtgcttttctcgtttccttcctcgccgccagcggcctggccgcggccaagtcgttttcccaccacgccgaggccccccagggctggcaggtccagaagaccgccaaggtcgcctccaacacgcagcacgtctttagcctcgccctgaccatgcagaacgtcgaccagctggagtcgaagctcctggacctgagctcccccgacagcgccaactacggcaactggctcagccacgacgagctgacctccacgttctcgcccagcaaggaggccgtcgcctcggtcaccaagtggctgaagagcaagggcatcaagcactacaaggtcaacggcgccttcattgactttgccgccgacgtcgagaaggccaacaccctcctgggcggcgactaccagtactacacgaaggacggccagaccaagctgcgcacgctctcctactcgatccccgacgacgtcgccggccacgtccagttcgtcgaccccagcaccaacttcggcggcacggtcgcctttaaccccgtcccccacccctcccgcaccctccaggagcgcaaggtctccccctccaagtcgacggtcgacgcctcctgccagacctcgatcacgcccagctgcctgaagcagatgtacaacattggcgactacacccccgacgccaagagcggctccgagatcggcttcagcagcttcctcggccaggccgccatttacagcgacgtcttcaagtttgaggagctcttcggcatccccaagcagaactacaccacgatcctgattaacaacggcaccgacgaccagaacacggcccacggcaactttggcgaggccaacctcgacgccgagaacatcgtcggcattgcccaccccctgcccttcaagcagtacatcaccggcggcagccccccctttgtccccaacattgaccagcccacggagaaggacaaccagaacgagccctacgtccccttctttcgctacctcctgggccagaaggacctgcccgccgtcatctcgaccagctacggcgacgaggaggactccgtcccccgcgagtacgccaccctcacgtgcaacatgatcggcctcctgggcctgcgcggcatctccgtcattttctcctcgggcgacattggcgtcggctcgggctgcctcgcccccgactacaagaccgtcgagttcaacgccatctttcccgccacctgcccctacctgacgtccgtcggcggcaccgtcgacgtcacgcccgagattgcctgggagggcagctccggcggcttctccaagtactttccccgcccctcgtaccaggacaaggccatcaagaagtacatgaagaccgtctcgaaggagacgaagaagtactacggcccctacaccaactgggagggccgcggcttccccgacgtcgccggccactccgtcgcccccgactacgaggtcatctacaacggcaagcaggcccgatccggcggcaccagcgccgccgcccccgtctgggccgccatcgtcggcctcctgaacgacgcccgattcaaggccggcaagaagagcctgggctggctcaaccccctgatctacaagcacggccccaaggtcctcaccgacatcacgggcggctacgccattggctgcgacggcaacaacacccagagcggcaagcccgagcccgccggctccggcctggtccccggcgcccgatggaacgccaccgccggctgggaccccaccacgggctacggcacgcccaacttccagaagctcaaggacctcgtcctgtccctctaa

SEQ ID NO: 90 - DNA, 1788 bp, Trichoderma virens; identical to SEQ ID NO: 87

SEQ ID NO: 91 - DNA, 1770 bp, Trichoderma atroviride
atgcagaccttcggtgcttttctcgtttccttcctcgccgccagcggcctggccgcggccgctgtccttgtcgagtctctcaagcaggtccccaacggctggaacgccgtcagcacccctgaccccagcaccagcatcgtcctccagatcgccctcgcccagcagaacatcgacgagcttgagtggcgcctcgccgccgtgtctacccccaactctggcaactacggcaagtacctcgacatcggcgagatcgagggcatcttcgcccccagcaacgccagctacaaggccgtcgcttcctggctccagagccacggcgtcaagaacttcgtcaagcaggccggcagcatctggttctacaccaccgtcagcaccgccaacaagatgctcagcaccgacttcaagcactacagcgaccccgtcggcatcgagaagctccgcaccctccagtacagcatccccgaggaactcgtcggccacgtcgacctcatcagccccaccacctacttcggcaacaaccaccctgccaccgcccgcacccccaacatgaaggccatcaacgtcacctaccagatcttccaccccgactgcctcaagaccaagtacggcgtcgacggctacgccccctcacctcgatgcggcagccgaatcggcttcggcagcttcctcaacgagactgccagctacagcgacctcgcccagttcgagaagtacttcgacctccccaaccagaacctcagcaccctcctcatcaacggcgccatcgacgtccagccccccagcaacaagaacgacagcgaggccaacatggacgtccagaccatcctcaccttcgtccagcccctgcccatcaccgagttcgtcgtcgccggcatccccccctacattcccgatgccgccctccccattggcgaccccgttcagaacgagccctggcttgagtacttcgagttcctcatgagccgcaccaacgccgagctgccccaggtcattgccaacagctacggcgacgaggaacagaccgtcccccaggcctacgccgtccgcgtctgcaaccagattggcctcctcggcctccgcggcatcagcgtcattgcctctagcggcgacaccggcgtcggcatgtcttgcatggccagcaacagcaccaccccccagttcaaccccatgttccccgccagctgcccctacatcaccaccgtcggcggcacccagcacctcgacaacgagatcgcctgggagctgagcagcggcggcttcagcaactacttcacccgcccctggtatcaagaggacgccgccaagacctaccttgagcgccacgtcagcaccgagactaaggcctactacgagcgctacgccaacttcctgggccgaggctttcctgacgtcgccgccctcagcctcaaccccgactaccccgtcatcatcggcggcgagcttggccctaacggcggcacttctgctgccgcccctgtcgtcgccagcatcattgccctgctcaacgacgcccgcctctgcctcggcaagcctgccctcggctttctcaaccccctcatctaccagtacgccgacaagggcggcttcaccgacatcaccagcggccagtcttggggctgcgccggcaacaccactcagactggacctccccctcctggcgctggcgtcattcctggcgctcactggaacgccaccaagggctgggaccccgtcaccggctttggcacccccaacttcaagaagctcctcagcctcgccctcagcgtctaa

SEQ ID NO: 92 - DNA, 1758 bp, Agaricus bisporus
atgcagaccttcggtgcttttctcgtttccttcctcgccgccagcggcctggccgcggcctctcctcttgctcgacgctgggacgacttcgccgagaagcacgcctgggtcgaggttcctcgcggctgggagatggtcagcgaggcccctagcgaccacaccttcgacctccgcatcggcgtcaagagcagcggcatggaacagctcatcgagaacctcatgcagaccagcgaccccacccacagccgctacggccagcacctcagcaaggaagaactccacgacttcgtccagccccaccccgactctactggcgccgtcgaggcctggcttgaggacttcggcatcagcgacgacttcatcgaccgcaccggcagcggcaactgggtcaccgtccgagtctctgtcgcccaggccgagcgaatgctcggcaccaagtacaacgtctaccgccacagcgagagcggcgagtccgtcgtccgcaccatgagctacagcctccccagcgagctgcacagccacatcgacgtcgtcgcccccaccacctacttcggcaccatgaagtcgatgcgcgtcacctcgttcctccagcccgagatcgagcccgtcgacccctctgccaagccttctgctgctcccgccagctgcctcagcaccaccgtcattacccccgactgcctccgcgacctctacaacaccgccgactacgtccccagcgccaccagccgcaacgccattggcattgccggctacctcgaccgcagcaaccgagccgacctccagaccttcttccgccgctttcgccctgacgccgtcggcttcaactacaccaccgtccagctcaacggcggaggcgacgaccagaacgaccctggcgtcgaggccaacctcgacatccagtacgccgctggcattgccttccccacccccgccacctactggtctactggcggcagcccccccttcatccccgacacccagacccccaccaacaccaacgagccctacctcgactggatcaacttcgtcctcggccaggatgagatcccccaggtcatcagcaccagctacggcgacgacgagcagaccgtccccgaggactacgccaccagcgtctgcaacctcttcgcccagcttggctctcgcggcgtcaccgtctttttcagcagcggcgacttcggcgtcggcggtggcgactgcctcactaacgacggcagcaaccaggtcctcttccagcccgccttccctgccagctgcccctttgtcactgccgtcggcggcaccgtccgactcgaccctgagatcgccgtcagcttcagcggcggtggcttcagccgctacttcagccgccccagctaccagaaccagaccgtcgcccagttcgtcagcaacctcggcaacaccttcaacggcctctacaacaagaacggccgagcctaccccgacctcgccgctcagggcaacggcttccaggtcgtcatcgacggcatcgtccgatcggtcggcggcacttctgccagcagccctaccgtcgccggcatcttcgccctgctcaacgacttcaagctctctcgcggccagagcaccctcggcttcatcaaccccctcatctacagcagcgccacctccggcttcaacgacatccgagccggcaccaaccctggctgtggcacccgaggctttaccgccggcactggctgggaccctgtcaccggactcggcacccctgactttctccgcctccagggcctcatctaa

SEQ ID NO: 93 - DNA, 1812 bp, Magnaporthe oryzae
atgcagaccttcggtgcttttctcgtttccttcctcgccgccagcggcctggccgcggcccgcgtctttgattctctccctcacccccctcgcggctggtcctactctcacgccgctgagagcaccgagcccctcaccctccgaattgccctccgccagcagaacgccgctgcccttgagcaggtcgtcctccaggtcagcaacccccgccacgccaactacggccagcacctcacccgagatgagctgcgctcttacaccgcccctacccctcgcgctgtccgctctgtcactagctggctcgtcgacaacggcgtcgacgactacaccgtcgagcacgactgggtcaccctccgcaccactgtcggcgctgccgatcgactcctcggcgccgactttgcctggtacgctggccctggcgagactctccagctccgcactctcagctacggcgtggacgacagcgtcgcccctcacgtcgatctcgtccagcccaccacccgctttggcggccctgttggccaggccagccacatcttcaagcaggacgacttcgacgagcagcagctcaagaccctcagcgtcggcttccaggtcatggccgacctccctgctaacggccctggcagcattaaggccgcctgcaacgagagcggcgtcacccctctctgcctccgcaccctctaccgcgtcaactacaagcccgccaccaccggcaacctcgtcgccttcgccagcttccttgagcagtacgcccgctacagcgaccagcaggccttcacccagcgagtccttggccctggcgtcccgctccagaacttcagcgtcgagactgtcaacggcggagccaacgaccagcagagcaagctcgatagcggcgaggccaacctcgacctccagtacgtcatggccatgtcccaccccatccccatccttgagtacagcactggcggccgaggccccctcgtccctactctcgatcagcccaacgccaacaacagcagcaacgagccctaccttgagttcctcacctacctgctcgcccagcccgacagcgccattccccagactctcagcgtgagctacggcgaggaagaacagagcgtcccccgcgactacgccatcaaggtctgcaacatgttcatgcagctcggcgctcgcggcgtcagcgtcatgtttagcagcggcgatagcggccctggcaacgactgcgtccgagcctctgacaacgccaccttcttcggcagcaccttccctgccggctgcccctacgtcactagcgtcggcagcaccgtcggcttcgagcctgagcgagccgtcagctttagctccggcggcttcagcatctaccacgcccgacccgactaccagaacgaggtcgtccccaagtacatcgagagcatcaaggccagcggctacgagaagttcttcgacggcaacggccgaggcatccccgatgtcgctgctcagggcgctcgcttcgtcgtcatcgacaagggccgcgtcagcctcatcagcggcactagcgcttccagccccgccttcgctggcatggtcgccctcgtcaacgccgctcgcaagagcaaggatatgcccgccctcggcttcctcaaccccatgctctaccagaacgctgccgccatgaccgacatcgtcaacggcgctggcatcggctgccgcaagcagcgcaccgagtttcccaacggtgcccgcttcaacgccaccgccggatgggaccctgtcactggccttggcacccccctgttcgacaagctcctcgccgttggcgctcccggcgtccctaacgcctaa

SEQ ID NO: 94 - DNA, 1845 bp, Togninia minima
atgcagaccttcggtgcttttctcgtttccttcctcgccgccagcggcctggccgcggcctccgatgtcgtccttgagtctctccgcgaggtcccccagggctggaagcgactccgagatgccgaccccgagcagagcatcaagctccgcattgcccttgagcagcccaacctcgacctcttcgagcagaccctctacgacatcagcagccccgaccaccccaagtacggccagcacctcaagagccacgagctgcgcgacatcatggcccctcgcgaggaatccactgccgccgtcattgcctggctccaggatgctggcctcagcggcagccagatcgaggacgacagcgactggatcaacatccagaccaccgtcgcccaggccaacgacatgctcaacaccaccttcggcctcttcgcccaagagggcaccgaggtcaaccgcattcgcgccctcgcctacagcgtccccgaggaaattgtcccccacgtcaagatgatcgcccccatcatccgcttcggccagctccgccctcagatgagccacatcttcagccacgagaaggtcgaggaaacccccagcatcggcaccatcaaggccgctgccatccccagcgtcgacctcaacgtcaccgcctgcaacgccagcatcacccccgagtgcctccgcgccctctacaacgtcggcgactacgaggccgaccccagcaagaagtccctcttcggcgtctgcggctaccttgagcagtacgccaagcacgaccagctcgccaagttcgagcagacgtacgccccctacgccatcggcgccgacttcagcgtcgtcaccatcaacggcggaggcgacaaccagaccagcaccatcgacgacggcgaggccaacctcgacatgcagtacgccgtcagcatggcctacaagacccccatcacctactacagcactggcggccgaggccccctcgtccctgatctcgatcagcccgaccccaacgacgtcagcaacgagccctacctcgacttcgtcagctacctcctcaagctccccgacagcaagctcccccagaccatcaccaccagctacggcgaggacgagcagagcgtcccccgcagctacgtcgagaaggtctgcaccatgttcggcgcccttggcgcccgaggcgtcagcgtcattttcagctctggcgacaccggcgtcggcagcgcctgccagactaacgacggcaagaacaccacccgctttctgcccatcttccctgccgcctgcccctacgtcactagcgtcggcggcacccgctacgtcgatcctgaggtcgccgtcagcttcagcagcggcggcttcagcgacatcttccccacccccctgtaccagaagggcgccgtcagcggctacctcaagatcctcggcgaccgctggaagggcctctacaaccctcacggccgaggcttccctgacgtcagcggccagtctgtccgctaccacgtctttgactacggcaaggacgtcatgtacagcggcaccagcgccagcgcccccatgtttgctgctctcgtcagcctcctcaacaacgcccgcctcgccaagaagctcccccctatgggcttcctcaacccctggctctacaccgtcggcttcaacggcctcaccgacatcgtccacggcggctctactggctgcaccggcaccgatgtctacagcggcctgcctacccccttcgtcccctacgcctcttggaacgccaccgtcggctgggaccctgtcactggccttggcacccccctgttcgacaagctcctcaacctcagcacccccaacttccacctcccccacatcggcggccactaa

SEQ ID NO: 95 - DNA, 1848 bp, Bipolaris maydis
atgcagaccttcggtgcttttctcgtttccttcctcgccgccagcggcctggccgcggcctctaccacttctcacgtcgagggcgaggtcgtcgagcgccttcatggcgtccctgagggctggtcacaggtcggcgctcccaaccccgaccagaagctccgcttccgcattgccgtccgcagcgccgacagcgagctgttcgagcgcaccctcatggaagtcagcagccccagccacccccgctacggccagcacctcaagcgccacgagctgaaggacctcatcaagcctcgcgccaagagcaccagcaacatcctcaactggctccaagagagcggcatcgaggcccgcgacatccagaacgacggcgagtggatcagcttctacgcccccgtcaagcgagccgagcagatgatgagcaccaccttcaagacctaccagaacgaggcccgagccaacatcaagaagatccgcagcctcgactacagcgtccccaagcacatccgcgacgacatcgacatcatccagcccaccacgcgcttcggccagatccagcctgagcgcagccaggtctttagccaagaggaagtccccttcagcgccctcgtcgtcaacgccacgtgcaacaagaagatcacccccgactgcctcgccaacctctacaacttcaaggactacgacgccagcgacgccaacgtcacgatcggcgtcagcggcttccttgagcagtacgcccgcttcgacgacctcaagcagttcatcagcaccttccagcccaaggccgctggctccaccttccaggtcaccagcgtcaacgctggccccttcgaccagaacagcaccgcctctagcgtcgaggccaacctcgacatccagtacaccaccggcctcgtcgcccccgacatcgagactcgctacttcaccgtccccggacgcggcatcctcatccccgacctcgaccagcctaccgagagcgacaacgccaacgagccctacctcgactacttcacctacctcaacaaccttgaggacgaggaactccccgacgtcctcaccaccagctacggcgagagcgagcagagcgtccctgccgagtacgccaagaaggtctgcaacctcatcggccagctcggcgctcgcggcgtcagcgtcattttcagcagcggcgacaccggccctggcagcgcctgccagactaacgacggcaagaacaccacccgctttctgcccatcttccccgccagctgcccctacgtcactagcgtcggcggcactgtcggcgtcgagcctgagaaggccgtcagctttagcagcggcggcttcagcgacctctggccccgacctgcctaccaagagaaggccgtgagcgagtaccttgagaagctcggcgaccgctggaacggcctctacaaccctcagggccgaggcttccctgacgtcgctgctcagggccagggcttccaggtctttgacaagggccgcctcatctcggtcggcggcacatctgcttccgcccctgtctttgccagcgtcgtcgccctcctcaacaacgcccgaaaggctgccggaatgagcagcctcggcttcctcaacccctggatctacgagcagggctacaagggcctcaccgacatcgtcgctggcggctctactggctgcaccggccgctctatctacagcggcctccctgcccccctggtcccttacgcttcttggaacgccaccgagggctgggaccccgtcactggctatggcacccccgacttcaagcagctcctcaccctcgccaccgcccccaagtctggcgagcgacgagttcgacgaggcggccttggaggccaggcttaa

SEQ ID NO: 96 - DNA, 1791 bp, Arthroderma benhamiae; identical to SEQ ID NO: 60

SEQ ID NO: 97 - DNA, 1803 bp, Artificial Sequence; identical to SEQ ID NO: 83
  TRI045 synthetic gene optimized for expression

SEQ ID NO: 98 - protein, 580 aa, Arthroderma benhamiae; identical to SEQ ID NO: 6

SEQ ID NO: 99 - protein, 390 aa, Arthroderma benhamiae
CRSLVTTACLRELYGLGDRVTQARDDNRIGVSGFLEEYAQYRDLELFLSRFEPSAKGFNFSEGLIAGGKNTQGGPGSSTEANLDMQYVVGLSHKAKVTYYSTAGRGPLIPDLSQPSQASNNNEPYLEQLRYLVKLPKNQLPSVLTTSYGDTEQSLPASYTKATCDLFAQLGTMGVSVIFSSGDTGPGSSCQTNDGKNATRFNPIYPASCPFVTSIGGTVGTGPERAVSFSSGGFSDRFPRPQYQDNAVKDYLKILGNQWSGLFDPNGRAFPDIAAQGSNYAVYDKGRMTGVSGTSASAPAMAAIIAQLNDFRLAKGSPVLGFLNPWIYSKGFSGFTDIVDGGSRGCTGYDIYSGLKAKKVPYASWNATKGWDPVTGFGTPNFQALTKVLP

SEQ ID NO: 100 - protein, 5 aa, Artificial Sequence
  Peptide substrate sequence
AAPPA

SEQ ID NO: 101 - protein, 5 aa, Artificial Sequence
  Peptide fragment of beta-lactoglobulin
IIAEK

SEQ ID NO: 102 - protein, 7 aa, Artificial Sequence
  Peptide fragment of beta-lactoglobulin
ALPMHIR

SEQ ID NO: 103 - protein, 9 aa, Artificial Sequence
  Peptide fragment of beta-lactoglobulin
WENGECAQK

SEQ ID NO: 104 - protein, 20 aa, Artificial Sequence
  Peptide fragment of beta-lactoglobulin
VYVEELKPTPEGDLEILLQK

SEQ ID NO: 105 - protein, 17 aa, Artificial Sequence
  Peptide fragment of beta-lactoglobulin
EELKPTPEGDLEILLQK

SEQ ID NO: 106 - protein, 26 aa, Artificial Sequence
  Peptide fragment of beta-lactoglobulin
VAGTWYSLAMAASDISLLDAQSAPLR

SEQ ID NO: 107 - protein, 7 aa, Artificial Sequence
  tryptic fragment of beta-casein
GPFPIIV

SEQ ID NO: 108 - protein, 8 aa, Artificial Sequence
  tryptic fragment of beta-casein
HKEMPFPK

SEQ ID NO: 109 - protein, 6 aa, Artificial Sequence
  Sequence motif
  MISC_FEATURE (1)..(1): Xaa may be Gly, Thr, Ser or Val
XEANLD

SEQ ID NO: 110 - protein, 5 aa, Artificial Sequence
  Sequence motif
  MISC_FEATURE (1)..(1): Xaa may be Ile, Leu or Val
  MISC_FEATURE (3)..(3): Xaa may be Ser or Thr
  MISC_FEATURE (4)..(4): Xaa may be Ile or Val
XTXXG

SEQ ID NO: 111 - protein, 5 aa, Artificial Sequence
  Sequence motif
QNFSV

SEQ ID NO: 112 - protein, 844 aa, Lactobacillus helveticus
MAVKRFYKTFHPEHYDLRINVNRKNKTINGTSTITGDVIENPVFINQKFMTIDSVKVDGKNVDFDVIEKDEAIKIKTGVTGKAVIEIAYSAPLTDTMMGIYPSYYELEGKKKQIIGTQFETTFARQAFPCVDEPEAKATFSLALKWDEQDGEVALANMPEVEVDKDGYHHFEETVRMSSYLVAFAFGELQSKTTHTKDGVLIGVYATKAHKPKELDFALDIAKRAIEFYEEFYQTKYPLPQSLQLALPDFSAGAMENWGLVTYREAYLLLDPDNTSLEMKKLVATVITHELAHQWFGDLVTMKWWDNLWLNESFANMMEYLSVDGLEPDWHIWEMFQTSEAASALNRDATDGVQPIQMEINDPADIDSVFDGAIVYAKGSRMLVMVRSLLGDDALRKGLKYYFDHHKFGNATGDDLWDALSTATDLDIGKIMHSWLKQPGYPVVNAFVAEDGHLKLTQKQFFIGEGEDKGRQWQIPLNANFDAPKIMSDKEIDLGNYKVLREEAGHPLRLNVGNNSHFIVEYDKTLLDDILSDVNELDPIDKLQLLQDLRLLAEGKQISYASIVPLLVKFADSKSSLVINALYTTAAKLRQFVEPESNEEKNLKKLYDLLSKDQVARLGWEVKPGESDEDVQIRPYELSASLYAENADSIKAAHQIFTENEDNLEALNADIRPYVLINEVKNFGNAELVDKLIKEYQRTADPSYKVDLRSAVTSTKDLAAIKAIVGDFENADVVKPQDLCDWYRGLLANHYGQQAAWDWIREDWDWLDKTVGGDMEFAKFITVTAGVFHTPERLKEFKEFFEPKINVPLLSREIKMDVKVIESKVNLIEAEKDAVNDAVAKAID

SEQ ID NO: 113 - protein, 7 aa, Artificial Sequence
  Protein sequence cleaved by tripeptidyl peptidase
VIPVPQK

SEQ ID NO: 114 - protein, 6 aa, Artificial Sequence
  Protein sequence cleaved by tripeptidyl peptidase
GPFPVI

SEQ ID NO: 115 - protein, 8 aa, Artificial Sequence
  Peptide substrate
RGPFPIIV

SEQ ID NO: 116 - protein, 5 aa, Artificial Sequence
  Peptide substrate
AAFPA

SEQ ID NO: 117 - protein, 525 aa, Artificial Sequence
  Enzyme polypetide sequence (not to be combined with)
MRTAAASLTLAATCLFELASALMPRAPLIPAMKAKVALPSGNATFEQYIDHNNPGLGTFPQRYWYNPEFWAGPGSPVLLFTPGESDAADYDGFLTNKTIVGRFAEEIGGAVILLEHRYWGASSPYPELTTETLQYLTLEQSIADLVHFAKTVNLPFDEIHSSNADNAPWVMTGGSYSGALAAWTASIAPGTFWAYHASSAPVQAIYDFWQYFVPVVEGMPKNCSKDLNRVVEYIDHVYESGDIERQQEIKEMFGLGALKHFDDFAAAITNGPWLWQDMNFVSGYSRFYKFCDAVENVTPGAKSVPGPEGVGLEKALQGYASWFNSTYLPGSCAEYKYWTDKDAVDCYDSYETNSPIYTDKAVNNTSNKQWTWFLCNEPLFYWQDGAPKDESTIVSRIVSAEYWQRQCHAYFPEVNGYTFGSANGKTAEDVNKWTKGWDLTNTTRLIWANGQFDPWRDASVSSKTRPGGPLQSTEQAPVHVIPGGFHCSDQWLVYGEANAGVQKVIDEEVAQIKAWVAEYPKYRKP

SEQ ID NO: 118 - protein, 33 aa, Artificial Sequence
  alpha-2-gliadin sequence
LQLQPFPQPQLPYPQPQLPYPQPQLPYPQPQPF

SEQ ID NO: 119 - protein, 21 aa, Artificial Sequence
  Intermediate alpha-2-gliadin peptide product
YPQPQLPYPQPQLPYPQPQPF

SEQ ID NO: 120 - protein, 178 aa, Bos taurus
MKCLLLALALTCGAQALIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 121 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 122 - protein, 9 aa, Artificial Sequence
  sequence of bradykinin peptide
RPPGFSPFR

SEQ ID NO: 123 - protein, 4 aa, Artificial Sequence
  Peptide substrate sequence
  MISC_FEATURE (3)..(3): Xaa may be any amino acid except Cys and Met
AAXF